(19) United States
(12) Patent Application Publication
(10) Pub. No.: US 2026/0265375 A1
(43) Pub. Date: Sep. 10, 2026

(54) INHIBITORY CHIMERIC RECEPTOR ARCHITECTURES

(71) Applicants: SENTI BIOSCIENCES, INC., South San Francisco, CA (US); TRUSTEES OF BOSTON UNIVERSITY, Boston, MA (US)

(72) Inventors: Wilson Wong, Brookline, MA (US); Seunghee Lee, Boston, MA (US); Russell Morrison Gordley, San Francisco, CA (US); Marcela Guzman Ayala, San Francisco, CA (US); Gary Lee, Castro Valley, CA (US); Nicholas Frankel, San Francisco, CA (US); Xi Kang, Boston, MA (US)

(21) Appl. No.: 19/686,478

(22) Filed: May 22, 2026

Related U.S. Application Data
(62) Division of application No. 17/820,525, filed on Aug. 17, 2022, now abandoned.
Application No. 17/820,525 is a continuation of application No. PCT/US2021/018868, filed on Feb. 19, 2021.
(60) Provisional application No. 62/979,309, filed on Feb. 20, 2020.
(60) Provisional application No. 63/044,597, filed on Jun. 26, 2020.
(60) Provisional application No. 63/136,134, filed on Jan. 11, 2021.

(51) Int. Cl. C07K 16/28; A61K 40/11; A61K 40/15; A61K 40/22; A61K 40/31; A61K 40/41; A61K 40/42; C07K 14/705; C07K 16/32

(52) U.S. Cl. CPC C07K 16/2803; A61K 40/11; A61K 40/15; A61K 40/22; A61K 40/31; A61K 40/41; A61K 40/4205; A61K 40/4211; A61K 40/4221; C07K 14/7051; C07K 14/70517; C07K 14/70521; C07K 16/32; A61K 2239/13; A61K 2239/21; A61K 2239/22; C07K 2317/622; C07K 2319/02; C07K 2319/03; C07K 2319/43

(57) ABSTRACT

Provided herein are inhibitory chimeric antigen receptor compositions and cells comprising such compositions. Also provided are methods of using inhibitory chimeric antigen receptors and cells.

### CROSS REFERENCE TO RELATED APPLICATIONS

This application is a divisional of pending U.S. application Ser. No. 17/820,525 filed Aug. 17, 2022, which is a continuation of International Application No. PCT/US2021/018868 filed Feb. 19, 2021, which claims the benefit of U.S. Provisional Application No. 62/979,309 filed Feb. 20, 2020; 63/044,597 filed Jun. 26, 2020; and 63/136,134 filed Jan. 11, 2021, each of which is hereby incorporated by reference in their entirety for all purposes.

### SEQUENCE LISTING

The instant application contains a Sequence Listing which has been submitted electronically in XML format and is hereby incorporated by reference in its entirety. Said Sequence Listing XML, created on May 22, 2026, is named SENTI1230-4_ST26.xml, and is 189,516 bytes in size.

### BACKGROUND

Chimeric antigen receptors (CARs) enable targeted in vivo activation of immunomodulatory cells, such as T cell. These recombinant membrane receptors have an antigen-binding domain and one or more signaling domains (e.g., T cell activation domains). These special receptors allow the T cells to recognize a specific protein antigen on tumor cells and induce T cell activation and signaling pathways. Recent results of clinical trials with chimeric receptor-expressing T cells have provided compelling support of their utility as agents for cancer immunotherapy. However, despite these promising results, a number of side effects associated the CAR T-cell therapeutics were identified, raising significant safety concerns. One side effect is “on-target but off-tissue” adverse events from TCR and CAR engineered T cells, in which a CAR T cell binds to its ligand outside of the target tumor tissue and induces an immune response. Therefore, the ability to identify appropriate CAR targets is important to effectively targeting and treating the tumor without damaging normal cells that express the same target antigen.

Inhibitory chimeric antigen receptors (also known as iCARs) are protein constructions that inhibit or reduce immunomodulatory cell activity after binding their cognate ligands on a target cell. Current iCAR designs leverage PD-1 intracellular domains for inhibition, but have proven difficult to reproduce. Thus, alternative inhibitory domains for use in iCARs are needed.

### SUMMARY

Provided herein are chimeric inhibitory receptors comprising: an extracellular protein binding domain; a transmembrane domain, wherein the transmembrane domain is operably linked to the extracellular protein binding domain; and an intracellular signaling domain, wherein the intracellular signaling domain is operably linked to the transmembrane domain, and wherein the intracellular signaling domain is capable of preventing, attenuating, or inhibiting activation of a tumor-targeting chimeric receptor expressed on an immunomodulatory cell.

In some aspects, the intracellular signaling domain is derived from a protein selected from the group consisting of: BTLA, PD-1, CTLA4, TIM3, KIR3DL1, LIR1, NKG2A, TIGIT, and LAG3.

In some aspects, the transmembrane domain and the intracellular signaling domain are derived from the same protein.

In some aspects, the transmembrane domain further comprises at least a portion of the protein extracellular domain.

In some aspects, the transmembrane domain is derived from a first protein and the intracellular signaling domain is derived from a second protein that is distinct from the first protein.

In some aspects, the intracellular signaling domain is derived from BTLA. In some aspects, the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RRHQGKQNELSDTAGREINLVDAHLKSEQTEASTRQNSQVLLSETGIYDNDPDLCFR MQEGSEVYSNPCLEENKPGIVYASLNHSVIGPNSRLARNVKEAPTEYASICVRS (SEQ ID NO: 3). In some aspects, the intracellular signaling domain comprises the amino acid sequence of

(SEQ ID NO: 3)
RRHQGKQNELSDTAGREINLVDAHLKSEQTEASTRQNSQVLLSETGIYDN
DPDLCFRMQEGSEVYSNPCLEENKPGIVYASLNHSVIGPNSRLARNVKEA
PTEYASICVRS.

In some aspects, the intracellular signaling domain is derived from LIR1. In some aspects, the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LRHRRQGKHWTSTQRKADFQHPAGAVGPEPTDRGLQWRSSPAADAQEENLYAAVK HTQPEDGVEMDTRSPHDEDPQAVTYAEVKHSRPRREMASPPSPLSGEFLDTKDRQAE EDRQMDTEAAASEAPQDVTYAQLHSLTLRREATEPPPSQEGPSPAVPSIYATLAIH (SEQ ID NO: 50). In some aspects, the intracellular signaling domain comprises the amino acid sequence of

(SEQ ID NO: 50)
LRHRRQGKHWTSTQRKADFQHPAGAVGPEPTDRGLQWRSSPAADAQEENL
YAAVKHTQPEDGVEMDTRSPHDEDPQAVTYAEVKHSRPRREMASPPSPLS
GEFLDTKDRQAEEDRQMDTEAAASEAPQDVTYAQLHSLTLRREATEPPPS
QEGPSPAVPSIYATLAIH.

In some aspects, the intracellular signaling domain is derived from PD-1. In some aspects, the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to CSRAARGTIGARRTGQPLKEDPSAVPVFSVDYGELDFQWREKTPEPPVPCVPEQTEY ATIVFPSGMGTSSPARRGSADGPRSAQPLRPEDGHCSWPL (SEQ ID NO: 1). In some aspects, the intracellular signaling domain comprises the amino acid sequence of

(SEQ ID NO: 1)
CSRAARGTIGARRTGQPLKEDPSAVPVFSVDYGELDFQWREKTPEPPVPC
VPEQTEYATIVFPSGMGTSSPARRGSADGPRSAQPLRPEDGHCSWPL.

In some aspects, the intracellular signaling domain is derived from KIR3DL1. In some aspects, the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to In some aspects, one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to HLWCSNKKNAAVMDQEPAGNRTANSEDSDEQDPEEVTYAQLDHCVFTQRKITRPSQ RPKTPPTDTILYTELPNAKPRSKVVSCP (SEQ ID NO: 66). In some aspects, one of the one or more intracellular signaling domains comprises the amino acid sequence of

(SEQ ID NO: 66)
HLWCSNKKNAAVMDQEPAGNRTANSEDSDEQDPEEVTYAQLDHCVFTQRK
ITRPSQRPKTPPTDTILYTELPNAKPRSKVVSCP.

In some aspects, the intracellular signaling domain is derived from CTLA4. In some aspects, one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to AVSLSKMLKKRSPLTTGVGVKMPPTEPECEKQFQPYFIPIN (SEQ ID NO: 67). In some aspects, one of the one or more intracellular signaling domains comprises the amino acid sequence of AVSLSKMLKKRSPLTTGVGVKMPPTEPECEKQFQPYFIPIN (SEQ ID NO: 67).

In some aspects, the transmembrane domain is derived from a protein selected from the group consisting of: BTLA, CD8, CD28, CD3zeta, CD4, 4-IBB, OX40, ICOS, 2B4, CD25, CD7, LAX, LAT, PD-1, CTLA4, TIM3, KIR3DL1, LIR1, NKG2A, TIGIT, and LAG3.

In some aspects, the chimeric inhibitory receptor comprises a transmembrane domain derived from BTLA. In some aspects, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LLPLGGLPLLITTCFCLFCCL (SEQ ID NO: 12). In some aspects, the transmembrane domain comprises the amino acid sequence of LLPLGGLPLLITTCFCLFCCL (SEQ ID NO: 12). In some aspects, the transmembrane domain further comprises at least a portion of the BTLA extracellular domain.

In some aspects, the chimeric inhibitory receptor comprises a transmembrane domain derived from LIR1. In some aspects, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VIGILVAVILLLLLLLLLFLI (SEQ ID NO: 59). In some aspects, the transmembrane domain comprises the amino acid sequence of VIGILVAVILLLLLLLLLFLI (SEQ ID NO: 59). In some aspects, the transmembrane domain further comprises at least a portion of the LIR1 extracellular domain.

In some aspects, the chimeric inhibitory receptor comprises a transmembrane domain derived from PD-1. In some aspects, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VGVVGGLLGSLVLLVWVLAVI (SEQ ID NO: 60). In some aspects, the transmembrane domain comprises the amino acid sequence of VGVVGGLLGSLVLLVWVLAVI (SEQ ID NO: 60). In some aspects, the transmembrane domain further comprises at least a portion of the PD1 extracellular domain.

In some aspects, the chimeric inhibitory receptor comprises a transmembrane domain derived from CTLA4. In some aspects, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to DFLLWILAAVSSGLFFYSFLLT (SEQ ID NO: 68). In some aspects, the transmembrane domain comprises the amino acid sequence of DFLLWILAAVSSGLFFYSFLLT (SEQ ID NO: 68). In some aspects, the transmembrane domain further comprises at least a portion of the CTLA4 extracellular domain.

In some aspects, the chimeric inhibitory receptor comprises a transmembrane domain derived from KIR3DL1. In some aspects, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to ILIGTSVVIILFILLLFFLL (SEQ ID NO: 69). In some aspects, the transmembrane domain comprises the amino acid sequence of ILIGTSVVIILFILLLFFLL (SEQ ID NO: 69). In some aspects, the transmembrane domain further comprises at least a portion of the KIR3DL1 extracellular domain.

In some aspects, the chimeric inhibitory receptor comprises a transmembrane domain derived from CD28. In some aspects, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to FWVLVVVGGVLACYSLLVTVAFIIFWV (SEQ ID NO: 11). In some aspects, the transmembrane domain comprises the amino acid sequence of FWVLVVVGGVLACYSLLVTVAFIIFWV (SEQ ID NO: 11). In some aspects, the transmembrane domain further comprises at least a portion of the CD28 extracellular domain.

In some aspects, the protein is not expressed on the target tumor.

In some aspects, the protein is expressed on a non-tumor cell.

In some aspects, the protein is expressed on a non-tumor cell derived from a tissue selected from the group consisting of: brain, neuronal tissue, endocrine, endothelial, bone, bone marrow, immune system, muscle, lung, liver, gallbladder, pancreas, gastrointestinal tract, kidney, urinary bladder, male reproductive organs, female reproductive organs, adipose, soft tissue, and skin.

In some aspects, the extracellular protein binding domain comprises a ligand-binding domain.

In some aspects, the extracellular protein binding domain comprises a receptor-binding domain.

In some aspects, the extracellular protein binding domain comprises an antigen-binding domain.

In some aspects, the antigen-binding domain comprises an antibody, an antigen-binding fragment of an antibody, a F(ab) fragment, a F(ab′) fragment, a single chain variable fragment (scFv), or a single-domain antibody (sdAb).

In some aspects, the antigen-binding domain comprises a single chain variable fragment (scFv).

In some aspects, each scFv comprises a heavy chain variable domain (VH) and a light chain variable domain (VL).

In some aspects, the VH and VL are separated by a peptide linker.

In some aspects, the peptide linker comprises an amino acid sequence selected from the group consisting of: GGS (SEQ ID NO: 15), GGSGGS (SEQ ID NO: 16), GGSGGSGGS (SEQ ID NO: 17), GGSGGSGGSGGS (SEQ ID NO: 18), GGSGGSGGSGGSGGS (SEQ ID NO: 19), GGGS (SEQ ID NO: 20), GGGSGGGS (SEQ ID NO: 21), GGGSGGGSGGGS (SEQ ID NO: 22), GGGSGGGSGGGSGGGS (SEQ ID NO: 23), GGGSGGGSGGGSGGGSGGGS (SEQ ID NO: 24), GGGGS (SEQ ID NO: 25), GGGGSGGGGS (SEQ ID NO: 26), GGGGSGGGGSGGGGS (SEQ ID NO: 27), GGGGSGGGGSGGGGSGGGGS (SEQ ID NO: 28), and GGGGSGGGGSGGGGSGGGGSGGGGS (SEQ ID NO: 29).

In some aspects, the scFv comprises the structure VH-L-VL or VL-L-VH, wherein VH is the heavy chain variable domain, L is the peptide linker, and VL is the light chain variable domain.

In some aspects, the transmembrane domain is physically linked to the extracellular protein binding domain.

In some aspects, the intracellular signaling domain is physically linked to the transmembrane domain.

In some aspects, the transmembrane domain is physically linked to the extracellular protein binding domain and the intracellular signaling domain is physically linked to the transmembrane domain.

In some aspects, the protein binding domain has a high binding affinity.

In some aspects, the protein binding domain has a low binding affinity.

In some aspects, the chimeric inhibitory receptor is capable of suppressing cytokine production by an activated immunomodulatory cell.

In some aspects, the chimeric inhibitory receptor is capable of suppressing a cell-mediated immune response to a target cell, wherein the immune response is induced by activation of the immunomodulatory cell.

In some aspects, the target cell is a tumor cell.

In some aspects, the intracellular signaling domain comprises one or more modifications.

In some aspects, the one or more modifications modulate sensitivity of the chimeric inhibitory receptor relative to the otherwise identical, unmodified receptor.

In some aspects, the one or more modifications increase sensitivity of the chimeric inhibitory receptor relative to the otherwise identical, unmodified receptor.

In some aspects, the one or more modifications reduce sensitivity of the chimeric inhibitory receptor relative to the otherwise identical, unmodified receptor.

In some aspects, the one or more modifications modulate potency of the chimeric inhibitory receptor relative to the otherwise identical, unmodified receptor.

In some aspects, the one or more modifications increase potency of the chimeric inhibitory receptor relative to the otherwise identical, unmodified receptor.

In some aspects, the one or more modifications reduce potency of the chimeric inhibitory receptor relative to the otherwise identical, unmodified receptor.

In some aspects, the one or more modifications modulate basal prevention, attenuation, or inhibition of activation of the tumor-targeting chimeric receptor when expressed on an immunomodulatory cell relative to the otherwise identical, unmodified receptor.

In some aspects, the one or more modifications reduce basal prevention, attenuation, or inhibition relative to the otherwise identical, unmodified receptor.

In some aspects, the one or more modifications increase basal prevention, attenuation, or inhibition relative to the otherwise identical, unmodified receptor.

In some aspects, the chimeric inhibitory receptor further comprises a spacer region positioned between the protein binding domain and the transmembrane domain and operably linked to each of the protein binding domain and the transmembrane domain.

In some aspects, the chimeric inhibitory receptor further comprises a spacer region positioned between the protein binding domain and the transmembrane domain and physically linked to each of the protein binding domain and the transmembrane domain.

In some aspects, the spacer region is derived from a protein selected from the group consisting of: CD8alpha, CD4, CD7, CD28, IgG1, IgG4, FcgammaRIIIalpha, LNGFR, and PDGFR.

In some aspects, the spacer region comprises an amino acid sequence selected from the group consisting of: AAAIEVMYPPPYLDNEKSNGTIIHVKGKHLCPSPLFPGPSKP (SEQ ID NO: 31), ESKYGPPCPSCP (SEQ ID NO: 32), ESKYGPPAPSAP (SEQ ID NO: 33), ESKYGPPCPPCP (SEQ ID NO: 34), EPKSCDKTHTCP (SEQ ID NO: 35), AAAFVPVFLPAKPTTTPAPRPPTPAPTIASQPLSLRPEACRPAAGGAVHTRGLDFACDI YIWAPLAGTCGVLLLSLVITLYCNHRN (SEQ ID NO: 36), TTTPAPRPPTPAPTIALQPLSLRPEACRPAAGGAVHTRGLDFACD (SEQ ID NO: 37) ACPTGLYTHSGECCKACNLGEGVAQPCGANQTVCEPCLDSVTFSDVVSATEPCKPCT ECVGLQSMSAPCVEADDAVCRCAYGYYQDETTGRCEACRVCEAGSGLVFSCQDKQ NTVCEECPDGTYSDEADAEC (SEQ ID NO: 38), ACPTGLYTHSGECCKACNLGEGVAQPCGANQTVC (SEQ ID NO: 39), AVGQDTQEVIVVPHSLPFKV (SEQ ID NO: 40), and TTTPAPRPPTPAPTIALQPLSLRPEACRPAAGGAVHTRGLDFACDQTTPGERSSLPAFY PGTSGSCSGCGSLSLP (SEQ ID NO: 70).

In some aspects, the spacer region modulates sensitivity of the chimeric inhibitory receptor relative to an otherwise identical chimeric inhibitory receptor lacking the spacer region.

In some aspects, the spacer region increases sensitivity of the chimeric inhibitory receptor relative to an otherwise identical chimeric inhibitory receptor lacking the spacer region.

In some aspects, the spacer region reduces sensitivity of the chimeric inhibitory receptor relative to an otherwise identical chimeric inhibitory receptor lacking the spacer region.

In some aspects, the spacer region modulates potency of the chimeric inhibitory receptor relative to an otherwise identical chimeric inhibitory receptor lacking the spacer region.

In some aspects, the spacer region increases potency of the chimeric inhibitory receptor relative to an otherwise identical chimeric inhibitory receptor lacking the spacer region.

In some aspects, the spacer region reduces potency of the chimeric inhibitory receptor relative to an otherwise identical chimeric inhibitory receptor lacking the spacer region.

In some aspects, the spacer region modulates basal prevention, attenuation, or inhibition of activation of the tumor-targeting chimeric receptor when expressed on an immunomodulatory cell relative to an otherwise identical chimeric inhibitory receptor lacking the spacer region.

In some aspects, the spacer region reduces basal prevention, attenuation, or inhibition relative to an otherwise identical chimeric inhibitory receptor lacking the spacer region.

In some aspects, the spacer region increases basal prevention, attenuation, or inhibition relative to an otherwise identical chimeric inhibitory receptor lacking the spacer region.

In some aspects, the chimeric inhibitory receptor further comprises an intracellular spacer region positioned between the transmembrane domain and the intracellular signaling domain and operably linked to each of the transmembrane domain and the intracellular signaling domain.

In some aspects, the chimeric inhibitory receptor further comprises an intracellular spacer region positioned between the transmembrane domain and the intracellular signaling domain and physically linked to each of the transmembrane domain and the intracellular signaling domain.

In some aspects, the intracellular spacer region modulates sensitivity of the chimeric inhibitory receptor relative to an otherwise identical chimeric inhibitory receptor lacking the intracellular spacer region.

In some aspects, the intracellular spacer region increases sensitivity of the chimeric inhibitory receptor relative to an otherwise identical chimeric inhibitory receptor lacking the intracellular spacer region.

In some aspects, the intracellular spacer region reduces sensitivity of the chimeric inhibitory receptor relative to an otherwise identical chimeric inhibitory receptor lacking the intracellular spacer region.

In some aspects, the intracellular spacer region modulates potency of the chimeric inhibitory receptor relative to an otherwise identical chimeric inhibitory receptor lacking the intracellular spacer region.

In some aspects, the intracellular spacer region increases potency of the chimeric inhibitory receptor relative to an otherwise identical chimeric inhibitory receptor lacking the intracellular spacer region.

In some aspects, the intracellular spacer region reduces potency of the chimeric inhibitory receptor relative to an otherwise identical chimeric inhibitory receptor lacking the intracellular spacer region.

In some aspects, the intracellular spacer region modulates basal prevention, attenuation, or inhibition of activation of the tumor-targeting chimeric receptor when expressed on an immunomodulatory cell relative to an otherwise identical chimeric inhibitory receptor lacking the intracellular spacer region.

In some aspects, the intracellular spacer region reduces basal prevention, attenuation, or inhibition relative to an otherwise identical chimeric inhibitory receptor lacking the intracellular spacer region.

In some aspects, the intracellular spacer region increases basal prevention, attenuation, or inhibition relative to an otherwise identical chimeric inhibitory receptor lacking the intracellular spacer region.

In some aspects, the inhibitory chimeric receptor further comprises an enzymatic inhibitory domain.

In some aspects, the enzymatic inhibitory domain is capable of preventing, attenuating, or inhibiting activation of a tumor-targeting chimeric receptor when expressed on an immunomodulatory cell relative to an otherwise identical chimeric inhibitory receptor lacking the enzymatic inhibitory domain.

In some aspects, the enzymatic inhibitory domain comprises an enzyme catalytic domain.

In some aspects, the enzyme catalytic domain is derived from an enzyme selected from the group consisting of CSK, SUP-1, PTEN, CD45, CD148, PTP-MEG1, PTP-PEST, c-CBL, CBL-b, PTPN22, LAR, PTPH1, SHIP-1, and RasGAP.

In some aspects, the enzymatic inhibitory domain comprises one or more modifications that modulate basal prevention, attenuation, or inhibition relative to an otherwise identical enzymatic inhibitory domain lacking the one or more modifications.

In some aspects, the one or more modifications reduce basal prevention, attenuation, or inhibition of the chimeric inhibitory receptor relative to an otherwise identical chimeric inhibitory receptor lacking the enzymatic inhibitory domain.

In some aspects, the one or more modifications increase basal prevention, attenuation, or inhibition of the chimeric inhibitory receptor relative to an otherwise identical chimeric inhibitory receptor lacking the enzymatic inhibitory domain.

In some aspects, the tumor-targeting chimeric receptor is a tumor-targeting chimeric antigen receptor (CAR) or an engineered T cell receptor (TCR).

In some aspects, the immunomodulatory cell is selected from the group consisting of a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an ESC-derived cell, and an iPSC-derived cell.

Also provided herein are chimeric inhibitory receptors comprising: an extracellular protein binding domain, a transmembrane domain, wherein the transmembrane domain is operably linked to the extracellular protein binding domain, and two or more intracellular signaling domains, wherein the two or more intracellular signaling domains are operably linked to the transmembrane domain; and wherein at least one of the two or more intracellular signaling domains is capable of preventing, attenuating, or inhibiting activation of a tumor-targeting chimeric receptor expressed on an immunomodulatory cell.

In some aspects, the two or more intracellular signaling domains are each derived from a protein selected from the group consisting of: BTLA, PD-1, CTLA4, TIM3, KIR3DL1, LIR1, NKG2A, TIGIT, and LAG3.

In some aspects, the transmembrane domain is derived from the same protein as one of the two or more intracellular signaling domains.

In some aspects, the transmembrane domain further comprises at least a portion of an extracellular domain of the same protein.

In some aspects, the transmembrane domain is derived from a first protein and the two or more intracellular signaling domains are derived from proteins that are distinct from the first protein.

In some aspects, at least one of the two or more intracellular signaling domains is derived from BTLA. In some aspects, the at least one of the two or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RRHQGKQNELSDTAGREINLVDAHLKSEQTEASTRQNSQVLLSETGIYDNDPDLCFR MQEGSEVYSNPCLEENKPGIVYASLNHSVIGPNSRLARNVKEAPTEYASICVRS (SEQ ID NO: 3). In some aspects, the at least one of the two or more intracellular signaling domains comprises the amino acid sequence of

(SEQ ID NO: 3)
RRHQGKQNELSDTAGREINLVDAHLKSEQTEASTRQNSQVLLSETGIYDN
DPDLCFRMQEGSEVYSNPCLEENKPGIVYASLNHSVIGPNSRLARNVKEA
PTEYASICVRS.

In some aspects, at least one of the two or more intracellular signaling domains is derived from LIR1. In some aspects, the at least one of the two or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LRHRRQGKHWTSTQRKADFQHPAGAVGPEPTDRGLQWRSSPAADAQEENLYAAVK HTQPEDGVEMDTRSPHDEDPQAVTYAEVKHSRPRREMASPPSPLSGEFLDTKDRQAE EDRQMDTEAAASEAPQDVTYAQLHSLTLRREATEPPPSQEGPSPAVPSIYATLAIH (SEQ ID NO: 50). In some aspects, the at least one of the two or more intracellular signaling domains comprises the amino acid sequence of

(SEQ ID NO: 50)
LRHRRQGKHWTSTQRKADFQHPAGAVGPEPTDRGLQWRSSPAADAQEEN
LYAAVKHTQPEDGVEMDTRSPHDEDPQAVTYAEVKHSRPRREMASPPSP
LSGEFLDTKDRQAEEDRQMDTEAAASEAPQDVTYAQLHSLTLRREATEP
PPSQEGPSPAVPSIYATLAIH.

In some aspects, at least one of the two or more intracellular signaling domains is derived from PD-1. In some aspects, the at least one of the two or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to CSRAARGTIGARRTGQPLKEDPSAVPVFSVDYGELDFQWREKTPEPPVPCVPEQTEY ATIVFPSGMGTSSPARRGSADGPRSAQPLRPEDGHCSWPL (SEQ ID NO: 1). In some aspects, the at least one of the two or more intracellular signaling domains comprises the amino acid sequence of

(SEQ ID NO: 1)
CSRAARGTIGARRTGQPLKEDPSAVPVFSVDYGELDFQWREKTPEPPVPC
VPEQTEYATIVFPSGMGTSSPARRGSADGPRSAQPLRPEDGHCSWPL.

In some aspects, at least one of the two or more intracellular signaling domains is derived from KIR3DL1. In some aspects, the at least one of the two or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to HLWCSNKKNAAVMDQEPAGNRTANSEDSDEQDPEEVTYAQLDHCVFTQRKITRPSQ RPKTPPTDTILYTELPNAKPRSKVVSCP (SEQ ID NO: 66). In some aspects, the at least one of the two or more intracellular signaling domains comprises the amino acid sequence of

(SEQ ID NO: 66)
HLWCSNKKNAAVMDQEPAGNRTANSEDSDEQDPEEVTYAQLDHCVFTQRK
ITRPSQRPKTPPTDTILYTELPNAKPRSKVVSCP.

In some aspects, at least one of the two or more intracellular signaling domains is derived from CTLA4. In some aspects, the at least one of the two or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to AVSLSKMLKKRSPLTTGVGVKMPPTEPECEKQFQPYFIPIN (SEQ ID NO: 67). In some aspects, the at least one of the two or more intracellular signaling domains comprises the amino acid sequence of AVSLSKMLKKRSPLTTGVGVKMPPTEPECEKQFQPYFIPIN (SEQ ID NO: 67).

In some aspects, the transmembrane domain is derived from a protein selected from the group consisting of: BTLA, CD8, CD28, CD3zeta, CD4, 4-IBB, OX40, ICOS, 2B4, CD25, CD7, LAX, LAT, PD-1, CTLA4, TIM3, KIR3DL1, LIR1, NKG2A, TIGIT, and LAG3.

In some aspects, the chimeric inhibitory receptor comprises a transmembrane domain derived from BTLA. In some aspects, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LLPLGGLPLLITTCFCLFCCL (SEQ ID NO: 12). In some aspects, the transmembrane domain comprises the amino acid sequence of LLPLGGLPLLITTCFCLFCCL (SEQ ID NO: 12). In some aspects, the transmembrane domain further comprises at least a portion of the BTLA extracellular domain.

In some aspects, the chimeric inhibitory receptor comprises a transmembrane domain derived from LIR1. In some aspects, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VIGILVAVILLLLLLLLLFLI (SEQ ID NO: 59). In some aspects, the transmembrane domain comprises the amino acid sequence of VIGILVAVILLLLLLLLLFLI (SEQ ID NO: 59). In some aspects, the transmembrane domain further comprises at least a portion of the LIR1 extracellular domain.

In some aspects, the chimeric inhibitory receptor comprises a transmembrane domain derived from PD-1. In some aspects, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VGVVGGLLGSLVLLVWVLAVI (SEQ ID NO: 60). In some aspects, the transmembrane domain comprises the amino acid sequence of VGVVGGLLGSLVLLVWVLAVI (SEQ ID NO: 60). In some aspects, the transmembrane domain further comprises at least a portion of the PD-1 extracellular domain.

In some aspects, the chimeric inhibitory receptor comprises a transmembrane domain derived from CTLA4. In some aspects, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to DFLLWILAAVSSGLFFYSFLLT (SEQ ID NO: 68). In some aspects, the transmembrane domain comprises the amino acid sequence of DFLLWILAAVSSGLFFYSFLLT (SEQ ID NO: 68). In some aspects, the transmembrane domain further comprises at least a portion of the CTLA4 extracellular domain.

In some aspects, the chimeric inhibitory receptor comprises a transmembrane domain derived from KIR3DL1. In some aspects, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to ILIGTSVVIILFILLLFFLL (SEQ ID NO: 69). In some aspects, the transmembrane domain comprises the amino acid sequence of ILIGTSVVIILFILLLFFLL (SEQ ID NO: 69). In some aspects, the transmembrane domain further comprises at least a portion of the KIR3DL1 extracellular domain.

In some aspects, the chimeric inhibitory receptor comprises a transmembrane domain derived from CD28. In some aspects, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to FWVLVVVGGVLACYSLLVTVAFIIFWV (SEQ ID NO: 11). In some aspects, the transmembrane domain comprises the amino acid sequence of FWVLVVVGGVLACYSLLVTVAFIIFWV (SEQ ID NO: 11). In some aspects, the transmembrane domain further comprises at least a portion of the CD28 extracellular domain.

In some aspects, the chimeric inhibitory receptor comprises a first intracellular signaling domain derived from LIR1 and a second intracellular signaling domain derived from BTLA.

In some aspects, the chimeric inhibitory receptor comprises a first intracellular signaling domain derived from LIR1 and a second intracellular signaling domain derived from PD-1.

In some aspects, the chimeric inhibitory receptor comprises a first intracellular signaling domain derived from LIR1 and a second intracellular signaling domain derived from KIR3DL1.

In some aspects, the chimeric inhibitory receptor comprises a first intracellular signaling domain derived from LIR1 and a second intracellular signaling domain derived from LIR1.

In some aspects, the chimeric inhibitory receptor comprises a first intracellular signaling domain derived from LIR1 and a second intracellular signaling domain derived from KIR3DL1.

In some aspects, the first intracellular signaling domain further comprises a transmembrane domain derived from LIR1.

In some aspects, the chimeric inhibitory receptor comprises a first intracellular signaling domain derived from BTLA and a second intracellular signaling domain derived from LIR1.

In some aspects, the chimeric inhibitory receptor comprises a first intracellular signaling domain derived from BTLA and a second intracellular signaling domain derived from PD-1.

In some aspects, the first intracellular signaling domain further comprises a transmembrane domain derived from BTLA.

In some aspects, the chimeric inhibitory receptor comprises a first intracellular signaling domain derived from PD-1 and a second intracellular signaling domain derived from LIR1.

In some aspects, the chimeric inhibitory receptor comprises a first intracellular signaling domain derived from PD-1 and a second intracellular signaling domain derived from BTLA.

In some aspects, the first intracellular signaling domain further comprises a transmembrane domain derived from PD-1.

In some aspects, the protein is not expressed on the target tumor.

In some aspects, the protein is expressed on a non-tumor cell.

In some aspects, the protein is expressed on a non-tumor cell derived from a tissue selected from the group consisting of: brain, neuronal tissue, endocrine, endothelial, bone, bone marrow, immune system, muscle, lung, liver, gallbladder, pancreas, gastrointestinal tract, kidney, urinary bladder, male reproductive organs, female reproductive organs, adipose, soft tissue, and skin.

In some aspects, the extracellular protein binding domain comprises a ligand-binding domain.

In some aspects, the extracellular protein binding domain comprises a receptor-binding domain.

In some aspects, the extracellular protein binding domain comprises an antigen-binding domain.

In some aspects, the antigen-binding domain comprises an antibody, an antigen-binding fragment of an antibody, a F(ab) fragment, a F(ab′) fragment, a single chain variable fragment (scFv), or a single-domain antibody (sdAb).

In some aspects, the antigen-binding domain comprises a single chain variable fragment (scFv).

In some aspects, each scFv comprises a heavy chain variable domain (VH) and a light chain variable domain (VL).

In some aspects, the VH and VL are separated by a peptide linker.

In some aspects, the peptide linker comprises an amino acid sequence selected from the group consisting of: GGS (SEQ ID NO: 15), GGSGGS (SEQ ID NO: 16), GGSGGSGGS (SEQ ID NO: 17), GGSGGSGGSGGS (SEQ ID NO: 18), GGSGGSGGSGGSGGS (SEQ ID NO: 19), GGGS (SEQ ID NO: 20), GGGSGGGS (SEQ ID NO: 21), GGGSGGGSGGGS (SEQ ID NO: 22), GGGSGGGSGGGSGGGS (SEQ ID NO: 23), GGGSGGGSGGGSGGGSGGGS (SEQ ID NO: 24), GGGGS (SEQ ID NO: 25), GGGGSGGGGS (SEQ ID NO: 26), GGGGSGGGGSGGGGS (SEQ ID NO: 27), GGGGSGGGGSGGGGSGGGGS (SEQ ID NO: 28), and GGGGSGGGGSGGGGSGGGGSGGGGS (SEQ ID NO: 29).

In some aspects, the scFv comprises the structure VH-L-VL or VL-L-VH, wherein VH is the heavy chain variable domain, L is the peptide linker, and VL is the light chain variable domain.

In some aspects, the transmembrane domain is physically linked to the extracellular protein binding domain.

In some aspects, one of the two or more intracellular signaling domains is physically linked to the transmembrane domain.

In some aspects, the transmembrane domain is physically linked to the extracellular protein binding domain and one of the two or more intracellular signaling domains is physically linked to the transmembrane domain.

In some aspects, the protein binding domain has a high binding affinity.

In some aspects, the protein binding domain has a low binding affinity.

In some aspects, the chimeric inhibitory receptor is capable of suppressing cytokine production by an activated immunomodulatory cell.

In some aspects, the chimeric inhibitory receptor is capable of suppressing a cell-mediated immune response to a target cell, wherein the immune response is induced by activation of the immunomodulatory cell.

In some aspects, the target cell is a tumor cell.

In some aspects, at least one of the two or more intracellular signaling domains comprises one or more modifications.

In some aspects, the one or more modifications modulate sensitivity of the chimeric inhibitory receptor relative to the otherwise identical, unmodified receptor.

In some aspects, the one or more modifications increase sensitivity of the chimeric inhibitory receptor relative to the otherwise identical, unmodified receptor.

In some aspects, the one or more modifications reduce sensitivity of the chimeric inhibitory receptor relative to the otherwise identical, unmodified receptor.

In some aspects, the one or more modifications modulate potency of the chimeric inhibitory receptor relative to the otherwise identical, unmodified receptor.

In some aspects, the one or more modifications increase potency of the chimeric inhibitory receptor relative to the otherwise identical, unmodified receptor.

In some aspects, the one or more modifications reduce potency of the chimeric inhibitory receptor relative to the otherwise identical, unmodified receptor.

In some aspects, the one or more modifications modulate basal prevention, attenuation, or inhibition of activation of the tumor-targeting chimeric receptor when expressed on an immunomodulatory cell relative to the otherwise identical, unmodified receptor.

In some aspects, the one or more modifications reduce basal prevention, attenuation, or inhibition relative to the otherwise identical, unmodified receptor.

In some aspects, the one or more modifications increase basal prevention, attenuation, or inhibition relative to the otherwise identical, unmodified receptor.

In some aspects, the chimeric inhibitory receptor further comprises a spacer region positioned between the protein binding domain and the transmembrane domain and operably linked to each of the protein binding domain and the transmembrane domain.

In some aspects, the chimeric inhibitory receptor further comprises a spacer region positioned between the protein binding domain and the transmembrane domain and physically linked to each of the protein binding domain and the transmembrane domain.

In some aspects, the spacer region is derived from a protein selected from the group consisting of: CD8alpha, CD4, CD7, CD28, IgG1, IgG4, FcgammaRIIIalpha, LNGFR, and PDGFR.

In some aspects, the spacer region comprises an amino acid sequence selected from the group consisting of:

(SEQ ID NO: 31)
AAAIEVMYPPPYLDNEKSNGTIIHVKGKHLCPSPLFPGPSKP,
(SEQ ID NO: 32)
ESKYGPPCPSCP,
(SEQ ID NO: 33)
ESKYGPPAPSAP,
(SEQ ID NO: 34)
ESKYGPPCPPCP,
(SEQ ID NO: 35)
EPKSCDKTHTCP,
(SEQ ID NO: 36)
AAAFVPVFLPAKPTTTPAPRPPTPAPTIASQPLSLRPEACRPAAGGAVHT
RGLDFACDIYIWAPLAGTCGVLLLSLVITLYCNHRN,
(SEQ ID NO: 37)
TTTPAPRPPTPAPTIALQPLSLRPEACRPAAGGAVHTRGLDFACD
(SEQ ID NO: 38)
ACPTGLYTHSGECCKACNLGEGVAQPCGANQTVCEPCLDSVTFSDVVSAT
EPCKPCTECVGLQSMSAPCVEADDAVCRCAYGYYQDETTGRCEACRVCEA
GSGLVFSCQDKQNTVCEECPDGTYSDEADAEC,
(SEQ ID NO: 39)
ACPTGLYTHSGECCKACNLGEGVAQPCGANQTVC,
(SEQ ID NO: 40)
AVGQDTQEVIVVPHSLPFKV,
and
(SEQ ID NO: 70)
TTTPAPRPPTPAPTIALQPLSLRPEACRPAAGGAVHTRGLDFACDQTTPG
ERSSLPAFYPGTSGSCSGCGSLSLP.

In some aspects, the spacer region modulates sensitivity of the chimeric inhibitory receptor relative to an otherwise identical chimeric inhibitory receptor lacking the spacer region.

In some aspects, the spacer region increases sensitivity of the chimeric inhibitory receptor relative to an otherwise identical chimeric inhibitory receptor lacking the spacer region.

In some aspects, the spacer region reduces sensitivity of the chimeric inhibitory receptor relative to an otherwise identical chimeric inhibitory receptor lacking the spacer region.

In some aspects, the spacer region modulates potency of the chimeric inhibitory receptor relative to an otherwise identical chimeric inhibitory receptor lacking the spacer region.

In some aspects, the spacer region increases potency of the chimeric inhibitory receptor relative to an otherwise identical chimeric inhibitory receptor lacking the spacer region.

In some aspects, the spacer region reduces potency of the chimeric inhibitory receptor relative to an otherwise identical chimeric inhibitory receptor lacking the spacer region.

In some aspects, the spacer region modulates basal prevention, attenuation, or inhibition of activation of the tumor-targeting chimeric receptor when expressed on an immunomodulatory cell relative to an otherwise identical chimeric inhibitory receptor lacking the spacer region.

In some aspects, the spacer region reduces basal prevention, attenuation, or inhibition relative to an otherwise identical chimeric inhibitory receptor lacking the spacer region.

In some aspects, the spacer region increases basal prevention, attenuation, or inhibition relative to an otherwise identical chimeric inhibitory receptor lacking the spacer region.

In some aspects, the chimeric inhibitory receptor further comprises an intracellular spacer region positioned between the transmembrane domain and one of the two or more intracellular signaling domains and operably linked to each of the transmembrane domain and the intracellular signaling domain.

In some aspects, the chimeric inhibitory receptor further comprises an intracellular spacer region positioned between the transmembrane domain and one of the two or more intracellular signaling domains and physically linked to each of the transmembrane domain and the intracellular signaling domain.

In some aspects, the intracellular spacer region modulates sensitivity of the chimeric inhibitory receptor relative to an otherwise identical chimeric inhibitory receptor lacking the intracellular spacer region.

In some aspects, the intracellular spacer region increases sensitivity of the chimeric inhibitory receptor relative to an otherwise identical chimeric inhibitory receptor lacking the intracellular spacer region.

In some aspects, the intracellular spacer region reduces sensitivity of the chimeric inhibitory receptor relative to an otherwise identical chimeric inhibitory receptor lacking the intracellular spacer region.

In some aspects, the intracellular spacer region modulates potency of the chimeric inhibitory receptor relative to an otherwise identical chimeric inhibitory receptor lacking the intracellular spacer region.

In some aspects, the intracellular spacer region increases potency of the chimeric inhibitory receptor relative to an otherwise identical chimeric inhibitory receptor lacking the intracellular spacer region.

In some aspects, the intracellular spacer region reduces potency of the chimeric inhibitory receptor relative to an otherwise identical chimeric inhibitory receptor lacking the intracellular spacer region.

In some aspects, the intracellular spacer region modulates basal prevention, attenuation, or inhibition of activation of the tumor-targeting chimeric receptor when expressed on an immunomodulatory cell relative to an otherwise identical chimeric inhibitory receptor lacking the intracellular spacer region.

In some aspects, the intracellular spacer region reduces basal prevention, attenuation, or inhibition relative to an otherwise identical chimeric inhibitory receptor lacking the intracellular spacer region.

In some aspects, the intracellular spacer region increases basal prevention, attenuation, or inhibition relative to an otherwise identical chimeric inhibitory receptor lacking the intracellular spacer region.

In some aspects, the inhibitory chimeric receptor further comprises an enzymatic inhibitory domain.

In some aspects, the enzymatic inhibitory domain is capable of preventing, attenuating, or inhibiting activation of a tumor-targeting chimeric receptor when expressed on an immunomodulatory cell relative to an otherwise identical chimeric inhibitory receptor lacking the enzymatic inhibitory domain.

In some aspects, the enzymatic inhibitory domain comprises an enzyme catalytic domain.

In some aspects, the enzyme catalytic domain is derived from an enzyme selected from the group consisting of: CSK, SUP-1, PTEN, CD45, CD148, PTP-MEG1, PTP-PEST, c-CBL, CBL-b, PTPN22, LAR, PTPH1, SHIP-1, and RasGAP.

In some aspects, the enzymatic inhibitory domain comprises one or more modifications that modulate basal prevention, attenuation, or inhibition relative to an otherwise identical enzymatic inhibitory domain lacking the one or more modifications.

In some aspects, the one or more modifications reduce basal prevention, attenuation, or inhibition of the chimeric inhibitory receptor relative to an otherwise identical chimeric inhibitory receptor lacking the enzymatic inhibitory domain.

In some aspects, the one or more modifications increase basal prevention, attenuation, or inhibition of the chimeric inhibitory receptor relative to an otherwise identical chimeric inhibitory receptor lacking the enzymatic inhibitory domain.

In some aspects, the tumor-targeting chimeric receptor is a tumor-targeting chimeric antigen receptor (CAR) or an engineered T cell receptor (TCR).

In some aspects, the immunomodulatory cell is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an ESC-derived cell, and an iPSC-derived cell. In some aspects, the immunomodulatory cell is a Natural Killer (NK) cell.

Also provided herein are compositions comprising the chimeric inhibitory receptor of as described herein and a pharmaceutically acceptable carrier.

Also provided herein are engineered nucleic acids encoding the chimeric inhibitory receptor as described herein.

Also provided herein are expression vectors comprising the engineered nucleic acids described herein.

Also provided herein are isolated immunomodulatory cells comprising the engineered nucleic acid encoding the chimeric inhibitory receptor as described herein or the expression vector of as described herein.

Also provided herein are compositions comprising the engineered nucleic acid as described herein or the expression vector as described herein, and a pharmaceutically acceptable carrier

Also provided herein are isolated immunomodulatory cells comprising the chimeric inhibitory receptor as described herein.

In some aspects, the cell further comprises a tumor-targeting chimeric receptor expressed on the surface of the cell.

In some aspects, upon binding of the protein to the chimeric inhibitory receptor, the chimeric inhibitory receptor prevents, attenuates, or inhibits activation of the tumor-targeting chimeric receptor relative to an otherwise identical cell lacking a chimeric inhibitory receptor.

Also provided herein are isolated immunomodulatory cells comprising a chimeric inhibitory receptor, wherein the chimeric inhibitory receptor comprises: an extracellular protein binding domain; a transmembrane domain, wherein the transmembrane domain is operably linked to the extracellular protein binding domain; and an intracellular signaling domain, wherein the intracellular signaling domain is operably linked to the transmembrane domain, and wherein upon binding of the protein to the chimeric inhibitory receptor, the chimeric inhibitory receptor prevents, attenuates, or inhibits activation of a tumor-targeting chimeric receptor expressed on the surface of the cell relative to an otherwise identical cell lacking a chimeric inhibitory receptor.

In some aspects, the cell further comprises a tumor-targeting chimeric receptor expressed on the surface of the cell.

Also provided herein are isolated immunomodulatory cells comprising: a chimeric inhibitory receptor, wherein the chimeric inhibitory receptor comprises: an extracellular protein binding domain, a transmembrane domain, wherein the transmembrane domain is operably linked to the extracellular protein binding domain, and an intracellular signaling domain, wherein the intracellular signaling domain is operably linked to the transmembrane domain; and a tumor-targeting chimeric receptor expressed on the surface of the cell, wherein upon binding of the protein to the chimeric inhibitory receptor, the chimeric inhibitory receptor prevents, attenuates, or inhibits activation of the tumor-targeting chimeric receptor relative to an otherwise identical cell lacking a chimeric inhibitory receptor.

In some aspects, the chimeric inhibitory receptor is recombinantly expressed.

In some aspects, the chimeric inhibitory receptor is expressed from a vector or a selected locus from the genome of the cell.

In some aspects, the tumor-targeting chimeric receptor is a chimeric antigen receptor (CAR) or an engineered T cell receptor.

In some aspects, prior to binding of the protein to the chimeric inhibitory receptor, the tumor-targeting chimeric receptor is capable of activating the cell.

In some aspects, upon binding of the protein to the chimeric inhibitory receptor, the chimeric inhibitory receptor suppresses cytokine production from the activated cell.

In some aspects, upon binding of the protein to the chimeric inhibitory receptor, the chimeric inhibitory receptor suppresses a cell-mediated immune response to a target cell, wherein the immune response is induced by activation of the immunomodulatory cell.

In some aspects, the transmembrane domain is physically linked to the extracellular protein binding domain.

In some aspects, the intracellular signaling domain is physically linked to the transmembrane domain.

In some aspects, the transmembrane domain is physically linked to the extracellular protein binding domain and the intracellular signaling domain is physically linked to the transmembrane domain.

Also provided herein are isolated immunomodulatory cells comprising a chimeric inhibitory receptor, wherein the chimeric inhibitory receptor comprises: an extracellular protein binding domain; a transmembrane domain, wherein the transmembrane domain is operably linked to the extracellular protein binding domain, and two or more intracellular signaling domains, wherein the two or more intracellular signaling domains are operably linked to the transmembrane domain; and wherein upon binding of the protein to the chimeric inhibitory receptor, the chimeric inhibitory receptor prevents, attenuates, or inhibits activation of a tumor-targeting chimeric receptor expressed on the surface of the cell relative to an otherwise identical cell lacking a chimeric inhibitory receptor.

In some aspects, the cell further comprises a tumor-targeting chimeric receptor expressed on the surface of the cell.

Also provided herein are isolated immunomodulatory cells comprising: (a) a chimeric inhibitory receptor, wherein the chimeric inhibitory receptor comprises: an extracellular protein binding domain, a transmembrane domain, wherein the transmembrane domain is operably linked to the extracellular protein binding domain, and two or more intracellular signaling domains, wherein the two or more intracellular signaling domains are operably linked to the transmembrane domain; and (b) a tumor-targeting chimeric receptor expressed on the surface of the cell, wherein upon binding of the protein to the chimeric inhibitory receptor, the chimeric inhibitory receptor prevents, attenuates, or inhibits activation of the tumor-targeting chimeric receptor relative to an otherwise identical cell lacking a chimeric inhibitory receptor.

In some aspects, the chimeric inhibitory receptor is recombinantly expressed.

In some aspects, the chimeric inhibitory receptor is expressed from a vector or a selected locus from the genome of the cell.

In some aspects, the tumor-targeting chimeric receptor is a chimeric antigen receptor (CAR) or an engineered T cell receptor.

In some aspects, prior to binding of the protein to the chimeric inhibitory receptor, the tumor-targeting chimeric receptor is capable of activating the cell.

In some aspects, upon binding of the protein to the chimeric inhibitory receptor, the chimeric inhibitory receptor suppresses cytokine production from the activated cell.

In some aspects, upon binding of the protein to the chimeric inhibitory receptor, the chimeric inhibitory receptor suppresses a cell-mediated immune response to a target cell, wherein the immune response is induced by activation of the immunomodulatory cell.

In some aspects, the transmembrane domain is physically linked to the extracellular protein binding domain.

In some aspects, one of the two or more intracellular signaling domains is physically linked to the transmembrane domain.

In some aspects, the transmembrane domain is physically linked to the extracellular protein binding domain and one of the two or more intracellular signaling domains is physically linked to the transmembrane domain.

In some aspects, the target cell is a tumor cell.

In some aspects, the cell is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an ESC-derived cell, and an iPSC-derived cell. In some aspects, the immunomodulatory cell is a Natural Killer (NK) cell.

In some aspects, the cell is autologous.

In some aspects, the cell is allogeneic.

Also provided herein are compositions comprising the isolated cell as described herein and a pharmaceutically acceptable carrier.

Also provided herein are methods of preventing, attenuating, or inhibiting a cell-mediated immune response induced by a tumor-targeting chimeric receptor expressed of the surface of an immunomodulatory cell, comprising: engineering the immunomodulatory cell to express the chimeric inhibitory receptor as described herein on the surface of the immunomodulatory cell, wherein upon binding of a cognate protein to the chimeric inhibitory receptor, the intracellular signaling domain prevents, attenuates, or inhibits activation of the tumor-targeting chimeric receptor.

Also provided herein are methods of preventing, attenuating, or inhibiting activation of a tumor-targeting chimeric receptor expressed on the surface of an immunomodulatory cell, comprising: contacting the isolated cell as described herein or the compositions as described herein with a cognate protein of the chimeric inhibitory receptor under conditions suitable for the chimeric inhibitory receptor to bind the cognate protein, wherein upon binding of the protein to the chimeric inhibitory receptor, the intracellular signaling domain prevents, attenuates, or inhibits activation of the tumor-targeting chimeric receptor.

In some aspects, the tumor-targeting chimeric receptor is a chimeric antigen receptor (CAR) or an engineered T cell receptor (TCR).

In some aspects, the CAR binds one or more antigens expressed on the surface of a tumor cell.

In some aspects, upon binding of the protein to the chimeric inhibitory receptor, the chimeric inhibitory receptor prevents, attenuates, or inhibits activation of the tumor-targeting chimeric receptor relative to an otherwise identical cell lacking a chimeric inhibitory receptor.

Also provided herein are isolated immunomodulatory cells comprising a chimeric inhibitory receptor, wherein the chimeric inhibitory receptor comprises: —an extracellular protein binding domain; —a transmembrane domain, wherein the transmembrane domain is operably linked to the extracellular protein binding domain, and —an intracellular signaling domain, wherein the intracellular signaling domain is operably linked to the transmembrane domain; and wherein upon binding of the protein to the chimeric inhibitory receptor, the chimeric inhibitory receptor prevents, attenuates, or inhibits activation of a tumor-targeting chimeric receptor expressed on the surface of the cell relative to an otherwise identical cell lacking a chimeric inhibitory receptor.

In some aspects, the cell further comprises a tumor-targeting chimeric receptor expressed on the surface of the cell.

Also provided herein are isolated immunomodulatory cells comprising: (a) a chimeric inhibitory receptor, wherein the chimeric inhibitory receptor comprises: —an extracellular protein binding domain, —a transmembrane domain, wherein the transmembrane domain is operably linked to the extracellular protein binding domain, and —an intracellular signaling domain, wherein the intracellular signaling domain is operably linked to the transmembrane domain; and (b) a tumor-targeting chimeric receptor expressed on the surface of the cell, wherein upon binding of the protein to the chimeric inhibitory receptor, the chimeric inhibitory receptor prevents, attenuates, or inhibits activation of the tumor-targeting chimeric receptor relative to an otherwise identical cell lacking a chimeric inhibitory receptor.

In some aspects, the chimeric inhibitory receptor is recombinantly expressed.

In some aspects, the chimeric inhibitory receptor is expressed from a vector or a selected locus from the genome of the cell.

In some aspects, the tumor-targeting chimeric receptor is a chimeric antigen receptor (CAR) or an engineered T cell receptor.

In some aspects, prior to binding of the protein to the chimeric inhibitory receptor, the tumor-targeting chimeric receptor is capable of activating the cell.

In some aspects, upon binding of the protein to the chimeric inhibitory receptor, the chimeric inhibitory receptor suppresses cytokine production from the activated cell.

In some aspects, upon binding of the protein to the chimeric inhibitory receptor, the chimeric inhibitory receptor suppresses a cell-mediated immune response to a target cell, wherein the immune response is induced by activation of the immunomodulatory cell.

In some aspects, the transmembrane domain is physically linked to the extracellular protein binding domain.

In some aspects, the intracellular signaling domain is physically linked to the transmembrane domain.

In some aspects, the transmembrane domain is physically linked to the extracellular protein binding domain and the intracellular signaling domain is physically linked to the transmembrane domain.

Also provided herein are isolated immunomodulatory cells comprising a chimeric inhibitory receptor, wherein the chimeric inhibitory receptor comprises: —an extracellular protein binding domain; —a transmembrane domain, wherein the transmembrane domain is operably linked to the extracellular protein binding domain, and —two or more intracellular signaling domains, wherein the two or more intracellular signaling domains are operably linked to the transmembrane domain; and wherein upon binding of the protein to the chimeric inhibitory receptor, the chimeric inhibitory receptor prevents, attenuates, or inhibits activation of a tumor-targeting chimeric receptor expressed on the surface of the cell relative to an otherwise identical cell lacking a chimeric inhibitory receptor.

In some aspects, the cell further comprises a tumor-targeting chimeric receptor expressed on the surface of the cell.

Also provided herein are isolated immunomodulatory cell comprising: (a) a chimeric inhibitory receptor, wherein the chimeric inhibitory receptor comprises: —an extracellular protein binding domain, —a transmembrane domain, wherein the transmembrane domain is operably linked to the extracellular protein binding domain, and —two or more intracellular signaling domains, wherein the two or more intracellular signaling domains are operably linked to the transmembrane domain; and (b) a tumor-targeting chimeric receptor expressed on the surface of the cell, wherein upon binding of the protein to the chimeric inhibitory receptor, the chimeric inhibitory receptor prevents, attenuates, or inhibits activation of the tumor-targeting chimeric receptor relative to an otherwise identical cell lacking a chimeric inhibitory receptor.

In some aspects, the chimeric inhibitory receptor is recombinantly expressed.

In some aspects, the chimeric inhibitory receptor is expressed from a vector or a selected locus from the genome of the cell.

In some aspects, the tumor-targeting chimeric receptor is a chimeric antigen receptor (CAR) or an engineered T cell receptor.

In some aspects, prior to binding of the protein to the chimeric inhibitory receptor, the tumor-targeting chimeric receptor is capable of activating the cell.

In some aspects, upon binding of the protein to the chimeric inhibitory receptor, the chimeric inhibitory receptor suppresses cytokine production from the activated cell.

In some aspects, upon binding of the protein to the chimeric inhibitory receptor, the chimeric inhibitory receptor suppresses a cell-mediated immune response to a target cell, wherein the immune response is induced by activation of the immunomodulatory cell.

In some aspects, the transmembrane domain is physically linked to the extracellular protein binding domain.

In some aspects, one of the two or more intracellular signaling domains is physically linked to the transmembrane domain.

In some aspects, the transmembrane domain is physically linked to the extracellular protein binding domain and one of the two or more intracellular signaling domains is physically linked to the transmembrane domain.

In some aspects, the target cell is a tumor cell.

In some aspects, the cell is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an ESC-derived cell, and an iPSC-derived cell. In some aspects, the immunomodulatory cell is a Natural Killer (NK) cell.

In some aspects, the cell is autologous.

The isolated cell as described herein, wherein the cell is allogeneic.

Also provided herein are compositions comprising an isolated cell as described herein and a pharmaceutically acceptable carrier.

Also provided herein are methods of preventing, attenuating, or inhibiting a cell-mediated immune response induced by a tumor-targeting chimeric receptor expressed of the surface of an immunomodulatory cell, comprising: engineering the immunomodulatory cell to express the chimeric inhibitory receptor as described herein on the surface of the immunomodulatory cell, wherein upon binding of a cognate protein to the chimeric inhibitory receptor, the intracellular signaling domain prevents, attenuates, or inhibits activation of the tumor-targeting chimeric receptor.

Also provided herein are methods of preventing, attenuating, or inhibiting activation of a tumor-targeting chimeric receptor expressed on the surface of an immunomodulatory cell, comprising: contacting an isolated cell as described herein or the compositions as described herein with a cognate protein of the chimeric inhibitory receptor under conditions suitable for the chimeric inhibitory receptor to bind the cognate protein, wherein upon binding of the protein to the chimeric inhibitory receptor, the intracellular signaling domain prevents, attenuates, or inhibits activation of the tumor-targeting chimeric receptor.

In some aspects, the tumor-targeting chimeric receptor is a chimeric antigen receptor (CAR) or an engineered T cell receptor (TCR).

In some aspects, the CAR binds one or more antigens expressed on the surface of a tumor cell.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1A shows an exemplary diagram of a T cell co-expressing an anti-CD19-BTLA iCAR and an anti-CD19-CD28/CD3ζ aCAR contacting a target cell expressing CD19. FIG. 1B shows negative control cells with no expression of either CAR construct. FIG. 1C shows anti-CD19-CD28/CD3ζ aCAR expression in transduced T cells. FIG. 1D shows anti-CD19-CD28/CD3ζ aCAR and anti-CD19-BTLA iCAR expression in transduced T cells.

FIG. 2A shows TNF-α production by T cells is reduced by co-expression of an anti-CD19 aCAR and an anti-CD19 iCAR as compared to an anti-CD19 aCAR alone. FIG. 2B shows IFN-γ production by T cells is reduced by co-expression of an anti-CD19 aCAR and an anti-CD19 iCAR as compared to an anti-CD19 aCAR alone. FIG. 2C shows IL-2 production by T cells is reduced by co-expression of an anti-CD19 aCAR and an anti-CD19 iCAR as compared to an anti-CD19 aCAR alone.

FIG. 3 shows T cell cytotoxicity is reduced by co-expression of an anti-CD19 aCAR and an anti-CD19 iCAR as compared to an anti-CD19 aCAR alone.

FIG. 4A shows an exemplary diagram of a T cell co-expressing an anti-CD19-BTLA iCAR and an anti-CD20-CD28/CD3ζ aCAR contacting a target cell expressing CD19 and CD20. FIG. 4B shows negative control cells with no expression of either CAR construct.

FIG. 4C shows anti-CD20-CD28/CD3ζ aCAR expression in transduced T cells. FIG. 4D shows anti-CD20-CD28/CD3ζ aCAR and anti-CD19-BTLA iCAR expression in transduced T cells.

FIG. 5A shows TNF-α production by T cells is reduced by co-expression of an anti-CD20 aCAR and an anti-CD19 iCAR as compared to an anti-CD20 aCAR alone. FIG. 5B shows IFN-7 production by T cells is reduced by co-expression of an anti-CD20 aCAR and an anti-CD19 iCAR as compared to an anti-CD20 aCAR alone. FIG. 5C shows IL-2 production by T cells is reduced by co-expression of an anti-CD20 aCAR and an anti-CD19 iCAR as compared to an anti-CD20 aCAR alone.

FIG. 6 shows anti-Axl-CD3ζ-mCherry aCAR expression in puromycin-selected T cells co-expressing the indicated anti-Her2-inhibitory domain iCAR.

FIG. 7A shows an exemplary diagram of a T cell co-expressing an anti-Axl-CD3ζ aCAR and an anti-Her2-inhibitory domain iCAR contacting target cells expressing Axl, Her2, Axl and Her2, or neither protein. FIG. 7B shows IL-2 secretion by T cells co-expressing the anti-Axl-CD3ζ aCAR and the indicated anti-Her2-inhibitory domain iCAR after contacting the indicated target cells. FIG. 7C shows IFN-7 secretion by T cells co-expressing the anti-Axl-CD3ζ aCAR and the indicated anti-Her2-inhibitory domain iCAR after contacting the indicated target cells.

FIG. 8A shows untransduced NK cells, and expression of anti-Her2-BTLA-GFP iCAR in transduced NK cells. FIG. 8B shows fluorescent microscopy images of expression of anti-Her2-BTLA-GFP iCAR and anti-Axl-CD3ζ-mCherry aCAR in singly or dual transduced NK cells.

FIG. 9A shows the percent lysis of target cells after incubation for 4 hours with NK cells expressing an anti-Axl aCAR, an anti-Her2 iCAR, or both the aCAR and the iCAR.

FIG. 9B shows the percent lysis of target cells after incubation for 8 hours with NK cells expressing an anti-Axl aCAR, an anti-Her2 iCAR, or both the aCAR and the iCAR.

FIG. 10 shows expression of aCARs and various iCAR formats, including co-expression, following transduction of NK cells as assessed by flow cytometry.

FIG. 11 shows NK cell mediated killing of parental target cells (column 1), target cells only expressing the aCAR antigen (column 2), or target cells expressing the aCAR antigen and iCAR antigen (column 3). Killing is shown for the various NK cells engineered to express aCAR only or engineered to co-express aCARs and the indicated iCARs.

FIG. 12 shows NK cell mediated killing of target cells only expressing the aCAR antigen in a mixed population (column 1) or target cells expressing the aCAR antigen and iCAR antigen in a mixed population (column 2). Killing is shown for the various NK cells engineered to express aCAR only or engineered to co-express aCARs and the indicated iCARs.

FIG. 13 shows NK cell mediated production of TNFα (top left), Granzyme B (bottom left), and IFNγ (top right) following co-culturing with parental target cells (column 1), target cells only expressing the aCAR antigen (column 2), target cells expressing the aCAR antigen and iCAR antigen (column 3), or a mixed population of target cells either only expressing the aCAR antigen or expressing the aCAR antigen and iCAR antigen. Cytokine production is shown for the various NK cells engineered to express aCAR only or engineered to co-express aCARs and the indicated iCARs.

FIG. 14 shows NK cell mediated killing of parental target cells (column 1), target cells only expressing the aCAR antigen (column 2), target cells expressing the aCAR antigen and iCAR antigen (column 3), target cells only expressing the aCAR antigen in a mixed population (column 4), or target cells expressing the aCAR antigen and iCAR antigen in a mixed population (column 5). Killing is shown for the various NK cells engineered to express aCAR only or engineered to co-express aCARs and the indicated iCARs.

FIG. 15 shows expression of aCARs and various iCAR formats, including co-expression, following transduction of NK cells as assessed by flow cytometry.

FIG. 16 shows NK cell mediated killing of parental target cells (column 1), target cells only expressing the aCAR antigen (column 2), or target cells expressing the aCAR antigen and iCAR antigen (column 3). Killing is shown for the various NK cells engineered to express aCAR only or engineered to co-express aCARs and the indicated iCARs.

FIG. 17 shows expression of aCARs and various iCAR formats, including co-expression, following transduction of T cells as assessed by flow cytometry.

FIG. 18 shows T cell mediated killing of parental target cells (column 1), target cells only expressing the iCAR antigen (column 2), target cells only expressing the aCAR antigen (column 3), or target cells expressing the aCAR antigen and iCAR antigen (column 4). Killing is shown for the various T cells engineered to express aCAR only or engineered to co-express aCARs and the indicated iCARs.

FIG. 19 shows T cell mediated IL-2 secretion of parental target cells (column 1), target cells only expressing the iCAR antigen (column 2), target cells only expressing the aCAR antigen (column 3), or target cells expressing the aCAR antigen and iCAR antigen (column 4). Killing is shown for the various T cells engineered to express aCAR only or engineered to co-express aCARs and the indicated iCARs.

FIG. 20 shows expression profiles of aCARs and various iCAR formats, including co-expression, following transduction of NK cells as assessed by flow cytometry. Between 1 and 3 biological replicates per condition (indicated as separate points).

FIG. 21 shows NK cell mediated killing (top panels) and cytokine secretion (bottom panel). Shown are for the various NK cells engineered to co-express an aCAR and the indicated iCARs. “Separate”=each type of SEM cell presented separately. “Mixed”=both types of SEM cells mixed together in the same culture. Between 1 and 3 biological replicates per condition (indicated as separate points). 3 technical replicates per measurement, X and Y SEM plotted where relevant.

### DETAILED DESCRIPTION

### Definitions

Terms used in the claims and specification are defined as set forth below unless otherwise specified.

The term “inhibitory chimeric receptor” or “chimeric inhibitory receptor” as used herein refers to a polypeptide or a set of polypeptides, which when expressed in an immune effector cell, provides the cell with specificity for a target cell, and with inhibitory intracellular signal generation. Inhibitory chimeric receptors typically include an extracellular protein binding domain (e.g., a ligand-binding domain, receptor-binding domain, antigen-binding domain, antibody fragment as an antigen-binding domain), a spacer domain, a transmembrane domain, and one or more intracellular signaling/co-signaling domains. An inhibitory chimeric receptor may also be called an “iCAR.”

The term “inhibitory chimeric antigen receptor” or “iCAR” as used herein refers to a polypeptide or a set of polypeptides, which when expressed in an immune effector cell, provides the cell with specificity for a target cell, and with inhibitory intracellular signal generation. Inhibitory chimeric antigen receptors typically include an extracellular antigen-binding domain (e.g., an antibody, or antigen-binding domain or fragment thereof), a spacer domain, a transmembrane domain, and one or more intracellular signaling/co-signaling domains.

The term “tumor targeting chimeric receptor” refers to activating chimeric receptors, tumor-targeting chimeric antigen receptors (CARs), or engineered T cell receptors. A tumor targeting chimeric receptor may also be called an “aCAR” or “activating CAR”

The term “chimeric antigen receptor” or alternatively a “CAR” as used herein refers to a polypeptide or a set of polypeptides, which when expressed in an immune effector cell, provides the cell with specificity for a target cell, and with intracellular signal generation. CARs typically include an extracellular protein binding domain (e.g., antibody fragment as an antigen-binding domain), a spacer domain, a transmembrane domain, and one or more intracellular signaling/co-signaling domains. In some embodiments, a CAR comprises at least an extracellular antigen binding domain, a transmembrane domain and a cytoplasmic signaling domain (also referred to herein as “an intracellular signaling domain”) comprising a functional signaling domain derived from a inhibitory molecule or a stimulatory molecule and/or costimulatory molecule. In some aspects, the set of polypeptides that comprise the inhibitory chimeric receptor or tumor targeting chimeric receptor are contiguous with each other. In some embodiments, the inhibitory chimeric receptor or tumor targeting chimeric receptor further comprises a spacer domain between the extracellular antigen binding domain and the transmembrane domain. In some embodiments, the set of polypeptides include recruitment domains, such as dimerization or multimerization domains, that can couple the polypeptides to one another. In some embodiments, an inhibitory chimeric receptor comprises a chimeric fusion protein comprising an extracellular antigen binding domain, a transmembrane domain and an intracellular signaling domain comprising a functional signaling domain derived from an inhibitory molecule or a stimulatory molecule. In one aspect, an inhibitory chimeric receptor comprises a chimeric fusion protein comprising an extracellular antigen binding domain, a transmembrane domain and an intracellular signaling domain comprising a functional inhibitory domain derived from an inhibitory molecule. In one aspect, a tumor targeting chimeric receptor comprises a chimeric fusion protein comprising an extracellular antigen binding domain, a transmembrane domain and an intracellular signaling domain comprising a functional signaling domain derived from a costimulatory molecule and a functional signaling domain derived from a stimulatory molecule.

The term, “intracellular signaling domain” as used herein, refers to a functional domain of the inhibitory chimeric receptor or the tumor targeting chimeric receptor located inside the cell. In some embodiments, the intracellular signaling domain is an inhibitory signaling domain. Following binding of the molecular binding domain to a protein, such as an antigen or ligand, for example, an inhibitory signaling domain represses receptor signaling while an activation signaling domain transmits a signal (e.g., proliferative/survival signal) to the cell.

The term, “transmembrane domain” as used herein, refers to a domain that spans a cellular membrane. In some embodiments, a transmembrane domain comprises a hydrophobic alpha helix.

The term, “extracellular protein binding domain” as used herein, refers to a molecular binding domain which is typically a ligand or ligand-binding domain, an ectodomain of a cell receptor, or the antigen binding domains of an antibody and is located outside the cell, exposed to the extracellular space. An extracellular antigen binding domain can include any molecule (e.g., protein or peptide) capable of binding to another protein or peptide, including a ligand, a ligand-binding domain, a receptor-binding domain, or an antigen-binding domain or antibody fragment as an antigen-binding domain. In some embodiments, an extracellular protein or antigen binding domain comprises a ligand, a ligand-binding domain, or a receptor-binding domain. In some embodiments, an extracellular protein or antigen binding domain comprises an antibody, an antigen-binding fragment thereof, F(ab), F(ab′), a single chain variable fragment (scFv), or a single-domain antibody (sdAb). In some embodiments, an extracellular protein or antigen binding domain binds to a cell-surface ligand (e.g., an antigen, such as a cancer antigen, or a protein expressed on the surface of a cell).

The term “extracellular antigen binding domain” as used herein, refers to a molecular antigen binding domain which is typically the antigen binding domains of an antibody and is located outside the cell, exposed to the extracellular space. An extracellular antigen binding domain can include any molecule (e.g., protein or peptide) capable of binding to an antigen protein or peptide. In some embodiments, an extracellular protein or antigen binding domain comprises an antibody, an antigen-binding fragment thereof, F(ab), F(ab′), a single chain variable fragment (scFv), or a single-domain antibody (sdAb). In some embodiments, an extracellular antigen binding domain binds to a cell-surface ligand (e.g., an antigen, such as a cancer antigen or a protein expressed on the surface of a cell).

The term “tumor” refers to tumor cells and the associated tumor microenvironment (TME). In some embodiments, tumor refers to a tumor cell or tumor mass. In some embodiments, tumor refers to the tumor microenvironment.

The term “not expressed” refers to expression that is at least 2-fold lower than the level of expression in non-tumor cells that would result in activation of the tumor-targeting chimeric antigen receptor. In some embodiments, the expression is at least 2-fold, at least 3-fold, at least 4-fold, at least 5-fold, at least 6-fold, at least 7-fold, at least 8-fold, at least 9-fold, or at least 10-fold or more lower than the level of expression in non-tumor cells that would result in activation of the tumor-targeting chimeric antigen receptor.

The term “ameliorating” refers to any therapeutically beneficial result in the treatment of a disease state, e.g., a cancer disease state, including prophylaxis, lessening in the severity or progression, remission, or cure thereof.

The term “in situ” refers to processes that occur in a living cell growing separate from a living organism, e.g., growing in tissue culture.

The term “in vivo” refers to processes that occur in a living organism.

The term “mammal” as used herein includes both humans and non-humans and include but is not limited to humans, non-human primates, canines, felines, murines, bovines, equines, and porcines.

The term percent “identity,” in the context of two or more nucleic acid or polypeptide sequences, refer to two or more sequences or subsequences that have a specified percentage of nucleic acid or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (ncbi.nlm.nih.gov/).

The term “sufficient amount” means an amount sufficient to produce a desired effect, e.g., an amount sufficient to modulate protein aggregation in a cell.

The term “therapeutically effective amount” is an amount that is effective to ameliorate a symptom of a disease. A therapeutically effective amount can be a “prophylactically effective amount” as prophylaxis can be considered therapy.

It must be noted that, as used in the specification and the appended claims, the singular forms “a,” “an” and “the” include plural referents unless the context clearly dictates otherwise.

### Chimeric Inhibitory Receptors

In one aspect, provided herein are chimeric inhibitory receptors comprising (i) an extracellular protein binding domain (e.g., an antigen-binding domain, ligand-binding domain, receptor-binding domain, etc.); (ii) a transmembrane domain, wherein the transmembrane domain is operably linked to the extracellular protein binding domain; and (iii) one or more intracellular signaling domains, wherein the one or more intracellular signaling domains are operably linked to the transmembrane domain, and wherein at least one of the one or more intracellular signaling domains is capable of preventing, attenuating, or inhibiting activation of a tumor-targeting chimeric receptor expressed on an immunomodulatory cell. In some embodiments, a chimeric inhibitory receptor of the present disclosure comprises two or more, three or more, four or more, or five or more intracellular signaling domains. In some embodiments, a chimeric inhibitory receptor of the present disclosure comprises one intracellular signaling domain. In some embodiments, a chimeric inhibitory receptor of the present disclosure comprises two intracellular signaling domains. In some embodiments, a chimeric inhibitory receptor of the present disclosure comprises three intracellular signaling domains. In some embodiments, a chimeric inhibitory receptor of the present disclosure comprises four intracellular signaling domains. In some embodiments, a chimeric inhibitory receptor of the present disclosure comprises five intracellular signaling domains.

The two, three, four, five or more intracellular signaling domains can be the same intracellular domain or different intracellular domains. For instance, one intracellular domain can be derived from one protein (e.g., BTLA) and a second intracellular domain can be derived from a different protein (e.g., LIR1). In instances with three or more intracellular domains, each of the three intracellular domains can be derived from the same protein, from three different proteins, or from two proteins. For example, in instance where the intracellular domains are derived from two proteins, the chimeric inhibitory receptor can have two domains from BTLA and one domain from LIR1, or any other combination of intracellular domains disclosed herein. In another example, in instances where the intracellular domains are derived from three proteins, the chimeric inhibitory receptor can one domain from BTLA, one domain from LIR1, and one domain from PD-1.

Generally, an inhibitory or tumor targeting chimeric receptor is designed for a T cell or NK cell, and is a chimera of an intracellular signaling domain and a protein-recognizing domain (e.g., a receptor-binding domain, a ligand-binding domain, or an antigen-binding domain, such as a single chain fragment (scFv) of an antibody) (Enblad et al., Human Gene Therapy. 2015; 26(8):498-505). A T cell that expresses a chimeric antigen receptor (CAR) is known in the art as a CAR T cell. An activating or tumor targeting CAR generally induces T cell signaling pathways upon binding to its cognate ligand via an intracellular signaling domain that results in activation of the T cell and an immune response. Activation CAR, activating CAR, and tumor-targeting CAR are interchangeable terms.

An inhibitory chimeric receptor, generally, is an artificial immune cell receptor engineered to recognize and bind to proteins, such as antigens, ligands, or receptors expressed by cells. Inhibitory chimeric receptors generally recognize proteins (e.g., antigens, ligands, receptors, etc.) that are not expressed on tumor cells, while activating or tumor targeting chimeric receptors (e.g., aCARs) generally recognize antigens that are expressed on tumor cells. Chimeric receptors in general typically include an antibody fragment as an antigen-binding domain, a spacer or hinge domains, a hydrophobic alpha helix transmembrane domain, and one or more intracellular signaling/co-signaling domains.

An inhibitory chimeric receptor generally follows the structure of activating CARs (aCARs) but uses an inhibitory domain for the intracellular signaling domain, instead of an activation signaling domain derived from a T-cell receptor (TCR). The intracellular signaling/co-signaling domain are inhibitory domains that reduce or inhibit signaling by other receptor proteins in the same cell. An inhibitory chimeric receptor cell can contain a protein-specific inhibitory receptor (e.g., an antigen-specific inhibitory receptor, a ligand-specific inhibitory receptor, receptor-specific inhibitory receptor, etc.), for example, to block nonspecific immunoactivation, which may result from extra-tumor target expression. In some embodiments, an inhibitory chimeric receptor blocks T cell responses in T cells activated by either their endogenous T cell receptor or an activating or tumor-targeting CAR. For example, an immunomodulatory cell can express both an inhibitory chimeric receptor that recognizes a non-tumor antigen target and a tumor-targeting chimeric receptor that recognizes a tumor antigen. When such an immunomodulatory cell contacts a tumor cell, only the tumor-targeting receptor recognizes and binds its cognate ligand and is activated, resulting in induction of cell signaling pathways and immune cell activation. In contrast, when the immunomodulatory cell contacts a non-tumor target, the inhibitory chimeric receptor binds to its cognate protein (e.g., cognate ligand, receptor, antigen, etc.) and represses or inhibits any signaling induced by the activation of the tumor-targeting chimeric receptor. Thus, the immunomodulatory cell can be constructed so that immune signaling only occurs when the cell contacts tumor cells.

In some embodiments, the protein (e.g., ligand, receptor, antigen, etc.) bound by the inhibitory chimeric receptor is not expressed on the target tumor. In some embodiments, the expression is at least 2-fold, at least 3-fold, at least 4-fold, at least 5-fold, at least 6-fold, at least 7-fold, at least 8-fold, at least 9-fold, or at least 10-fold or more lower than the level of expression in non-tumor cells that would result in activation of the tumor-targeting chimeric antigen receptor.

In some embodiments, the protein (e.g., ligand, receptor, antigen, etc.) bound by the inhibitory chimeric receptor is expressed on a non-tumor cell.

In some embodiments, the protein (e.g., ligand, receptor, antigen, etc.) bound by the inhibitory chimeric receptor is expressed on a non-tumor cell derived from a tissue selected from the group consisting of brain, neuronal tissue, endocrine, endothelial, bone, bone marrow, immune system, muscle, lung, liver, gallbladder, pancreas, gastrointestinal tract, kidney, urinary bladder, male reproductive organs, female reproductive organs, adipose, soft tissue, and skin.

In some embodiments, the inhibitory chimeric receptor comprises the sequence shown in SEQ ID NO: 56.

### Intracellular Signaling Domains

The inhibitory chimeric receptors of the present disclosure comprise one or more intracellular signaling domains that are capable of preventing, attenuating, or inhibiting activation of a tumor-targeting chimeric receptor expressed on an immunomodulatory cell.

In some embodiments, the one or more intracellular signaling domains comprise one or more modifications. In some embodiments, the one or more modifications modulate sensitivity of the chimeric inhibitory receptor relative to the otherwise identical, unmodified receptor. In some embodiments, the one or more modifications increase sensitivity of the chimeric inhibitory receptor relative to the otherwise identical, unmodified receptor. In some embodiments, the one or more modifications reduce sensitivity of the chimeric inhibitory receptor relative to the otherwise identical, unmodified receptor. In some embodiments, the one or more modifications modulate potency of the chimeric inhibitory receptor relative to the otherwise identical, unmodified receptor. In some embodiments, the one or more modifications increase potency of the chimeric inhibitory receptor relative to the otherwise identical, unmodified receptor. In some embodiments, the one or more modifications reduce potency of the chimeric inhibitory receptor relative to the otherwise identical, unmodified receptor.

In some embodiments, the one or more modifications modulate basal prevention, attenuation, or inhibition of activation of the tumor-targeting chimeric receptor expressed on an immunomodulatory cell relative to the otherwise identical, unmodified receptor. In some embodiments, the one or more modifications reduce basal prevention, attenuation, or inhibition relative to the otherwise identical, unmodified receptor. In some embodiments, the one or more modifications increase basal prevention, attenuation, or inhibition relative to the otherwise identical, unmodified receptor.

### Inhibitory Domains

In some embodiments, the CAR described herein comprises one or more inhibitory intracellular domains. In some embodiments, the CAR described herein comprises two or more inhibitory intracellular domains. In some embodiments, the CAR described herein comprises three or more inhibitory intracellular domains. In some embodiments, the CAR described herein comprises four or more inhibitory intracellular domains. In some embodiments, the CAR described herein comprises five or more inhibitory intracellular domains. In some embodiments, the CAR described herein comprises one inhibitory intracellular domain. In some embodiments, the CAR described herein comprises two inhibitory intracellular domains. In some embodiments, the CAR described herein comprises three inhibitory intracellular domains. In some embodiments, the CAR described herein comprises four inhibitory intracellular domains. In some embodiments, the CAR described herein comprises five inhibitory intracellular domains.

In some embodiments, for CARs having two or more inhibitory intracellular domains, two or more of the inhibitory intracellular domains are different domains. In some embodiments, for CARs having two or more inhibitory intracellular domains, each of the inhibitory intracellular domains are different domains. As an illustrative non-limiting example, a CAR can have a KIR3DL1 inhibitory intracellular domain linked to a LIR1 inhibitory intracellular domain. In some embodiments, for CARs having two or more inhibitory intracellular domains, two or more of the inhibitory intracellular domains are the same domain (i.e., a concatemer of the same domain). In some embodiments, for CARs having two or more inhibitory intracellular domains, each of the inhibitory intracellular domains are the same domain. As illustrative non-limiting examples, a CAR can have a first KIR3DL1 inhibitory intracellular domain linked to a second KIR3DL1 inhibitory intracellular domain or have a first LIR1 inhibitory intracellular domain linked to a second LIR1 inhibitory intracellular domain.

In some embodiments, one of the one or more inhibitory intracellular domains is a B- and T-lymphocyte attenuator (BTLA) domain. In some embodiments, one of the one or more inhibitory intracellular domains is a BTLA intracellular domain. BTLA (UNIPROT Q7Z6A9) is a transmembrane protein expressed on B cells, dendritic cells and naive T cells, and activated CD4+ T cells. The BTLA receptor's intracellular domain contains an immunoreceptor tyrosine-based inhibitory motif (ITIM) sequence that can bind to both SHP-1 and SHP-2. When BTLA's extracellular domain binds its ligand HVEM, the SUP-1 and SHP-2 phosphatases inhibit signaling through the TCR and may also block co-activators such as CD28.

In some embodiments, one of the one or more inhibitory intracellular domains is a LIR1 domain. In some embodiments, one of the one or more inhibitory intracellular domains is a LIR1 intracellular domain. LIR1 is also known as Leukocyte immunoglobulin-like receptor subfamily B member 1 (LILRB1, UNIPROT Q8NHL6). LIR1 is a transmembrane protein expressed on immune cells and binds to MHC class I molecules on antigen presenting cells. Binding of LIR1 to its cognate MHC I ligand induces inhibitory signaling that suppresses stimulation of an immune response. LIR family receptors contain two to four extracellular immunoglobulin domains, a transmembrane domain, and two to four intracellular domains with ITIM sequences.

In some embodiments, one of the one or more inhibitory intracellular domains is a PD-1 domain. In some embodiments, one of the one or more inhibitory intracellular domains is a PD-1 intracellular domain. PD-1 (Programmed cell death protein 1, UNIPROT Q15116) is expressed on T cell, B cells, and macrophages, and is a member of the CD28/CTLA-4 family of T cell regulators and the immunoglobulin superfamily. PD-1 is a transmembrane protein with an extracellular IgV ligand-binding domain and an intracellular domain with an ITIM sequence and an immunoreceptor tyrosine-based switch motif sequence. After binding of one of PD-1's two ligands, PD-L1 or PD-L2, SUP-1 and SHP-2 bind to the intracellular domain of PD-1 and negatively regulate TCR signaling.

In some embodiments, each of the one or more inhibitory intracellular signaling domains is derived from a protein selected from the group consisting of BTLA, PD-1, CTLA4, TIM3, KIR3DL1, LIR1, NKG2A, TIGIT, and LAG3. In some embodiments, the inhibitory chimeric receptor described herein comprises one or more inhibitory intracellular signaling domains. In some embodiments, one of the one or more inhibitory intracellular signaling domains is a BTLA domain. In some embodiments, one of the one or more intracellular signaling domains is derived from BTLA. In some embodiments, one of the one or more intracellular signaling domains is a CTLA4 domain. In some embodiments, one of the one or more intracellular signaling domains is derived from CTLA4. In some embodiments, one of the one or more intracellular signaling domains is a PD-1 domain. In some embodiments, one of the one or more intracellular signaling domains is derived from PD-1. In some embodiments, one of the one or more intracellular signaling domains is a TIM3 domain. In some embodiments, one of the one or more intracellular signaling domains is derived from TIM3. In some embodiments, one of the one or more intracellular signaling domains is a KIR3DL1 domain. In some embodiments, one of the one or more intracellular signaling domains is derived from KIR3DL1. In some embodiments, one of the one or more intracellular signaling domains is a LIR1 domain. In some embodiments, one of the one or more intracellular signaling domains is derived from LIR1. In some embodiments, one of the one or more intracellular signaling domains is an NKG2A domain. In some embodiments, one of the one or more intracellular signaling domains is derived from NKG2A. In some embodiments, one of the one or more intracellular signaling domains is a TIGIT domain. In some embodiments, one of the one or more intracellular signaling domains is derived from TIGIT. In some embodiments, one of the one or more intracellular signaling domains is a LAG3 domain. In some embodiments, one of the one or more intracellular signaling domains is derived from LAG3.

Exemplary inhibitory intracellular signaling domain amino acid sequences are shown in Table 1. Exemplary inhibitory intracellular signaling domain nucleic acid sequences are shown in Table 2.

TABLE 1
Exemplary inhibitory intracellular signaling domain amino acid sequences

| Amino Acid Sequence | SEQ ID NO: | Description |
|---|---|---|
| CSRAARGTIGARRTGQPLKEDPSAVPVFSVDYGELDF | 1 | PD-1 intracellular signaling |
| QWREKTPEPPVPCVPEQTEYATIVFPSGMGTSSPARR | | domain |
| GSADGPRSAQPLRPEDGHCSWPL | | |
| AVSLSKMLKKRSPLTTGVYVKMPPTEPECEKQFQPY | 2 | CTLA4 intracellular signaling |
| FIPIN | | domain |
| RRHQGKQNELSDTAGREINLVDAHLKSEQTEASTRQ | 3 | BTLA intracellular signaling |
| NSQVLLSETGIYDNDPDLCFRMQEGSEVYSNPCLEEN | | domain |
| KPGIVYASLNHSVIGPNSRLARNVKEAPTEYASICVR | | |
| S | | |
| LRHRRQGKHWTSTQRKADFQHPAGAVGPEPTDRGL | 50 | LIR1 intracellular signaling |
| QWRSSPAADAQEENLYAAVKHTQPEDGVEMDTRSP | | domain |
| HDEDPQAVTYAEVKHSRPRREMASPPSPLSGEFLDT | | |
| KDRQAEEDRQMDTEAAASEAPQDVTYAQLHSLTLR | | |
| REATEPPPSQEGPSPAVPSIYATLAIH | | |
| HLWCSNKKNAAVMDQEPAGNRTANSEDSDEQDPEE | 66 | KIR3DL1 intracellular signaling |
| VTYAQLDHCVFTQRKITRPSQRPKTPPTDTILYTELPN | | domain |
| AKPRSKVVSCP | | |
| AVSLSKMLKKRSPLTTGVGVKMPPTEPECEKQFQPY | 67 | CTLA4 intracellular signaling |
| FIPIN | | domain |
| KEPASPLDKCHYTKDNGQFDQSAKQLNLEAYTIEQE | 93 | NKG2A (reversed) intracellular |
| TALISNKNGKPKRQQRKPNPPLNLDSYIVGQNDM | | signaling domain |
| LTRKKKALRIHSVEGDLRRKSAGQEEWSPSAPSPPGS | 95 | TIGIT intracellular signaling |
| CVQAEAAPAGLCGEQRGEDCAELHDYFNVLSYRSL | | domain |
| GNCSFFTETG | | |
| MDNQGVIYSDLNLPPNPKRQQRKPKGNKNSILATEQ | 105 | NKG2A intracellular signaling |
| EITYAELNLQKASQDFQGNDKTYHCKDLPSAPEK | | domain |

TABLE 2
Exemplary inhibitory intracellular signaling domain nucleic acid sequences

| Nucleic Acid Sequence | SEQ ID NO: | Description |
|---|---|---|
| TGTAGCAGAGCCGCCAGAGGAACAATCGGCGCCA | 4 | PD-1 intracellular signaling |
| GAAGAACAGGCCAGCCTCTGAAAGAGGACCCCTC | | domain |
| TGCCGTTCCTGTGTTCAGCGTGGACTATGGCGAGC | | |
| TGGATTTCCAGTGGCGGGAAAAGACACCCGAGCC | | |
| TCCAGTGCCTTGTGTGCCTGAGCAGACAGAGTACG | | |
| CCACCATCGTGTTCCCTAGCGGCATGGGCACATCT | | |
| AGCCCTGCCAGAAGAGGATCTGCCGACGGACCTA | | |
| GATCTGCCCAGCCTCTTAGACCTGAGGACGGCCAC | | |
| TGTTCTTGGCCTCTT | | |
| TGTAGCCGAGCGGCCAGAGGCACAATCGGGGCAA | 51 | PD-1 intracellular signaling |
| GACGAACAGGACAGCCGCTCAAAGAGGACCCCAG | | domain |
| TGCGGTCCCCGTTTTCTCCGTGGATTACGGAGAAC | | |
| TGGATTTCCAGTGGCGGGAGAAGACACCAGAGCC | | |
| CCCGGTGCCCTGCGTGCCGGAGCAGACTGAGTACG | | |
| CCACGATTGTGTTTCCCTCTGGAATGGGGACTTCA | | |
| TCCCCCGCTAGGCGCGGCTCAGCTGATGGCCCAAG | | |
| ATCCGCTCAACCGTTGCGGCCAGAGGACGGGCATT | | |
| GCAGTTGGCCTCTG | | |
| GCCGTGTCTCTGAGCAAGATGCTGAAGAAGCGGA | 5 | CTLA4 intracellular signaling |
| GCCCTCTGACCACCGGCGTGTACGTGAAAATGCCT | | domain |
| CCTACCGAGCCTGAGTGCGAGAAGCAGTTCCAGCC | | |
| TTACTTCATCCCCATCAAC | | |
| AGGAGACATCAGGGGAAGCAGAATGAACTCAGCG | 6 | BTLA intracellular signaling |
| ATACAGCAGGGCGAGAAATTAATTTGGTAGACGC | | domain |
| GCATCTGAAGTCCGAACAGACAGAGGCTTCTACTA | | |
| GACAGAACTCCCAAGTTTTGTTGAGTGAGACGGGG | | |
| ATCTATGATAATGATCCCGATCTGTGTTTTAGAAT | | |
| GCAGGAGGGTAGTGAAGTCTACTCAAACCCGTGC | | |
| CTGGAAGAAAATAAGCCCGGCATTGTTTACGCTAG | | |
| TTTGAATCATTCTGTAATAGGCCCGAACTCCAGAC | | |
| TGGCTCGCAATGTGAAGGAGGCCCCAACTGAGTAT | | |
| GCGTCCATTTGCGTGCGGTCT | | |
| AGAAGACATCAGGGGAAGCAGAATGAACTCAGCG | 52 | BTLA intracellular signaling |
| ATACAGCAGGGCGAGAAATTAATTTGGTAGACGC | | domain |
| GCATCTGAAGTCCGAACAGACAGAGGCTTCTACTA | | |
| GACAGAACTCCCAAGTTTTGTTGAGTGAGACGGGG | | |
| ATCTATGATAATGATCCCGATCTGTGTTTTAGAAT | | |
| GCAGGAGGGTAGTGAAGTCTACTCAAACCCGTGC | | |
| CTGGAAGAAAATAAGCCCGGCATTGTTTACGCTAG | | |
| TTTGAATCATTCTGTAATAGGCCCGAACTCCAGAC | | |
| TGGCTCGCAATGTGAAGGAGGCCCCAACTGAGTAT | | |
| GCGTCCATTTGCGTGCGGTCT | | |
| AGAAGGCACCAGGGAAAGCAGAACGAGCTGAGCG | 53 | BTLA intracellular signaling |
| ATACCGCCGGCAGAGAAATCAACCTGGTGGACGC | | domain |
| CCACCTGAAAAGCGAGCAGACAGAGGCCAGCACC | | |
| AGACAGAATAGCCAGGTGCTGCTGAGCGAGACAG | | |
| GCATCTACGACAACGACCCCGACCTGTGCTTCCGG | | |
| ATGCAAGAGGGAAGCGAGGTGTACAGCAACCCCT | | |
| GCCTGGAAGAGAACAAGCCCGGCATCGTGTACGC | | |
| TAGCCTGAACCACTCTGTGATCGGCCCCAATTCCA | | |
| GACTGGCCCGGAACGTGAAAGAGGCCCCTACAGA | | |
| GTACGCCAGCATCTGCGTCAGAAGC | | |
| TTGCGCCACAGACGGCAGGGAAAGCACTGGACTA | 54 | LIR1 intracellular signaling |
| GTACGCAGAGGAAAGCGGACTTCCAGCATCCCGC | | domain |
| AGGAGCCGTGGGGCCTGAACCCACTGATCGCGGC | | |
| CTTCAATGGAGGTCTAGCCCGGCGGCAGACGCAC | | |
| AAGAGGAAAACTTGTACGCAGCCGTTAAGCACAC | | |
| CCAACCGGAGGACGGCGTTGAGATGGATACCCGC | | |
| TCCCCTCACGATGAAGACCCTCAAGCAGTCACTTA | | |
| CGCGGAAGTAAAGCATAGCCGCCCCAGACGGGAA | | |
| ATGGCTAGCCCGCCGTCCCCCCTTAGCGGGGAATT | | |
| TCTGGACACTAAAGATAGGCAGGCGGAAGAGGAC | | |
| CGCCAAATGGATACAGAGGCGGCGGCAAGTGAAG | | |
| CACCTCAAGACGTTACTTACGCTCAACTTCACAGC | | |
| CTTACCCTCAGGCGAGAAGCGACTGAACCACCCCC | | |
| TTCCCAAGAAGGGCCAAGCCCAGCGGTTCCTTCTA | | |
| TCTATGCTACTCTTGCTATTCAC | | |
| CTGCGGCACAGACGGCAGGGCAAGCACTGGACAA | 55 | LIR1 intracellular signaling |
| GCACACAGAGAAAGGCCGACTTTCAGCACCCTGCT | | domain |
| GGTGCCGTTGGACCTGAGCCTACAGATAGAGGACT | | |
| GCAGTGGCGGTCTAGCCCTGCCGCTGATGCTCAAG | | |
| AGGAAAACCTGTACGCCGCCGTGAAGCACACCCA | | |
| ACCTGAAGATGGCGTGGAAATGGACACCAGATCT | | |
| CCCCACGATGAGGACCCTCAGGCCGTGACATATGC | | |
| CGAAGTGAAGCACTCCCGGCCTCGGAGAGAAATG | | |
| GCTAGCCCTCCAAGTCCTCTGAGCGGCGAGTTCCT | | |
| GGACACCAAGGATAGACAGGCCGAAGAGGACCGG | | |
| CAGATGGATACAGAAGCTGCCGCATCTGAGGCCC | | |
| CACAGGATGTGACTTATGCCCAGCTGCACAGCCTG | | |
| ACACTGCGGAGAGAAGCCACAGAGCCTCCACCTT | | |
| CTCAAGAGGGCCCATCTCCAGCCGTGCCTAGCATC | | |
| TATGCCACACTGGCCATCCAC | | |
| GCCGTGTCACTTAGTAAGATGCTGAAGAAGAGGTC | 84 | CTLA4 intracellular signaling |
| ACCACTGACGACAGGGGTTGGAGTGAAGATGCCA | | domain |
| CCCACAGAACCCGAATGTGAGAAGCAATTCCAGC | | |
| CTTATTTCATTCCAATAAAT | | |
| CATCTGTGGTGTTCTAATAAGAAGAATGCTGCTGT | 85 | KIR3DL1 intracellular signaling |
| GATGGATCAAGAGCCCGCTGGTAACAGAACGGCC | | domain |
| AACAGTGAAGATAGCGATGAGCAGGACCCAGAAG | | |
| AAGTGACCTACGCCCAACTCGACCACTGTGTTTTT | | |
| ACGCAGCGGAAAATCACTCGACCCTCTCAACGACC | | |
| CAAAACGCCGCCTACGGACACCATACTCTACACCG | | |
| AACTGCCGAACGCCAAACCACGGTCCAAGGTGGT | | |
| ATCATGTCCG | | |
| CTGCGGCACAGAAGGCAGGGCAAGCACTGGACAA | 86 | LIR1 intracellular signaling |
| GCACCCAGAGAAAGGCCGATTTTCAGCACCCTGCT | | domain |
| GGCGCCGTTGGACCTGAGCCTACAGATAGAGGAC | | |
| TGCAGTGGCGGTCTAGCCCTGCTGCCGATGCTCAA | | |
| GAGGAAAACCTGTACGCCGCCGTGAAGCACACCC | | |
| AACCTGAAGATGGCGTGGAAATGGACACCAGATC | | |
| TCCCCACGATGAGGACCCTCAGGCCGTGACATACG | | |
| CTGAAGTGAAGCACTCCCGGCCTCGGAGAGAAAT | | |
| GGCTAGCCCTCCAAGTCCTCTGAGCGGCGAGTTCC | | |
| TGGACACCAAGGATAGACAGGCCGAAGAGGACCG | | |
| GCAGATGGATACAGAAGCTGCCGCCTCTGAAGCC | | |
| CCACAGGATGTGACATATGCCCAGCTGCATAGCCT | | |
| GACACTGCGGAGAGAAGCCACAGAGCCTCCACCT | | |
| TCTCAAGAGGGCCCATCTCCAGCCGTGCCTAGCAT | | |
| CTATGCCACACTGGCCATTCAC | | |
| AAGGAGCCTGCGTCCCCGTTGGATAAATGCCACTA | 94 | NKG2A (reversed) intracellular |
| TACTAAGGATAACGGTCAGTTCGATCAGAGTGCAA | | signaling domain |
| AGCAACTTAACTTGGAGGCTTACACTATAGAGCAA | | |
| GAAACAGCGCTGATAAGTAATAAGAACGGTAAGC | | |
| CAAAGCGACAGCAGAGGAAACCCAATCCTCCGCT | | |
| TAACTTGGATAGCTACATCGTCGGGCAAAATGACA | | |
| TG | | |
| CTGACCAGAAAGAAGAAGGCCCTGAGAATCCACA | 96 | TIGIT intracellular signaling |
| GCGTGGAAGGCGACCTGCGGAGAAAGTCTGCCGG | | domain |
| ACAAGAAGAGTGGTCCCCTAGCGCTCCATCTCCAC | | |
| CTGGATCTTGTGTGCAGGCCGAAGCAGCTCCTGCT | | |
| GGACTGTGTGGCGAACAGAGAGGCGAAGATTGCG | | |
| CCGAGCTGCACGACTACTTCAACGTGCTGAGCTAC | | |
| AGAAGCCTGGGCAACTGCAGCTTCTTCACCGAGAC | | |
| AGGA | | |
| ATGGACAACCAGGGCGTGATCTACAGCGACCTGA | 106 | NKG2A intracellular signaling |
| ACCTGCCTCCTAATCCTAAGCGGCAGCAGAGAAA | | domain |
| GCCCAAGGGCAACAAGAACAGCATCCTGGCCACC | | |
| GAGCAAGAGATCACCTACGCCGAGCTGAATCTGC | | |
| AGAAGGCCAGCCAGGACTTCCAGGGCAACGACAA | | |
| GACCTACCACTGCAAGGACCTGCCTAGCGCTCCCG | | |
| AGAAG | | |
| ATGGACAACCAGGGCGTCATCTACAGCGACCTGA | 130 | NKG2A intracellular signaling |
| ACCTGCCTCCTAATCCAAAGCGGCAGCAGCGGAA | | domain (codon optimized) |
| GCCCAAGGGCAACAAGAATAGCATCCTGGCCACC | | |
| GAGCAAGAGATCACCTACGCCGAGCTGAATCTGC | | |
| AGAAGGCCAGCCAGGATTTCCAGGGCAACGACAA | | |
| GACCTACCACTGCAAGGACCTGCCTAGCGCTCCTG | | |
| AGAAA | | |

In some embodiments, one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to RRHQGKQNELSDTAGREINLVDAHLKSEQTEASTRQNSQVLLSETGIYDNDPDLCFR MQEGSEVYSNPCLEENKPGIVYASLNHSVIGPNSRLARNVKEAPTEYASICVRS (SEQ ID NO: 3). In some embodiments, one of the one or more intracellular signaling domains comprises the amino acid sequence of

(SEQ ID NO: 3)
RRHQGKQNELSDTAGREINLVDAHLKSEQTEASTRQNSQVLLSETGIYDN
DPDLCFRMQEGSEVYSNPCLEENKPGIVYASLNHSVIGPNSRLARNVKEA
PTEYASICVRS.

In some embodiments, one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 1. In some embodiments, one of the one or more intracellular signaling domains comprises the amino acid sequence of SEQ ID NO: 1.

In some embodiments, one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 2. In some embodiments, one of the one or more intracellular signaling domains comprises the amino acid sequence of SEQ ID NO: 2.

In some embodiments, one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LRHRRQGKHWTSTQRKADFQHPAGAVGPEPTDRGLQWRSSPAADAQEENLYAAVK HTQPEDGVEMDTRSPHDEDPQAVTYAEVKHSRPRREMASPPSPLSGEFLDTKDRQAE EDRQMDTEAAASEAPQDVTYAQLHSLTLRREATEPPPSQEGPSPAVPSIYATLAIH (SEQ ID NO: 50). In some embodiments, one of the one or more intracellular signaling domains comprises the amino acid sequence of

(SEQ ID NO: 50)
LRHRRQGKHWTSTQRKADFQHPAGAVGPEPTDRGLQWRSSPAADAQEENL
YAAVKHTQPEDGVEMDTRSPHDEDPQAVTYAEVKHSRPRREMASPPSPLS
GEFLDTKDRQAEEDRQMDTEAAASEAPQDVTYAQLHSLTLRREATEPPPS
QEGPSPAVPSIYATLAIH.

In some embodiments, one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to HLWCSNKKNAAVMDQEPAGNRTANSEDSDEQDPEEVTYAQLDHCVFTQRKITRPSQ RPKTPPTDTILYTELPNAKPRSKVVSCP (SEQ ID NO: 66). In some embodiments, one of the one or more intracellular signaling domains comprises the amino acid sequence of

(SEQ ID NO: 66)
HLWCSNKKNAAVMDQEPAGNRTANSEDSDEQDPEEVTYAQLDHCVFTQRK
ITRPSQRPKTPPTDTILYTELPNAKPRSKVVSCP.

In some embodiments, one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to AVSLSKMLKKRSPLTTGVGVKMPPTEPECEKQFQPYFIPIN (SEQ ID NO: 67). In some embodiments, one of the one or more intracellular signaling domains comprises the amino acid sequence of AVSLSKMLKKRSPLTTGVGVKMPPTEPECEKQFQPYFIPIN (SEQ ID NO: 67).

In some embodiments, one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 93. In some embodiments, one of the one or more intracellular signaling domains comprises the amino acid sequence of SEQ ID NO: 93.

In some embodiments, one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 95. In some embodiments, one of the one or more intracellular signaling domains comprises the amino acid sequence of SEQ ID NO: 95.

In some embodiments, one of the one or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 105. In some embodiments, one of the one or more intracellular signaling domains comprises the amino acid sequence of SEQ ID NO: 105.

In some embodiments, the transmembrane domain and at least one of the one or more intracellular signaling domains are derived from the same protein. In some embodiments, the transmembrane domain is derived from a first protein and each of the one or more intracellular signaling domains is derived from a protein that is distinct from the first protein.

In some embodiments, an inhibitory chimeric receptor of the present disclosure comprises two intracellular signaling domains. In some embodiments, the first intracellular signaling domain is derived LIR1 and the second intracellular signaling domain is derived from BTLA. In some embodiments, the first intracellular signaling domain is derived LIR1 and the second intracellular signaling domain is derived from PD-1. In some embodiments, the first intracellular signaling domain further comprises a transmembrane domain derived from LIR1.

In some embodiments, an inhibitory chimeric receptor of the present disclosure comprises two intracellular signaling domains. In some embodiments, the first intracellular signaling domain is derived from BTLA and the second intracellular signaling domain is derived from LIR1. In some embodiments, the first intracellular signaling domain is derived from BTLA and the second intracellular signaling domain is derived from PD-1. In some embodiments, the first intracellular signaling domain further comprises a transmembrane domain derived from BTLA.

In some embodiments, an inhibitory chimeric receptor of the present disclosure comprises two intracellular signaling domains. In some embodiments, the two intracellular signaling domains are selected from the group consisting of BTLA, PD-1, CTLA4, TIM3, KIR3DL1, LIR1, NKG2A, TIGIT, and LAG3. In some embodiments, the first intracellular signaling domain is derived from PD-1 and the second intracellular signaling domain is derived from LIR1. In some embodiments, the first intracellular signaling domain is derived from PD-1 and the second intracellular signaling domain is derived from BTLA. In some embodiments, the first intracellular signaling domain further comprises a transmembrane domain derived from PD-1. The first and second intracellular signaling domains may be in any order.

In some embodiments, an inhibitory chimeric receptor of the present disclosure comprises three intracellular signaling domains. In some embodiments, the three intracellular signaling domains are selected from the group consisting of BTLA, PD-1, CTLA4, TIM3, KIR3DL1, LIR1, NKG2A, TIGIT, and LAG3. In some embodiments, the first intracellular signaling domain is derived from PD-1, the second intracellular signaling domain is derived from LIR1, and the third intracellular signaling domain is derived from BTLA. In some embodiments, the first intracellular signaling domain further comprises a transmembrane domain derived from PD-1. In some embodiments, the first intracellular signaling domain further comprises a transmembrane domain derived from LIR1. In some embodiments, the first intracellular signaling domain further comprises a transmembrane domain derived from BTLA. The first, second, and third intracellular signaling domains may be in any order. For instance, in an inhibitory chimeric receptor comprising the three signaling domains from PD-1, LIR1, and BTLA, the order of the intracellular signaling domains can be PD-1-LIR1-BTLA, or PD-1-BTLA-LIR1, or LIR1-PD-1-BTLA, or LIR1-BTLA-PD-1, or BTLA-PD-1-LIR1, or BTLA-LIR1-PD-1.

In some embodiments, an inhibitory chimeric receptor of the present disclosure comprises four intracellular signaling domains. In some embodiments, the four intracellular signaling domains are selected from the group consisting of BTLA, PD-1, CTLA4, TIM3, KIR3DL1, LIR1, NKG2A, TIGIT, and LAG3. The first, second, third, and fourth intracellular signaling domains may be in any order.

In some embodiments, an inhibitory chimeric receptor of the present disclosure comprises five intracellular signaling domains. In some embodiments, the five intracellular signaling domains are selected from the group consisting of BTLA, PD-1, CTLA4, TIM3, KIR3DL1, LIR1, NKG2A, TIGIT, and LAG3. The first, second, third, fourth, and fifth intracellular signaling domains may be in any order. In some embodiments, an inhibitory chimeric receptor of the present disclosure comprises more than five intracellular signaling domains. In some embodiments, the more than five intracellular signaling domains are selected from the group consisting of BTLA, PD-1, CTLA4, TIM3, KIR3DL1, LIR1, NKG2A, TIGIT, and LAG3. The first, second, third, fourth, fifth, and additional intracellular signaling domains may be in any order.

In some embodiments, one of the one or more intracellular signaling domains comprises a nucleic acid comprising SEQ ID NO: 4. In some embodiments, one of the one or more intracellular signaling domain polypeptides comprises a nucleic acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 4.

In some embodiments, one of the one or more intracellular signaling domains comprises a nucleic acid comprising SEQ ID NO: 5. In some embodiments, one of the one or more intracellular signaling domains comprises a nucleic acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 5.

In some embodiments, one of the one or more intracellular signaling domains comprises a nucleic acid comprising SEQ ID NO: 6. In some embodiments, one of the one or more intracellular signaling domains comprises a nucleic acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 6.

In some embodiments, one of the one or more intracellular signaling domains comprises a nucleic acid comprising SEQ ID NO: 51. In some embodiments, one of the one or more intracellular signaling domains comprises a nucleic acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 51.

In some embodiments, one of the one or more intracellular signaling domains comprises a nucleic acid comprising SEQ ID NO: 52. In some embodiments, one of the one or more intracellular signaling domains comprises a nucleic acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 52.

In some embodiments, one of the one or more intracellular signaling domains comprises a nucleic acid comprising SEQ ID NO: 53. In some embodiments, one of the one or more intracellular signaling domains comprises a nucleic acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 53.

In some embodiments, one of the one or more intracellular signaling domains comprises a nucleic acid comprising SEQ ID NO: 54. In some embodiments, one of the one or more intracellular signaling domains comprises a nucleic acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 54.

In some embodiments, one of the one or more intracellular signaling domains comprises a nucleic acid comprising SEQ ID NO: 55. In some embodiments, one of the one or more intracellular signaling domains comprises a nucleic acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 55.

In some embodiments, one of the one or more intracellular signaling domains comprises a nucleic acid comprising SEQ ID NO: 84. In some embodiments, one of the one or more intracellular signaling domains comprises a nucleic acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 84.

In some embodiments, one of the one or more intracellular signaling domains comprises a nucleic acid comprising SEQ ID NO: 85. In some embodiments, one of the one or more intracellular signaling domains comprises a nucleic acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 85.

In some embodiments, one of the one or more intracellular signaling domains comprises a nucleic acid comprising SEQ ID NO: 86. In some embodiments, one of the one or more intracellular signaling domains comprises a nucleic acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 86.

In some embodiments, one of the one or more intracellular signaling domains comprises a nucleic acid comprising SEQ ID NO: 94. In some embodiments, one of the one or more intracellular signaling domains comprises a nucleic acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 94.

In some embodiments, one of the one or more intracellular signaling domains comprises a nucleic acid comprising SEQ ID NO: 96. In some embodiments, one of the one or more intracellular signaling domains comprises a nucleic acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 96.

In some embodiments, one of the one or more intracellular signaling domains comprises a nucleic acid comprising SEQ ID NO: 106. In some embodiments, one of the one or more intracellular signaling domains comprises a nucleic acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 106.

In some embodiments, one of the one or more intracellular signaling domains comprises a nucleic acid comprising SEQ ID NO: 130. In some embodiments, one of the one or more intracellular signaling domains comprises a nucleic acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 130.

### Enzymatic Inhibitory Domains

In some embodiments, the inhibitory chimeric receptor comprises an enzymatic inhibitory domain. In some embodiments, the enzymatic inhibitory domain is also capable of preventing, attenuating, or inhibiting activation of a chimeric receptor when expressed on an immunomodulatory cell relative to an otherwise identical chimeric inhibitory receptor lacking the enzymatic inhibitory domain.

In some embodiments, the enzymatic inhibitory domain comprises an enzyme catalytic domain. In some embodiments, the enzyme catalytic domain is derived from an enzyme selected from the group consisting of: CSK, SUP-1, PTEN, CD45, CD148, PTP-MEG1, PTP-PEST, c-CBL, CBL-b, PTPN22, LAR, PTPH1, SHIP-1, and RasGAP.

In some embodiments, the enzymatic inhibitory domain comprises one or more modifications that modulate basal prevention, attenuation, or inhibition relative to an otherwise identical enzymatic inhibitory domain lacking the one or more modifications. In some embodiments, the one or more modifications reduce basal prevention, attenuation, or inhibition relative to an otherwise identical enzymatic inhibitory domain lacking the one or more modifications. In some embodiments, the one or more modifications increase basal prevention, attenuation, or inhibition relative to an otherwise identical enzymatic inhibitory domain lacking the one or more modifications.

### Activation and Co-Stimulatory Domains

In some embodiments, a cell disclosed herein can further comprise at least one tumor-targeting chimeric receptor or T cell receptor comprising an activating intracellular domain or a co-stimulatory intracellular domain. In some embodiments, the cell comprises at least one inhibitory chimeric receptor and at least one tumor-targeting chimeric receptor. The cell can comprise at least 1, at least 2, at least 3, at least 4, at least 5, at least 6, at least 7, at least 8, at least 9, or at least 10 or more tumor-targeting CARs and at least 1, at least 2, at least 3, at least 4, at least 5, at least 6, at least 7, at least 8, at least 9, or at least 10 or more inhibitory chimeric receptors.

In some embodiments, the activating signaling domain is a CD3-zeta protein, which includes three immunoreceptor tyrosine-based activation motifs (ITAMs). Other examples of activating signaling domains include CD28, 4-1BB, and OX40. In some embodiments, a cell receptor comprises more than one activating signaling domain, each referred to as a co-stimulatory domain.

In some embodiments, the tumor-targeting chimeric receptor is a chimeric antigen receptor (CAR) or an engineered T cell receptor. In some embodiments, the CAR binds one or more antigens expressed on the surface of a tumor cell.

In some embodiments, prior to binding of the antigen to the chimeric inhibitory receptor, the tumor-targeting chimeric receptor is capable of activating the cell.

In some embodiments, the tumor-targeting chimeric antibody comprises the sequence shown in SEQ ID NO: 51. In some embodiments, the tumor-targeting chimeric antibody comprises the sequence shown in SEQ ID NO: 52.

### Transmembrane Domains

The inhibitory chimeric receptors can contain transmembrane domains that link the protein binding domain to the intracellular domain. Different transmembrane domains result in different receptor stability. Suitable transmembrane domains include, but are not limited to, BTLA, CD8, CD28, CD3zeta, CD4, 4-IBB, OX40, ICOS, 2B4, CD25, CD7, LAX, LAT, PD-1, CTLA4, TIM3, KIR3DL1, LIR1, NKG2A, TIGIT, and LAG3.

In some embodiments, the transmembrane domain is derived from a protein selected from the group consisting of BTLA, CD8, CD28, CD3zeta, CD4, 4-IBB, OX40, ICOS, 2B4, CD25, CD7, LAX, LAT, PD-1, CTLA4, TIM3, KIR3DL1, LIR1, NKG2A, TIGIT, and LAG3. In some embodiments, a transmembrane domain of a cell receptor is a BTLA transmembrane domain. In some embodiments, a transmembrane domain of a cell receptor is a CD8 transmembrane domain. In some embodiments, a transmembrane domain of a cell receptor is a CD28 transmembrane domain. In some embodiments, a transmembrane domain of a cell receptor is a CD3zeta transmembrane domain. In some embodiments, a transmembrane domain of a cell receptor is a CD4 transmembrane domain. In some embodiments, a transmembrane domain of a cell receptor is a 4-1BB transmembrane domain. In some embodiments, a transmembrane domain of a cell receptor is an OX40 transmembrane domain. In some embodiments, a transmembrane domain of a cell receptor is an ICOS transmembrane domain. In some embodiments, a transmembrane domain of a cell receptor is a 2B4 transmembrane domain. In some embodiments, a transmembrane domain of a cell receptor is a CD25 transmembrane domain. In some embodiments, a transmembrane domain of a cell receptor is a CD7 transmembrane domain. In some embodiments, a transmembrane domain of a cell receptor is a n LAX transmembrane domain. In some embodiments, a transmembrane domain of a cell receptor is an LAT transmembrane domain. In some embodiments, a transmembrane domain of a cell receptor is a PD-1 transmembrane domain. In some embodiments, a transmembrane domain of a cell receptor is a CLTA4 transmembrane domain. In some embodiments, a transmembrane domain of a cell receptor is a TIM3 transmembrane domain. In some embodiments, a transmembrane domain of a cell receptor is a KIR3DL transmembrane domain. In some embodiments, a transmembrane domain of a cell receptor is a LIR1 transmembrane domain. In some embodiments, a transmembrane domain of a cell receptor is a NKG2A transmembrane domain. In some embodiments, a transmembrane domain of a cell receptor is a TIGIT transmembrane domain. In some embodiments, a transmembrane domain of a cell receptor is a LAG3 transmembrane domain.

In some embodiments, the transmembrane domain further comprises at least a portion of an extracellular domain of the same protein. In some embodiments, the transmembrane domain further comprises at least a portion of the extracellular domain of BTLA. In some embodiments, the transmembrane domain further comprises at least a portion of the extracellular domain of PD-1. In some embodiments, the transmembrane domain further comprises at least a portion of the extracellular domain of CTLA4. In some embodiments, the transmembrane domain further comprises at least a portion of the extracellular domain of TIM3. In some embodiments, the transmembrane domain further comprises at least a portion of the extracellular domain of KIR3DL1. In some embodiments, the transmembrane domain further comprises at least a portion of the extracellular domain of LIR1. In some embodiments, the transmembrane domain further comprises at least a portion of the extracellular domain of NKG2A. In some embodiments, the transmembrane domain further comprises at least a portion of the extracellular domain of TIGIT. In some embodiments, the transmembrane domain further comprises at least a portion of the extracellular domain of LAG3.

In some embodiments, the transmembrane domain further comprises at least a portion of the BTLA extracellular domain. In some embodiments, the transmembrane domain comprises at least 1, 5, 10, 15, 20, 25, 30, 35, 40, 45, 50, 55, 60, 65, 70, 75, 80, 85, 90, 95, or 100 or more amino acids of the BTLA extracellular domain. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to a portion of the BTLA extracellular domain.

In some embodiments, the transmembrane domain further comprises at least a portion of the LIR1 extracellular domain. In some embodiments, the transmembrane domain comprises at least 1, 5, 10, 15, 20, 25, 30, 35, 40, 45, 50, 55, 60, 65, 70, 75, 80, 85, 90, 95, or 100 or more amino acids of the LIR1 extracellular domain. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to a portion of the LIR1 extracellular domain.

In some embodiments, the transmembrane domain further comprises at least a portion of the PD-1 extracellular domain. In some embodiments, the transmembrane domain comprises at least 1, 5, 10, 15, 20, 25, 30, 35, 40, 45, 50, 55, 60, 65, 70, 75, 80, 85, 90, 95, or 100 or more amino acids of the PD-1 extracellular domain. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to a portion of the PD-1 extracellular domain.

In some embodiments, the transmembrane domain further comprises at least a portion of the CTLA4 extracellular domain. In some embodiments, the transmembrane domain comprises at least 1, 5, 10, 15, 20, 25, 30, 35, 40, 45, 50, 55, 60, 65, 70, 75, 80, 85, 90, 95, or 100 or more amino acids of the CTLA4 extracellular domain. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to a portion of the CTLA4 extracellular domain.

In some embodiments, the transmembrane domain further comprises at least a portion of the KIR3DL1 extracellular domain. In some embodiments, the transmembrane domain comprises at least 1, 5, 10, 15, 20, 25, 30, 35, 40, 45, 50, 55, 60, 65, 70, 75, 80, 85, 90, 95, or 100 or more amino acids of the KIR3DL1 extracellular domain. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to a portion of the KIR3DL1 extracellular domain.

In some embodiments, the transmembrane domain further comprises at least a portion of the CD28 extracellular domain. In some embodiments, the transmembrane domain comprises at least 1, 5, 10, 15, 20, 25, 30, 35, 40, 45, 50, 55, 60, 65, 70, 75, 80, 85, 90, 95, or 100 or more amino acids of the CD28 extracellular domain. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to a portion of the CD28 extracellular domain.

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 7. In some embodiments, the transmembrane domain comprises the amino acid sequence of SEQ ID NO: 7. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 8. In some embodiments, the transmembrane domain comprises the amino acid sequence of SEQ ID NO: 8. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 9. In some embodiments, the transmembrane domain comprises the amino acid sequence of SEQ ID NO: 9. In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 10. In some embodiments, the transmembrane domain comprises the amino acid sequence of SEQ ID NO: 10.

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 11. In some embodiments, the transmembrane domain comprises the amino acid sequence of FWVLVVVGGVLACYSLLVTVAFIIFWV (SEQ ID NO: 11).

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 12. In some embodiments, the transmembrane domain comprises the amino acid sequence of LLPLGGLPLLITTCFCLFCCL (SEQ ID NO: 12).

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 59. In some embodiments, the transmembrane domain comprises the amino acid sequence of VIGILVAVILLLLLLLLLFLI (SEQ ID NO: 59).

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 60. In some embodiments, the transmembrane domain comprises the amino acid sequence of VGVVGGLLGSLVLLVWVLAVI (SEQ ID NO: 60).

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 68. In some embodiments, the transmembrane domain comprises the amino acid sequence of DFLLWILAAVSSGLFFYSFLLT (SEQ ID NO: 68).

In some embodiments, the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 69. In some embodiments, the transmembrane domain comprises the amino acid sequence of ILIGTSVVIILFILLLFFLL (SEQ ID NO: 69).

Exemplary transmembrane domain amino acid sequences are shown in Table 3. Exemplary transmembrane domain nucleic acid sequences are shown in Table 4.

TABLE 3
Exemplary transmembrane domain amino acid sequences

| Amino Acid Sequence | SEQ ID NO: | Description |
|---|---|---|
| IFSGFAGLLAILLVVAVFCIL | 7 | LAX transmembrane domain |
| VAVAGCVFLLISVLLLSGL | 8 | CD25 transmembrane domain |
| AALAVISFLLGLGLGVACVLA | 9 | CD7 transmembrane domain |
| MEADALSPVGLGLLLLPFLVTLLAALAVRARELPVS | 10 | LAT transmembrane domain |
| FWVLVVVGGVLACYSLLVTVAFIIFWV | 11 | CD28 transmembrane domain |
| LLPLGGLPLLITTCFCLFCCL | 12 | BTLA transmembrane domain |
| VIGILVAVILLLLLLLLLFLI | 59 | LIR1 transmembrane domain |
| VGVVGGLLGSLVLLVWVLAVI | 60 | PD-1 transmembrane domain |
| DFLLWILAAVSSGLFFYSFLLT | 68 | CTLA4 transmembrane domain |
| ILIGTSWIILFILLLFFLL | 69 | KIR3DL1 transmembrane |
| | | domain |
| IVVITVVSAMLILCIIGLIGVIL | 89 | NKG2A (reversed) |
| | | transmembrane domain |
| LLGAMAATLVVICTAVIVVVA | 91 | TIGIT transmembrane domain |
| LIVGILGIICLILMASVVTIVVI | 107 | NKG2A transmembrane domain |

TABLE 4
Exemplary transmembrane domain nucleic acid sequences

| Nucleic Acid Sequence | SEQ ID NO: | Description |
|---|---|---|
| TTTTGGGTGCTGGTGGTGGTTGGTGGAGTCCTGGC | 13 | CD28 transmembrane domain |
| TTGCTATAGCTTGCTAGTAACAGTGGCCTTTATTAT | | |
| TTTCTGGGTG | | |
| CTCTTGCCGTTGGGGGGTCTGCCACTTCTCATAAC | 14 | BTLA transmembrane domain |
| AACTTGCTTCTGCCTTTTTTGCTGTTTG | | |
| CTGCTGCCTCTTGGAGGACTGCCCCTCCTGATCAC | 61 | BTLA tranmembrane domain |
| CACATGCTTTTGCCTGTTCTGCTGTCTG | | |
| GTTATAGGGATCCTGGTGGCTGTCATACTCCTCTT | 62 | LIR1 transmembrane domain |
| GCTCCTCTTGTTGCTGCTTTTTTTGATA | | |
| GTGATCGGAATTCTGGTGGCCGTGATTCTGCTGCT | 63 | LIR1 transmembrane domain |
| GCTCCTTCTGCTCCTGCTGTTTCTGATT | | |
| GTTGGGGTTGTAGGTGGTCTGCTCGGCAGCCTGGT | 64 | PD-1 transmembrane domain |
| CTTGTTGGTGTGGGTCTTGGCTGTGATC | | |
| GTGGGAGTTGTTGGCGGCCTGCTGGGATCTCTGGT | 65 | PD-1 transmembrane domain |
| GCTGCTTGTTTGGGTGCTCGCCGTGATC | | |
| GATTTTCTGCTGTGGATTCTGGCAGCTGTGAGCTCT | 80 | CTLA-4 transmembrane domain |
| GGCTTGTTTTTCTACAGCTTCCTCCTGACC | | |
| ATCCTGATCGGGACAAGTGTAGTAATCATACTTTT | 81 | KIR3DL1 transmembrane domain |
| CATACTCCTGCTCTTTTTTCTCTTG | | |
| GTGATCGGAATTCTGGTGGCCGTGATCCTGCTGCT | 82 | LIR1 transmembrane domain |
| CCTGCTTCTCCTCCTGCTGTTTCTGATC | | (codon optimized #1) |
| GTGATCGGCATTCTGGTGGCCGTGATTCTGCTGCT | 131 | LIR1 transmembrane domain |
| CCTGCTGTTGCTGCTGCTGTTCCTGATC | | (codon optimized #2) |
| FWVLVVVGGVLACYSLLVTVAFIIFWV | 83 | CD28 transmembrane domain |
| ATAGTGGTCATCACTGTAGTTAGTGCAATGCTTAT | 90 | NKG2A transmembrane domain |
| TCTTTGTATCATAGGGCTCATAGGGGTAATCCTG | | |
| CTGCTGGGCGCCATGGCCGCCACACTGGTTGTTAT | 92 | TIGIT transmembrane domain |
| CTGTACCGCCGTGATCGTGGTGGTGGCC | | |
| CTGATCGTGGGAATCCTGGGCATCATCTGCCTGAT | 108 | NKG2A transmembrane domain |
| CCTGATGGCCAGCGTGGTCACCATCGTGGTCATC | | |
| CTGATCGTGGGCATCCTGGGCATCATCTGTCTGAT | 132 | NKG2A transmembrane domain |
| CCTGATGGCCAGCGTGGTCACCATCGTGGTCATC | | (codon optimized) |

In some embodiments, the transmembrane domain is physically linked to the extracellular protein binding domain. In some embodiments, one of the one or more intracellular signaling domains is physically linked to the transmembrane domain. In some embodiments, the transmembrane domain is physically linked to the extracellular protein binding domain and one of the one or more intracellular signaling domains is physically linked to the transmembrane domain.

In some embodiments, the transmembrane domain comprises a amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 7. In some embodiments, the transmembrane domain comprises the amino acid sequence of SEQ ID NO: 7.

In some embodiments, the transmembrane domain comprises a nucleic acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 13. In some embodiments, the transmembrane domain comprises the nucleic acid sequence of SEQ ID NO: 13.

In some embodiments, the transmembrane domain comprises a nucleic acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 14. In some embodiments, the transmembrane domain comprises the nucleic acid sequence of SEQ ID NO: 14.

In some embodiments, the transmembrane domain comprises a nucleic acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 61. In some embodiments, the transmembrane domain comprises the nucleic acid sequence of SEQ ID NO: 61.

In some embodiments, the transmembrane domain comprises a nucleic acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 62. In some embodiments, the transmembrane domain comprises the nucleic acid sequence of SEQ ID NO: 62.

In some embodiments, the transmembrane domain comprises a nucleic acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 63. In some embodiments, the transmembrane domain comprises the nucleic acid sequence of SEQ ID NO: 63.

In some embodiments, the transmembrane domain comprises a nucleic acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 64. In some embodiments, the transmembrane domain comprises the nucleic acid sequence of SEQ ID NO: 64.

In some embodiments, the transmembrane domain comprises a nucleic acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 65. In some embodiments, the transmembrane domain comprises the nucleic acid sequence of SEQ ID NO: 65.

In some embodiments, the transmembrane domain comprises a nucleic acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 80. In some embodiments, the transmembrane domain comprises the nucleic acid sequence of SEQ ID NO: 80.

In some embodiments, the transmembrane domain comprises a nucleic acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 81. In some embodiments, the transmembrane domain comprises the nucleic acid sequence of SEQ ID NO: 81.

In some embodiments, the transmembrane domain comprises a nucleic acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 82. In some embodiments, the transmembrane domain comprises the nucleic acid sequence of SEQ ID NO: 82.

In some embodiments, the transmembrane domain comprises a nucleic acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 83. In some embodiments, the transmembrane domain comprises the nucleic acid sequence of SEQ ID NO: 83.

In some embodiments, the transmembrane domain comprises a nucleic acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 90. In some embodiments, the transmembrane domain comprises the nucleic acid sequence of SEQ ID NO: 90.

In some embodiments, the transmembrane domain comprises a nucleic acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 92. In some embodiments, the transmembrane domain comprises the nucleic acid sequence of SEQ ID NO: 92.

In some embodiments, the transmembrane domain comprises a nucleic acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 108. In some embodiments, the transmembrane domain comprises the nucleic acid sequence of SEQ ID NO: 108.

In some embodiments, the transmembrane domain comprises a nucleic acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 131. In some embodiments, the transmembrane domain comprises the nucleic acid sequence of SEQ ID NO: 131.

In some embodiments, the transmembrane domain comprises a nucleic acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 132. In some embodiments, the transmembrane domain comprises the nucleic acid sequence of SEQ ID NO: 132.

### Extracellular Protein Binding Domains

The inhibitory chimeric receptors described herein further comprise extracellular protein binding domains, such as ligand-binding domains, receptor-binding domains, antigen-binding domains, etc.

In some embodiments, immune cells expressing an inhibitory chimeric receptor are genetically modified to recognize multiple targets or proteins (e.g., ligands, receptors, antigens, etc.), which permits the recognition of unique target or protein (e.g., ligand, receptor, antigen, etc.) expression patterns on tumor cells.

In some embodiments, the protein (e.g., ligand, receptor, antigen, etc.) is not expressed on the target tumor. In some embodiments, the expression in non-tumor cells is at least 2-fold, at least 3-fold, at least 4-fold, at least 5-fold, at least 6-fold, at least 7-fold, at least 8-fold, at least 9-fold, or at least 10-fold or more lower than the level of expression that would result in activation of the tumor-targeting chimeric antigen receptor.

In some embodiments, the protein (e.g., ligand, receptor, antigen, etc.) is expressed on a non-tumor cell.

In some embodiments, the protein (e.g., ligand, receptor, antigen, etc.) is expressed on a non-tumor cell derived from a tissue selected from the group consisting of brain, neuronal tissue, endocrine, endothelial, bone, bone marrow, immune system, muscle, lung, liver, gallbladder, pancreas, gastrointestinal tract, kidney, urinary bladder, male reproductive organs, female reproductive organs, adipose, soft tissue, and skin.

In some embodiments, an extracellular protein binding domain of a inhibitory chimeric receptor of the disclosure comprises an antigen binding domain, such as a single chain Fv (scFv) specific for a tumor antigen. In some embodiments, an extracellular protein binding domain comprises an antibody, an antigen-binding fragment thereof, F(ab), F(ab′), a single chain variable fragment (scFv), or a single-domain antibody (sdAb).

The term “single-chain” refers to a molecule comprising amino acid monomers linearly linked by peptide bonds. In a particular such embodiment, the C-terminus of the Fab light chain is connected to the N-terminus of the Fab heavy chain in the single-chain Fab molecule. As described in more detail herein, an scFv has a variable domain of light chain (VL) connected from its C-terminus to the N-terminal end of a variable domain of heavy chain (VH) by a polypeptide chain. Alternately the scFv comprises of polypeptide chain where in the C-terminal end of the VH is connected to the N-terminal end of VL by a polypeptide chain.

The “Fab fragment” (also referred to as fragment antigen-binding) contains the constant domain (CL) of the light chain and the first constant domain (CH1) of the heavy chain along with the variable domains VL and VH on the light and heavy chains respectively. The variable domains comprise the complementarity determining loops (CDR, also referred to as hypervariable region) that are involved in antigen-binding. Fab′ fragments differ from Fab fragments by the addition of a few residues at the carboxy terminus of the heavy chain CH1 domain including one or more cysteines from the antibody hinge region.

“F(ab′)2” fragments contain two Fab′ fragments joined, near the hinge region, by disulfide bonds. F(ab′)2 fragments may be generated, for example, by recombinant methods or by pepsin digestion of an intact antibody. The F(ab′) fragments can be dissociated, for example, by treatment with 8-mercaptoethanol.

“Fv” fragments comprise a non-covalently-linked dimer of one heavy chain variable domain and one light chain variable domain.

“Single-chain Fv” or “sFv” or “scFv” includes the VH and VL domains of an antibody, wherein these domains are present in a single polypeptide chain. In one embodiment, the Fv polypeptide further comprises a polypeptide linker between the VH and VL domains which enables the scFv to form the desired structure for antigen-binding.

The term “single domain antibody” or “sdAb” refers to a molecule in which one variable domain of an antibody specifically binds to an antigen without the presence of the other variable domain. Single domain antibodies, and fragments thereof, are described in Arabi Ghahroudi et al., FEBS Letters, 1998, 414:521-526 and Muyldermans et al., Trends in Biochem. Sci., 2001, 26:230-245, each of which is incorporated by reference in its entirety. Single domain antibodies are also known as sdAbs or nanobodies. Sdabs are fairly stable and easy to express as fusion partner with the Fc chain of an antibody (Harmsen M M, De Haard H J (2007). “Properties, production, and applications of camelid single-domain antibody fragments”. Appl. Microbiol Biotechnol. 77(1): 13-22).

An “antibody fragment” comprises a portion of an intact antibody, such as the antigen-binding or variable region of an intact antibody. Antibody fragments include, for example, Fv fragments, Fab fragments, F(ab′)2 fragments, Fab′ fragments, scFv (sFv) fragments, and scFv-Fc fragments.

In some embodiments, the protein binding domain is an antigen-binding domain that comprises an antibody, an antigen-binding fragment of an antibody, a F(ab) fragment, a F(ab′) fragment, a single chain variable fragment (scFv), or a single-domain antibody (sdAb). In some embodiments, the antigen-binding domain comprises a single chain variable fragment (scFv). In some embodiments, each scFv comprises a heavy chain variable domain (VH) and a light chain variable domain (VL). In some embodiments, the VH and VL are separated by a peptide linker.

In some embodiments, the extracellular protein binding domain comprises a ligand-binding domain. The ligand-binding domain can be a domain from a receptor, wherein the receptor is selected from the group consisting of TCR, BCR, a cytokine receptor, RTK receptors, serine/threonine kinase receptors, hormone receptors, immunoglobulin superfamily receptors, and TNFR-superfamily of receptors.

The choice of binding domain depends upon the type and number of ligands that define the surface of a target cell. For example, the protein binding domain may be chosen to recognize a ligand that acts as a cell surface marker on target cells associated with non-disease states, such as “self” or normal tissue. Or the protein-binding domain may be chosen to recognize a ligand that acts as a cell surface marker on targets associated with a particular disease state, such as cancer or an autoimmune disease. In general, an inhibitory chimeric receptor binding domain may be selected from a non-disease state cell surface marker, while a tumor-targeting chimeric receptor binding domain may be selected from a disease state cell surface marker. Thus, examples of cell surface markers that may act as ligands for the protein binding domain in the inhibitory chimeric receptor of the present disclosure include those associated with normal tissue and examples of cell surface markers that may act as ligands for the protein binding domain in a tumor-targeting chimeric receptor include those associated with cancer cells and/or other forms of diseased cells. In some embodiments, an inhibitory chimeric receptor is engineered to target a non-tumor antigen or protein of interest by way of engineering a desired antigen or protein binding domain that specifically binds to an antigen or protein on a non-tumor cell encoded by an engineered nucleic acid.

In some embodiments, the extracellular protein binding domain comprises a receptor-binding domain. In some embodiments, the extracellular protein binding domain comprises an antigen-binding domain.

A protein binding domain (e.g., a ligand-binding domain, a receptor-binding domain, or an antigen binding domain such as an scFv) that specifically binds to a target or an epitope is a term understood in the art, and methods to determine such specific binding are also known in the art. A molecule is said to exhibit specific binding if it reacts or associates more frequently, more rapidly, with greater duration and/or with greater affinity with a particular target antigen than it does with alternative targets. A protein binding domain (e.g., a ligand-binding domain, a receptor-binding domain, or an antigen binding domain such as an scFv) that specifically binds to a first target antigen may or may not specifically bind to a second target antigen. As such, specific binding does not necessarily require (although it can include) exclusive binding.

In some embodiments, the protein binding domain has a high binding affinity.

In some embodiments, the protein binding domain has a low binding affinity.

### Linkers

In some embodiments, the inhibitory chimeric receptor comprises a peptide linker. A linker is generally used to link two peptides of a protein binding domain (e.g., an antigen-binding domain, ligand-binding domain, receptor-binding domain, etc.), such as the peptides of an scFv or sdAb. Any appropriate linker known in the art may be used, including glycerin-serine based linkers. In some embodiments, the heavy chain variable domain (VH) and light chain variable domain (VL) of an scFv are separated by a peptide linker. In some embodiments, the scFv comprises the structure VH-L-VL or VL-L-VH, wherein VH is the heavy chain variable domain, L is the peptide linker, and VL is the light chain variable domain.

In some embodiments, the inhibitory chimeric receptor comprises a peptide linker. A linker is generally used to link two peptides of a protein binding domain (e.g., an antigen-binding domain, ligand-binding domain, receptor-binding domain, etc.), such as the peptides of an scFv or sdAb. Any appropriate linker known in the art may be used, including glycerin-serine based linkers. In some embodiments, the heavy chain variable domain (VH) and light chain variable domain (VL) of an scFv are separated by a peptide linker. In some embodiments, the scFv comprises the structure VH-L-VL or VL-L-VH, wherein VH is the heavy chain variable domain, L is the peptide linker, and VL is the light chain variable domain. In some embodiments, the peptide linker comprises an amino acid sequence selected from the group consisting of GGS (SEQ ID NO: 15), GGSGGS (SEQ ID NO: 16), GGSGGSGGS (SEQ ID NO: 17), GGSGGSGGSGGS (SEQ ID NO: 18), GGSGGSGGSGGSGGS (SEQ ID NO: 19), GGGS (SEQ ID NO: 20), GGGSGGGS (SEQ ID NO: 21), GGGSGGGSGGGS (SEQ ID NO: 22), GGGSGGGSGGGSGGGS (SEQ ID NO: 23), GGGSGGGSGGGSGGGSGGGS (SEQ ID NO: 24), GGGGS (SEQ ID NO: 25), GGGGSGGGGS (SEQ ID NO: 26), GGGGSGGGGSGGGGS (SEQ ID NO: 27), GGGGSGGGGSGGGGSGGGGS (SEQ ID NO: 28), and GGGGSGGGGSGGGGSGGGGSGGGGS (SEQ ID NO: 29). In some embodiments, the peptide linker comprises a nucleic acid sequence comprising the sequence shown in SEQ ID NO: 30.

Exemplary linker amino acid sequences are shown in Table 5. An exemplary linker nucleic acid sequence is shown in Table 6.

TABLE 5
Exemplary linker amino acid sequences

| | SEQ | |
|---|---|---|
| | ID | |
| Amino Acid Sequence | NO: | Description |
| GGS | 15 | (G₂S)₁ scFv linker |
| GGSGGS | 16 | (G₂S)₂ scFv linker |
| GGSGGSGGS | 17 | (G₂S)₃ scFv linker |
| GGSGGSGGSGGS | 18 | (G₂S)₄ scFv linker |
| GGSGGSGGSGGSGGS | 19 | (G₂S)₅ scFv linker |
| GGGS | 20 | (G₃S)₁ scFv linker |
| GGGSGGGS | 21 | (G₃S)₂ scFv linker |
| GGGSGGGSGGGS | 22 | (G₃S)₃ scFv linker |
| GGGSGGGSGGGSGGGS | 23 | (G₃S)₄ scFv linker |
| GGGSGGGSGGGSGGGSGGGS | 24 | (G₃S)₅ scFv linker |
| GGGGS | 25 | (G₄S)₁ scFv linker |
| GGGGSGGGGS | 26 | (G₄S)₂ scFv linker |
| GGGGSGGGGSGGGGS | 27 | (G₄S)₃ scFv linker |
| GGGGSGGGGSGGGGSGGGGS | 28 | (G₄S)₄ scFv linker |
| GGGGSGGGGSGGGGSGGGGSGGGGS | 29 | (G₄S)₅ scFv linker |

TABLE 6
Exemplary linker nucleic acid sequence

| | SEQ | |
|---|---|---|
| | ID | |
| Nucleic Acid Sequence | NO: | Description |
| GGAGGCGGAGGATCTGGTGGCGGAGGAAGTG | 30 | (G₄S)₃ scFv |
| GCGGAGGCGGTTCT | | linker |

### Spacers Hinges

Chimer receptors can also contain spacer or hinge domains in the polypeptide. In some embodiments, a spacer domain or a hinge domain is located between an extracellular domain (e.g., comprising the protein binding domain) and a transmembrane domain of an inhibitory chimeric receptor or tumor-targeting chimeric receptor, or between an intracellular signaling domain and a transmembrane domain of the inhibitory chimeric receptor or tumor-targeting chimeric receptor. A spacer or hinge domain is any oligopeptide or polypeptide that functions to link the transmembrane domain to the extracellular domain and/or the intracellular signaling domain in the polypeptide chain. Spacer or hinge domains provide flexibility to the inhibitory chimeric receptor or tumor-targeting chimeric receptor, or domains thereof, or prevent steric hindrance of the inhibitory chimeric receptor or tumor-targeting chimeric receptor, or domains thereof. In some embodiments, a spacer domain or hinge domain may comprise up to 300 amino acids (e.g., 10 to 100 amino acids, or 5 to 20 amino acids). In some embodiments, one or more spacer domain(s) may be included in other regions of an inhibitory chimeric receptor or tumor-targeting chimeric receptor.

Exemplary spacer or hinge domain amino acid sequences are shown in Table 7. Exemplary spacer or hinge domain nucleic acid sequences are shown in Table 8.

TABLE 7
Exemplary spacer or hinge domain amino acid sequences

| Amino Acid Sequence | SEQ ID NO: | Description |
|---|---|---|
| AAAIEVMYPPPYLDNEKSNGTIIHVKGKHLCPSPLFP | 31 | CD28 hinge |
| GPSKP | | |
| ESKYGPPCPSCP | 32 | IgG4 minimal hinge |
| ESKYGPPAPSAP | 33 | IgG4 minimal hinge, no |
| | | disulfides |
| ESKYGPPCPPCP | 34 | IgG4 S228P minimal hinge, |
| | | enhanced disulfide formation |
| EPKSCDKTHTCP | 35 | IgG1 minimal hinge |
| AAAFVPVFLPAKPTTTPAPRPPTPAPTIASQPLSLRPE | 36 | Extended CD8a hinge |
| ACRPAAGGAVHTRGLDFACDIYIWAPLAGTCGVLLL | | |
| SLVITLYCNHRN | | |
| TTTPAPRPPTPAPTIALQPLSLRPEACRPAAGGAVHTR | 37 | CD8a hinge |
| GLDFACD | | |
| ACPTGLYTHSGECCKACNLGEGVAQPCGANQTVCE | 38 | LNGFR hinge |
| PCLDSVTFSDVVSATEPCKPCTECVGLQSMSAPCVEA | | |
| DDAVCRCAYGYYQDETTGRCEACRVCEAGSGLVFS | | |
| CQDKQNTVCEECPDGTYSDEADAEC | | |
| ACPTGLYTHSGECCKACNLGEGVAQPCGANQTVC | 39 | Truncated LNGFR hinge (TNFR- |
| | | Cys1) |
| AVGQDTQEVIVVPHSLPFKV | 40 | PDGFR-beta extracellular linker |
| TTTPAPRPPTPAPTIALQPLSLRPEACRPAAGGAVHTR | 70 | CD8a-DAP10e hinge |
| GLDFACDQTTPGERSSLPAFYPGTSGSCSGCGSLSLP | | |

TABLE 8
Exemplary spacer or hinge domain nucleic acid sequences

| Nucleic Acid Sequence | SEQ ID NO: | Description |
|---|---|---|
| GCAGCAGCTATCGAGGTGATGTATCCTCCGCCCTA | 41 | CD28 hinge |
| CCTGGATAATGAAAAGAGTAATGGGACTATCATTC | | |
| ATGTAAAAGGGAAGCATCTTTGTCCTTCTCCCCTTT | | |
| TCCCCGGTCCGTCTAAACCT | | |
| GAAAGCAAGTACGGTCCACCTTGCCCTAGCTGTCC | 42 | IgG4 minimal hinge |
| G | | |
| GAATCCAAGTACGGCCCCCCAGCGCCTAGTGCCCC | 43 | IgG4 minimal hinge, no |
| A | | disulfides |
| GAATCTAAATATGGCCCGCCATGCCCGCCTTGCCC | 44 | IgG4 S228P minimal hinge, |
| A | | enhanced disulfide formation |
| GAACCGAAGTCTTGTGATAAAACTCATACGTGCCC | 45 | IgG1 minimal hinge |
| G | | |
| GCTGCTGCTTTCGTACCCGTGTTCCTCCCTGCTAAG | 46 | Extended CD8a hinge |
| CCTACGACTACCCCCGCACCGAGACCACCCACGCC | | |
| AGCACCCACGATTGCTAGCCAGCCCCTTAGTTTGC | | |
| GACCAGAAGCTTGTCGGCCTGCTGCTGGTGGCGCG | | |
| GTACATACCCGCGGCCTTGATTTTGCTTGCGATAT | | |
| ATATATCTGGGCGCCTCTGGCCGGAACATGCGGGG | | |
| TCCTCCTCCTTTCTCTGGTTATTACTCTCTACTGTA | | |
| ATCACAGGAAT | | |
| GCCTGCCCGACCGGGCTCTACACTCATAGCGGGGA | 47 | LNGFR hinge |
| ATGTTGTAAGGCATGTAACTTGGGTGAGGGCGTCG | | |
| CACAGCCCTGCGGAGCTAACCAAACAGTGTGCGA | | |
| ACCCTGCCTCGATAGTGTGACGTTCTCTGATGTTGT | | |
| ATCAGCTACAGAGCCTTGCAAACCATGTACTGAGT | | |
| GCGTTGGACTTCAGTCAATGAGCGCTCCATGTGTG | | |
| GAGGCAGATGATGCGGTCTGTCGATGTGCTTACGG | | |
| ATACTACCAAGACGAGACAACAGGGCGGTGCGAG | | |
| GCCTGTAGAGTTTGTGAGGCGGGCTCCGGGCTGGT | | |
| GTTTTCATGTCAAGACAAGCAAAATACGGTCTGTG | | |
| AAGAGTGCCCTGATGGCACCTACTCAGACGAAGC | | |
| AGATGCAGAATGC | | |
| GCCTGCCCTACAGGACTCTACACGCATAGCGGTGA | 48 | Truncated LNGFR hinge (TNFR- |
| GTGTTGTAAAGCATGCAACCTCGGGGAAGGTGTA | | Cys1) |
| GCCCAGCCATGCGGGGCTAACCAAACCGTTTGC | | |
| GCTGTGGGCCAGGACACGCAGGAGGTCATCGTGG | 49 | PDGFR-beta extracellular linker |
| TGCCACACTCCTTGCCCTTTAAGGTG | | |
| ACCACGACGCCAGCGCCGCGACCACCAACACCGG | 79 | CD8 hinge |
| CGCCCACCATCGCGTTGCAGCCCCTGTCCCTGCGC | | |
| CCAGAGGCGTGCCGGCCAGCGGCGGGGGGCGCAG | | |
| TGCACACGAGGGGGCTGGACTTCGCCTGTGAT | | |
| ACCACGACGCCAGCGCCGCGACCACCAACACCGG | 87 | CD8a-DAP10e hinge |
| CGCCCACCATCGCGTTGCAGCCCCTGTCCCTGCGC | | |
| CCAGAGGCGTGCCGGCCAGCGGCGGGGGGCGCAG | | |
| TGCACACGAGGGGGCTGGACTTCGCCTGTGATCAG | | |
| ACCACACCTGGCGAGAGATCTTCCCTGCCTGCCTT | | |
| CTATCCTGGCACCAGCGGCTCTTGTTCTGGCTGTG | | |
| GATCACTGAGCCTGCCT | | |
| GCCGCTGCTATCGAAGTGATGTACCCTCCTCCTTA | 88 | CD28 hinge |
| CCTGGACAACGAGAAGTCCAACGGCACCATCATC | | |
| CACGTGAAGGGCAAGCACCTGTGTCCTTCTCCACT | | |
| GTTCCCCGGACCTAGCAAGCCT | | |

In some embodiments, the chimeric inhibitory receptor further comprises a spacer region positioned between the protein binding domain and the transmembrane domain and operably linked to each of the protein binding domain and the transmembrane domain. In some embodiments, the chimeric inhibitory receptor further comprises a spacer region positioned between the protein binding domain and the transmembrane domain and physically linked to each of the protein binding domain and the transmembrane domain.

In some embodiments, the chimeric inhibitory receptor further comprises a spacer region between the protein binding domain and the transmembrane domain.

In some embodiments, the spacer region is derived from a protein selected from the group consisting of: CD8a, CD4, CD7, CD28, IgG1, IgG4, FcγRIIIa, LNGFR, and PDGFR. In some embodiments, the spacer region comprises an amino acid sequence selected from the group consisting of:

(SEQ ID NO: 31)
AAAIEVMYPPPYLDNEKSNGTIIHVKGKHLCPSPLFPGPSKP,
(SEQ ID NO: 32)
ESKYGPPCPSCP,
(SEQ ID NO: 33)
ESKYGPPAPSAP,
(SEQ ID NO: 34)
ESKYGPPCPPCP,
(SEQ ID NO: 35)
EPKSCDKTHTCP,
(SEQ ID NO: 36)
AAAFVPVFLPAKPTTTPAPRPPTPAPTIASQPLSLRPEACRPAAGGAVHT
RGLDFACDIYIWAPLAGTCGVLLLSLVITLYCNHRN,
(SEQ ID NO: 37)
TTTPAPRPPTPAPTIALQPLSLRPEACRPAAGGAVHTRGLDFACD,
(SEQ ID NO: 38)
ACPTGLYTHSGECCKACNLGEGVAQPCGANQTVCEPCLDSVTFSDVVSAT
EPCKPCTECVGLQSMSAPCVEADDAVCRCAYGYYQDETTGRCEACRVCEA
GSGLVFSCQDKQNTVCEECPDGTYSDEADAEC,
(SEQ ID NO: 39)
ACPTGLYTHSGECCKACNLGEGVAQPCGANQTVC,
(SEQ ID NO: 40)
AVGQDTQEVIVVPHSLPFKV,
and
(SEQ ID NO: 70)
TTTPAPRPPTPAPTIALQPLSLRPEACRPAAGGAVHTRGLDFACDQTTPG
ERSSLPAFYPGTSGSCSGCGSLSLP.

In some embodiments, the spacer region comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 31. In some embodiments, the spacer region comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 32. In some embodiments, the spacer region comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 33. In some embodiments, the spacer region comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 34. In some embodiments, the spacer region comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 35. In some embodiments, the spacer region comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 36. In some embodiments, the spacer region comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 37. In some embodiments, the spacer region comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 38. In some embodiments, the spacer region comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 39. In some embodiments, the spacer region comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 40. In some embodiments, the spacer region comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO: 70.

In some embodiments, the spacer region modulates sensitivity of the chimeric inhibitory receptor. In some embodiments, the spacer region increases sensitivity of the chimeric inhibitory receptor relative to an otherwise identical chimeric inhibitory receptor lacking the spacer region. In some embodiments, the spacer region reduces sensitivity of the chimeric inhibitory receptor relative to an otherwise identical chimeric inhibitory receptor lacking the spacer region. In some embodiments, the spacer region modulates potency of the chimeric inhibitory receptor relative to an otherwise identical chimeric inhibitory receptor lacking the spacer region. In some embodiments, the spacer region increases potency of the chimeric inhibitory receptor relative to an otherwise identical chimeric inhibitory receptor lacking the spacer region. In some embodiments, the spacer region reduces potency of the chimeric inhibitory receptor relative to an otherwise identical chimeric inhibitory receptor lacking the spacer region. In some embodiments, the spacer region modulates basal prevention, attenuation, or inhibition of activation of the tumor-targeting chimeric receptor expressed on the immunomodulatory cell relative to an otherwise identical chimeric inhibitory receptor lacking the spacer region. In some embodiments, the spacer region reduces basal prevention, attenuation, or inhibition relative to an otherwise identical chimeric inhibitory receptor lacking the spacer region. In some embodiments, the spacer region increases basal prevention, attenuation, or inhibition relative to an otherwise identical chimeric inhibitory receptor lacking the spacer region.

In some embodiments, wherein the chimeric inhibitory receptor further comprises an intracellular spacer region positioned between the transmembrane domain and one of the one or more intracellular signaling domains and operably linked to each of the transmembrane domain and the intracellular signaling domain. In some embodiments, the chimeric inhibitory receptor further comprises an intracellular spacer region positioned between the transmembrane domain and one of the one or more intracellular signaling domains and physically linked to each of the transmembrane domain and the intracellular signaling domain.

In some embodiments, the intracellular spacer region modulates sensitivity of the chimeric inhibitory receptor relative to an otherwise identical chimeric inhibitory receptor lacking the intracellular spacer region. In some embodiments, the intracellular spacer region increases sensitivity of the chimeric inhibitory receptor relative to an otherwise identical chimeric inhibitory receptor lacking the intracellular spacer region. In some embodiments, the intracellular spacer region reduces sensitivity of the chimeric inhibitory receptor relative to an otherwise identical chimeric inhibitory receptor lacking the intracellular spacer region. In some embodiments, the intracellular spacer region modulates potency of the chimeric inhibitory receptor relative to an otherwise identical chimeric inhibitory receptor lacking the intracellular spacer region.

In some embodiments, the intracellular spacer region increases potency of the chimeric inhibitory receptor relative to an otherwise identical chimeric inhibitory receptor lacking the intracellular spacer region. In some embodiments, the intracellular spacer region reduces potency of the chimeric inhibitory receptor relative to an otherwise identical chimeric inhibitory receptor lacking the intracellular spacer region. In some embodiments, the intracellular spacer region modulates basal prevention, attenuation, or inhibition of activation of the tumor-targeting chimeric receptor expressed on the immunomodulatory cell when expressed on an immunomodulatory cell relative to an otherwise identical chimeric inhibitory receptor lacking the intracellular spacer region. In some embodiments, the intracellular spacer region reduces basal prevention, attenuation, or inhibition relative to an otherwise identical chimeric inhibitory receptor lacking the intracellular spacer region. In some embodiments, the intracellular spacer region increases basal prevention, attenuation, or inhibition relative to an otherwise identical chimeric inhibitory receptor lacking the intracellular spacer region.

### Polynucleotides Encoding Inhibitory Chimeric Receptors

Also presented herein are a polynucleotide or set of polynucleotides encoding an inhibitory chimeric receptor, and a vector comprising such a polynucleotide. When the inhibitory chimeric receptor is a multichain receptor, a set of polynucleotides is used. In this case, the set of polynucleotides can be cloned into a single vector or a plurality of vectors. In some embodiments, the polynucleotide comprises a sequence encoding an inhibitory chimeric receptor, wherein the sequence encoding an extracellular protein binding domain is contiguous with and in the same reading frame as a sequence encoding an intracellular signaling domain and a transmembrane domain.

The polynucleotide can be codon optimized for expression in a mammalian cell. In some embodiments, the entire sequence of the polynucleotide has been codon optimized for expression in a mammalian cell. Codon optimization refers to the discovery that the frequency of occurrence of synonymous codons (i.e., codons that code for the same amino acid) in coding DNA is biased in different species. Such codon degeneracy allows an identical polypeptide to be encoded by a variety of nucleic acid sequences. A variety of codon optimization methods is known in the art, and include, e.g., methods disclosed in at least U.S. Pat. Nos. 5,786,464 and 6,114,148.

The polynucleotide encoding an inhibitory chimeric receptor can be obtained using recombinant methods known in the art, such as, for example by screening libraries from cells expressing the polynucleotide, by deriving it from a vector known to include the same, or by isolating directly from cells and tissues containing the same, using standard techniques. Alternatively, the polynucleotide can be produced synthetically, rather than cloned.

The polynucleotide can be cloned into a vector. In some embodiments, an expression vector known in the art is used. Accordingly, the present disclosure includes retroviral and lentiviral vector constructs expressing an inhibitory chimeric receptor that can be directly transduced into a cell.

The present disclosure also includes an RNA construct that can be directly transfected into a cell. A method for generating mRNA for use in transfection involves in vitro transcription (IVT) of a template with specially designed primers, followed by polyA addition, to produce a construct containing 3′ and 5′ untranslated sequence (“UTR”) (e.g., a 3′ and/or 5′ UTR described herein), a 5′ cap (e.g., a 5′ cap described herein) and/or Internal Ribosome Entry Site (IRES) (e.g., an IRES described herein), the nucleic acid to be expressed, and a polyA tail. RNA so produced can efficiently transfect different kinds of cells. In some embodiments, an RNA inhibitory chimeric receptor vector is transduced into a cell, e.g., a T cell or a NK cell, by electroporation.

### Cells

In one aspect, the present disclosure provides inhibitory chimeric receptor-modified cells. The cells can be stem cells, progenitor cells, and/or immune cells modified to express an inhibitory chimeric receptor described herein. In some embodiments, a cell line derived from an immune cell is used. Non-limiting examples of cells, as provided herein, include mesenchymal stem cells (MSCs), natural killer (NK) cells, NKT cells, innate lymphoid cells, mast cells, eosinophils, basophils, macrophages, neutrophils, mesenchymal stem cells, dendritic cells, T cells (e.g., CD8+ T cells, CD4+ T cells, gamma-delta T cells, and T regulatory cells (CD4+, FOXP3+, CD25+)), and B cells. In some embodiments, the cell a stem cell, such as pluripotent stem cell, embryonic stem cell, adult stem cell, bone-marrow stem cell, umbilical cord stem cells, or other stem cell.

The cells can be modified to express an inhibitory chimeric receptor provided herein. Accordingly, the present disclosure provides a cell (e.g., a population of cells) engineered to express an inhibitory chimeric receptor, wherein the inhibitory chimeric receptor comprises a protein binding domain (e.g., an antigen-binding domain, a ligand-binding domain, a receptor-binding domain, etc.), a transmembrane domain, and one or more inhibitory intracellular signaling domains. In some embodiments, the inhibitory chimeric receptor comprises two or more intracellular signaling domains. In some embodiments, the inhibitory chimeric receptor comprises three or more intracellular signaling domains. In some embodiments, the inhibitory chimeric receptor comprises four or more intracellular signaling domains. In some embodiments, the inhibitory chimeric receptor comprises five or more intracellular signaling domains. In some embodiments, the inhibitory chimeric receptor comprises one intracellular signaling domain. In some embodiments, the inhibitory chimeric receptor comprises two intracellular signaling domains. In some embodiments, the inhibitory chimeric receptor comprises three intracellular signaling domains. In some embodiments, the inhibitory chimeric receptor comprises four intracellular signaling domains. In some embodiments, the inhibitory chimeric receptor comprises five intracellular signaling domains.

In some embodiments, the immunomodulatory cell is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an ESC-derived cell, and an iPSC-derived cell. In some embodiments, the immunomodulatory cell is a Natural Killer (NK) cell.

In some embodiments, the cell is autologous. In some embodiments, the cell is allogeneic.

In some embodiments, an immunomodulatory cell comprises a chimeric inhibitory receptor, wherein the chimeric inhibitory receptor comprises: an extracellular protein binding domain (e.g., an extracellular antigen-binding domain, an extracellular ligand-binding domain, an extracellular receptor-binding domain, etc.); a transmembrane domain, wherein the transmembrane domain is operably linked to the extracellular protein binding domain; and one or more intracellular signaling domains, wherein the one or more intracellular signaling domains are operably linked to the transmembrane domain, and wherein upon binding of the protein (e.g., ligand, receptor, antigen, etc.) to the chimeric inhibitory receptor, the chimeric inhibitory receptor prevents, attenuates, or inhibits activation of a tumor-targeting chimeric receptor expressed on the surface of the cell. In some embodiments, the inhibitory chimeric receptor comprises two or more intracellular signaling domains. In some embodiments, the inhibitory chimeric receptor comprises three or more intracellular signaling domains. In some embodiments, the inhibitory chimeric receptor comprises four or more intracellular signaling domains. In some embodiments, the inhibitory chimeric receptor comprises five or more intracellular signaling domains. In some embodiments, the inhibitory chimeric receptor comprises one intracellular signaling domain. In some embodiments, the inhibitory chimeric receptor comprises two intracellular signaling domains. In some embodiments, the inhibitory chimeric receptor comprises three intracellular signaling domains. In some embodiments, the inhibitory chimeric receptor comprises four intracellular signaling domains. In some embodiments, the inhibitory chimeric receptor comprises five intracellular signaling domains.

In some embodiments, the cell further comprises a tumor-targeting chimeric receptor expressed on the surface of the cell. In some embodiments, the chimeric inhibitory receptor is recombinantly expressed.

In some embodiments, prior to binding of the protein (e.g., ligand, receptor, antigen, etc.) to the chimeric inhibitory receptor, the tumor-targeting chimeric receptor is capable of activating the cell. In some embodiments, upon binding of the protein (e.g., ligand, receptor, antigen, etc.) to the chimeric inhibitory receptor, the chimeric inhibitory receptor suppresses cytokine production from the activated cell. In some embodiments, upon binding of the protein (e.g., ligand, receptor, antigen, etc.) to the chimeric inhibitory receptor, the chimeric inhibitory receptor suppresses a cell-mediated immune response to a target cell, wherein the immune response is induced by activation of the immunomodulatory cell. In some embodiments, the target cell is a tumor cell. In some embodiments, the target cell is a non-tumor cell.

### Cells Expressing Multiple Chimeric Receptors

The cells can be modified to express an inhibitory chimeric receptor provided herein. The cells can also be modified to express an inhibitory chimeric receptor (e.g., an iCAR) and a tumor-targeting CAR (e.g., an aCAR). If a cell is modified to express at least one inhibitory chimeric receptor and at least one tumor-targeting CAR, the cells can express multiple inhibitory and/or tumor-targeting chimeric receptor proteins and/or polynucleotides. In some embodiments, the cell expresses at least 1, at least 2, at least 3, at least 4, at least 5, at least 6, at least 7, at least 8, at least 9, or at least 10 or more inhibitory chimeric receptor polynucleotide and/or polypeptide. In some embodiments, the cell contains at least 1, at least 2, at least 3, at least 4, at least 5, at least 6, at least 7, at least 8, at least 9, or at least 10 or more tumor-targeting chimeric receptor polynucleotide and/or polypeptide.

### Methods of Preparing Inhibitory Chimeric Receptor-Modified Cells

In one aspect, the present disclosure provides a method of preparing a modified immune cells comprising an inhibitory chimeric receptor for experimental or therapeutic use.

Ex vivo procedures for making therapeutic inhibitory chimeric receptor-modified cells are well known in the art. For example, cells are isolated from a mammal (e.g., a human) and genetically modified (i.e., transduced or transfected in vitro) with a vector expressing a inhibitory chimeric receptor disclosed herein. The inhibitory chimeric receptor-modified cell can be administered to a mammalian recipient to provide a therapeutic benefit. The mammalian recipient may be a human and the inhibitory chimeric receptor-modified cell can be autologous with respect to the recipient. Alternatively, the cells can be allogeneic, syngeneic or xenogeneic with respect to the recipient. The procedure for ex vivo expansion of hematopoietic stem and progenitor cells is described in U.S. Pat. No. 5,199,942, incorporated herein by reference, can be applied to the cells of the present disclosure. Other suitable methods are known in the art, therefore the present disclosure is not limited to any particular method of ex vivo expansion of the cells. Briefly, ex vivo culture and expansion of immune effector cells (e.g., T cells, NK cells) comprises: (1) collecting CD34+ hematopoietic stem and progenitor cells from a mammal from peripheral blood harvest or bone marrow explants; and (2) expanding such cells ex vivo. In addition to the cellular growth factors described in U.S. Pat. No. 5,199,942, other factors such as flt3-L, IL-1, IL-3 and c-kit ligand, can be used for culturing and expansion of the cells.

In some embodiments, the methods comprise culturing the population of cells (e.g. in cell culture media) to a desired cell density (e.g., a cell density sufficient for a particular cell-based therapy). In some embodiments, the population of cells are cultured in the absence of an agent that represses activity of the repressible protease or in the presence of an agent that represses activity of the repressible protease.

In some embodiments, the population of cells is cultured for a period of time that results in the production of an expanded cell population that comprises at least 2-fold the number of cells of the starting population. In some embodiments, the population of cells is cultured for a period of time that results in the production of an expanded cell population that comprises at least 4-fold the number of cells of the starting population. In some embodiments, the population of cells is cultured for a period of time that results in the production of an expanded cell population that comprises at least 16-fold the number of cells of the starting population.

### Methods of Use

Methods for treatment of immune-related disorders, such as cancers, are also encompassed. Said methods include administering an inhibitory chimeric receptor or immunoresponsive inhibitory chimeric receptor-modified cell as described herein. In some embodiments, compositions comprising chimeric receptors or genetically modified immunoresponsive cells that express such chimeric receptors can be provided systemically or directly to a subject for the treatment of a proliferative disorder, such as a cancer.

In one aspect, the present disclosure provides a method of preparing a modified immune cells comprising at least one inhibitory chimeric receptor (e.g., inhibitory chimeric receptor (iCAR)-modified cells) for experimental or therapeutic use. In some embodiments, the modified immune cells further comprise at least one tumor-targeting chimeric receptor (e.g., iCAR and aCAR-modified cells).

In some aspects, methods of use encompass methods of preventing, attenuating, or inhibiting a cell-mediated immune response induced by a chimeric receptor expressed of the surface of an immunomodulatory cell, comprising: engineering the immunomodulatory cell to express the chimeric inhibitory receptor described herein on the surface of the immunomodulatory cell, wherein upon binding of a cognate protein (e.g., ligand, receptor, antigen, etc.) to the chimeric inhibitory receptor, the intracellular signaling domain prevents, attenuates, or inhibits activation of the chimeric receptor. In other aspects, methods of use encompass methods of preventing, attenuating, or inhibiting activation of a chimeric receptor expressed on the surface of an immunomodulatory cell, comprising: contacting an isolated cell or a composition as described herein with a cognate protein (e.g., ligand, receptor, antigen, etc.) of the chimeric inhibitory receptor under conditions suitable for the chimeric inhibitory receptor to bind the cognate protein (e.g., ligand, receptor, antigen, etc.), wherein upon binding of the protein (e.g., ligand, receptor, antigen, etc.) to the chimeric inhibitory receptor, the intracellular signaling domain prevents, attenuates, or inhibits activation of the chimeric receptor.

In general, the inhibitory chimeric receptor is used to prevent, attenuate, inhibit, or suppress an immune response initiated by a tumor targeting chimeric receptor (e.g., an activating CAR). For example, an immunomodulator cell expresses an inhibitory chimeric receptor that recognizes a protein target 1 (e.g., a non-tumor target ligand, receptor, antigen, etc.) and a tumor-targeting chimeric receptor that recognizes a protein target 2 (e.g., a tumor target antigen). When the exemplary immunomodulatory cell contacts a target cell, the inhibitory and tumor targeting chimeric receptors may or may not bind to their cognate protein. In exemplary instances where the target cell is a non-tumor cell that expresses both protein target 1 and protein target 2, both the inhibitory chimeric receptor and the tumor-targeting receptor can be activated. In such cases, the activation of the inhibitory chimeric receptor results in the prevention, attenuation, or inhibition of the tumor targeting chimeric receptor signaling and the immunomodulatory cell is not activated. Similarly, in exemplary instances where the target cell is a non-tumor cell that expresses only protein target 1, only the inhibitory chimeric receptor can be activated. In contrast, in exemplary instances where the target cell is a tumor cell that expresses only protein target 2, the inhibitory chimeric receptor cannot be activated while the tumor-targeting chimeric receptor can be activated, resulting in signal transduction that results in activation of the immunomodulatory cell.

Attenuation of an immune response initiated by a tumor targeting chimeric receptor can be a decrease or reduction in the activation of the tumor targeting chimeric receptor, a decrease or reduction in the signal transduction of a tumor targeting chimeric receptor, or a decrease or reduction in the activation of the immunomodulatory cell. The inhibitory chimeric receptor can attenuate activation of the tumor targeting chimeric receptor, signal transduction by the tumor targeting chimeric receptor, or activation of the immunomodulatory cell by the tumor targeting chimeric receptor 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold or more as compared to the activation of the tumor targeting chimeric receptor, signal transduction, or activation of the immunomodulatory cell as compared to an immunomodulatory cell lacking an inhibitory chimeric receptor. In some embodiments, attenuation refers to a decrease or reduction of the activity of a tumor targeting chimeric receptor after it has been activated.

Prevention of an immune response initiated by a tumor targeting chimeric receptor can be an inhibition or reduction in the activation of the tumor targeting chimeric receptor, an inhibition or reduction in the signal transduction of a tumor targeting chimeric receptor, or an inhibition or reduction in the activation of the immunomodulatory cell. The inhibitory chimeric receptor can prevent activation of the tumor targeting chimeric receptor, signal transduction by the tumor targeting chimeric receptor, or activation of the immunomodulatory cell by the tumor targeting chimeric receptor by about 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold or more as compared to the activation of the tumor targeting chimeric receptor, signal transduction, or activation of the immunomodulatory cell as compared to an immunomodulatory cell lacking an inhibitory chimeric receptor. In some embodiments, prevention refers to a blockage of the activity of a tumor targeting chimeric receptor before it has been activated.

Inhibition of an immune response initiated by a tumor targeting chimeric receptor can be an inhibition or reduction in the activation of the tumor targeting chimeric receptor, an inhibition or reduction in the signal transduction of a tumor targeting chimeric receptor, or an inhibition or reduction in the activation of the immunomodulatory cell. The inhibitory chimeric receptor can inhibit activation of the tumor targeting chimeric receptor, signal transduction by the tumor targeting chimeric receptor, or activation of the immunomodulatory cell by the tumor targeting chimeric receptor by about 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold or more as compared to the activation of the tumor targeting chimeric receptor, signal transduction, or activation of the immunomodulatory cell as compared to an immunomodulatory cell lacking an inhibitory chimeric receptor. In some embodiments, inhibition refers to a decrease or reduction of the activity of a tumor targeting chimeric receptor before or after it has been activated.

Suppression of an immune response initiated by a tumor targeting chimeric receptor can be an inhibition or reduction in the activation of the tumor targeting chimeric receptor, an inhibition or reduction in the signal transduction of a tumor targeting chimeric receptor, or an inhibition or reduction in the activation of the immunomodulatory cell. The inhibitory chimeric receptor can suppress activation of the tumor targeting chimeric receptor, signal transduction by the tumor targeting chimeric receptor, or activation of the immunomodulatory cell by the tumor targeting chimeric receptor by about 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold or more as compared to the activation of the tumor targeting chimeric receptor, signal transduction, or activation of the immunomodulatory cell as compared to an immunomodulatory cell lacking an inhibitory chimeric receptor. In some embodiments, suppression refers to a decrease or reduction of the activity of a tumor targeting chimeric receptor before or after it has been activated.

The immune response can be cytokine or chemokine production and secretion from an activated immunomodulatory cell. The immune response can be a cell-mediated immune response to a target cell.

In some embodiments, the chimeric inhibitory receptor is capable of suppressing cytokine production from an activated immunomodulatory cell. In some embodiments, the chimeric inhibitory receptor is capable of suppressing a cell-mediated immune response to a target cell, wherein the immune response is induced by activation of the immunomodulatory cell.

In one aspect, the present disclosure provides a type of cell therapy where immune cells are genetically modified to express an inhibitory chimeric receptor provided herein and the modified immune cells are administered to a subject in need thereof.

Thus, in some embodiments, the methods comprise delivering cells of the expanded population of cells to a subject in need of a cell-based therapy to treat a condition or disorder. In some embodiments, the subject is a human subject. In some embodiments, the condition or disorder is an autoimmune condition. In some embodiments, the condition or disorder is an immune related condition. In some embodiments, the condition or disorder is a cancer (e.g., a primary cancer or a metastatic cancer). In some embodiments, the cancer is a solid cancer. In some embodiments, the cancer is a liquid cancer, such as a myeloid disorder.

### Pharmaceutical Compositions

The inhibitory chimeric receptor or immunoresponsive cell can be formulated in pharmaceutical compositions. Pharmaceutical compositions of the present disclosure can comprise an inhibitory chimeric receptor (e.g., an iCAR) or immunoresponsive cell (e.g., a plurality of inhibitory chimeric receptor-expressing cells), as described herein, in combination with one or more pharmaceutically or physiologically acceptable carriers, diluents or excipients. Such materials should be non-toxic and should not interfere with the efficacy of the active ingredient. The precise nature of the carrier or other material can depend on the route of administration, e.g. oral, intravenous, cutaneous or subcutaneous, nasal, intramuscular, intraperitoneal routes. In certain embodiments, the composition is directly injected into an organ of interest (e.g., an organ affected by a disorder). Alternatively, the composition may be provided indirectly to the organ of interest, for example, by administration into the circulatory system (e.g., the tumor vasculature). Expansion and differentiation agents can be provided prior to, during, or after administration of the composition to increase production of T cells, NK cells, or CTL cells in vitro or in vivo.

In certain embodiments, the compositions are pharmaceutical compositions comprising genetically modified cells, such as immunoresponsive cells or their progenitors and a pharmaceutically acceptable carrier. Administration can be autologous or heterologous. For example, immunoresponsive cells, or progenitors can be obtained from one subject, and administered to the same subject or a different, compatible subject. In some embodiments, immunoresponsive cells of the present disclosure or their progeny may be derived from peripheral blood cells (e.g., in vivo, ex vivo, or in vitro derived) and may be administered via localized injection, including catheter administration, systemic injection, localized injection, intravenous injection, or parenteral administration. When administering a therapeutic composition of the present disclosure (e.g., a pharmaceutical composition containing a genetically modified cell of the present disclosure), it will generally be formulated in a unit dosage injectable form (solution, suspension, emulsion).

Certain aspects of the present disclosure relate to formulations of compositions comprising chimeric receptors of the present disclosure or genetically modified cells (e.g., immunoresponsive cells of the present disclosure) expressing such chimeric receptors. In some embodiments, compositions of the present disclosure comprising genetically modified cells may be provided as sterile liquid preparations, including without limitation isotonic aqueous solutions, suspensions, emulsions, dispersions, and viscous compositions, which may be buffered to a selected pH. Liquid preparations are typically easier to prepare than gels, other viscous compositions, and solid compositions. Additionally, liquid compositions may be more convenient to administer, especially by injection. In some embodiments, viscous compositions can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (e.g., glycerol, propylene glycol, liquid polyethylene glycol, etc.) and suitable mixtures thereof.

Pharmaceutical compositions for oral administration can be in tablet, capsule, powder or liquid form. A tablet can include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol can be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required. In some embodiments, compositions of the present disclosure can be isotonic, i.e., having the same osmotic pressure as blood and lacrimal fluid. In some embodiments, the desired isotonicity may be achieved using, for example, sodium chloride, dextrose, boric acid, sodium tartrate, propylene glycol, or other inorganic or organic solutes.

In some embodiments, compositions of the present disclosure may further include various additives that may enhance the stability and sterility of the compositions. Examples of such additives include, without limitation, antimicrobial preservatives, antioxidants, chelating agents, and buffers. In some embodiments, microbial contamination may be prevented by the inclusions of any of various antibacterial and antifungal agents, including without limitation parabens, chlorobutanol, phenol, sorbic acid, and the like. Prolonged absorption of an injectable pharmaceutical formulation of the; present disclosure can be brought about by the use of suitable agents that delay absorption, such as aluminum monostearate and gelatin. In some embodiments, sterile injectable solutions can be prepared by incorporating genetically modified cells of the present disclosure in a sufficient amount of the appropriate solvent with various amounts of any other ingredients, as desired. Such compositions may be in admixture with a suitable carrier, diluent, or excipient such as sterile water, physiological saline, glucose, dextrose, or the like. In some embodiments, the compositions can also be lyophilized. The compositions can contain auxiliary substances such as wetting, dispersing agents, pH buffering agents, and antimicrobials depending upon the route of administration and the preparation desired.

In some embodiments, the components of the formulations of the present disclosure are selected to be chemically inert and to not affect the viability or efficacy of the genetically modified cells of the present disclosure.

One consideration concerning the therapeutic use of the genetically modified cells of the present disclosure is the quantity of cells needed to achieve optimal efficacy. In some embodiments, the quantity of cells to be administered will vary for the subject being treated. In certain embodiments, the quantity of genetically modified cells that are administered to a subject in need thereof may range from 1×10⁴ cells to 1×10¹⁰ cells. In some embodiments, the precise quantity of cells that would be considered an effective dose may be based on factors individual to each subject, including their size, age, sex, weight, and condition of the particular subject. Dosages can be readily ascertained by those skilled in the art based on the present disclosure and the knowledge in the art.

Whether it is a polypeptide, antibody, nucleic acid, small molecule or other pharmaceutically useful compound according to the present invention that is to be given to an individual, administration is preferably in a “therapeutically effective amount” or “prophylactically effective amount” (as the case can be, although prophylaxis can be considered therapy), this being sufficient to show benefit to the individual. The actual amount administered, and rate and time-course of administration, will depend on the nature and severity of protein aggregation disease being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

A composition can be administered alone or in combination with other treatments, either simultaneously or sequentially dependent upon the condition to be treated.

### Kits

Certain aspects of the present disclosure relate to kits for the treatment and/or prevention of a cancer or other diseases (e.g., immune-related or autoimmune disorders). In certain embodiments, the kit includes a therapeutic or prophylactic composition comprising an effective amount of one or more chimeric receptors of the present disclosure, isolated nucleic acids of the present disclosure, vectors of the present disclosure, and/or cells of the present disclosure (e.g., immunoresponsive cells). In some embodiments, the kit comprises a sterile container. In some embodiments, such containers can be boxes, ampules, bottles, vials, tubes, bags, pouches, blister-packs, or other suitable container forms known in the art. The container may be made of plastic, glass, laminated paper, metal foil, or other materials suitable for holding medicaments.

In some embodiments, therapeutic or prophylactic composition is provided together with instructions for administering the therapeutic or prophylactic composition to a subject having or at risk of developing a cancer or immune-related disorder. In some embodiments, the instructions may include information about the use of the composition for the treatment and/or prevention of the disorder. In some embodiments, the instructions include, without limitation, a description of the therapeutic or prophylactic composition, a dosage schedule, an administration schedule for treatment or prevention of the disorder or a symptom thereof, precautions, warnings, indications, counter-indications, over-dosage information, adverse reactions, animal pharmacology, clinical studies, and/or references. In some embodiments, the instructions can be printed directly on the container (when present), or as a label applied to the container, or as a separate sheet, pamphlet, card, or folder supplied in or with the container.

### ADDITIONAL EMBODIMENTS

Provided below are enumerated embodiments describing specific embodiments of the invention:

Embodiment 1: A chimeric inhibitory receptor comprising:
an extracellular protein binding domain,
a transmembrane domain, wherein the transmembrane domain is operably linked to the extracellular protein binding domain, and
an intracellular signaling domain, wherein the intracellular signaling domain is operably linked to the transmembrane domain; and
   wherein the intracellular signaling domain is capable of preventing, attenuating, or inhibiting activation of a tumor-targeting chimeric receptor expressed on an immunomodulatory cell.

Embodiment 2: The chimeric inhibitory receptor of embodiment 1, wherein the intracellular signaling domain is derived from a protein selected from the group consisting of: BTLA, PD-1, CTLA4, TIM3, KIR3DL1, LIR1, NKG2A, TIGIT, and LAG3.

Embodiment 3: The chimeric inhibitory receptor of embodiments 1 or embodiment, wherein the transmembrane domain and the intracellular signaling domain are derived from the same protein.

Embodiment 4: The chimeric inhibitory receptor of embodiment, wherein the transmembrane domain further comprises at least a portion of an extracellular domain of the same protein.

Embodiment 5: The chimeric inhibitory receptor of embodiment 1 or embodiment, wherein the transmembrane domain is derived from a first protein and the intracellular signaling domain is derived from a second protein that is distinct from the first protein.

Embodiment 6: The chimeric inhibitory receptor of any one of embodiments 1-, wherein the intracellular signaling domain is derived from BTLA.

Embodiment 7: The chimeric inhibitory receptor of embodiment, wherein the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 3)
RRHQGKQNELSDTAGREINLVDAHLKSEQTEASTRQNSQVLLSETGIYDN
DPDLCFRMQEGSEVYSNPCLEENKPGIVYASLNHSVIGPNSRLARNVKEA
PTEYASICVRS.

Embodiment 8: The chimeric inhibitory receptor of embodiment, wherein the intracellular signaling domain comprises the amino acid sequence of

(SEQ ID NO: 3)
RRHQGKQNELSDTAGREINLVDAHLKSEQTEASTRQNSQVLLSETGIYDN
DPDLCFRMQEGSEVYSNPCLEENKPGIVYASLNHSVIGPNSRLARNVKEA
PTEYASICVRS.

Embodiment 9: The chimeric inhibitory receptor of any one of embodiments 1-, wherein the intracellular signaling domain is derived from LIR1.

Embodiment 10: The chimeric inhibitory receptor of embodiment, wherein the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 50)
LRHRRQGKHWTSTQRKADFQHPAGAVGPEPTDRGLQWRSSPAADAQEENL
YAAVKHTQPEDGVEMDTRSPHDEDPQAVTYAEVKHSRPRREMASPPSPLS
GEFLDTKDRQAEEDRQMDTEAAASEAPQDVTYAQLHSLTLRREATEPPPS
QEGPSPAVPSIYATLAIH.

Embodiment 11: The chimeric inhibitory receptor of embodiment, wherein the intracellular signaling domain comprises the amino acid sequence of

(SEQ ID NO: 50)
LRHRRQGKHWTSTQRKADFQHPAGAVGPEPTDRGLQWRSSPAADAQEENL
YAAVKHTQPEDGVEMDTRSPHDEDPQAVTYAEVKHSRPRREMASPPSPLS
GEFLDTKDRQAEEDRQMDTEAAASEAPQDVTYAQLHSLTLRREATEPPPS
QEGPSPAVPSIYATLAIH.

Embodiment 12: The chimeric inhibitory receptor of any one of embodiments 1-, wherein the intracellular signaling domain is derived from KIR3DL1.

Embodiment 13: The chimeric inhibitory receptor of embodiment, wherein the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 66)
HLWCSNKKNAAVMDQEPAGNRTANSEDSDEQDPEEVTYAQLDHCVFTQRK
ITRPSQRPKTPPTDTILYTELPNAKPRSKVVSCP.

Embodiment 14: The chimeric inhibitory receptor of embodiment, wherein the intracellular signaling domain comprises the amino acid sequence of

(SEQ ID NO: 66)
HLWCSNKKNAAVMDQEPAGNRTANSEDSDEQDPEEVTYAQLDHCVFTQRK
ITRPSQRPKTPPTDTILYTELPNAKPRSKVVSCP.

Embodiment 15: The chimeric inhibitory receptor of any one of embodiments 1-, wherein the intracellular signaling domain is derived from PD-1.

Embodiment 16: The chimeric inhibitory receptor of embodiment, wherein the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 1)
CSRAARGTIGARRTGQPLKEDPSAVPVFSVDYGELDFQWREKTPEPPVPC
VPEQTEYATIVFPSGMGTSSPARRGSADGPRSAQPLRPEDGHCSWPL.

Embodiment 17: The chimeric inhibitory receptor of embodiment, wherein the intracellular signaling domain comprises the amino acid sequence of

(SEQ ID NO: 1)
CSRAARGTIGARRTGQPLKEDPSAVPVFSVDYGELDFQWREKTPEPPVPC
VPEQTEYATIVFPSGMGTSSPARRGSADGPRSAQPLRPEDGHCSWPL.

Embodiment 18: The chimeric inhibitory receptor of any one of embodiments 1-, wherein the intracellular signaling domain is derived from CTLA4.

Embodiment 19: The chimeric inhibitory receptor of embodiment, wherein the intracellular signaling domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 67)
AVSLSKMLKKRSPLTTGVGVKMPPTEPECEKQFQPYFIPIN.

Embodiment 20: The chimeric inhibitory receptor of embodiment, wherein the intracellular signaling domain comprises the amino acid sequence of

(SEQ ID NO: 67)
AVSLSKMLKKRSPLTTGVGVKMPPTEPECEKQFQPYFIPIN.

Embodiment 21: The chimeric inhibitory receptor of any one of embodiments 1-, wherein the transmembrane domain is derived from a protein selected from the group consisting of: BTLA, CD8, CD28, CD3zeta, CD4, 4-IBB, OX40, ICOS, 2B4, CD25, CD7, LAX, LAT, PD-1, CTLA4, TIM3, KIR3DL1, LIR1, NKG2A, TIGIT, and LAG3.

Embodiment 22: The chimeric inhibitory receptor of any one of embodiments 1-, wherein the chimeric inhibitory receptor comprises a transmembrane domain derived from BTLA.

Embodiment 23: The chimeric inhibitory receptor of embodiment, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LLPLGGLPLLITTCFCLFCCL (SEQ ID NO: 12).

Embodiment 24: The chimeric inhibitory receptor of embodiment, wherein the transmembrane domain comprises the amino acid sequence of

(SEQ ID NO: 12)
LLPLGGLPLLITTCFCLFCCL.

Embodiment 25: The chimeric inhibitory receptor of any one of embodiments —, wherein the transmembrane domain further comprises at least a portion of the BTLA extracellular domain.

Embodiment 26: The chimeric inhibitory receptor of any one of embodiments 1-, wherein the chimeric inhibitory receptor comprises a transmembrane domain derived from PD-1.

Embodiment 27: The chimeric inhibitory receptor of embodiment, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VGVVGGLLGSLVLLVWVLAVI (SEQ ID NO: 60).

Embodiment 28: The chimeric inhibitory receptor of embodiment, wherein the transmembrane domain comprises the amino acid sequence of

(SEQ ID NO: 60)
VGVVGGLLGSLVLLVWVLAVI.

Embodiment 29: The chimeric inhibitory receptor of any one of embodiments —, wherein the transmembrane domain further comprises at least a portion of the PD-1 extracellular domain.

Embodiment 30: The chimeric inhibitory receptor of any one of embodiments 1-, wherein the chimeric inhibitory receptor comprises a transmembrane domain derived from CTLA4.

Embodiment 31: The chimeric inhibitory receptor of embodiment, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to DFLLWILAAVSSGLFFYSFLLT (SEQ ID NO: 68).

Embodiment 32: The chimeric inhibitory receptor of embodiment, wherein the transmembrane domain comprises the amino acid sequence of

(SEQ ID NO: 68)
DFLLWILAAVSSGLFFYSFLLT.

Embodiment 33: The chimeric inhibitory receptor of any one of embodiments—, wherein the transmembrane domain further comprises at least a portion of the CTLA4 extracellular domain.

Embodiment 34: The chimeric inhibitory receptor of any one of embodiments 1-, wherein the chimeric inhibitory receptor comprises a transmembrane domain derived from KIR3DL1.

Embodiment 35: The chimeric inhibitory receptor of embodiment, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to ILIGTSVVIILFILLLFFLL (SEQ ID NO: 69).

Embodiment 36: The chimeric inhibitory receptor of embodiment, wherein the transmembrane domain comprises the amino acid sequence of ILIGTSVVIILFILLLFFLL (SEQ ID NO: 69).

Embodiment 37: The chimeric inhibitory receptor of any one of embodiments—, wherein the transmembrane domain further comprises at least a portion of the KIR3DL1 extracellular domain.

Embodiment 38: The chimeric inhibitory receptor of any one of embodiments 1-, wherein the chimeric inhibitory receptor comprises a transmembrane domain derived from LIR1.

Embodiment 39: The chimeric inhibitory receptor of embodiment, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VIGILVAVILLLLLLLLLFLI (SEQ ID NO: 59).

Embodiment 40: The chimeric inhibitory receptor of embodiment, wherein the transmembrane domain comprises the amino acid sequence of VIGILVAVILLLLLLLLLFLI (SEQ ID NO: 59).

Embodiment 41: The chimeric inhibitory receptor of any one of embodiments—, wherein the transmembrane domain further comprises at least a portion of the LIR1 extracellular domain.

Embodiment 42: The chimeric inhibitory receptor of any one of embodiments 1-, wherein the chimeric inhibitory receptor comprises a transmembrane domain derived from CD28.

Embodiment 43: The chimeric inhibitory receptor of embodiment, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 11)
FWVLVVVGGVLACYSLLVTVAFIIFWV.

Embodiment 44: The chimeric inhibitory receptor of embodiment, wherein the transmembrane domain comprises the amino acid sequence of

(SEQ ID NO: 11)
FWVLVVVGGVLACYSLLVTVAFIIFWV.

Embodiment 45: The chimeric inhibitory receptor of any one of embodiments—, wherein the transmembrane domain further comprises at least a portion of the CD28 extracellular domain.

Embodiment 46: The chimeric inhibitory receptor of any one of embodiments 1-, wherein the protein is not expressed on the target tumor.

Embodiment 47: The chimeric inhibitory receptor of any one of embodiments 1-, wherein the protein is expressed on a non-tumor cell.

Embodiment 48: The chimeric inhibitory receptor of embodiment, wherein the protein is expressed on a non-tumor cell derived from a tissue selected from the group consisting of: brain, neuronal tissue, endocrine, endothelial, bone, bone marrow, immune system, muscle, lung, liver, gallbladder, pancreas, gastrointestinal tract, kidney, urinary bladder, male reproductive organs, female reproductive organs, adipose, soft tissue, and skin.

Embodiment 49: The chimeric inhibitory receptor of any one of embodiments 1-, wherein the extracellular protein binding domain comprises a ligand-binding domain.

Embodiment 50: The chimeric inhibitory receptor of any one of embodiments 1-, wherein the extracellular protein binding domain comprises a receptor-binding domain.

Embodiment 51: The chimeric inhibitory receptor of any one of embodiments 1-, wherein the extracellular protein binding domain comprises an antigen-binding domain.

Embodiment 52: The chimeric inhibitory receptor of embodiment, wherein in the antigen-binding domain comprises an antibody, an antigen-binding fragment of an antibody, a F(ab) fragment, a F(ab′) fragment, a single chain variable fragment (scFv), or a single-domain antibody (sdAb).

Embodiment 53: The chimeric inhibitory receptor of embodiment, wherein the antigen-binding domain comprises a single chain variable fragment (scFv).

Embodiment 54: The chimeric inhibitory receptor of embodiment, wherein each scFv comprises a heavy chain variable domain (VH) and a light chain variable domain (VL).

Embodiment 55: The chimeric inhibitory receptor of embodiment, wherein the VH and VL are separated by a peptide linker.

Embodiment 56: The chimeric inhibitory receptor of embodiment, wherein the peptide linker comprises an amino acid sequence selected from the group consisting of: GGS (SEQ ID NO: 15), GGSGGS (SEQ ID NO: 16), GGSGGSGGS (SEQ ID NO: 17), GGSGGSGGSGGS (SEQ ID NO: 18), GGSGGSGGSGGSGGS (SEQ ID NO: 19), GGGS (SEQ ID NO: 20), GGGSGGGS (SEQ ID NO: 21), GGGSGGGSGGGS (SEQ ID NO: 22), GGGSGGGSGGGSGGGS (SEQ ID NO: 23), GGGSGGGSGGGSGGGSGGGS (SEQ ID NO: 24), GGGGS (SEQ ID NO: 25), GGGGSGGGGS (SEQ ID NO: 26), GGGGSGGGGSGGGGS (SEQ ID NO: 27), GGGGSGGGGSGGGGSGGGGS (SEQ ID NO: 28), and GGGGSGGGGSGGGGSGGGGSGGGGS (SEQ ID NO: 29).

Embodiment 57: The chimeric inhibitory receptor of any one of embodiments—, wherein the scFv comprises the structure VH-L-VL or VL-L-VH, wherein VH is the heavy chain variable domain, L is the peptide linker, and VL is the light chain variable domain.

Embodiment 58: The chimeric inhibitory receptor of any one of embodiments 1-, wherein the transmembrane domain is physically linked to the extracellular protein binding domain.

Embodiment 59: The chimeric inhibitory receptor of any one of embodiments 1-, wherein the intracellular signaling domain is physically linked to the transmembrane domain.

Embodiment 60: The chimeric inhibitory receptor of any one of embodiments 1-, wherein the transmembrane domain is physically linked to the extracellular protein binding domain and the intracellular signaling domain is physically linked to the transmembrane domain.

Embodiment 61: The chimeric inhibitory receptor of any one of embodiments 1-, wherein the extracellular protein binding domain has a high binding affinity.

Embodiment 62: The chimeric inhibitory receptor of any one of embodiments 1-, wherein the extracellular protein binding domain has a low binding affinity.

Embodiment 63: The chimeric inhibitory receptor of any one of embodiments 1-, wherein the chimeric inhibitory receptor is capable of suppressing cytokine production by an activated immunomodulatory cell.

Embodiment 64: The chimeric inhibitory receptor of any one of embodiments 1-, wherein the chimeric inhibitory receptor is capable of suppressing a cell-mediated immune response to a target cell, wherein the immune response is induced by activation of the immunomodulatory cell.

Embodiment 65: The chimeric inhibitory receptor of any one of embodiments 1-, wherein the target cell is a tumor cell.

Embodiment 66: The chimeric inhibitory receptor of any one of embodiments 1-, wherein the intracellular signaling domain comprises one or more modifications.

Embodiment 67: The chimeric inhibitory receptor of embodiment, wherein the one or more modifications modulate sensitivity of the chimeric inhibitory receptor relative to the otherwise identical, unmodified receptor.

Embodiment 68: The chimeric inhibitory receptor of embodiment, wherein the one or more modifications increase sensitivity of the chimeric inhibitory receptor relative to the otherwise identical, unmodified receptor.

Embodiment 69: The chimeric inhibitory receptor of embodiment, wherein the one or more modifications reduce sensitivity of the chimeric inhibitory receptor relative to the otherwise identical, unmodified receptor.

Embodiment 70: The chimeric inhibitory receptor of any one embodiments—, wherein the one or more modifications modulate potency of the chimeric inhibitory receptor relative to the otherwise identical, unmodified receptor.

Embodiment 71: The chimeric inhibitory receptor of embodiment, wherein the one or more modifications increase potency of the chimeric inhibitory receptor relative to the otherwise identical, unmodified receptor.

Embodiment 72: The chimeric inhibitory receptor of embodiment, wherein the one or more modifications reduce potency of the chimeric inhibitory receptor relative to the otherwise identical, unmodified receptor.

Embodiment 73: The chimeric inhibitory receptor of any one of embodiments—, wherein the one or more modifications modulate basal prevention, attenuation, or inhibition of activation of the tumor-targeting chimeric receptor when expressed on an immunomodulatory cell relative to the otherwise identical, unmodified receptor.

Embodiment 74: The chimeric inhibitory receptor of embodiment, wherein the one or more modifications reduce basal prevention, attenuation, or inhibition relative to the otherwise identical, unmodified receptor.

Embodiment 75: The chimeric inhibitory receptor of embodiment, wherein the one or more modifications increase basal prevention, attenuation, or inhibition relative to the otherwise identical, unmodified receptor.

Embodiment 76: The chimeric inhibitory receptor of any one of embodiments 1-, wherein the chimeric inhibitory receptor further comprises a spacer region positioned between the extracellular protein binding domain and the transmembrane domain and operably linked to each of the extracellular protein binding domain and the transmembrane domain.

Embodiment 77: The chimeric inhibitory receptor of any one of embodiments 1-, wherein the chimeric inhibitory receptor further comprises a spacer region positioned between the extracellular protein binding domain and the transmembrane domain and physically linked to each of the extracellular protein binding domain and the transmembrane domain.

Embodiment 78: The chimeric inhibitory receptor of embodiment or embodiment, wherein the spacer region is derived from a protein selected from the group consisting of: CD8alpha, CD4, CD7, CD28, IgG1, IgG4, FcgammaRIIIalpha, LNGFR, and PDGFR.

Embodiment 79: The chimeric inhibitory receptor of embodiment or embodiment, wherein the spacer region comprises an amino acid sequence selected from the group consisting of: AAAIEVMYPPPYLDNEKSNGTIIHVKGKHLCPSPLFPGPSKP (SEQ ID NO: 31), ESKYGPPCPSCP (SEQ ID NO: 32), ESKYGPPAPSAP (SEQ ID NO: 33), ESKYGPPCPPCP (SEQ ID NO: 34), EPKSCDKTHTCP (SEQ ID NO: 35), AAAFVPVFLPAKPTTTPAPRPPTPAPTIASQPLSLRPEACRPAAGGAVHTRGLDFACDI YIWAPLAGTCGVLLLSLVITLYCNHRN (SEQ ID NO: 36), TTTPAPRPPTPAPTIALQPLSLRPEACRPAAGGAVHTRGLDFACD (SEQ ID NO: 37) ACPTGLYTHSGECCKACNLGEGVAQPCGANQTVCEPCLDSVTFSDVVSATEPCKPCT ECVGLQSMSAPCVEADDAVCRCAYGYYQDETTGRCEACRVCEAGSGLVFSCQDKQ NTVCEECPDGTYSDEADAEC (SEQ ID NO: 38), ACPTGLYTHSGECCKACNLGEGVAQPCGANQTVC (SEQ ID NO: 39), AVGQDTQEVIVVPHSLPFKV (SEQ ID NO: 40), and TTTPAPRPPTPAPTIALQPLSLRPEACRPAAGGAVHTRGLDFACDQTTPGERSSLPAFY PGTSGSCSGCGSLSLP (SEQ ID NO: 70).

Embodiment 80: The chimeric inhibitory receptor of any one of embodiments—, wherein the spacer region modulates sensitivity of the chimeric inhibitory receptor relative to an otherwise identical chimeric inhibitory receptor lacking the spacer region.

Embodiment 81: The chimeric inhibitory receptor of embodiment, wherein the spacer region increases sensitivity of the chimeric inhibitory receptor relative to an otherwise identical chimeric inhibitory receptor lacking the spacer region.

Embodiment 82: The chimeric inhibitory receptor of embodiment, wherein the spacer region reduces sensitivity of the chimeric inhibitory receptor relative to an otherwise identical chimeric inhibitory receptor lacking the spacer region.

Embodiment 83: The chimeric inhibitory receptor of any one of embodiments—, wherein the spacer region modulates potency of the chimeric inhibitory receptor relative to an otherwise identical chimeric inhibitory receptor lacking the spacer region.

Embodiment 84: The chimeric inhibitory receptor of embodiment, wherein the spacer region increases potency of the chimeric inhibitory receptor relative to an otherwise identical chimeric inhibitory receptor lacking the spacer region.

Embodiment 85: The chimeric inhibitory receptor of embodiment, wherein the spacer region reduces potency of the chimeric inhibitory receptor relative to an otherwise identical chimeric inhibitory receptor lacking the spacer region.

Embodiment 86: The chimeric inhibitory receptor of any one of embodiments—, wherein the spacer region modulates basal prevention, attenuation, or inhibition of activation of the tumor-targeting chimeric receptor when expressed on an immunomodulatory cell relative to an otherwise identical chimeric inhibitory receptor lacking the spacer region.

Embodiment 87: The chimeric inhibitory receptor of embodiment, wherein the spacer region reduces basal prevention, attenuation, or inhibition relative to an otherwise identical chimeric inhibitory receptor lacking the spacer region.

Embodiment 88: The chimeric inhibitory receptor of embodiment, wherein the spacer region increases basal prevention, attenuation, or inhibition relative to an otherwise identical chimeric inhibitory receptor lacking the spacer region.

Embodiment 89: The chimeric inhibitory receptor of any one of embodiments 1-, wherein the chimeric inhibitory receptor further comprises an intracellular spacer region positioned between the transmembrane domain and the intracellular signaling domain and operably linked to each of the transmembrane domain and the intracellular signaling domain.

Embodiment 90: The chimeric inhibitory receptor of any one of embodiments 1-, wherein the chimeric inhibitory receptor further comprises an intracellular spacer region positioned between the transmembrane domain and the intracellular signaling domain and physically linked to each of the transmembrane domain and the intracellular signaling domain.

Embodiment 91: The chimeric inhibitory receptor of embodiment or embodiment, wherein the intracellular spacer region modulates sensitivity of the chimeric inhibitory receptor relative to an otherwise identical chimeric inhibitory receptor lacking the intracellular spacer region.

Embodiment 92: The chimeric inhibitory receptor of embodiment, wherein the intracellular spacer region increases sensitivity of the chimeric inhibitory receptor relative to an otherwise identical chimeric inhibitory receptor lacking the intracellular spacer region.

Embodiment 93: The chimeric inhibitory receptor of embodiment, wherein the intracellular spacer region reduces sensitivity of the chimeric inhibitory receptor relative to an otherwise identical chimeric inhibitory receptor lacking the intracellular spacer region.

Embodiment 94: The chimeric inhibitory receptor of any one of embodiments—, wherein the intracellular spacer region modulates potency of the chimeric inhibitory receptor relative to an otherwise identical chimeric inhibitory receptor lacking the intracellular spacer region.

Embodiment 95: The chimeric inhibitory receptor of embodiment, wherein the intracellular spacer region increases potency of the chimeric inhibitory receptor relative to an otherwise identical chimeric inhibitory receptor lacking the intracellular spacer region.

Embodiment 96: The chimeric inhibitory receptor of embodiment, wherein the intracellular spacer region reduces potency of the chimeric inhibitory receptor relative to an otherwise identical chimeric inhibitory receptor lacking the intracellular spacer region.

Embodiment 97: The chimeric inhibitory receptor of any one of embodiments—, wherein the intracellular spacer region modulates basal prevention, attenuation, or inhibition of activation of the tumor-targeting chimeric receptor when expressed on an immunomodulatory cell relative to an otherwise identical chimeric inhibitory receptor lacking the intracellular spacer region.

Embodiment 98: The chimeric inhibitory receptor of embodiment, wherein the intracellular spacer region reduces basal prevention, attenuation, or inhibition relative to an otherwise identical chimeric inhibitory receptor lacking the intracellular spacer region.

Embodiment 99: The chimeric inhibitory receptor of embodiment, wherein the intracellular spacer region increases basal prevention, attenuation, or inhibition relative to an otherwise identical chimeric inhibitory receptor lacking the intracellular spacer region.

Embodiment 100: The chimeric inhibitory receptor of any one of embodiments 1-, wherein the inhibitory chimeric receptor further comprises an enzymatic inhibitory domain.

Embodiment 101: The chimeric inhibitory receptor of embodiment, wherein the enzymatic inhibitory domain is capable of preventing, attenuating, or inhibiting activation of a tumor-targeting chimeric receptor when expressed on an immunomodulatory cell relative to an otherwise identical chimeric inhibitory receptor lacking the enzymatic inhibitory domain.

Embodiment 102: The chimeric inhibitory receptor of embodiment or embodiment, wherein the enzymatic inhibitory domain comprises an enzyme catalytic domain.

Embodiment 103: The chimeric inhibitory receptor of embodiment, wherein the enzyme catalytic domain is derived from an enzyme selected from the group consisting of: CSK, SHP-1, PTEN, CD45, CD148, PTP-MEG1, PTP-PEST, c-CBL, CBL-b, PTPN22, LAR, PTPH1, SHIP-1, and RasGAP.

Embodiment 104: The chimeric inhibitory receptor of any one of embodiments—, wherein the enzymatic inhibitory domain comprises one or more modifications that modulate basal prevention, attenuation, or inhibition relative to an otherwise identical enzymatic inhibitory domain lacking the one or more modifications.

Embodiment 105: The chimeric inhibitory receptor of embodiment, wherein the one or more modifications reduce basal prevention, attenuation, or inhibition of the chimeric inhibitory receptor relative to an otherwise identical chimeric inhibitory receptor lacking the enzymatic inhibitory domain.

Embodiment 106: The chimeric inhibitory receptor of embodiment, wherein the one or more modifications increase basal prevention, attenuation, or inhibition of the chimeric inhibitory receptor relative to an otherwise identical chimeric inhibitory receptor lacking the enzymatic inhibitory domain.

Embodiment 107: The chimeric inhibitory receptor of any one of embodiments 1-, wherein the tumor-targeting chimeric receptor is a tumor-targeting chimeric antigen receptor (CAR) or an engineered T cell receptor (TCR).

Embodiment 108: The chimeric inhibitory receptor of any one of embodiments 1-, wherein the immunomodulatory cell is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an ESC-derived cell, and an iPSC-derived cell.

Embodiment 109: The chimeric inhibitory receptor of any one of embodiments 1-, wherein the immunomodulatory cell is a Natural Killer (NK) cell.

Embodiment 110: A chimeric inhibitory receptor comprising:
an extracellular protein binding domain,
a transmembrane domain, wherein the transmembrane domain is operably linked to the extracellular protein binding domain, and
two or more intracellular signaling domains, wherein the two or more intracellular signaling domains are operably linked to the transmembrane domain; and
   wherein at least one of the two or more intracellular signaling domains is capable of preventing, attenuating, or inhibiting activation of a tumor-targeting chimeric receptor expressed on an immunomodulatory cell.

Embodiment 111: The chimeric inhibitory receptor of embodiment, wherein the two or more intracellular signaling domains are each derived from a protein selected from the group consisting of: BTLA, PD-1, CTLA4, TIM3, KIR3DL1, LIR1, NKG2A, TIGIT, and LAG3.

Embodiment 112: The chimeric inhibitory receptor of embodiment or embodiment, wherein the transmembrane domain is derived from the same protein as one of the two or more intracellular signaling domains.

Embodiment 113: The chimeric inhibitory receptor of embodiment, wherein the transmembrane domain further comprises at least a portion of an extracellular domain of the same protein.

Embodiment 114: The chimeric inhibitory receptor of embodiment or embodiment, wherein the transmembrane domain is derived from a first protein and the two or more intracellular signaling domains are derived from proteins that are distinct from the first protein.

Embodiment 115: The chimeric inhibitory receptor of any one of embodiments—, wherein at least one of the two or more intracellular signaling domains is derived from BTLA.

Embodiment 116: The chimeric inhibitory receptor of embodiment, wherein the at least one of the two or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 3)
RRHQGKQNELSDTAGREINLVDAHLKSEQTEASTRQNSQVLLSETGIYDN
DPDLCFRMQEGSEVYSNPCLEENKPGIVYASLNHSVIGPNSRLARNVKEA
PTEYASICVRS.

Embodiment 117: The chimeric inhibitory receptor of embodiment, wherein the at least one of the two or more intracellular signaling domains comprises the amino acid sequence of

(SEQ ID NO: 3)
RRHQGKQNELSDTAGREINLVDAHLKSEQTEASTRQNSQVLLSETGIYDN
DPDLCFRMQEGSEVYSNPCLEENKPGIVYASLNHSVIGPNSRLARNVKEA
PTEYASICVRS.

Embodiment 118: The chimeric inhibitory receptor of any one of embodiments—, wherein at least one of the two or more intracellular signaling domains is derived from LIR1.

Embodiment 119: The chimeric inhibitory receptor of embodiment, wherein the at least one of the two or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 50)
LRHRRQGKHWTSTQRKADFQHPAGAVGPEPTDRGLQWRSSPAADAQEENL
YAAVKHTQPEDGVEMDTRSPHDEDPQAVTYAEVKHSRPRREMASPPSPLS
GEFLDTKDRQAEEDRQMDTEAAASEAPQDVTYAQLHSLTLRREATEPPPS
QEGPSPAVPSIYATLAIH.

Embodiment 120: The chimeric inhibitory receptor of embodiment, wherein the at least one of the two or more intracellular signaling domains comprises the amino acid sequence of

(SEQ ID NO: 50)
LRHRRQGKHWTSTQRKADFQHPAGAVGPEPTDRGLQWRSSPAADAQEENL
YAAVKHTQPEDGVEMDTRSPHDEDPQAVTYAEVKHSRPRREMASPPSPLS
GEFLDTKDRQAEEDRQMDTEAAASEAPQDVTYAQLHSLTLRREATEPPPS
QEGPSPAVPSIYATLAIH.

Embodiment 121: The chimeric inhibitory receptor of any one of embodiments—, wherein at least one of the two or more intracellular signaling domains is derived from PD-1.

Embodiment 122: The chimeric inhibitory receptor of embodiment, wherein the at least one of the two or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 1)
CSRAARGTIGARRTGQPLKEDPSAVPVFSVDYGELDFQWREKTPEPPVPC
VPEQTEYATIVFPSGMGTSSPARRGSADGPRSAQPLRPEDGHCSWPL.

Embodiment 123: The chimeric inhibitory receptor of embodiment, wherein the at least one of the two or more intracellular signaling domains comprises the amino acid sequence of

(SEQ ID NO: 1)
CSRAARGTIGARRTGQPLKEDPSAVPVFSVDYGELDFQWREKTPEPPVPC
VPEQTEYATIVFPSGMGTSSPARRGSADGPRSAQPLRPEDGHCSWPL.

Embodiment 124: The chimeric inhibitory receptor of any one of embodiments—, wherein at least one of the two or more intracellular signaling domains is derived from KIR3DL1.

Embodiment 125: The chimeric inhibitory receptor of embodiment 124, wherein the at least one of the two or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 66)
HLWCSNKKNAAVMDQEPAGNRTANSEDSDEQDPEEVTYAQLDHCVFTQRK
ITRPSQRPKTPPTDTILYTELPNAKPRSKVVSCP.

Embodiment 126: The chimeric inhibitory receptor of embodiment 124, wherein the at least one of the two or more intracellular signaling domains comprises the amino acid sequence of

(SEQ ID NO: 66)
HLWCSNKKNAAVMDQEPAGNRTANSEDSDEQDPEEVTYAQLDHCVFTQRK
ITRPSQRPKTPPTDTILYTELPNAKPRSKVVSCP.

Embodiment 127: The chimeric inhibitory receptor of any one of embodiments—, wherein at least one of the two or more intracellular signaling domains is derived from CTLA4.

Embodiment 128: The chimeric inhibitory receptor of embodiment, wherein the at least one of the two or more intracellular signaling domains comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 67)
AVSLSKMLKKRSPLTTGVGVKMPPTEPECEKQFQPYFIPIN.

Embodiment 129: The chimeric inhibitory receptor of embodiment, wherein the intracellular signaling domain comprises the amino acid sequence of

(SEQ ID NO: 67)
AVSLSKMLKKRSPLTTGVGVKMPPTEPECEKQFQPYFIPIN.

Embodiment 130: The chimeric inhibitory receptor of any one of embodiments—, wherein the transmembrane domain is derived from a protein selected from the group consisting of: BTLA, CD8, CD28, CD3zeta, CD4, 4-IBB, OX40, ICOS, 2B4, CD25, CD7, LAX, LAT, PD-1, CTLA4, TIM3, KIR3DL1, LIR1, NKG2A, TIGIT, and LAG3.

Embodiment 131: The chimeric inhibitory receptor of any one of embodiments—, wherein the chimeric inhibitory receptor comprises a transmembrane domain derived from BTLA.

Embodiment 132: The chimeric inhibitory receptor of embodiment, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to LLPLGGLPLLITTCFCLFCCL (SEQ ID NO: 12).

Embodiment 133: The chimeric inhibitory receptor of embodiment, wherein the transmembrane domain comprises the amino acid sequence of

(SEQ ID NO: 12)
LLPLGGLPLLITTCFCLFCCL.

Embodiment 134: The chimeric inhibitory receptor of any one of embodiments—, wherein the transmembrane domain further comprises at least a portion of the BTLA extracellular domain.

Embodiment 135: The chimeric inhibitory receptor of any one of embodiments—, wherein the chimeric inhibitory receptor comprises a transmembrane domain derived from LIR1.

Embodiment 136: The chimeric inhibitory receptor of embodiment, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VIGILVAVILLLLLLLLLFLI (SEQ ID NO: 59).

Embodiment 137: The chimeric inhibitory receptor of embodiment, wherein the transmembrane domain comprises the amino acid sequence of VIGILVAVILLLLLLLLLFLI (SEQ ID NO: 59).

Embodiment 138: The chimeric inhibitory receptor of any one of embodiments—, wherein the transmembrane domain further comprises at least a portion of the LIR1 extracellular domain.

Embodiment 139: The chimeric inhibitory receptor of any one of embodiments—, wherein the chimeric inhibitory receptor comprises a transmembrane domain derived from PD-1.

Embodiment 140: The chimeric inhibitory receptor of embodiment, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to VGVVGGLLGSLVLLVWVLAVI (SEQ ID NO: 60).

Embodiment 141: The chimeric inhibitory receptor of embodiment, wherein the transmembrane domain comprises the amino acid sequence of

(SEQ ID NO: 60)
VGVVGGLLGSLVLLVWVLAVI.

Embodiment 142: The chimeric inhibitory receptor of any one of embodiments—, wherein the transmembrane domain further comprises at least a portion of the PD-1 extracellular domain.

Embodiment 143: The chimeric inhibitory receptor of any one of embodiments—, wherein the chimeric inhibitory receptor comprises a transmembrane domain derived from CTLA4.

Embodiment 144: The chimeric inhibitory receptor of embodiment, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to DFLLWILAAVSSGLFFYSFLLT (SEQ ID NO: 68).

Embodiment 145: The chimeric inhibitory receptor of embodiment, wherein the transmembrane domain comprises the amino acid sequence of

(SEQ ID NO: 68)
DFLLWILAAVSSGLFFYSFLLT.

Embodiment 146: The chimeric inhibitory receptor of any one of embodiments—, wherein the transmembrane domain further comprises at least a portion of the CTLA4 extracellular domain.

Embodiment 147: The chimeric inhibitory receptor of any one of embodiments—, wherein the chimeric inhibitory receptor comprises a transmembrane domain derived from KIR3DL1.

Embodiment 148: The chimeric inhibitory receptor of embodiment 147, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to ILIGTSVVIILFILLLFFLL (SEQ ID NO: 69).

Embodiment 149: The chimeric inhibitory receptor of embodiment, wherein the transmembrane domain comprises the amino acid sequence of ILIGTSVVIILFILLLFFLL (SEQ ID NO: 69).

Embodiment 150: The chimeric inhibitory receptor of any one of embodiments—, wherein the transmembrane domain further comprises at least a portion of the KIR3DL1 extracellular domain.

Embodiment 151: The chimeric inhibitory receptor of any one of embodiments—, wherein the chimeric inhibitory receptor comprises a transmembrane domain derived from CD28.

Embodiment 152: The chimeric inhibitory receptor of embodiment, wherein the transmembrane domain comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to

(SEQ ID NO: 11)
FWVLVVVGGVLACYSLLVTVAFIIFWV.

Embodiment 153: The chimeric inhibitory receptor of embodiment, wherein the transmembrane domain comprises the amino acid sequence of

(SEQ ID NO: 11)
FWVLVVVGGVLACYSLLVTVAFIIFWV.

Embodiment 154: The chimeric inhibitory receptor of any one of embodiments—, wherein the transmembrane domain further comprises at least a portion of the CD28 extracellular domain.

Embodiment 155: The chimeric inhibitory receptor of any one of embodiments—, wherein the chimeric inhibitory receptor comprises a first intracellular signaling domain derived from LIR1 and a second intracellular signaling domain derived from BTLA.

Embodiment 156: The chimeric inhibitory receptor of any one of embodiments—, wherein the chimeric inhibitory receptor comprises a first intracellular signaling domain derived from LIR1 and a second intracellular signaling domain derived from PD-1.

Embodiment 157: The chimeric inhibitory receptor of any one of embodiments—, wherein the chimeric inhibitory receptor comprises a first intracellular signaling domain derived from LIR1 and a second intracellular signaling domain derived from KIR3DL1.

Embodiment 158: The chimeric inhibitory receptor of any one of embodiments—, wherein the chimeric inhibitory receptor comprises a first intracellular signaling domain derived from LIR1 and a second intracellular signaling domain derived from LIR1.

Embodiment 159: The chimeric inhibitory receptor of any one of embodiments—, wherein the first intracellular signaling domain further comprises a transmembrane domain derived from LIR1.

Embodiment 160: The chimeric inhibitory receptor of any one of embodiments—, wherein the chimeric inhibitory receptor comprises a first intracellular signaling domain derived from BTLA and a second intracellular signaling domain derived from LIR1.

Embodiment 161: The chimeric inhibitory receptor of any one of embodiments—, wherein the chimeric inhibitory receptor comprises a first intracellular signaling domain derived from BTLA and a second intracellular signaling domain derived from PD-1.

Embodiment 162: The chimeric inhibitory receptor of embodiment or embodiment, wherein the first intracellular signaling domain further comprises a transmembrane domain derived from BTLA.

Embodiment 163: The chimeric inhibitory receptor of any one of embodiments—, wherein the chimeric inhibitory receptor comprises a first intracellular signaling domain derived from PD-1 and a second intracellular signaling domain derived from LIR1.

Embodiment 164: The chimeric inhibitory receptor of any one of embodiments—, wherein the chimeric inhibitory receptor comprises a first intracellular signaling domain derived from PD-1 and a second intracellular signaling domain derived from BTLA.

Embodiment 165: The chimeric inhibitory receptor of embodiment or embodiment, wherein the first intracellular signaling domain further comprises a transmembrane domain derived from PD-1.

Embodiment 166: The chimeric inhibitory receptor of any one of embodiments—, wherein the chimeric inhibitory receptor comprises a first intracellular signaling domain derived from KIR3DL1 and a second intracellular signaling domain derived from LIR1.

Embodiment 167: The chimeric inhibitory receptor of any one of embodiments—, wherein the chimeric inhibitory receptor comprises a first intracellular signaling domain derived from KIR3DL1 and a second intracellular signaling domain derived from KIR3DL1.

Embodiment 168: The chimeric inhibitory receptor of embodiment or embodiment, wherein the first intracellular signaling domain further comprises a transmembrane domain derived from KIR3DL1.

Embodiment 169: The chimeric inhibitory receptor of any one of embodiments—, wherein the protein is not expressed on the target tumor.

Embodiment 170: The chimeric inhibitory receptor of any one of embodiments—, wherein the protein is expressed on a non-tumor cell.

Embodiment 171: The chimeric inhibitory receptor of embodiment, wherein the protein is expressed on a non-tumor cell derived from a tissue selected from the group consisting of: brain, neuronal tissue, endocrine, endothelial, bone, bone marrow, immune system, muscle, lung, liver, gallbladder, pancreas, gastrointestinal tract, kidney, urinary bladder, male reproductive organs, female reproductive organs, adipose, soft tissue, and skin.

Embodiment 172: The chimeric inhibitory receptor of any one of embodiments—, wherein the extracellular protein binding domain comprises a ligand-binding domain.

Embodiment 173: The chimeric inhibitory receptor of any one of embodiments—, wherein the extracellular protein binding domain comprises a receptor-binding domain.

Embodiment 174: The chimeric inhibitory receptor of any one of embodiments—, wherein the extracellular protein binding domain comprises an antigen-binding domain.

Embodiment 175: The chimeric inhibitory receptor of embodiment, wherein the antigen-binding domain comprises an antibody, an antigen-binding fragment of an antibody, a F(ab) fragment, a F(ab′) fragment, a single chain variable fragment (scFv), or a single-domain antibody (sdAb).

Embodiment 176: The chimeric inhibitory receptor of embodiment, wherein the antigen-binding domain comprises a single chain variable fragment (scFv).

Embodiment 177: The chimeric inhibitory receptor of embodiment, wherein each scFv comprises a heavy chain variable domain (VH) and a light chain variable domain (VL).

Embodiment 178: The chimeric inhibitory receptor of embodiment, wherein the VH and VL are separated by a peptide linker.

Embodiment 179: The chimeric inhibitory receptor of embodiment, wherein the peptide linker comprises an amino acid sequence selected from the group consisting of: GGS (SEQ ID NO: 15), GGSGGS (SEQ ID NO: 16), GGSGGSGGS (SEQ ID NO: 17), GGSGGSGGSGGS (SEQ ID NO: 18), GGSGGSGGSGGSGGS (SEQ ID NO: 19), GGGS (SEQ ID NO: 20), GGGSGGGS (SEQ ID NO: 21), GGGSGGGSGGGS (SEQ ID NO: 22), GGGSGGGSGGGSGGGS (SEQ ID NO: 23), GGGSGGGSGGGSGGGSGGGS (SEQ ID NO: 24), GGGGS (SEQ ID NO: 25), GGGGSGGGGS (SEQ ID NO: 26), GGGGSGGGGSGGGGS (SEQ ID NO: 27), GGGGSGGGGSGGGGSGGGGS (SEQ ID NO: 28), and GGGGSGGGGSGGGGSGGGGSGGGGS (SEQ ID NO: 29).

Embodiment 180: The chimeric inhibitory receptor of any one of embodiments—, wherein the scFv comprises the structure VH-L-VL or VL-L-VH, wherein VH is the heavy chain variable domain, L is the peptide linker, and VL is the light chain variable domain.

Embodiment 181: The chimeric inhibitory receptor of any one of embodiments—, wherein the transmembrane domain is physically linked to the extracellular protein binding domain.

Embodiment 182: The chimeric inhibitory receptor of any one of embodiments—, wherein one of the two or more intracellular signaling domains is physically linked to the transmembrane domain.

Embodiment 183: The chimeric inhibitory receptor of any one of embodiments—, wherein the transmembrane domain is physically linked to the extracellular protein binding domain and one of the two or more intracellular signaling domains is physically linked to the transmembrane domain.

Embodiment 184: The chimeric inhibitory receptor of any one of embodiments—, wherein the extracellular protein binding domain has a high binding affinity.

Embodiment 185: The chimeric inhibitory receptor of any one of embodiments—, wherein extracellular protein binding domain has a low binding affinity.

Embodiment 186: The chimeric inhibitory receptor of any one of embodiments—, wherein the chimeric inhibitory receptor is capable of suppressing cytokine production by an activated immunomodulatory cell.

Embodiment 187: The chimeric inhibitory receptor of any one of embodiments—, wherein the chimeric inhibitory receptor is capable of suppressing a cell-mediated immune response to a target cell, wherein the immune response is induced by activation of the immunomodulatory cell.

Embodiment 188: The chimeric inhibitory receptor of any one of embodiments—, wherein the target cell is a tumor cell.

Embodiment 189: The chimeric inhibitory receptor of any one of embodiments—, wherein at least one of the two or more intracellular signaling domains comprises one or more modifications.

Embodiment 190: The chimeric inhibitory receptor of embodiment, wherein the one or more modifications modulate sensitivity of the chimeric inhibitory receptor relative to the otherwise identical, unmodified receptor.

Embodiment 191: The chimeric inhibitory receptor of embodiment, wherein the one or more modifications increase sensitivity of the chimeric inhibitory receptor relative to the otherwise identical, unmodified receptor.

Embodiment 192: The chimeric inhibitory receptor of embodiment, wherein the one or more modifications reduce sensitivity of the chimeric inhibitory receptor relative to the otherwise identical, unmodified receptor.

Embodiment 193: The chimeric inhibitory receptor of any one of embodiments—, wherein the one or more modifications modulate potency of the chimeric inhibitory receptor relative to the otherwise identical, unmodified receptor.

Embodiment 194: The chimeric inhibitory receptor of embodiment, wherein the one or more modifications increase potency of the chimeric inhibitory receptor relative to the otherwise identical, unmodified receptor.

Embodiment 195: The chimeric inhibitory receptor of embodiment, wherein the one or more modifications reduce potency of the chimeric inhibitory receptor relative to the otherwise identical, unmodified receptor.

Embodiment 196: The chimeric inhibitory receptor of any one of embodiments—, wherein the one or more modifications modulate basal prevention, attenuation, or inhibition of activation of the tumor-targeting chimeric receptor when expressed on an immunomodulatory cell relative to the otherwise identical, unmodified receptor.

Embodiment 197: The chimeric inhibitory receptor of embodiment, wherein the one or more modifications reduce basal prevention, attenuation, or inhibition relative to the otherwise identical, unmodified receptor.

Embodiment 198: The chimeric inhibitory receptor of embodiment, wherein the one or more modifications increase basal prevention, attenuation, or inhibition relative to the otherwise identical, unmodified receptor.

Embodiment 199: The chimeric inhibitory receptor of any one of embodiments—, wherein the chimeric inhibitory receptor further comprises a spacer region positioned between the extracellular protein binding domain and the transmembrane domain and operably linked to each of the extracellular protein binding domain and the transmembrane domain.

Embodiment 200: The chimeric inhibitory receptor of any one of embodiments—, wherein the chimeric inhibitory receptor further comprises a spacer region positioned between the extracellular protein binding domain and the transmembrane domain and physically linked to each of the extracellular protein binding domain and the transmembrane domain.

Embodiment 201: The chimeric inhibitory receptor of embodiment or embodiment, wherein the spacer region is derived from a protein selected from the group consisting of: CD8alpha, CD4, CD7, CD28, IgG1, IgG4, FcgammaRIIIalpha, LNGFR, and PDGFR.

Embodiment 202: The chimeric inhibitory receptor of embodiment or embodiment, wherein the spacer region comprises an amino acid sequence selected from the group consisting of: AAAIEVMYPPPYLDNEKSNGTIIHVKGKHLCPSPLFPGPSKP (SEQ ID NO: 31), ESKYGPPCPSCP (SEQ ID NO: 32), ESKYGPPAPSAP (SEQ ID NO: 33), ESKYGPPCPPCP (SEQ ID NO: 34), EPKSCDKTHTCP (SEQ ID NO: 35), AAAFVPVFLPAKPTTTPAPRPPTPAPTIASQPLSLRPEACRPAAGGAVHTRGLDFACDI YIWAPLAGTCGVLLLSLVITLYCNHRN (SEQ ID NO: 36), TTTPAPRPPTPAPTIALQPLSLRPEACRPAAGGAVHTRGLDFACD (SEQ ID NO: 37) ACPTGLYTHSGECCKACNLGEGVAQPCGANQTVCEPCLDSVTFSDVVSATEPCKPCT ECVGLQSMSAPCVEADDAVCRCAYGYYQDETTGRCEACRVCEAGSGLVFSCQDKQ NTVCEECPDGTYSDEADAEC (SEQ ID NO: 38), ACPTGLYTHSGECCKACNLGEGVAQPCGANQTVC (SEQ ID NO: 39), AVGQDTQEVIVVPHSLPFKV (SEQ ID NO: 40), and TTTPAPRPPTPAPTIALQPLSLRPEACRPAAGGAVHTRGLDFACDQTTPGERSSLPAFY PGTSGSCSGCGSLSLP (SEQ ID NO: 70).

Embodiment 203: The chimeric inhibitory receptor of any one of embodiments—, wherein the spacer region modulates sensitivity of the chimeric inhibitory receptor relative to an otherwise identical chimeric inhibitory receptor lacking the spacer region.

Embodiment 204: The chimeric inhibitory receptor of embodiment, wherein the spacer region increases sensitivity of the chimeric inhibitory receptor relative to an otherwise identical chimeric inhibitory receptor lacking the spacer region.

Embodiment 205: The chimeric inhibitory receptor of embodiment, wherein the spacer region reduces sensitivity of the chimeric inhibitory receptor relative to an otherwise identical chimeric inhibitory receptor lacking the spacer region.

Embodiment 206: The chimeric inhibitory receptor of any one of embodiments—, wherein the spacer region modulates potency of the chimeric inhibitory receptor relative to an otherwise identical chimeric inhibitory receptor lacking the spacer region.

Embodiment 207: The chimeric inhibitory receptor of embodiment, wherein the spacer region increases potency of the chimeric inhibitory receptor relative to an otherwise identical chimeric inhibitory receptor lacking the spacer region.

Embodiment 208: The chimeric inhibitory receptor of embodiment, wherein the spacer region reduces potency of the chimeric inhibitory receptor relative to an otherwise identical chimeric inhibitory receptor lacking the spacer region.

Embodiment 209: The chimeric inhibitory receptor of any one of embodiments—, wherein the spacer region modulates basal prevention, attenuation, or inhibition of activation of the tumor-targeting chimeric receptor when expressed on an immunomodulatory cell relative to an otherwise identical chimeric inhibitory receptor lacking the spacer region.

Embodiment 210: The chimeric inhibitory receptor of embodiment, wherein the spacer region reduces basal prevention, attenuation, or inhibition relative to an otherwise identical chimeric inhibitory receptor lacking the spacer region.

Embodiment 211: The chimeric inhibitory receptor of embodiment, wherein the spacer region increases basal prevention, attenuation, or inhibition relative to an otherwise identical chimeric inhibitory receptor lacking the spacer region.

Embodiment 212: The chimeric inhibitory receptor of any one of embodiments—, wherein the chimeric inhibitory receptor further comprises an intracellular spacer region positioned between the transmembrane domain and one of the two or more intracellular signaling domains and operably linked to each of the transmembrane domain and the intracellular signaling domain.

Embodiment 213: The chimeric inhibitory receptor of any one of embodiments—, wherein the chimeric inhibitory receptor further comprises an intracellular spacer region positioned between the transmembrane domain and one of the two or more intracellular signaling domains and physically linked to each of the transmembrane domain and the intracellular signaling domain.

Embodiment 214: The chimeric inhibitory receptor of embodiment or embodiment, wherein the intracellular spacer region modulates sensitivity of the chimeric inhibitory receptor relative to an otherwise identical chimeric inhibitory receptor lacking the intracellular spacer region.

Embodiment 215: The chimeric inhibitory receptor of embodiment, wherein the intracellular spacer region increases sensitivity of the chimeric inhibitory receptor relative to an otherwise identical chimeric inhibitory receptor lacking the intracellular spacer region.

Embodiment 216: The chimeric inhibitory receptor of embodiment, wherein the intracellular spacer region reduces sensitivity of the chimeric inhibitory receptor relative to an otherwise identical chimeric inhibitory receptor lacking the intracellular spacer region.

Embodiment 217: The chimeric inhibitory receptor of any one of embodiments—, wherein the intracellular spacer region modulates potency of the chimeric inhibitory receptor relative to an otherwise identical chimeric inhibitory receptor lacking the intracellular spacer region.

Embodiment 218: The chimeric inhibitory receptor of embodiment, wherein the intracellular spacer region increases potency of the chimeric inhibitory receptor relative to an otherwise identical chimeric inhibitory receptor lacking the intracellular spacer region.

Embodiment 219: The chimeric inhibitory receptor of embodiment, wherein the intracellular spacer region reduces potency of the chimeric inhibitory receptor relative to an otherwise identical chimeric inhibitory receptor lacking the intracellular spacer region.

Embodiment 220: The chimeric inhibitory receptor of any one of embodiments—, wherein the intracellular spacer region modulates basal prevention, attenuation, or inhibition of activation of the tumor-targeting chimeric receptor when expressed on an immunomodulatory cell relative to an otherwise identical chimeric inhibitory receptor lacking the intracellular spacer region.

Embodiment 221: The chimeric inhibitory receptor of embodiment, wherein the intracellular spacer region reduces basal prevention, attenuation, or inhibition relative to an otherwise identical chimeric inhibitory receptor lacking the intracellular spacer region.

Embodiment 222: The chimeric inhibitory receptor of embodiment, wherein the intracellular spacer region increases basal prevention, attenuation, or inhibition relative to an otherwise identical chimeric inhibitory receptor lacking the intracellular spacer region.

Embodiment 223: The chimeric inhibitory receptor of any one of embodiments—, wherein the inhibitory chimeric receptor further comprises an enzymatic inhibitory domain.

Embodiment 224: The chimeric inhibitory receptor of embodiment, wherein the enzymatic inhibitory domain is capable of preventing, attenuating, or inhibiting activation of a tumor-targeting chimeric receptor when expressed on an immunomodulatory cell relative to an otherwise identical chimeric inhibitory receptor lacking the enzymatic inhibitory domain.

Embodiment 225: The chimeric inhibitory receptor of embodiment or embodiment, wherein the enzymatic inhibitory domain comprises an enzyme catalytic domain.

Embodiment 226: The chimeric inhibitory receptor of embodiment, wherein the enzyme catalytic domain is derived from an enzyme selected from the group consisting of: CSK, SHP-1, PTEN, CD45, CD148, PTP-MEG1, PTP-PEST, c-CBL, CBL-b, PTPN22, LAR, PTPH1, SHIP-1, and RasGAP.

Embodiment 227: The chimeric inhibitory receptor of any one of embodiments—, wherein the enzymatic inhibitory domain comprises one or more modifications that modulate basal prevention, attenuation, or inhibition relative to an otherwise identical enzymatic inhibitory domain lacking the one or more modifications.

Embodiment 228: The chimeric inhibitory receptor of embodiment, wherein the one or more modifications reduce basal prevention, attenuation, or inhibition of the chimeric inhibitory receptor relative to an otherwise identical chimeric inhibitory receptor lacking the enzymatic inhibitory domain.

Embodiment 229: The chimeric inhibitory receptor of embodiment, wherein the one or more modifications increase basal prevention, attenuation, or inhibition of the chimeric inhibitory receptor relative to an otherwise identical chimeric inhibitory receptor lacking the enzymatic inhibitory domain.

Embodiment 230: The chimeric inhibitory receptor of any one of embodiments—, wherein the tumor-targeting chimeric receptor is a tumor-targeting chimeric antigen receptor (CAR) or an engineered T cell receptor (TCR).

Embodiment 231: The chimeric inhibitory receptor of any one of embodiments—, wherein the immunomodulatory cell is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an ESC-derived cell, and an iPSC-derived cell.

Embodiment 232: The chimeric inhibitory receptor of any one of embodiments—, wherein the immunomodulatory cell is a Natural Killer (NK) cell.

Embodiment 233: A composition comprising the chimeric inhibitory receptor of any one of embodiments 1—and a pharmaceutically acceptable carrier.

Embodiment 234: An engineered nucleic acid encoding the chimeric inhibitory receptor of any one of embodiments 1-.

Embodiment 235: An expression vector comprising the engineered nucleic acid of embodiment.

Embodiment 236: An isolated immunomodulatory cell comprising the engineered nucleic acid of embodiment or the expression vector of embodiment.

Embodiment 237: A composition comprising the engineered nucleic acid of embodiment or the expression vector of embodiment, and a pharmaceutically acceptable carrier.

Embodiment 238: An isolated immunomodulatory cell comprising the chimeric inhibitory receptor of any one of embodiments 1-.

Embodiment 239: The isolated cell of embodiment, wherein the cell further comprises a tumor-targeting chimeric receptor expressed on the surface of the cell.

Embodiment 240: The isolated cell of embodiment, wherein upon binding of the protein to the chimeric inhibitory receptor, the chimeric inhibitory receptor prevents, attenuates, or inhibits activation of the tumor-targeting chimeric receptor relative to an otherwise identical cell lacking a chimeric inhibitory receptor.

Embodiment 241: An isolated immunomodulatory cell comprising a chimeric inhibitory receptor, wherein the chimeric inhibitory receptor comprises:
an extracellular protein binding domain;
a transmembrane domain, wherein the transmembrane domain is operably linked to the extracellular protein binding domain, and
an intracellular signaling domain, wherein the intracellular signaling domain is operably linked to the transmembrane domain; and
   wherein upon binding of the protein to the chimeric inhibitory receptor, the chimeric inhibitory receptor prevents, attenuates, or inhibits activation of a tumor-targeting chimeric receptor expressed on the surface of the cell relative to an otherwise identical cell lacking a chimeric inhibitory receptor.

Embodiment 242: The isolated cell of embodiment, wherein the cell further comprises a tumor-targeting chimeric receptor expressed on the surface of the cell.

Embodiment 243: An isolated immunomodulatory cell comprising:
(a) a chimeric inhibitory receptor, wherein the chimeric inhibitory receptor comprises:
   an extracellular protein binding domain,
   a transmembrane domain, wherein the transmembrane domain is operably linked to the extracellular protein binding domain, and
   an intracellular signaling domain, wherein the intracellular signaling domain is operably linked to the transmembrane domain; and
(b) a tumor-targeting chimeric receptor expressed on the surface of the cell,
   wherein upon binding of the protein to the chimeric inhibitory receptor, the chimeric inhibitory receptor prevents, attenuates, or inhibits activation of the tumor-targeting chimeric receptor relative to an otherwise identical cell lacking a chimeric inhibitory receptor.

Embodiment 244: The isolated cell of any one of embodiments—, wherein the intracellular signaling domain is derived from a protein selected from the group consisting of: BTLA, PD-1, CTLA4, TIM3, KIR3DL1, LIR1, NKG2A, TIGIT, and LAG3.

Embodiment 245: The isolated cell of any one of embodiments—, wherein the chimeric inhibitory receptor is recombinantly expressed.

Embodiment 246: The isolated cell of any one of embodiments—, wherein the chimeric inhibitory receptor is expressed from a vector or a selected locus from the genome of the cell.

Embodiment 247: The isolated cell of any one of embodiments—, wherein the tumor-targeting chimeric receptor is a chimeric antigen receptor (CAR) or an engineered T cell receptor.

Embodiment 248: The cell of any one of embodiments—, wherein prior to binding of the protein to the chimeric inhibitory receptor, the tumor-targeting chimeric receptor is capable of activating the cell.

Embodiment 249: The cell of any one of embodiments—, wherein upon binding of the protein to the chimeric inhibitory receptor, the chimeric inhibitory receptor suppresses cytokine production from the activated cell.

Embodiment 250: The cell of any one of embodiments—, wherein upon binding of the protein to the chimeric inhibitory receptor, the chimeric inhibitory receptor suppresses a cell-mediated immune response to a target cell, wherein the immune response is induced by activation of the immunomodulatory cell.

Embodiment 251: The cell of any one of embodiments—, wherein the transmembrane domain is physically linked to the extracellular protein binding domain.

Embodiment 252: The cell of any one of embodiments—, wherein the intracellular signaling domain is physically linked to the transmembrane domain.

Embodiment 253: The cell of any one of embodiments—, wherein the transmembrane domain is physically linked to the extracellular protein binding domain and the intracellular signaling domain is physically linked to the transmembrane domain.

Embodiment 254: An isolated immunomodulatory cell comprising a chimeric inhibitory receptor, wherein the chimeric inhibitory receptor comprises:
an extracellular protein binding domain;
a transmembrane domain, wherein the transmembrane domain is operably linked to the extracellular protein binding domain, and
two or more intracellular signaling domains, wherein the two or more intracellular signaling domains are operably linked to the transmembrane domain; and
   wherein upon binding of the protein to the chimeric inhibitory receptor, the chimeric inhibitory receptor prevents, attenuates, or inhibits activation of a tumor-targeting chimeric receptor expressed on the surface of the cell relative to an otherwise identical cell lacking a chimeric inhibitory receptor.

Embodiment 255: The isolated cell of embodiment, wherein the cell further comprises a tumor-targeting chimeric receptor expressed on the surface of the cell.

Embodiment 256: An isolated immunomodulatory cell comprising:
(a) a chimeric inhibitory receptor, wherein the chimeric inhibitory receptor comprises:
   an extracellular protein binding domain,
   a transmembrane domain, wherein the transmembrane domain is operably linked to the extracellular protein binding domain, and
   two or more intracellular signaling domains, wherein the two or more intracellular signaling domains are operably linked to the transmembrane domain; and
(b) a tumor-targeting chimeric receptor expressed on the surface of the cell,
   wherein upon binding of the protein to the chimeric inhibitory receptor, the chimeric inhibitory receptor prevents, attenuates, or inhibits activation of the tumor-targeting chimeric receptor relative to an otherwise identical cell lacking a chimeric inhibitory receptor. Embodiment 257: The isolated cell of any one of embodiments—, wherein each of the two or more intracellular signaling domains is derived from a protein selected from the group consisting of: BTLA, PD-1, CTLA4, TIM3, KIR3DL1, LIR1, NKG2A, TIGIT, and LAG3.

Embodiment 258: The isolated cell of any one of embodiments—, wherein the chimeric inhibitory receptor is recombinantly expressed.

Embodiment 259: The isolated cell of any one of embodiments—, wherein the chimeric inhibitory receptor is expressed from a vector or a selected locus from the genome of the cell.

Embodiment 260: The isolated cell of any one of embodiments—, wherein the tumor-targeting chimeric receptor is a chimeric antigen receptor (CAR) or an engineered T cell receptor.

Embodiment 261: The isolated cell of any one of embodiments—, wherein prior to binding of the protein to the chimeric inhibitory receptor, the tumor-targeting chimeric receptor is capable of activating the cell.

Embodiment 262: The isolated cell of any one of embodiments—, wherein upon binding of the protein to the chimeric inhibitory receptor, the chimeric inhibitory receptor suppresses cytokine production from the activated cell.

Embodiment 263: The isolated cell of any one of embodiments—, wherein upon binding of the protein to the chimeric inhibitory receptor, the chimeric inhibitory receptor suppresses a cell-mediated immune response to a target cell, wherein the immune response is induced by activation of the immunomodulatory cell.

Embodiment 264: The isolated cell of any one of embodiments—, wherein the transmembrane domain is physically linked to the extracellular protein binding domain.

Embodiment 265: The isolated cell of any one of embodiments—, wherein one of the two or more intracellular signaling domains is physically linked to the transmembrane domain.

Embodiment 266: The isolated cell of any one of embodiments—, wherein the transmembrane domain is physically linked to the extracellular protein binding domain and one of the two or more intracellular signaling domains is physically linked to the transmembrane domain.

Embodiment 267: The isolated cell of any one of embodiments—, wherein the target cell is a tumor cell.

Embodiment 268: The isolated cell of any one of embodiments—, wherein the cell is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an ESC-derived cell, and an iPSC-derived cell.

Embodiment 269: The isolated cell of any one of embodiments—, wherein the cell is a Natural Killer (NK) cell.

Embodiment 270: The isolated cell of any one of embodiments—, wherein the cell is autologous.

Embodiment 271: The isolated cell of any one of embodiments—, wherein the cell is allogeneic.

Embodiment 272: A composition comprising the isolated cell of any one of embodiments—and a pharmaceutically acceptable carrier.

Embodiment 273: A method of preventing, attenuating, or inhibiting a cell-mediated immune response induced by a tumor-targeting chimeric receptor expressed of the surface of an immunomodulatory cell, comprising:

engineering the immunomodulatory cell to express the chimeric inhibitory receptor of any one of embodiments 1—on the surface of the immunomodulatory cell,
wherein upon binding of a cognate protein to the chimeric inhibitory receptor, the intracellular signaling domain prevents, attenuates, or inhibits activation of the tumor-targeting chimeric receptor.

Embodiment 274: A method of preventing, attenuating, or inhibiting activation of a tumor-targeting chimeric receptor expressed on the surface of an immunomodulatory cell, comprising:

contacting the isolated cell of any one of embodiments—or the composition of embodiment 272 with a cognate protein of the chimeric inhibitory receptor under conditions suitable for the chimeric inhibitory receptor to bind the cognate protein,
wherein upon binding of the protein to the chimeric inhibitory receptor, the intracellular signaling domain prevents, attenuates, or inhibits activation of the tumor-targeting chimeric receptor.

Embodiment 275: The method of embodiment or embodiment, wherein the tumor-targeting chimeric receptor is a chimeric antigen receptor (CAR) or an engineered T cell receptor (TCR).

Embodiment 276: The method of embodiment, wherein the CAR binds one or more antigens expressed on the surface of a tumor cell.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W. H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B (1992).

### Example 1: Inhibitory CARs with a Various Signaling Domains Reduce T Cell Activation

### Methods and Materials

### Inhibitory Chimeric Receptor and Tumor-Targeting Chimeric Receptor Constructs

An inhibitory chimeric receptor (iCAR) with a BTLA intracellular signaling domain was synthesized. The iCAR comprised an IgGκ secretion signal, an anti-CD19 scFv with a FLAG tag, a CD8 hinge domain, a BTLA transmembrane domain, and a BTLA intracellular signaling domain. A FLAG tag was fused on the N-terminus of the scFv (after the signal sequence) in the iCAR. Two activating CARs (aCAR) were also constructed. One aCAR had a CD8 secretion signal, an anti-CD19 scFv with a Myc tag, a CD8 hinge domain, a CD28 transmembrane domain, and CD28 and CD3ζ intracellular signaling domains. The other aCAR had a CD8 secretion signal, an anti-CD20 scFv with a Myc tag, a CD8 hinge domain, a CD28 transmembrane domain, and CD28 and CD3ζ intracellular signaling domains. The MYC tag was fused on the C-terminus of the scFv (before the hinge) in the aCARs. In both cases a 3×(G4S) linker was used in the scFv and the CD8 hinge connecting the scFv to the transmembrane domain.

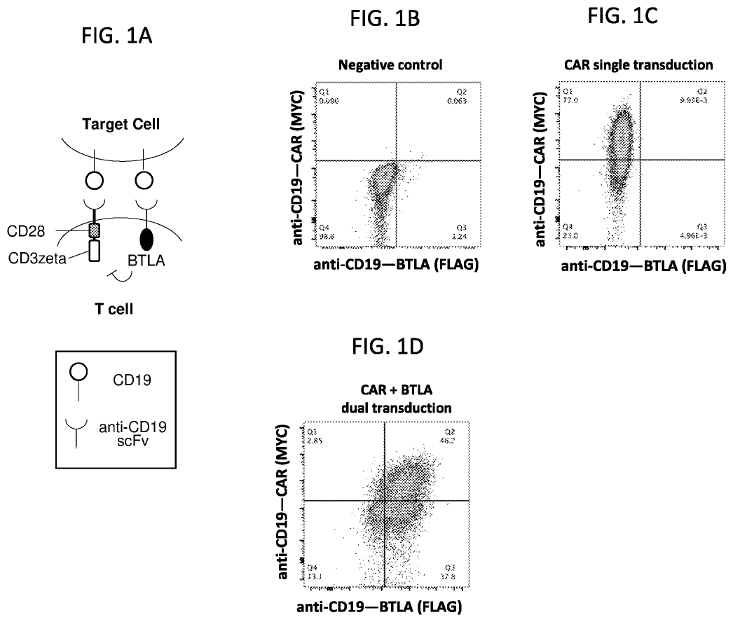

An exemplary diagram of a T cell co-expressing an anti-CD19-BTLA iCAR and an anti-CD19-CD28/CD3ζ aCAR contacting a target cell expressing CD19 is shown in FIG. 1A.

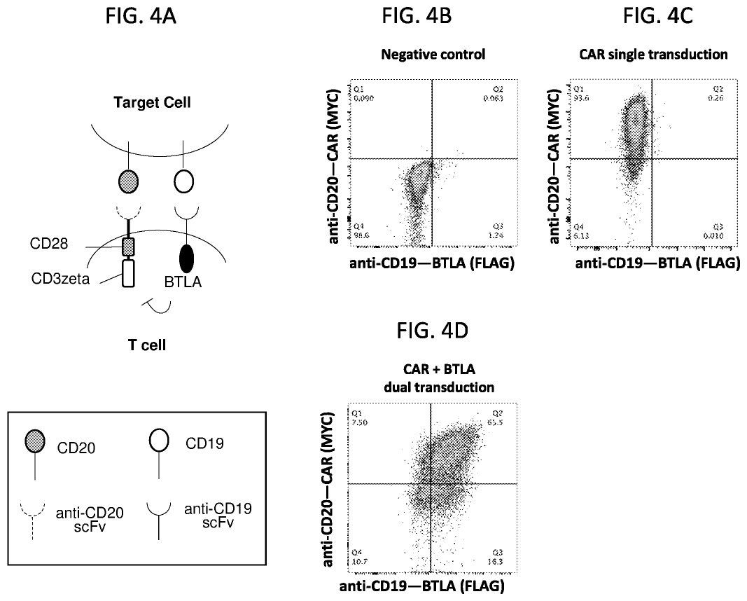

An exemplary diagram of a T cell co-expressing an anti-CD19-BTLA iCAR and an anti-CD20-CD28/CD3ζ aCAR contacting a target cell expressing CD19 and CD20 is shown in FIG. 4A.

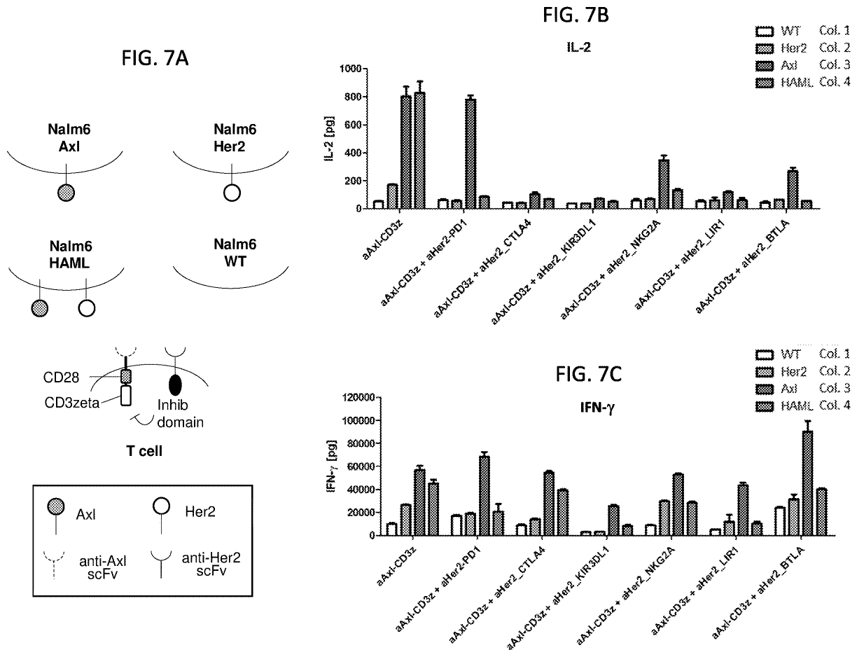

Additional inhibitory chimeric receptors with an anti-Her2 scFv fused to BTLA, PD1, CTLA4, KIR3DL1, NKG2A, or LIR1 intracellular signaling domains and GFP were also synthesized. These inhibitory chimeric receptors had and the FLAG tag and GFP fluorescence protein as described above. An additional aCAR comprising an anti-Axl scFv fused to a CD3ζ intracellular signaling domain and mCherry was also synthesized. An exemplary diagram of target cells expressing Axl, Her2, both Axl and Her2, or neither Axl and Her2, and a T cell co-expressing an anti-Her2 iCAR with a general intracellular inhibitory domain and an anti-Axl-CD3ζ aCAR is shown in FIG. 7A.

Table 9 provides the sequences of the inhibitory and tumor-targeting chimeric receptors synthesized.

TABLE 9
Inhibitory and tumor-targeting chimeric
receptors

| | SEQ | |
|---|---|---|
| | ID | Descrip- |
| Amino Acid Sequence | NO: | tion |
| METDTLLLWVLLLWVPGSTGAGGSDYKDDDDKGG | 56 | Anti- |
| SEVKLQESGPGLVAPSQSLSVTCTVSGVSLPDYG | | CD19 |
| VSWIRQPPRKGLEWLGVIWGSETTYYNSALKSRL | | BTLA |
| TIIKDNSKSQVFLKMNSLQTDDTAIYYCAKHYYY | | inhibi- |
| GGSYAMDYWGQGTSVTVSSGGGGSGGGGSGGGGS | | tory |
| DIQMTQTTSSLSASLGDRVTISCRASQDISKYLN | | chimeric |
| WYQQKPDGTVKLLIYHTSRLHSGVPSRFSGSGSG | | receptor |
| TDYSLTISNLEQEDIATYFCQQGNTLPYTFGGGT | | |
| KLEITTTTPAPRPPTPAPTIALQPLSLRPEACRP | | |
| AAGGAVHTRGLDFACDLLPLGGLPLLITTCFCLF | | |
| CCLRRHQGKQNELSDTAGREINLVDAHLKSEQTE | | |
| ASTRQNSQVLLSETGIYDNDPDLCFRMQEGSEVY | | |
| SNPCLEENKPGIVYASLNHSVIGPNSRLARNVKE | | |
| APTEYASICVRS | | |
| MALPVTALLLPLALLLHAARPEVKLQESGPGLVA | 57 | Anti- |
| PSQSLSVTCTVSGVSLPDYGVSWIRQPPRKGLEW | | CD19- |
| LGVIWGSETTYYNSALKSRLTIIKDNSKSQVFLK | | CD28/ |
| MNSLQTDDTAIYYCAKHYYYGGSYAMDYWGQGTS | | CD3 ζ |
| VTVSSGGGGSGGGGSGGGGSDIQMTQTTSSLSAS | | tumor |
| LGDRVTISCRASQDISKYLNWYQQKPDGTVKLLI | | target- |
| YHTSRLHSGVPSRFSGSGSGTDYSLTISNLEQED | | ing |
| IATYFCQQGNTLPYTFGGGTKLEITEQKLISEED | | CAR |
| LNGAATTTPAPRPPTPAPTIALQPLSLRPEACRP | | |
| AAGGAVHTRGLDFACDFWVLVVVGGVLACYSLLV | | |
| TVAFIIFWVRSKRSRLLHSDYMNMTPRRPGPTRK | | |
| HYQPYAPPRDFAAYRSRVKFSRSADAPAYKQGQN | | |
| QLYNELNLGRREEYDVLDKRRGRDPEMGGKPRRK | | |
| NPQEGLYNELQKDKMAEAYSEIGMKGERRRGKGH | | |
| DGLYQGLSTATKDTYDALHMQALPPR | | |
| MALPVTALLLPLALLLHAARPQVQLVQSGAEVKK | 58 | Anti- |
| PGASVKVSCKASGYTFTNYWMHWVRQAPGQGLEW | | CD20- |
| MGFITPTTGYPEYNQKFKDRVTMTADKSTSTAYM | | CD28/ |
| ELSSLRSEDTAVYYCARRKVGKGVYYALDYWGQG | | CD3 ζ |
| TTVTVSSGGGGSGGGGSGGGGSDIQMTQSPSSLS | | tumor |
| ASVGDRVTITCRASGNIHNYLAWYQQKPGKVPKL | | target- |
| LIYNTKTLADGVPSRFSGSGSGTDYTLTISSLQP | | ing |
| EDVATYYCQHFWSSPWTFGGGTKVEIKEQKLISE | | CAR |
| EDLNGAATTTPAPRPPTPAPTIALQPLSLRPEAC | | |
| RPAAGGAVHTRGLDFACDFWVLVVVGGVLACYSL | | |
| LVTVAFIIFWVRSKRSRLLHSDYMNMTPRRPGPT | | |
| RKHYQPYAPPRDFAAYRSRVKFSRSADAPAYKQG | | |
| QNQLYNELNLGRREEYDVLDKRRGRDPEMGGKPR | | |
| RKNPQEGLYNELQKDKMAEAYSEIGMKGERRRGK | | |
| GHDGLYQGLSTATKDTYDALHMQALPPR | | |

The sequence of the anti-CD19 BTLA inhibitory chimeric receptor with a BTLA intracellular signaling domain is shown as SEQ ID NO: 56. The sequence of the anti-CD19-CD328/CD33 tumor targeting CAR is shown as SEQ ID NO: 57. The sequence of the anti-CD20-CD28/CD3 tumor targeting CAR is shown as SEQ ID NO: 58.

T Cell Transduction and Expansion with Anti-CD19 or Anti-CD20 Activating CAR (aCAR) and/or the Anti-CD19 Inhibitory CAR (iCAR)

On day 1, 1×10⁶ purified CD4+/CD8+ T-cells were thawed and stimulated with 3×10⁶ Dynabeads, then cultured in 1 mL Optimizer CTS T-cell expansion media (Gibco) with 0.2 ug/mL IL-2. T cells were singly or co-transduced on day 2 with lentivirus (100K each, as quantified by GoStix (Tekara)) encoding constitutive expression of either the anti-CD19 or anti-CD20 activating CAR (aCAR) and/or the anti-CD19 inhibitory CAR (iCAR).

On day 3, the Dynabeads were removed by magnet. The T-cells were counted and passaged (0.5×10⁶ cells/mL). An aliquot of these cells was stained with PE conjugated anti-MYC and BV421 conjugated anti-FLAG antibodies (corresponding to the aCAR and the iCAR), and their transgene expression quantified using an LX CytoFlex Flow Cytometry machine. During subsequent expansion, cells were passaged every two days (0.5×10⁶ cells/mL).

T Cell Co-Culture Assay for Anti-CD19 CD20 iCARs and aCARs

On day 8, the T-cells were counted and distributed into a 96-well plate for co-culture assays. Each well contained 5×10⁵ Nalm6 target cells stained with cell trace violet dye (Invitrogen) and 5×10⁵ aCAR plus or minus iCAR T-cells. Co-cultures were incubated at 37° C. with 5% CO₂ for 18 hrs.

On day 9, cells in co-cultures were stained with NIR viability dye (Biolegend) and percent death of target cells was quantified using an LX CytoFlex Flow Cytometry machine. The percent killing was normalized to target cells only. Cytokines in the media from the same co-cultures were measured using a Human magnetic Luminex assay (R&D systems) and MAGPIX analyzer (Millipore Sigma).

T Cell Transduction and Expansion with Anti-Axl-CD3C Activating CAR (aCAR) and or the Anti-Her2 Inhibitory CARs (iCARs)

On day 1, 1×10⁶ purified CD4+ T-cells were thawed and stimulated with 3×10⁶ Dynabeads, then cultured in 1 mL Optimizer CTS T-cell expansion media (Gibco) with 0.2 ug/mL IL-2. T cells were singly or co-transduced on day 2 with lentivirus (100K each, as quantified by GoStix (Tekara)) encoding constitutive expression of either the anti-Axl-CD3ζ-mCherry activating CAR (aCAR) and/or the various anti-Her2 inhibitory CARs (iCAR) individually. The iCAR expression plasmid included a puromycin resistance gene.

On Day 4, the T cells were incubated with media containing puromycin to select for expression of the indicated iCAR. Control cells transduced with only the anti-Axl-CD3ζ-mCherry activating CAR were not selected with puromycin.

The Dynabeads were removed by magnet. The T-cells were counted and passaged (0.5×10⁶ cells/mL). Expression of the anti-Axl-CD3ζ-mCherry aCAR was checked by flow cytometry for mCherry expression. During subsequent expansion, cells were passaged every two days (0.5×10⁶ cells/mL).

T Cell Co-Culture Assay for Anti-Her2 iCARs and Anti-Axl aCARs

On day 7, the T-cells were counted and distributed into a 96-well plate with X-VIVO15 medium (Lonza) supplemented with human antibody for co-culture assays. Each well contained 1×10⁵ Nalm6 target cells expressing either Alx, Her2, both Axl and Her2, or neither Axl or Her2 (wt), and 1×105 CD4+ T-cells expressing both the anti-Axl activating CAR and the indicated anti-Her2 inhibitory CAR. CD4+ T cells expressing only the anti-Axl activating CAR were used as a control. Co-cultures were incubated at 37° C. with 5% CO₂ for 18 hrs.

On Day 8, supernatants were collected and cytokines analyzed via ELISA

### Results

### Inhibitory Chimeric Receptor and Tumor-Targeting Chimeric Receptor Bind Same Antigen

The ability of an iCAR to reduce or inhibit T cell activation in a cell expressing an iCAR and an aCAR that bind the same antigen was assessed. An exemplary diagram of a T cell co-expressing an anti-CD19-BTLA iCAR and an anti-CD19 aCAR contacting a target cell expressing CD19 is shown in FIG. 1A. The cells transduced with the anti-CD19-BTLA-iCAR and anti-CD19 aCAR showed high levels of surface expression in primary T cells. T cells transduced with only the aCAR showed high aCAR expression and no iCAR expression (FIG. 1C), while T cells co-transduced with both the aCAR and iCAR showed high levels of expression of both CAR proteins (FIG. 1D). The negative control cells showed no expression of either construct (FIG. 1B).

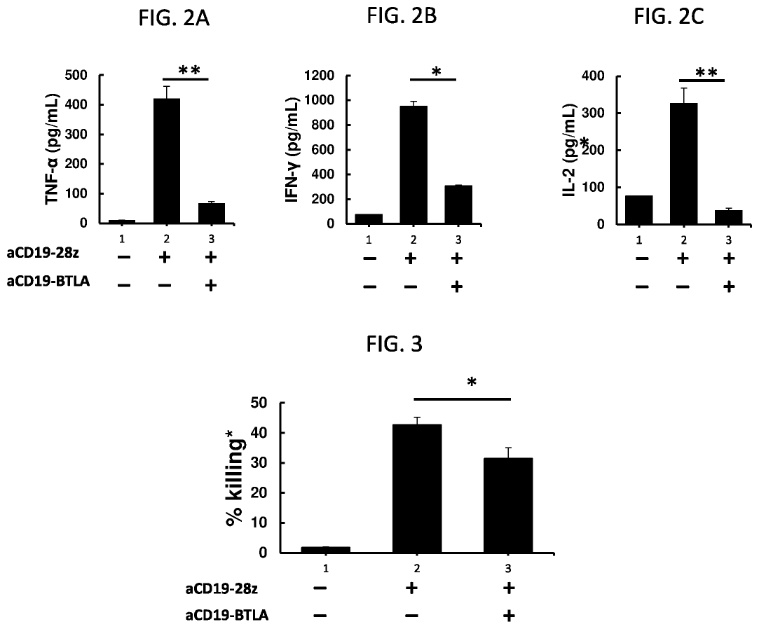

The anti-CD19-BTLA iCAR suppressed T cell cytokine production induced by the anti-CD19 aCAR (aCD19-28z) after co-culture with Nalm6 cells expressing CD19. Co-culture of the CD19-expressing Nalm6 cells with anti-CD19 aCAR T cells induced TNF-α, IFN-7, and IL-2 production (FIGS. 2A, 2B, and 2C, respectively). However, T cells expressing both the anti-CD19 aCAR and the anti-CD19 BTLA-iCAR had significantly reduced TNF-α, IFN-γ, and IL-2 production after co-culture with the Nalm6 target cells (*p>0.05, **p>0.01). Thus, binding of the iCAR to its cognate ligand on the target cell successfully reduced the aCAR-induced cytokine production.

In addition, the anti-CD19-BTLA iCAR suppressed the T cell cytotoxicity induced by the anti-CD19 aCAR after co-culture with Nalm6 cells expressing CD19. As shown in FIG. 3, co-culture of the target Nalm6 cells expressing CD19 with T cells expressing only the anti-CD19 aCAR resulted in significant killing of the target cells. However, T cells expressing both the anti-CD19 aCAR and the anti-CD19 BTLA iCAR had a statistically significant reduction in cytotoxicity when co-cultured with the Nalm6 target cells. Thus, binding of the iCAR to its cognate ligand on the target cell successfully reduced the aCAR-induced cytotoxicity activity of the T cells.

### Inhibitory Chimeric Receptor and Tumor-Targeting Chimeric Receptor Bind Different Antigens

Next, the ability of an iCAR to reduce or inhibit T cell activation in a T cell expressing an iCAR and an aCAR that each bind different antigens was assessed. An exemplary diagram of a T cell co-expressing an anti-CD20-BTLA iCAR and an anti-CD19 aCAR contacting a target cell expressing CD19 and CD20 is shown in FIG. 4A. The cells transduced with the anti-CD19-BTLA iCAR and anti-CD20 aCAR showed high levels of surface expression in primary T cells. T cells transduced with only the aCAR showed high aCAR expression and no iCAR expression (FIG. 4C), while T cells co-transduced with both the aCAR and iCAR showed high levels of expression of both CAR proteins (FIG. 4D). The negative control cells showed no expression of either construct (FIG. 4B).

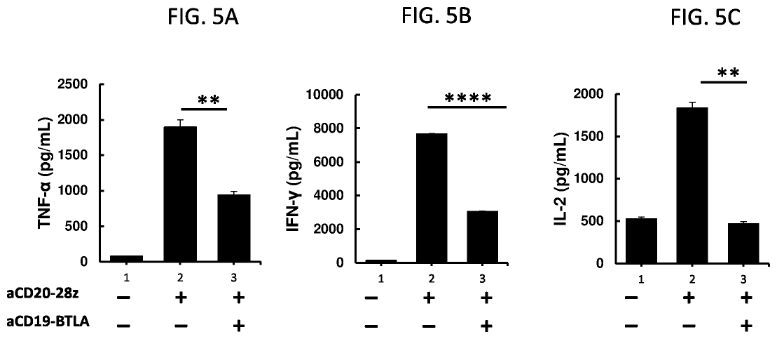

The anti-CD19-BTLA iCAR suppressed T cell cytokine production induced by the anti-CD20 aCAR (aCD20-28z) after co-culture with Raji cells expressing CD19 and CD20. Co-culture of the Raji cells with anti-CD20 aCAR T cells induced TNF-α, IFN-γ, and IL-2 production (FIGS. 5A, 5B, and 5C, respectively). However, T cells expressing both the anti-CD20 aCAR and the anti-CD19 BTLA iCAR had significantly reduced TNF-α, IFN-γ, and IL-2 production after co-culture with the Raji target cells (**p>0.01, ****p>0.0001). Thus, binding of the iCAR to its cognate ligand on the target cell successfully reduced the aCAR-induced cytokine production.

Thus, an anti-CD19-BTLA fusion (iCAR) was expressed at high levels in lentivirus transduced CD4+ and CD8+ T-cells without subsequent enrichment. Importantly, high levels of co-expression of iCAR and aCAR were observed after co-transduction. In addition, the CD19-BTLA iCAR suppressed multiple T-cell activation responses (cytotoxicity and production of cytokines TNF-α, IFN-γ, and IL-2) in two contexts: i) when the iCAR shares the same cell surface ligand as the aCAR (CD19 receptor), and ii) when the iCAR and aCAR target different cell surface ligands (CD19 and CD20, respectively).

Functionality of Additional iCAR Domains

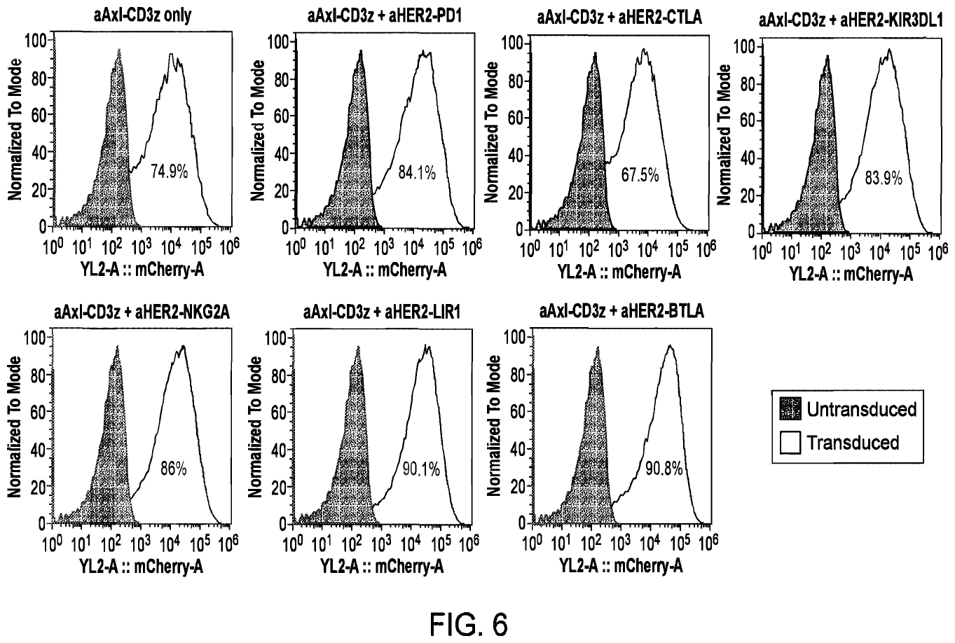

FIG. 6 shows expression of the anti-Axl-CD3ζ-mCherry aCAR in CD4+ T cells as determined by flow cytometry quantification of mCherry. Expression of the indicated anti-Her2 inhibitory CAR was determined via puromycin resistance selection prior to the mCherry flow cytometry quantification of the resistance-selected T cells. Control cells expressing only anti-Axl-CD3ζ aCAR were not incubated with puromycin. Thus, all dual transduced T cells in FIG. 6 express both the anti-Axl-CD3ζ aCAR and the indicated anti-Her2 inhibitory CAR.

IL-2 (FIG. 7B) and IFN-7 (FIG. 7C) secretion by the dual expression T cells was assessed after incubation with target Naml6 cells expressing Axl alone, Her2 alone, or Axl and Her2 (HAML cells). WT Nalm6 cells expressing either Axl or Her2 were used as a control.

As shown in FIG. 7B, cells expressing both the anti-Axl-CD3ζ aCAR and either the anti-Her2-PD-1 iCAR or the anti-Her2-BTLA iCAR had the highest specificity in the IL-2 secretion assay. In those samples, the Nalm6 cells expressing Axl induced IL-2 secretion by the T cells, while the Nalm6 cells HAML expressing Axl and Her2 did not induce IL-2 secretion, indicating the successful inhibitory activity of the anti-Her2-PD-1 iCAR or the anti-Her2-BTLA iCAR on the activation and signaling of the anti-Axl-CD3ζ aCAR in the T cell. The anti-Her2-NKG2A iCAR also successfully reduced the IL-2 secretion induced by the anti-Axl-CD3ζ aCAR in the T cell after contacting the HAML dual expressing target cells.

As shown in FIG. 7C, cells expressing both the anti-Axl-CD3ζ aCAR and either the anti-Her2-PD-1 iCAR, the anti-Her2-KIR3DL1 iCAR, or the anti-Her2-LIR1 iCAR had the highest specificity in the IFN-7 secretion assay. In those samples, the Nalm6 cells expressing Axl induced IFN-7 secretion by the T cells, while the Nalm6 cells HAML expressing Axl and Her2 did not induce IFN-7 secretion, indicating the successful inhibitory activity of the anti-Her2-PD-1 iCAR, the anti-Her2-KIR3DL1 iCAR, or the anti-Her2-LIR1 iCAR on the activation and signaling of the anti-Axl-CD3ζ aCAR in the T cell. The anti-Her2-BTLA iCAR and the anti-Her2-NKG2A iCARs also successfully reduced the IFN-7 secretion induced by the anti-Axl-CD3ζ aCAR in the T cell after contacting the HAML dual expressing target cells.

### Example 2: Inhibitory Chimeric Receptor with a BTLA Signaling Domain Reduces NK Cell Activation

### Materials and Methods

### Transduction and Expansion

NK cells were co-cultured at a 1:1 ratio with irradiated aAPC(K562 mIL-15/4-1BBL/CD86) to drive expansion on day 1. On day 7, an assay plate was prepared by coating the wells of 24 well plate with RetroNectin (Tekara, 7 ug/well) at 4° overnight.

NK cells were co-transduced with lentivirus encoding constitutive expression of either an activating CAR (aCAR) and/or an inhibitory CAR (iCAR) using RetroNectin (MOI: 5-10) according to the manufacturer's protocol on day 8. The aCAR was an anti-Axl scFv fused to a CD3ζ intracellular signaling domain and mCherry. The iCAR was an anti-Her2 scFv fused to a BTLA intracellular signaling domain and GFP. The transduction was repeated on day 9. Expression of the aCAR and iCAR transgenes was checked by fluorescent microscopy and flow cytometry.

### Co-Culture Assay

NK cells expressing the aCAR and/or the iCAR were incubated with engineered Nalm6 target cells (Her2+, Axl+) at increasing effector to target cell ratios (E:T). NK cell killing of the Nalm6 target cells was performed using the LDH-Glo™ Cytotoxicity Assay (Promega) according to manufacturer's instructions.

### Results

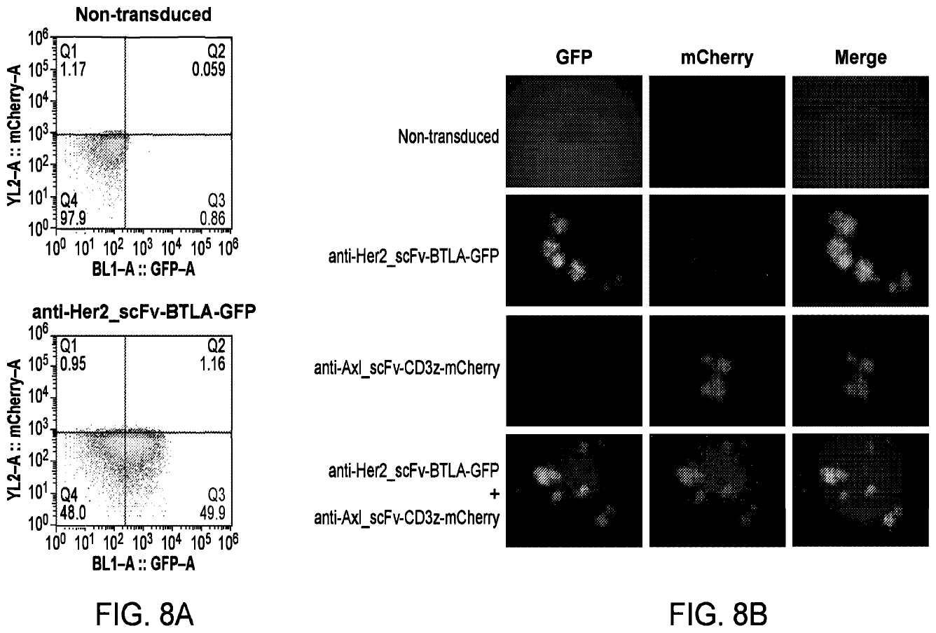

The anti-Her2 BTLA-iCAR showed high levels of surface expression in primary NK cells, in co-transduction with anti-Axl CD3zeta-aCAR. FIG. 8A shows the flow cytometry dot plots of the non-transduced NK cells (negative control, top panel) and the NK cells transduced with only the anti-Her2-BTLA iCAR expression construct (bottom panel). FIG. 8B shows the GFP, mCherry, and merged channels from immunofluorescent microscopy of non-transduced cells, cells transduced with the anti-Her2-BTLA iCAR, cells transduced with the anti-Axl-CD3ζ aCAR, and cells transduced with both the iCAR and the aCAR. The single and dual transduced cells both showed good expression of the CARs as shown by the expression of the fused mCherry or GFP reporter proteins. The non-transduced cells show no signal in the GFP, mCherry, or merge channels. The Her2-BTLA-GFP cells show signal in the GFP channel. The Axl-CD3ζ-mCherry cells show signal in the mCherry channel. The Her2-BTLA-GFP and Axl-CD3ζ-mCherry cells show GFP and mCherry expression in the corresponding channels that overlap in the merge channel, indicating that the dual transduces cells successfully express both the Her2-BTLA-GFP iCAR and the Axl-CD3ζ-mCherry aCAR constructs.

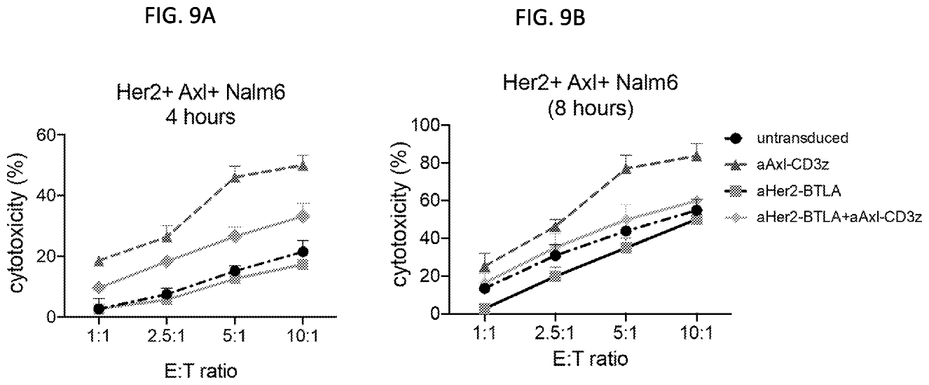

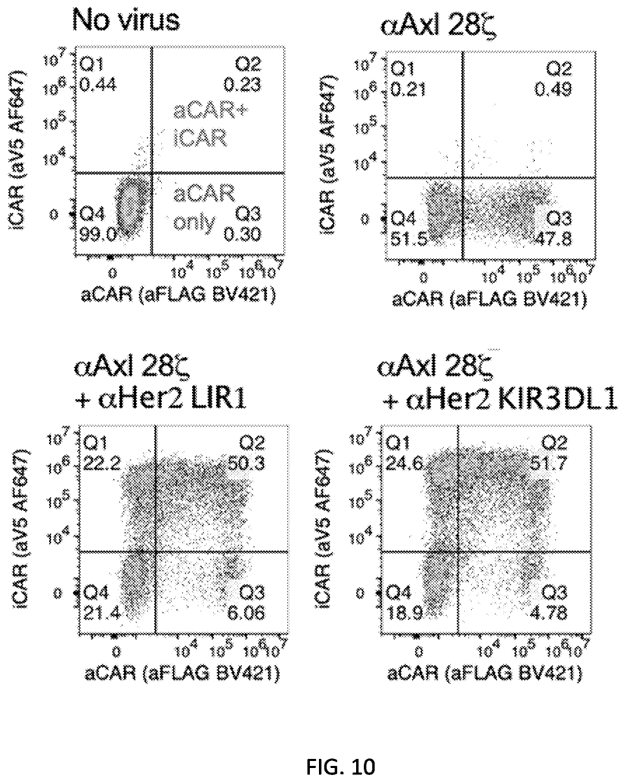

The anti-Her2-BTLA iCAR suppressed anti-Axl-CD3ζ aCAR cytotoxicity in primary NK cells. FIG. 9A shows the percent cell lysis of the target Her2+ Axl+ Nalm6 cells after a 4 hr incubation with NK cells singly or co-expressing the anti-Her2-BTLA iCAR and the anti-Axl-CD3ζ aCAR. NK cells expressing just the anti-Her2-BTLA iCAR did not induce cell lysis as compared to untransduced NK cells, while NK cells expressing just the anti-Axl-CD3ζ aCAR induced significant amounts of cell lysis as compared to untransduced NK cells. Importantly, the NK cells co-expressing the iCAR and the aCAR induced lower levels of target cell lysis than the NK cells expressing the aCAR alone. This indicates that the activation of the iCAR by its cognate ligand on the target cell inhibited the signaling of the aCAR, and thus inhibited the activation of the NK cell. Similar results were seen after 8 hours of incubation (FIG. 9B), with greater inhibitory activity of the iCAR on the aCAR signaling in the co-transduced NK cells.

Thus, an anti-Her2-BTLA fusion (iCAR) was expressed at high levels in lentivirus transduced NK cells without subsequent enrichment. Importantly, co-expression of the iCAR and the aCAR was seen after co-transduction. Furthermore, the scFv-BTLA iCAR suppressed the aCAR-mediated cytotoxicity of target cells.

### Example 3: Assessment of LIR1 and KIR3DL1 Inhibitory Chimeric Receptors in Reducing NK Cell Activation

### Materials and Methods

### Transduction and Expansion

NK cells were expanded for 10 days with mitomycin C-treated K562 feeder cells, followed by transduction with 7.5e5 pg of aCAR virus (SFFV FLAGtag aAxl CD28-CD3z) alone or with 7.5e5 pg of either iCAR1 or iCAR2 virus (SFFV aHer2 V5tag LIR1 P2A PuroR or SFFV aHer2 V5tag KIR3DL1 P2A PuroR, respectively). Sequences for the iCAR constructs assessed are shown in Table 10A. Sequences for the aCAR construct assessed are shown in Table 10B. Each iCAR construct format is from N to C terminal: signal sequence 1-signal sequence 2-scFv-tag-hinge-TM-inhibitory cytosolic domain 1-inhibitory cytosolic domain 2 (if present). The aAxl CD28-CD3z format is from N to C terminal: signal sequence-tag-scFv-hinge-TM-intracellular signaling domain 1-intracellular signaling domain 2. The After 4 days, puromycin was added to cells for selection.

After 3 more days, cytotoxicity assays were performed by co-incubating engineered NK cells and parental NALM6 targets (WT), or NALM6 targets engineered to overexpress Axl or both Axl and Her2 antigens. Each engineered NK cells were incubated either with (1) each target cell type separately at a ratio of 25,000 NK cells to 50,000 NALM6 cells in triplicate; or (2) as a mixture of 25,000 single antigen Axl+ only and 25,000 dual antigen Axl+Her2+ NALM6 cells co-incubated with 25,000 NK cells of the indicated type in a 1:1:1 ratio (dual antigen targets were stained with different membrane dyes allowing them to be distinguished by flow). After overnight incubation, cells were stained with viability dyes and counted via flow cytometry. The target cell reduction was quantified as 100%×(1−No. Targets/No. Targets (NV)). Supernatant was also collected from cytotoxicity assays and analyzed for the presence of NK cell-secreted cytotoxic factors, including TNFa, Granzyme B, and IFNg by ELISA (Luminex).

TABLE 10A
Anti-Her2 iCAR Formats and Domains

| Construct | | |
|---|---|---|
| (by ICD) | Domain | Sequence |
| KIR3DL1 | signal | MALPVTALLLPLALLLHAARP |
| LIR1 | sequence | (SEQ ID NO: 71) |
| | 1 (CD8) | |
| | amino | |
| | acid | |
| KIR3DL1 | signal | ATGGCCTTACCAGTGACCGCCTTGCTCCT |
| LIR1 | sequence | GCCGCTGGCCTTGCTGCTCCACGCCGCCA |
| | 1 (CD8) | GGCCG |
| | nucleic | (SEQ ID NO: 72) |
| | acid | |
| KIR3DL1 | signal | KYLLPTAAAGLLLLAAQPAMA |
| LIR1 | sequence | (SEQ ID NO: 73) |
| | 2 (pelB) | |
| | amino | |
| | acid | |
| KIR3DL1 | signal | AAATACCTATTGCCTACGGCAGCCGCTGG |
| LIR1 | sequence | ATTGTTATTACTCGCGGCCCAGCCGGCCA |
| | 2 (pelB) | TGGCC |
| | nucleic | (SEQ ID NO: 74) |
| | acid | |
| KIR3DL1 | scFv | QVQLVQSGAEVKKPGESLKISCKGSGYSF |
| LIR1 | (aHer2 | TSYWIAWVRQMPGKGLEYMGLIYPGDSDT |
| | H3B1 | KYSPSFQGQVTISVDKSVSTAYLQWSSLK |
| | with | PSDSAVYFCARHDVGYCTDRTCAKWPEYF |
| | (G4S)3 | QHWGQGTLVTVSSGGGGSGGGGSGGGGSQ |
| | linker) | SVLTQPPSVSAAPGQKVTISCSGSSSNIG |
| | amino | NNYVSWYQQLPGTAPKLLIYDHTNRPAGV |
| | acid | PDRFSGSKSGTSASLAISGFRSEDEADYY |
| | | CASWDYTLSGWVFGGGTKLTVLG |
| | | (SEQ ID NO: 75) |
| KIR3DL1 | scFv | CAGGTGCAGCTGGTGCAGTCTGGGGCAGA |
| LIR1 | (aHer2 | GGTGAAAAAGCCCGGGGAGTCTCTGAAGA |
| | H3B1 | TCTCCTGTAAGGGTTCTGGATACAGCTTT |
| | with | ACCAGCTACTGGATCGCCTGGGTGCGCCA |
| | (G4S)3 | GATGCCCGGGAAAGGCCTGGAGTACATGG |
| | linker) | GGCTCATCTATCCTGGTGACTCTGACACC |
| | nucleic | AAATACAGCCCGTCCTTCCAAGGCCAGGT |
| | acid | CACCATCTCAGTCGACAAGTCCGTCAGCA |
| | | CTGCCTACTTGCAATGGAGCAGTCTGAAG |
| | | CCCTCGGACAGCGCCGTGTATTTTTGTGC |
| | | GAGACATGACGTGGGATATTGCACCGACC |
| | | GGACTTGCGCAAAGTGGCCTGAATACTTC |
| | | CAGCATTGGGGCCAGGGCACCCTGGTCAC |
| | | CGTCTCCTCAGGTGGAGGCGGTTCAGGCG |
| | | GAGGTGGCTCTGGCGGTGGCGGATCGCAG |
| | | TCTGTGTTGACGCAGCCGCCCTCAGTGTC |
| | | TGCGGCCCCAGGACAGAAGGTCACCATCT |
| | | CCTGCTCTGGAAGCAGCTCCAACATTGGG |
| | | AATAATTATGTATCCTGGTACCAGCAGCT |
| | | CCCAGGAACAGCCCCCAAACTCCTCATCT |
| | | ATGATCACACCAATCGGCCCGCAGGGGTC |
| | | CCTGACCGATTCTCTGGCTCCAAGTCTGG |
| | | CACCTCAGCCTCCCTGGCCATCAGTGGGT |
| | | TCCGGTCCGAGGATGAGGCTGATTATTAC |
| | | TGTGCCTCCTGGGACTACACCCTCTCGGG |
| | | CTGGGTGTTCGGCGGAGGGACCAAGCTGA |
| | | CCGTCCTAGGT |
| | | (SEQ ID NO: 76) |
| KIR3DL1 | tag | GKPIPNPLLGLDSTNGAA |
| LIR1 | (V5 + | (SEQ ID NO: 77) |
| | NGAA | |
| | linker) | |
| | amino | |
| | acid | |
| KIR3DL1 | tag | GGGAAGCCTATCCCGAACCCTCTGTTGGG |
| LIR1 | (V5 + | TCTCGATAGTACCAATGGGGCCGCA |
| | NGAA | (SEQ ID NO: 78) |
| | linker) | |
| | nucleic | |
| | acid | |
| KIR3DL1 | hinge | TTTPAPRPPTPAPTIALQPLSLRPEACRP |
| LIR1 | (CD8) | AAGGAVHTRGLDFACD |
| | amino | (SEQ ID NO: 37) |
| | acid | |
| KIR3DL1 | hinge | ACCACGACGCCAGCGCCGCGACCACCAAC |
| LIR1 | (CD8) | ACCGGCGCCCACCATCGCGTTGCAGCCCC |
| | nucleic | TGTCCCTGCGCCCAGAGGCGTGCCGGCCA |
| | acid | GCGGCGGGGGGCGCAGTGCACACGAGGGG |
| | | GCTGGACTTCGCCTGTGAT |
| | | (SEQ ID NO: 79) |
| KIR3DL1 | TM | ILIGTSVVIILFILLLFFLL |
| | (KIR3 | (SEQ ID NO: 69) |
| | DL1) | |
| | amino | |
| | acid | |
| KIR3DL1 | TM | ATCCTGATCGGGACAAGTGTAGTAATCAT |
| | (KIR3) | ACTTTTCATACTCCTGCTCTTTTTTCTCT |
| | DL1 | TG |
| | nucleic | (SEQ ID NO: 81) |
| | acid | |
| LIR1 | TM | VIGILVAVILLLLLLLLLFLI |
| | (LIR1) | (SEQ ID NO: 59) |
| | amino | |
| | acid | |
| LIR1 | TM | GTTATAGGGATCCTGGTGGCTGTCATACT |
| | (LIR1) | CCTCTTGCTCCTCTTGTTGCTGCTTTTTT |
| | nucleic | TGATA |
| | acid | (SEQ ID NO: 62) |
| KIR3DL1 | inhibi- | HLWCSNKKNAAVMDQEPAGNRTANSEDSD |
| | tory | EQDPEEVTYAQLDHCVFTQRKITRPSQRP |
| | cyto- | KTPPTDTILYTELPNAKPRSKVVSCP |
| | solic | (SEQ ID NO: 66) |
| | domain | |
| | 1 | |
| | (KIR3 | |
| | DL1) | |
| | amino | |
| | acid | |
| KIR3DL1 | inhibi- | CATCTGTGGTGTTCTAATAAGAAGAATGC |
| | tory | TGCTGTGATGGATCAAGAGCCCGCTGGTA |
| | cyto- | ACAGAACGGCCAACAGTGAAGATAGCGAT |
| | solic | GAGCAGGACCCAGAAGAAGTGACCTACGC |
| | domain | CCAACTCGACCACTGTGTTTTTACGCAGC |
| | 1 | GGAAAATCACTCGACCCTCTCAACGACCC |
| | (KIR3 | AAAACGCCGCCTACGGACACCATACTCTA |
| | DL1) | CACCGAACTGCCGAACGCCAAACCACGGT |
| | nucleic | CCAAGGTGGTATCATGTCCG |
| | acid | (SEQ ID NO: 85) |
| LIR1 | inhibi- | LRHRRQGKHWTSTQRKADFQHPAGAVGPE |
| | tory | PTDRGLQWRSSPAADAQEENLYAAVKHTQ |
| | cyto- | PEDGVEMDTRSPHDEDPQAVTYAEVKHSR |
| | solic | PRREMASPPSPLSGEFLDTKDRQAEEDRQ |
| | domain | MDTEAAASEAPQDVTYAQLHSLTLRREAT |
| | 1 | EPPPSQEGPSPAVPSIYATLAIH |
| | (LIR1) | (SEQ ID NO: 50) |
| | amino | |
| | acid | |
| LIR1 | inhibi- | TTGCGCCACAGACGGCAGGGAAAGCACTG |
| | tory | GACTAGTACGCAGAGGAAAGCGGACTTCC |
| | cyto- | AGCATCCCGCAGGAGCCGTGGGGCCTGAA |
| | solic | CCCACTGATCGCGGCCTTCAATGGAGGTC |
| | domain | TAGCCCGGCGGCAGACGCACAAGAGGAAA |
| | 1 | ACTTGTACGCAGCCGTTAAGCACACCCAA |
| | (LIR1) | CCGGAGGACGGCGTTGAGATGGATACCCG |
| | nucleic | CTCCCCTCACGATGAAGACCCTCAAGCAG |
| | acid | TCACTTACGCGGAAGTAAAGCATAGCCGC |
| | | CCCAGACGGGAAATGGCTAGCCCGCCGTC |
| | | CCCCCTTAGCGGGGAATTTCTGGACACTA |
| | | AAGATAGGCAGGCGGAAGAGGACCGCCAA |
| | | ATGGATACAGAGGCGGCGGCAAGTGAAGC |
| | | ACCTCAAGACGTTACTTACGCTCAACTTC |
| | | ACAGCCTTACCCTCAGGCGAGAAGCGACT |
| | | GAACCACCCCCTTCCCAAGAAGGGCCAAG |
| | | CCCAGCGGTTCCTTCTATCTATGCTACTC |
| | | TTGCTATTCAC |
| | | (SEQ ID NO: 54) |

TABLE 10B
aAx1 CD28-CD3z aCAR Domains

| Con- | | |
|---|---|---|
| struct | Domain | Sequence |
| aAx1 | signal | METDTLLLWVLLLWVPGSTG |
| CD28- | sequence | (SEQ ID NO: 113) |
| CD3z | (IgK) | |
| | amino | |
| | acid | |
| aAx1 | signal | ATGGAAACCGACACACTGCTGCTGTGGGTG |
| CD28- | sequence | CTGCTTCTTTGGGTGCCCGGATCTACAGGT |
| CD3z | (IgK) | (SEQ ID NO: 114) |
| | nucleic | |
| | acid | |
| aAx1 | scFv | QVQLQESGPGLVKPSETLSLTCTVSGYSIT |
| CD28- | (aAx1 | SNYWGWIRQPPGKGLEWMGYITYSGSTSYN |
| CD3z | 1448 | PSLKSRITISRDTSKNQFSLKLSSVTAADT |
| | with | AVYYCAITTFYYWGQGTLVTVSSGGGGSGG |
| | (G4S)3 | GGSGGGGSDIQMTQSPSSLSASVGDRVTIT |
| | linker) | CRASQDIGNYLRWFQQKPGKAPKLLISGAT |
| | amino | NLAAGVPSRFSGSGSGSDFTLTISSLQPED |
| | acid | FATYYCLQSKESPWTFGQGTKVEIKRT |
| | | (SEQ ID NO: 115) |
| aAx1 | scFv | CAGGTCCAGCTGCAAGAATCTGGACCAGGC |
| CD28- | (aAx1 | CTCGTGAAGCCCAGCGAGACACTGTCTCTG |
| CD3z | 1448 | ACCTGTACCGTGTCCGGCTACAGCATCACC |
| | with | AGCAACTACTGGGGCTGGATCAGACAGCCT |
| | (G4S)3 | CCTGGCAAAGGCCTTGAGTGGATGGGCTAC |
| | linker) | ATCACCTACAGCGGCAGCACCAGCTACAAC |
| | nucleic | CCCAGCCTGAAGTCCCGGATCACCATCAGC |
| | acid | AGAGACACCAGCAAGAACCAGTTCTCCCTG |
| | | AAGCTGAGCAGCGTGACAGCCGCCGATACA |
| | | GCCGTGTACTACTGTGCCATCACCACCTTC |
| | | TACTATTGGGGCCAGGGCACCCTGGTCACA |
| | | GTTTCTAGCGGAGGCGGAGGATCTGGTGGC |
| | | GGAGGAAGTGGCGGAGGCGGTTCTGATATC |
| | | CAGATGACACAGAGCCCCAGCAGCCTGTCT |
| | | GCCTCTGTGGGAGACAGAGTGACCATCACC |
| | | TGTAGGGCCAGCCAGGACATCGGCAACTAC |
| | | CTGAGATGGTTCCAGCAGAAGCCTGGCAAG |
| | | GCCCCTAAGCTGCTGATTAGCGGCGCCACA |
| | | AATCTGGCTGCTGGCGTGCCAAGCAGATTT |
| | | TCCGGCTCTGGCAGCGGCTCCGATTTCACC |
| | | CTGACCATATCTAGCCTGCAGCCTGAGGAC |
| | | TTCGCCACCTACTACTGCCTGCAGAGCAAA |
| | | GAGAGCCCCTGGACATTTGGACAGGGCACC |
| | | AAGGTGGAAATCAAGCGGACC |
| | | (SEQ ID NO: 116) |
| aAx1 | tag | AGGSDYKDDDDKGGS |
| CD28- | (AGGS | (SEQ ID NO: 117) |
| CD3z | FLAGtag | |
| | GGS) | |
| | amino | |
| | acid | |
| aAx1 | tag | GCCGGCGGAAGCGACTACAAGGACGACGAT |
| CD28- | (AGGS | GACAAAGGCGGCAGC |
| CD3z | FLAGtag | (SEQ ID NO: 118) |
| | GGS) | |
| | nucleic | |
| | acid | |
| aAx1 | hinge | TTTPAPRPPTPAPTIALQPLSLRPEACRPA |
| CD28- | (CD8) | AGGAVHTRGLDFACD |
| CD3z | amino | (SEQ ID NO: 37) |
| | acid | |
| aAx1 | hinge | ACCACGACGCCAGCGCCGCGACCACCAACA |
| CD28- | (CD8) | CCGGCGCCCACCATCGCGTTGCAGCCCCTG |
| CD3z | nucleic | TCCCTGCGCCCAGAGGCGTGCCGGCCAGCG |
| | acid | GCGGGGGGCGCAGTGCACACGAGGGGGCTG |
| | | GACTTCGCCTGTGAT |
| | | (SEQ ID NO: 79) |
| aAx1 | TM | FWVLVVVGGVLACYSLLVTVAFIIFWV |
| CD28- | (CD28) | (SEQ ID NO: 11) |
| CD3z | amino | |
| | acid | |
| aAx1 | TM | TTCTGGGTGCTCGTTGTTGTTGGCGGCGTG |
| CD28- | (CD28) | CTGGCCTGTTATTCCCTGCTGGTTACCGTG |
| CD3z | nucleic | GCCTTCATCATCTTTTGGGTC |
| | acid | (SEQ ID NO: 83) |
| aAx1 | intra- | RSKRSRLLHSDYMNMTPRRPGPTRKHYQPY |
| CD28- | cellu- | APPRDFAAYRS |
| CD3z | lar | (SEQ ID NO: 119) |
| | signal- | |
| | ing | |
| | domain | |
| | 1 | |
| | (CD28) | |
| | amino | |
| | acid | |
| aAx1 | intra- | CGAAGCAAGCGGAGCCGGCTGCTGCACAGC |
| CD28- | cellu- | GATTACATGAACATGACCCCTCGGAGGCCC |
| CD3z | lar | GGACCTACCAGAAAGCACTACCAGCCTTAC |
| | signal- | GCTCCTCCTAGAGATTTCGCCGCCTACCGG |
| | ing | TCC |
| | domain | (SEQ ID NO: 120) |
| | 1 | |
| | (CD28) | |
| | nucleic | |
| | acid | |
| aAx1 | intra- | RVKFSRSADAPAYKQGQNQLYNELNLGRRE |
| CD28- | cellu- | EYDVLDKRRGRDPEMGGKPRRKNPQEGLYN |
| CD3z | lar | ELQKDKMAEAYSEIGMKGERRRGKGHDGLY |
| | signal- | QGLSTATKDTYDALHMQALPPR |
| | ing | (SEQ ID NO: 121) |
| | domain | |
| | 2 | |
| | (CD3ζ) | |
| | amino | |
| | acid | |
| aAx1 | intra- | AGAGTGAAGTTCAGCAGATCCGCCGATGCT |
| CD28- | cellu- | CCCGCCTATAAGCAGGGCCAGAACCAGCTG |
| CD3z | lar | TACAACGAGCTGAACCTGGGGAGAAGAGAA |
| | signal- | GAGTACGACGTGCTGGACAAGCGGAGAGGC |
| | ing | AGAGATCCTGAAATGGGCGGCAAGCCCAGA |
| | domain | CGGAAGAATCCTCAAGAGGGCCTGTATAAT |
| | 2 | GAGCTGCAGAAAGACAAGATGGCCGAGGCC |
| | (CD3ζ) | TACAGCGAGATCGGAATGAAGGGCGAGCGC |
| | nucleic | AGAAGAGGCAAGGGACACGATGGACTGTAC |
| | acid | CAGGGACTGAGCACCGCCACCAAGGATACC |
| | | TATGACGCCCTGCACATGCAGGCCCTGCCT |
| | | CCAAGA |
| | | (SEQ ID NO: 122) |

### Results

NK cells were engineered to express activating chimeric receptors (aCARS) and inhibitory chimeric receptors (iCARs) having LIR1 and KIR3DL1 inhibitory domains. Engineered NK cells were then assessed for iCARs reducing aCAR mediated activation of NK cells.

NK cells were virally transduced with aCAR only (anti-Axl-CD28/CD3ζ; “αAxl 28ζ”), or in combination with anti-Her2 iCAR1 (LIR1 inhibitory domain; “aHer2 LIR1”) or iCAR2 (KIR3DL1 inhibitory domain; “aHer2 KIR3DL1”). As shown in FIG. 10, the CARs were expressed in ~50% of NK cells for aCAR alone (top right panel). NK cells co-engineered with iCARS demonstrated co-expression (aCAR+iCAR+) in ~50% of cells (top right quadrant of each bottom panel). Notably, co-engineered NK cells only demonstrated ~5-6% of cells expressing the aCAR only (aCAR+iCAR−; bottom right quadrant of each bottom panel). The expression results demonstrate NK cells can be successfully engineered to co-express aCARs and iCARs.

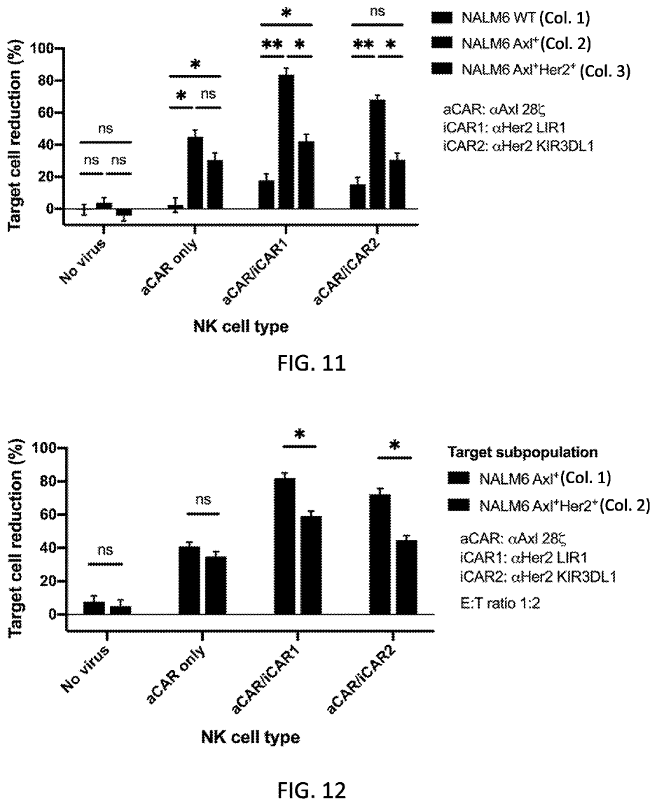

Engineered NK cells were then assessed for iCARs reducing aCAR induced NK cell mediated killing of target cells. As shown in FIG. 11, NK cells engineered to co-express the aCAR and iCAR killed target cells only expressing the aCAR antigen (NALM6 Axl+; column 2 each engineering condition) at least as well as NK cells transduced with aCAR only relative to killing of parental target cells not expressing the aCAR antigen (NALM6 WT; column 1 each engineering condition) demonstrating aCAR antigen dependent antigen-specific killing. When co-incubated with target cells expressing both aCAR and iCAR antigen (NALM6 Axl+Her2+; column 3 each engineering condition), NK cells engineered to co-express the aCAR and iCAR exhibited significantly reduced killing relative to killing of target cells only expressing the aCAR antigen (aCAR/iCAR1 and aCAR/iCAR2 comparing columns 3 to 2, respectively). In contrast, NK cells engineered to express aCAR only did not demonstrate a significant reduction in killing (aCAR only comparing columns 3 to 2, respectively). The results demonstrate NK cells engineered to co-express aCARs and iCARs successfully kill target cells in the absence of an iCAR ligand and successfully reduce NK-mediated killing in the presence of an iCAR ligand.

Engineered NK cells were then assessed for iCARs reducing aCAR induced NK cell mediated killing in the context of a mixed target population. As shown in FIG. 12, NK cells engineered to co-express the aCAR and iCAR exhibited significantly reduced killing of target cells expressing both aCAR and iCAR antigen relative to killing of target cells expressing only the aCAR ligand within a mixed population (aCAR/iCAR1 and aCAR/iCAR2 comparing columns 2 to 1, respectively), in contrast to NK cells engineered to express aCAR-only (aCAR only comparing columns 2 to 1, respectively). The results demonstrate NK cells engineered to co-express aCARs and iCARs successfully selectively kill target cells that do not express an iCAR ligand in a mixed population of cells.

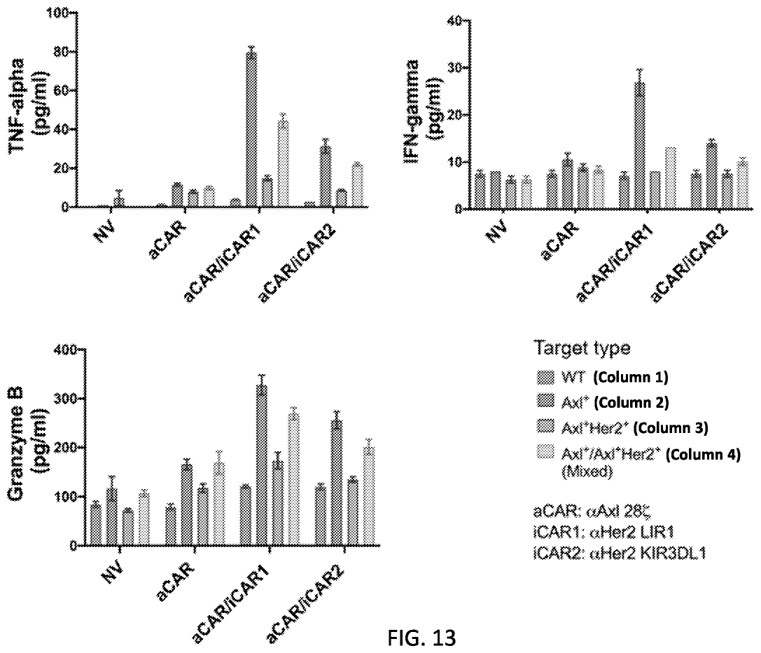

Engineered NK cells were then assessed for iCARs reducing aCAR mediated activation of NK cells as assessed by cytokine production. As shown in FIG. 13, NK cells engineered to co-express the aCAR and iCAR secreted cytokines TNFα, Granzyme B, and IFNγ when co-incubated with target cells expressing only the aCAR ligand (aCAR/iCAR1 and aCAR/iCAR2 column 2) or a mixed population of target cells with half expressing only the aCAR ligand (aCAR/iCAR1 and aCAR/iCAR2 comparing column 4), while cytokine secretion was reduced following co-incubation with target cells expressing both aCAR and iCAR antigens (aCAR/iCAR1 and aCAR/iCAR2 comparing column 3).

The results demonstrate NK cells can be successfully engineered to co-express aCARs and iCARs, NK cells engineered to co-express aCARs and iCARs successfully kill target cells and proinflammatory cytokine production in the absence of an iCAR ligand, and NK cells engineered to co-express aCARs and iCARs successfully reduce NK-mediated killing and proinflammatory cytokine production in an iCAR ligand dependent manner.

### Example 4: Assessment of Various Inhibitory Chimeric Receptors in Reducing NK Cell Activation

### Materials and Methods

### Transduction and Expansion

NK cells were expanded for 10 days with mitomycin C-treated K562 feeder cells, followed by transduction with 7.5e5 pg of each lentivirus for aCAR and iCAR constructs. Sequences for the iCAR constructs assessed are shown in Table 11. Sequences for the aCAR construct assessed are shown in Table 10B. Each iCAR construct format is from N to C terminal: signal sequence 1-signal sequence 2-scFv-tag-hinge-TM-inhibitory cytosolic domain 1-inhibitory cytosolic domain 2 (if present). The aAxl CD28-CD3z format is from N to C terminal: signal sequence-tag-scFv-hinge-TM-intracellular signaling domain 1-intracellular signaling domain 2. After 4 days, puromycin was added to cells for selection.

After 3 more days, cytotoxicity assays were performed by co-incubating engineered NK cells and parental SEM target cells (WT), or SEM targets engineered to overexpress Axl or both Axl and Her2 antigens. Each engineered NK cells were incubated either with (1) each target cell type separately at a ratio of 25,000 NK cells to 50,000 SEM cells in triplicate; or (2) as a mixture of 25,000 single antigen Axl+ only and 25,000 dual antigen Axl+Her2+ SEM cells co-incubated with 25,000 NK cells of the indicated type in a 1:1:1 ratio (dual antigen targets were stained with different membrane dyes allowing them to be distinguished by flow). After overnight incubation, cells were stained with viability dyes and counted via flow cytometry. The target cell reduction was quantified as 100%×(1−No. Targets/No. Targets (NV)).

TABLE 11
Anti-Her2 iCAR Formats and Domains

| Construct | | |
|---|---|---|
| (by ICD) | Domain | Sequence |
| PD-1 | signal | MALPVTALLLPLALLLHAARP |
| CTLA-4 | sequence 1 | (SEQ ID NO: 71) |
| KIR3DL1 | (CD8) | |
| LIR1 | amino acid | |
| BTLA | | |
| NKG2A | | |
| PD-1 | signal | ATGGCCTTACCAGTGACCGCCTTGCTCCTGCCGCTGGCCTTG |
| CTLA-4 | sequence 1 | CTGCTCCACGCCGCCAGGCCG |
| KIR3DL1 | (CD8) | (SEQ ID NO: 72) |
| LIR1 | nucleic acid | |
| BTLA | | |
| NKG2A | | |
| PD-1 | signal | KYLLPTAAAGLLLLAAQPAMA |
| CTLA-4 | sequence 2 | (SEQ ID NO: 73) |
| KIR3DL1 | (pelB) | |
| LIR1 | amino acid | |
| BTLA | | |
| NKG2A | | |
| PD-1 | signal | AAATACCTATTGCCTACGGCAGCCGCTGGATTGTTATTACTC |
| CTLA-4 | sequence 2 | GCGGCCCAGCCGGCCATGGCC |
| KIR3DL1 | (pelB) | (SEQ ID NO: 74) |
| LIR1 | nucleic acid | |
| BTLA | | |
| NKG2A | | |
| PD-1 | scFv | SEQ ID NO: 75 |
| CTLA-4 | (aHer2H3B1 | |
| KIR3DL1 | with (G4S)3 | |
| LIR1 | linker) | |
| BTLA | amino acid | |
| NKG2A | | |
| PD-1 | scFv | SEQ ID NO: 76 |
| CTLA-4 | (aHer2H3B1 | |
| KIR3DL1 | with (G4S)3 | |
| LIR1 | linker) | |
| BTLA | nucleic acid | |
| NKG2A | | |
| PD-1 | tag | GKPIPNPLLGLDSTNGAA |
| CTLA-4 | (V5 + NGAA | (SEQ ID NO: 77) |
| KIR3DL1 | linker) | |
| LIR1 | amino acid | |
| BTLA | | |
| NKG2A | | |
| PD-1 | tag | GGGAAGCCTATCCCGAACCCTCTGTTGGGTCTCGATAGTACC |
| CTLA-4 | (V5 + NGAA | AATGGGGCCGCA |
| KIR3DL1 | linker) | (SEQ ID NO: 78) |
| LIR1 | nucleic acid | |
| BTLA | | |
| NKG2A | | |
| PD-1 | hinge | TTTPAPRPPTPAPTIALQPLSLRPEACRPAAGGAVHTRGLDFAC |
| CTLA-4 | (CD8) | D |
| KIR3DL1 | amino acid | (SEQ ID NO: 37) |
| LIR1 | | |
| BTLA | | |
| NKG2A | | |
| PD-1 | hinge | ACCACGACGCCAGCGCCGCGACCACCAACACCGGCGCCCAC |
| CTLA-4 | (CD8) | CATCGCGTTGCAGCCCCTGTCCCTGCGCCCAGAGGCGTGCC |
| KIR3DL1 | nucleic acid | GGCCAGCGGCGGGGGGCGCAGTGCACACGAGGGGGCTGGA |
| LIR1 | | CTTCGCCTGTGAT |
| BTLA | | (SEQ ID NO: 79) |
| NKG2A | | |
| PD-1 | TM | VGVVGGLLGSLVLLVWVLAVI |
| | (PD-1) | (SEQ ID NO: 60) |
| | amino acid | |
| PD-1 | TM | GTTGGGGTTGTAGGTGGTCTGCTCGGCAGCCTGGTCTTGTTG |
| | (PD-1) | GTGTGGGTCTTGGCTGTGATC |
| | nucleic acid | (SEQ ID NO: 64) |
| CTLA-4 | TM | DFLLWILAAVSSGLFFYSFLLT |
| | (CTLA-4) | (SEQ ID NO: 68) |
| | amino acid | |
| CTLA-4 | TM | GATTTTCTGCTGTGGATTCTGGCAGCTGTGAGCTCTGGCTTG |
| | (CTLA-4) | TTTTTCTACAGCTTCCTCCTGACC |
| | nucleic acid | (SEQ ID NO: 80) |
| KIR3DL1 | TM | ILIGTSVVIILFILLLFFLL |
| | (KIR3DL1) | (SEQ ID NO: 69) |
| | amino acid | |
| KIR3DL1 | TM | ATCCTGATCGGGACAAGTGTAGTAATCATACTTTTCATACTC |
| | (KIR3DL1) | CTGCTCTTTTTTCTCTTG |
| | nucleic acid | (SEQ ID NO: 81) |
| LIR1 | TM | VIGILVAVILLLLLLLLLFLI |
| | (LIR1) | (SEQ ID NO: 59) |
| | amino acid | |
| LIR1 | TM | GTTATAGGGATCCTGGTGGCTGTCATACTCCTCTTGCTCCTC |
| | (LIR1) | TTGTTGCTGCTTTTTTTGATA |
| | nucleic acid | (SEQ ID NO: 62) |
| BTLA | TM | LLPLGGLPLLITTCFCLFCCL |
| | (BTLA) | (SEQ ID NO: 12) |
| | nucleic acid | |
| BTLA | TM | CTCTTGCCGTTGGGGGGTCTGCCACTTCTCATAACAACTTGC |
| | (BTLA) | TTCTGCCTTTTTTGCTGTTTG |
| | nucleic acid | (SEQ ID NO: 14) |
| CD28 | TM | FWVLVVVGGVLACYSLLVTVAFIIFWV |
| | (CD28) | (SEQ ID NO: 11) |
| | amino acid | |
| CD28 | TM | TTCTGGGTGCTCGTTGTTGTTGGCGGCGTGCTGGCCTGTTAT |
| | (CD28) | TCCCTGCTGGTTACCGTGGCCTTCATCATCTTTTGGGTC |
| | nucleic acid | (SEQ ID NO: 83) |
| NKG2A | TM | IVVITVVSAMLILCIIGLIGVIL |
| | (NKG2A- | (SEQ ID NO: 89) |
| | reversed) | |
| | amino acid | |
| NKG2A | TM | ATAGTGGTCATCACTGTAGTTAGTGCAATGCTTATTCTTTGT |
| | (NKG2A- | ATCATAGGGCTCATAGGGGTAATCCTG |
| | reversed) | (SEQ ID NO: 90) |
| | amino acid | |
| PD-1 | inhibitory | CSRAARGTIGARRTGQPLKEDPSAVPVFSVDYGELDFQWREKT |
| | cytosolic | PEPPVPCVPEQTEYATIVFPSGMGTSSPARRGSADGPRSAQPLR |
| | domain 1 | PEDGHCSWPL |
| | (PD-1) | (SEQ ID NO: 1) |
| | amino acid | |
| PD-1 | inhibitory | TGTAGCCGAGCGGCCAGAGGCACAATCGGGGCAAGACGAA |
| | cytosolic | CAGGACAGCCGCTCAAAGAGGACCCCAGTGCGGTCCCCGTT |
| | domain 1 | TTCTCCGTGGATTACGGAGAACTGGATTTCCAGTGGCGGGA |
| | (PD-1) | GAAGACACCAGAGCCCCCGGTGCCCTGCGTGCCGGAGCAGA |
| | nucleic acid | CTGAGTACGCCACGATTGTGTTTCCCTCTGGAATGGGGACTT |
| | | CATCCCCCGCTAGGCGCGGCTCAGCTGATGGCCCAAGATCC |
| | | GCTCAACCGTTGCGGCCAGAGGACGGGCATTGCAGTTGGCC |
| | | TCTG |
| | | (SEQ ID NO: 51) |
| CTLA-4 | inhibitory | AVSLSKMLKKRSPLTTGVGVKMPPTEPECEKQFQPYFIPIN |
| | cytosolic | (SEQ ID NO: 67) |
| | domain 1 | |
| | (CTLA-4) | |
| | amino acid | |
| CTLA-4 | inhibitory | GCCGTGTCACTTAGTAAGATGCTGAAGAAGAGGTCACCACT |
| | cytosolic | GACGACAGGGGTTGGAGTGAAGATGCCACCCACAGAACCC |
| | domain 1 | GAATGTGAGAAGCAATTCCAGCCTTATTTCATTCCAATAAAT |
| | (CTLA-4) | (SEQ ID NO: 84) |
| | nucleic acid | |
| KIR3DL1 | inhibitory | HLWCSNKKNAAVMDQEPAGNRTANSEDSDEQDPEEVTYAQL |
| | cytosolic | DHCVFTQRKITRPSQRPKTPPTDTILYTELPNAKPRSKVVSCP |
| | domain 1 | (SEQ ID NO: 66) |
| | (KIR3DL1) | |
| | amino acid | |
| KIR3DL1 | inhibitory | CATCTGTGGTGTTCTAATAAGAAGAATGCTGCTGTGATGGAT |
| | cytosolic | CAAGAGCCCGCTGGTAACAGAACGGCCAACAGTGAAGATA |
| | domain 1 | GCGATGAGCAGGACCCAGAAGAAGTGACCTACGCCCAACTC |
| | (KIR3DL1) | GACCACTGTGTTTTTACGCAGCGGAAAATCACTCGACCCTCT |
| | nucleic acid | CAACGACCCAAAACGCCGCCTACGGACACCATACTCTACAC |
| | | CGAACTGCCGAACGCCAAACCACGGTCCAAGGTGGTATCAT |
| | | GTCCG |
| | | (SEQ ID NO: 85) |
| LIR1 | inhibitory | LRHRRQGKHWTSTQRKADFQHPAGAVGPEPTDRGLQWRSSPA |
| | cytosolic | ADAQEENLYAAVKHTQPEDGVEMDTRSPHDEDPQAVTYAEV |
| | domain 1 | KHSRPRREMASPPSPLSGEFLDTKDRQAEEDRQMDTEAAASEA |
| | (LIR1) | PQDVTYAQLHSLTLRREATEPPPSQEGPSPAVPSIYATLAIH |
| | amino acid | (SEQ ID NO: 50) |
| LIR1 | inhibitory | TTGCGCCACAGACGGCAGGGAAAGCACTGGACTAGTACGCA |
| | cytosolic | GAGGAAAGCGGACTTCCAGCATCCCGCAGGAGCCGTGGGGC |
| | domain 1 | CTGAACCCACTGATCGCGGCCTTCAATGGAGGTCTAGCCCG |
| | (LIR1) | GCGGCAGACGCACAAGAGGAAAACTTGTACGCAGCCGTTAA |
| | nucleic acid | GCACACCCAACCGGAGGACGGCGTTGAGATGGATACCCGCT |
| | | CCCCTCACGATGAAGACCCTCAAGCAGTCACTTACGCGGAA |
| | | GTAAAGCATAGCCGCCCCAGACGGGAAATGGCTAGCCCGCC |
| | | GTCCCCCCTTAGCGGGGAATTTCTGGACACTAAAGATAGGC |
| | | AGGCGGAAGAGGACCGCCAAATGGATACAGAGGCGGCGGC |
| | | AAGTGAAGCACCTCAAGACGTTACTTACGCTCAACTTCACA |
| | | GCCTTACCCTCAGGCGAGAAGCGACTGAACCACCCCCTTCC |
| | | CAAGAAGGGCCAAGCCCAGCGGTTCCTTCTATCTATGCTACT |
| | | CTTGCTATTCAC |
| | | (SEQ ID NO: 54) |
| BTLA | inhibitory | RRHQGKQNELSDTAGREINLVDAHLKSEQTEASTRQNSQVLLS |
| | cytosolic | ETGIYDNDPDLCFRMQEGSEVYSNPCLEENKPGIVYASLNHSVI |
| | domain 1 | GPNSRLARNVKEAPTEYASICVRS |
| | (BTLA) | (SEQ ID NO: 3) |
| | amino acid | |
| BTLA | inhibitory | AGAAGACATCAGGGGAAGCAGAATGAACTCAGCGATACAG |
| | cytosolic | CAGGGCGAGAAATTAATTTGGTAGACGCGCATCTGAAGTCC |
| | domain 1 | GAACAGACAGAGGCTTCTACTAGACAGAACTCCCAAGTTTT |
| | (BTLA) | GTTGAGTGAGACGGGGATCTATGATAATGATCCCGATCTGT |
| | nucleic acid | GTTTTAGAATGCAGGAGGGTAGTGAAGTCTACTCAAACCCG |
| | | TGCCTGGAAGAAAATAAGCCCGGCATTGTTTACGCTAGTTT |
| | | GAATCATTCTGTAATAGGCCCGAACTCCAGACTGGCTCGCA |
| | | ATGTGAAGGAGGCCCCAACTGAGTATGCGTCCATTTGCGTG |
| | | CGGTCT |
| | | (SEQ ID NO: 52) |
| NKG2A | inhibitory | KEPASPLDKCHYTKDNGQFDQSAKQLNLEAYTIEQETALISNK |
| | cytosolic | NGKPKRQQRKPNPPLNLDSYIVGQNDM |
| | domain 1 | (SEQ ID NO: 93) |
| | (NKG2A- | |
| | reversed) | |
| | amino acid | |
| NKG2A | inhibitory | AAGGAGCCTGCGTCCCCGTTGGATAAATGCCACTATACTAA |
| | cytosolic | GGATAACGGTCAGTTCGATCAGAGTGCAAAGCAACTTAACT |
| | domain 1 | TGGAGGCTTACACTATAGAGCAAGAAACAGCGCTGATAAGT |
| | (NKG2A- | AATAAGAACGGTAAGCCAAAGCGACAGCAGAGGAAACCCA |
| | reversed) | ATCCTCCGCTTAACTTGGATAGCTACATCGTCGGGCAAAATG |
| | nucleic acid | ACATG |
| | | (SEQ ID NO: 94) |

### Results

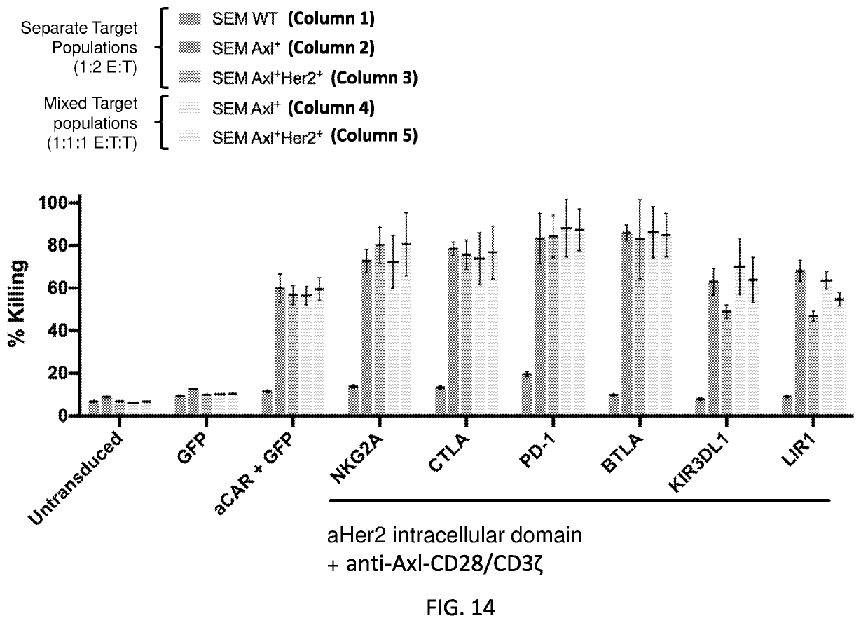

NK cells were engineered to express activating chimeric receptors (aCARS) and inhibitory chimeric receptors (iCARs) having various inhibitory domains. NK cells were virally transduced with aCAR only (anti-Axl-CD28/CD3ζ; “αAxl 28ζ”), or in combination with anti-Her2 iCARs having the various inhibitory domains indicated. Engineered NK cells were then assessed for iCARs reducing aCAR induced NK cell mediated killing of target cells. As shown in FIG. 14, NK cells engineered to co-express the aCAR and iCAR killed target cells expressing only the aCAR antigen (“Axl+”) as a separate target population (columns 2 each engineering condition) or in a mixed target population (column 4 each engineering condition) at least as well as NK cells transduced with aCAR only relative to killing of parental target cells not expressing the aCAR antigen (column 1 each engineering condition) demonstrating antigen-specific killing. Notably, NK cells engineered to co-express anti-Her2 iCARs having LIR1 and KIR3DL1 inhibitory domains demonstrated reduced killing of target cells expressing the aCAR antigen and iCAR antigen (“Axl+Her+”) as a separate target population (columns 3 each engineering condition) or in a mixed target population (column 5 each engineering condition) relative to target cells expressing only the aCAR antigen, while differences in NK cells engineered to co-express anti-Her2 iCARs having NKG2A, CTLA4, PD-1, or BTLA inhibitory domains were not observed. The results demonstrate NK cells engineered to co-express aCARs and select iCARs successfully kill target cells in the absence of an iCAR ligand and successfully reduce NK-mediated killing in an iCAR ligand dependent manner, while also indicating iCARs having inhibitory domains derived from different native inhibitory co-receptors can vary in iCAR antigen-dependent suppression of NK cell activation relative to one another.

### Example 5: Assessment of Tandem Inhibitory Chimeric Receptors in Reducing NK Cell Activation

### Materials and Methods

### Transduction and Expansion

NK cells were expanded for 10 days with mitomycin C-treated K562 feeder cells, followed by transduction with 7.5e5 pg of each lentivirus for aCAR and iCAR constructs having tandem inhibitory domains. Sequences for the iCAR constructs assessed are shown in Table 12. Sequences for the aCAR construct assessed are shown in Table 10B. Each iCAR construct format is from N to C terminal: signal sequence 1-signal sequence 2-scFv-tag-hinge-TM-inhibitory cytosolic domain 1-inhibitory cytosolic domain 2 (if present). The aAxl CD28-CD3z format is from N to C terminal: signal sequence-tag-scFv-hinge-TM-intracellular signaling domain 1-intracellular signaling domain 2. After 4 days, puromycin was added to cells for selection.

After 3 more days, cytotoxicity assays were performed by co-incubating engineered NK cells and parental SEM target cells (WT), or SEM targets engineered to overexpress Axl or both Axl and Her2 antigens. Each engineered NK cells were incubated either with (1) each target cell type separately at a ratio of 25,000 NK cells to 50,000 SEM cells in triplicate; or (2) as a mixture of 25,000 single antigen Axl+ only and 25,000 dual antigen Axl+Her2+ SEM cells co-incubated with 25,000 NK cells of the indicated type in a 1:1:1 ratio (dual antigen targets were stained with different membrane dyes allowing them to be distinguished by flow). After overnight incubation, cells were stained with viability dyes and counted via flow cytometry. The target cell reduction was quantified as 100%×(1−No. Targets/No. Targets (NV)).

TABLE 12
Anti-Her2 iCAR Formats and Domains

| Con- | | |
|---|---|---|
| struct | | |
| (by | | |
| ICD) | Domain | Sequence |
| LIR1- | signal | MALPVTALLLPLALLLHAARP |
| BTLA | sequence | (SEQ ID NO: 71) |
| LIR1- | 1 (CD8) | |
| PD1 | amino | |
| | acid | |
| LIR1- | signal | ATGGCCTTACCAGTGACCGCCTTGCTCCTGCCG |
| BTLA | sequence | CTGGCCTTGCTGCTCCACGCCGCCAGGCCG |
| LIR1- | 1 (CD8) | (SEQ ID NO: 72) |
| PD1 | nucleic | |
| | acid | |
| LIR1- | signal | KYLLPTAAAGLLLLAAQPAMA |
| BTLA | sequence | (SEQ ID NO: 73) |
| LIR1- | 2 (pelB) | |
| PD1 | amino | |
| | acid | |
| LIR1- | signal | AAATACCTATTGCCTACGGCAGCCGCTGGATT |
| BTLA | sequence | GTTATTACTCGCGGCCCAGCCGGCCATGGCC |
| LIR1- | 2 (pelB) | (SEQ ID NO: 74) |
| PD1 | nucleic | |
| | acid | |
| LIR1- | scFv | SEQ ID NO: 75 |
| BTLA | (aHer2 | |
| LIR1- | H3B1 | |
| PD1 | with | |
| | (G4S)3 | |
| | linker) | |
| | amino | |
| | acid | |
| LIR1- | scFv | SEQ ID NO: 76 |
| BTLA | (aHer2 | |
| LIR1- | H3B1 | |
| PD1 | with | |
| | (G4S)3 | |
| | linker) | |
| | nucleic | |
| | acid | |
| LIR1- | tag | GKPIPNPLLGLDSTNGAA |
| BTLA | (V5 + | (SEQ ID NO: 77) |
| LIR1- | NGAA | |
| PD1 | linker) | |
| | amino | |
| | acid | |
| LIR1- | tag | GGGAAGCCTATCCCGAACCCTCTGTTGGGTCT |
| BTLA | (V5 + | CGATAGTACCAATGGGGCCGCA |
| LIR1- | NGAA | (SEQ ID NO: 78) |
| PD1 | linker) | |
| | nucleic | |
| | acid | |
| LIR1- | hinge | TTTPAPRPPTPAPTIALQPLSLRPEACRPAAGG |
| BTLA | (CD8) | AVHTRGLDFACD |
| LIR1- | amino | (SEQ ID NO: 37) |
| PD1 | acid | |
| LIR1- | hinge | ACCACGACGCCAGCGCCGCGACCACCAACACC |
| BTLA | (CD8) | GGCGCCCACCATCGCGTTGCAGCCCCTGTCCC |
| LIR1- | nucleic | TGCGCCCAGAGGCGTGCCGGCCAGCGGCGGG |
| PD1 | acid | GGGCGCAGTGCACACGAGGGGGCTGGACTTCG |
| | | CCTGTGAT |
| | | (SEQ ID NO: 79) |
| LIR1- | TM | VIGILVAVILLLLLLLLLFLI |
| BTLA | (LIR1) | (SEQ ID NO: 59) |
| LIR1- | amino | |
| PD1 | acid | |
| LIR1- | TM | GTTATAGGGATCCTGGTGGCTGTCATACTCCTC |
| BTLA | (LIR1) | TTGCTCCTCTTGTTGCTGCTTTTTTTGATA |
| LIR1- | nucleic | (SEQ ID NO: 62) |
| PD1 | acid | |
| LIR1- | inhibi- | SEQ ID NO: 50 |
| BTLA | tory | |
| LIR1- | cyto- | |
| PD1 | solic | |
| | domain | |
| | 1 (LIR1) | |
| | amino | |
| | acid | |
| LIR1- | inhibi- | SEQ ID NO: 54 |
| BTLA | tory | |
| LIR1- | cyto- | |
| PD1 | solic | |
| | domain | |
| | 1 (LIR1) | |
| | nucleic | |
| | acid | |
| LIR1- | inhibi- | RRHQGKQNELSDTAGREINLVDAHLKSEQTEAS |
| BTLA | tory | TRQNSQVLLSETGIYDNDPDLCFRMQEGSEVYS |
| | cyto- | NPCLEENKPGIVYASLNHSVIGPNSRLARNVKE |
| | solic | APTEYASICVRS |
| | domain | (SEQ ID NO: 3) |
| | 2 (BTLA) | |
| | amino | |
| | acid | |
| LIR1- | inhibi- | AGAAGACATCAGGGGAAGCAGAATGAACTCA |
| BTLA | tory | GCGATACAGCAGGGCGAGAAATTAATTTGGTA |
| | cyto- | GACGCGCATCTGAAGTCCGAACAGACAGAGG |
| | solic | CTTCTACTAGACAGAACTCCCAAGTTTTGTTGA |
| | domain | GTGAGACGGGGATCTATGATAATGATCCCGAT |
| | 2 (BTLA) | CTGTGTTTTAGAATGCAGGAGGGTAGTGAAGT |
| | nucleic | CTACTCAAACCCGTGCCTGGAAGAAAATAAGC |
| | acid | CCGGCATTGTTTACGCTAGTTTGAATCATTCTG |
| | | TAATAGGCCCGAACTCCAGACTGGCTCGCAAT |
| | | GTGAAGGAGGCCCCAACTGAGTATGCGTCCAT |
| | | TTGCGTGCGGTCT |
| | | (SEQ ID NO: 52) |
| LIR1- | inhibi- | CSRAARGTIGARRTGQPLKEDPSAVPVFSVDYG |
| PD1 | tory | ELDFQWREKTPEPPVPCVPEQTEYATIVFPSGM |
| | cyto- | GTSSPARRGSADGPRSAQPLRPEDGHCSWPL |
| | solic | (SEQ ID NO: 1) |
| | domain | |
| | 2 (PD-1) | |
| | amino | |
| | acid | |
| LIR1- | inhibi- | TGTAGCCGAGCGGCCAGAGGCACAATCGGGG |
| PD1 | tory | CAAGACGAACAGGACAGCCGCTCAAAGAGGA |
| | cyto- | CCCCAGTGCGGTCCCCGTTTTCTCCGTGGATTA |
| | solic | CGGAGAACTGGATTTCCAGTGGCGGGAGAAG |
| | domain | ACACCAGAGCCCCCGGTGCCCTGCGTGCCGGA |
| | 2 (PD-1) | GCAGACTGAGTACGCCACGATTGTGTTTCCCT |
| | nucleic | CTGGAATGGGGACTTCATCCCCCGCTAGGCGC |
| | acid | GGCTCAGCTGATGGCCCAAGATCCGCTCAACC |
| | | GTTGCGGCCAGAGGACGGGCATTGCAGTTGGC |
| | | CTCTG |
| | | (SEQ ID NO: 51) |

### Results

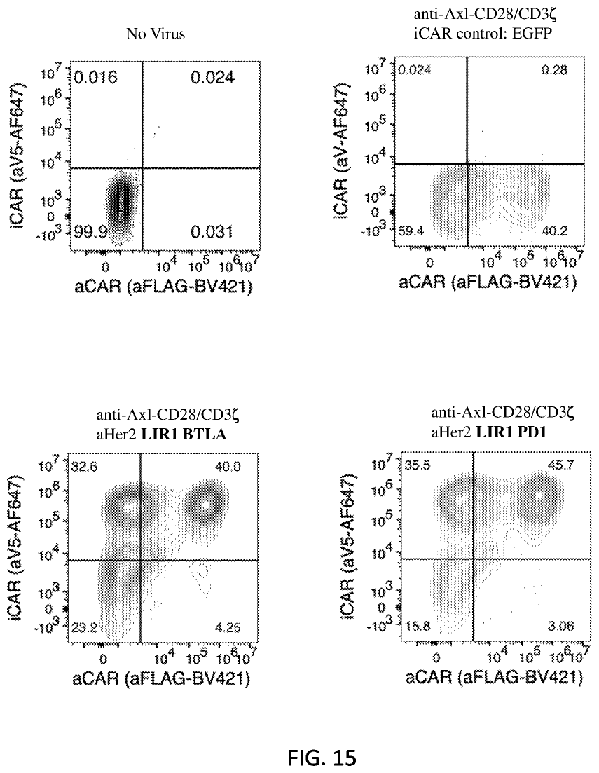

NK cells were engineered to express activating chimeric receptors (aCARS) and inhibitory chimeric receptors (iCARs) with intracellular domains having inhibitory domains in tandem. NK cells were virally transduced with aCAR only (anti-Axl-CD28/CD3ζ; “αAxl 28ζ”), or in combination with anti-Her2 iCARs having the various tandem inhibitory domains indicated. As shown in FIG. 15, the CARs were expressed in ~40% of NK cells for aCAR alone (top right panel). NK cells co-engineered with iCARS demonstrated co-expression (aCAR+iCAR+) in ~40-45% of cells (top right quadrant of each bottom panel). Notably, co-engineered NK cells only demonstrated less than 5% of cells expressing the aCAR only (aCAR+iCAR−; bottom right quadrant of each bottom panel). The expression results demonstrate NK cells can be successfully engineered to co-express aCARs and iCARs with tandem intracellular inhibitory domains.

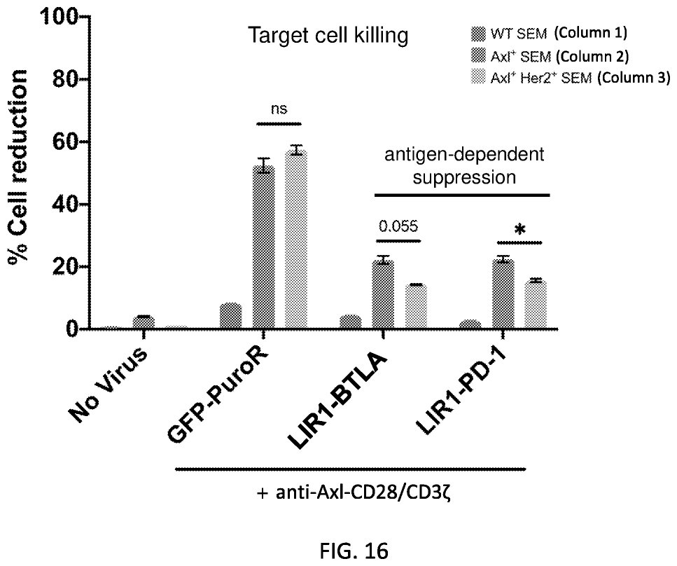

Engineered NK cells were then assessed for iCARs reducing aCAR induced NK cell mediated killing of target cells. As shown in FIG. 16, NK cells engineered to co-express the aCAR and iCAR killed Axl+ target cells (column 2 each engineering condition), though not as well as NK cells transduced with aCAR only (GFP-PuroR) relative to killing of parental cells (WT SEM) not expressing the aCAR antigen (column 1 each engineering condition) demonstrating aCAR antigen dependent antigen-specific killing. When co-incubated with target cells expressing both aCAR and iCAR antigen (Axl+Her2+ SEM cells; column 3 each engineering condition), NK cells engineered to co-express the aCAR and an iCAR with a tandem LIR1/PD-1 organization exhibited significantly reduced killing and an iCAR with a tandem LIR1/BTLA organization exhibited observably (p=0.055) reduced killing relative to killing of target cells expressing only the aCAR antigen (comparing columns 3 to 2). In contrast, NK cells engineered to express aCAR-only did not demonstrate an observable reduction in killing (GFP-PuroR comparing columns 3 to 2, respectively). The results demonstrate NK cells engineered to co-express aCARs and iCARs successfully kill target cells in the absence of an iCAR ligand and successfully reduce NK-mediated killing in an iCAR ligand dependent manner.

### Example 6: Assessment of Inhibitory Chimeric Receptors with or without Extracellular Domains from Inhibitory Domains in Reducing T Cell Activation

### Materials and Methods

### Transduction and Expansion

Primary T cells were isolated from human donor PBMCs and frozen. On day 1, 1×10⁶ purified CD4+/CD8+ T-cells were thawed and stimulated with 3×10⁶ Dynabeads, then cultured in 1 mL Optimizer CTS T-cell expansion media (Gibco) with 0.2 ug/mL IL-2. T cells were singly or co-transduced on day 2 with lentivirus (100K each, as quantified by GoStix (Tekara)). Sequences for the iCAR constructs assessed are shown in Table 13A. Each iCAR construct format is from N to C terminal (except those designated as “full”): signal sequence 1-signal sequence 2-scFv-tag-hinge-TM-inhibitory cytosolic domain 1-inhibitory cytosolic domain 2 (if present). Each iCAR construct format having an ECD (designated as “full”) is from N to C terminal (except NKG2A “full”): signal sequence 1-signal sequence 2-scFv-tag-hinge-ECD-TM-inhibitory cytosolic domain 1. The NKG2A “full” iCAR format is from N to C terminal: inhibitory cytosolic domain 1-TM-ECD-hinge-tag-signal sequence 1-scFv. Sequences for the aCAR construct aAxl CD3z are shown in Table 13B. The aAxl CD3z format is from N to C terminal: signal sequence-scFv-tag-hinge-TM-intracellular signaling domain.

Dynabeads were removed by magnet. Cells were expanded and treated with puromycin for 10 days. An aliquot of each condition was stained with PE conjugated anti-MYC and BV421 conjugated anti-FLAG antibodies (corresponding to the aCAR and the iCAR), and their transgene expression quantified using an LX CytoFlex Flow Cytometry machine.

### T Cell Co-Culture Killing Assay

T-cells were counted and distributed into a 96-well plate for co-culture assays. Cytotoxicity assays were performed by co-incubating engineered T cells and parental NALM6 targets (WT), or NALM6 targets engineered to overexpress Axl, Her2, or both Axl and Her2 antigens. Each well contained 1×105 Nalm6 target cells pre-stained with cell trace violet dye (Invitrogen) and 1×10⁵ engineered T-cells. Co-cultures were incubated at 37° C. with 5% CO₂ for 24 hrs. Cell were stained with 7-AAD viability dye and percent death of target cells was quantified by flow cytometry. The percent killing was normalized to target cells only. Cytokines in the media from the same co-cultures were measured using a Human magnetic Luminex assay (R&D systems) and MAGPIX analyzer (Millipore Sigma).

TABLE 13A
Anti-Her2 iCAR Formats and Domains

| Construct | | |
|---|---|---|
| (by ICD) | Domain | Sequence |
| LIR1 | signal | MALPVTALLLPLALLLHAARP (SEQ ID NO: 71) |
| PD-1 (full) | sequence 1 | |
| CTLA-4 (full) | (CD8) | |
| KIR3DL1 (full) | amino acid | |
| NKG2A | | |
| TIGIT | | |
| TIGIT (full) | | |
| LIR1 | signal | ATGGCCTTACCAGTGACCGCCTTGCTCCTGCCGCTGGCCTTG |
| PD-1 (full) | sequence 1 | CTGCTCCACGCCGCCAGGCCG (SEQ ID NO: 72) |
| CTLA-4 (full) | (CD8) | |
| KIR3DL1 (full) | nucleic acid | |
| NKG2A | | |
| TIGIT | | |
| TIGIT (full) | | |
| LIR1 | signal | KYLLPTAAAGLLLLAAQPAMA (SEQ ID NO: 73) |
| PD-1 (full) | sequence 2 | |
| CTLA-4 (full) | (pelB) | |
| KIR3DL1 (full) | amino acid | |
| NKG2A | | |
| TIGIT | | |
| TIGIT (full) | | |
| NKG2A full no ss1 | | |
| LIR1 | signal | AAATACCTATTGCCTACGGCAGCCGCTGGATTGTTATTACTC |
| PD-1 (full) | sequence 2 | GCGGCCCAGCCGGCCATGGCC (SEQ ID NO: 74) |
| CTLA-4 (full) | (pelB) | |
| KIR3DL1 (full) | nucleic acid | |
| NKG2A | | |
| TIGIT | | |
| TIGIT (full) | | |
| NKG2A full no ss1 | | |
| LIR1 | scFv | SEQ ID NO: 75 |
| PD-1 (full) | (aHer2H3B1 | |
| CTLA-4 (full) | with (G4S)3 | |
| KIR3DL1 (full) | linker) | |
| NKG2A | amino acid | |
| NKG2A (full) | | |
| TIGIT | | |
| TIGIT (full) | | |
| LIR1 | scFv | SEQ ID NO: 76 |
| PD-1 (full) | (aHer2H3B1 | |
| CTLA-4 (full) | with (G4S)3 | |
| KIR3DL1 (full) | linker) | |
| NKG2A | nucleic acid | |
| NKG2A (full) | | |
| TIGIT | | |
| TIGIT (full) | | |
| LIR1 | tag | GKPIPNPLLGLDSTNGAA (SEQ ID NO: 77) |
| PD-1 (full) | (V5 + NGAA | |
| CTLA-4 (full) | linker) | |
| KIR3DL1 (full) | amino acid | |
| NKG2A | | |
| NKG2A (full) | | |
| TIGIT | | |
| TIGIT (full) | | |
| LIR1 | tag | GGGAAGCCTATCCCGAACCCTCTGTTGGGTCTCGATAGTACC |
| PD-1 (full) | (V5 + NGAA | AATGGGGCCGCA (SEQ ID NO: 78) |
| CTLA-4 (full) | linker) | |
| KIR3DL1 (full) | nucleic acid | |
| NKG2A | | |
| TIGIT | | |
| TIGIT (full) | | |
| LIR1 | hinge | TTTPAPRPPTPAPTIALQPLSLRPEACRPAAGGAVHTRGLDFAC |
| PD-1 (full) | (CD8) | D (SEQ ID NO: 37) |
| CTLA-4 (full) | amino acid | |
| KIR3DL1 (full) | | |
| NKG2A | | |
| NKG2A (full) | | |
| TIGIT | | |
| TIGIT (full) | | |
| LIR1 | hinge | ACCACGACGCCAGCGCCGCGACCACCAACACCGGCGCCCAC |
| PD-1 (full) | (CD8) | CATCGCGTTGCAGCCCCTGTCCCTGCGCCCAGAGGCGTGCC |
| CTLA-4 (full) | nucleic acid | GGCCAGCGGCGGGGGGCGCAGTGCACACGAGGGGGCTGGA |
| KIR3DL1 (full) | | CTTCGCCTGTGAT (SEQ ID NO: 79) |
| NKG2A | | |
| NKG2A (full) | | |
| TIGIT | | |
| TIGIT (full) | | |
| PD-1 (full) | TM | VGVVGGLLGSLVLLVWVLAVI (SEQ ID NO: 60) |
| | (PD-1) | |
| | amino acid | |
| PD-1 (full) | TM | GTTGGGGTTGTAGGTGGTCTGCTCGGCAGCCTGGTCTTGTTG |
| | (PD-1) | GTGTGGGTCTTGGCTGTGATC (SEQ ID NO: 64) |
| | nucleic acid | |
| CTLA-4 (full) | TM | DFLLWILAAVSSGLFFYSFLLT (SEQ ID NO: 68) |
| | (CTLA-4) | |
| | amino acid | |
| CTLA-4 (full) | TM | GATTTTCTGCTGTGGATTCTGGCAGCTGTGAGCTCTGGCTTG |
| | (CTLA-4) | TTTTTCTACAGCTTCCTCCTGACC (SEQ ID NO: 80) |
| | nucleic acid | |
| KIR3DL1 (full) | TM | ILIGTSVVIILFILLLFFLL (SEQ ID NO: 69) |
| | (KIR3DL1) | |
| | amino acid | |
| KIR3DL1 (full) | TM | ATCCTGATCGGGACAAGTGTAGTAATCATACTTTTCATACTC |
| | (KIR3DL1) | CTGCTCTTTTTTCTCTTG (SEQ ID NO: 81) |
| | nucleic acid | |
| LIR1 | TM | VIGIL VAVILLLLLLLLLFLI (SEQ ID NO: 59) |
| | (LIR1) | |
| | amino acid | |
| LIR1 | TM | GTTATAGGGATCCTGGTGGCTGTCATACTCCTCTTGCTCCTC |
| | (LIR1) | TTGTTGCTGCTTTTTTTGATA (SEQ ID NO: 62) |
| | nucleic acid | |
| NKG2A | TM | IVVITVVSAMLILCIIGLIGVIL (SEQ ID NO: 89) |
| | (NKG2A- | |
| | reversed) | |
| | amino acid | |
| NKG2A | TM | ATAGTGGTCATCACTGTAGTTAGTGCAATGCTTATTCTTTGT |
| | (NKG2A- | ATCATAGGGCTCATAGGGGTAATCCTG (SEQ ID NO: 90) |
| | reversed) | |
| | amino acid | |
| TIGIT | TM | LLGAMAATLVVICTAVIVVVA (SEQ ID NO: 91) |
| TIGIT (full) | (TIGIT) | |
| | amino acid | |
| TIGIT | TM | CTGCTGGGCGCCATGGCCGCCACACTGGTTGTTATCTGTACC |
| TIGIT (full) | (TIGIT) | GCCGTGATCGTGGTGGTGGCC (SEQ ID NO: 92) |
| | nucleic acid | |
| PD-1 (full) | inhibitory | CSRAARGTIGARRTGQPLKEDPSAVPVFSVDYGELDFQWREKT |
| | cytosolic | PEPPVPCVPEQTEYATIVFPSGMGTSSPARRGSADGPRSAQPLR |
| | domain 1 | PEDGHCSWPL (SEQ ID NO: 1) |
| | (PD-1) | |
| | amino acid | |
| PD-1 (full) | inhibitory | TGTAGCCGAGCGGCCAGAGGCACAATCGGGGCAAGACGAA |
| | cytosolic | CAGGACAGCCGCTCAAAGAGGACCCCAGTGCGGTCCCCGTT |
| | domain 1 | TTCTCCGTGGATTACGGAGAACTGGATTTCCAGTGGCGGGA |
| | (PD-1) | GAAGACACCAGAGCCCCCGGTGCCCTGCGTGCCGGAGCAGA |
| | nucleic acid | CTGAGTACGCCACGATTGTGTTTCCCTCTGGAATGGGGACTT |
| | | CATCCCCCGCTAGGCGCGGCTCAGCTGATGGCCCAAGATCC |
| | | GCTCAACCGTTGCGGCCAGAGGACGGGCATTGCAGTTGGCC |
| | | TCTG (SEQ ID NO: 51) |
| CTLA-4 (full) | inhibitory | AVSLSKMLKKRSPLTTGVGVKMPPTEPECEKQFQPYFIPIN |
| | cytosolic | (SEQ ID NO: 67) |
| | domain 1 | |
| | (CTLA-4) | |
| | amino acid | |
| CTLA-4 (full) | inhibitory | GCCGTGTCACTTAGTAAGATGCTGAAGAAGAGGTCACCACT |
| | cytosolic | GACGACAGGGGTTGGAGTGAAGATGCCACCCACAGAACCC |
| | domain 1 | GAATGTGAGAAGCAATTCCAGCCTTATTTCATTCCAATAAAT |
| | (CTLA-4) | (SEQ ID NO: 84) |
| | nucleic acid | |
| KIR3DL1 (full) | inhibitory | HLWCSNKKNAAVMDQEPAGNRTANSEDSDEQDPEEVTYAQL |
| | cytosolic | DHCVFTQRKITRPSQRPKTPPTDTILYTELPNAKPRSKVVSCP |
| | domain 1 | (SEQ ID NO: 66) |
| | (KIR3DL1) | |
| | amino acid | |
| KIR3DL1 (full) | inhibitory | CATCTGTGGTGTTCTAATAAGAAGAATGCTGCTGTGATGGAT |
| | cytosolic | CAAGAGCCCGCTGGTAACAGAACGGCCAACAGTGAAGATA |
| | domain 1 | GCGATGAGCAGGACCCAGAAGAAGTGACCTACGCCCAACTC |
| | (KIR3DL1) | GACCACTGTGTTTTTACGCAGCGGAAAATCACTCGACCCTCT |
| | nucleic acid | CAACGACCCAAAACGCCGCCTACGGACACCATACTCTACAC |
| | | CGAACTGCCGAACGCCAAACCACGGTCCAAGGTGGTATCAT |
| | | GTCCG (SEQ ID NO: 85) |
| LIR1 | inhibitory | LRHRRQGKHWTSTQRKADFQHPAGAVGPEPTDRGLQWRSSPA |
| | cytosolic | ADAQEENLYAAVKHTQPEDGVEMDTRSPHDEDPQAVTYAEV |
| | domain 1 | KHSRPRREMASPPSPLSGEFLDTKDRQAEEDRQMDTEAAASEA |
| | (LIR1) | PQDVTYAQLHSLTLRREATEPPPSQEGPSPAVPSIYATLAIH |
| | amino acid | (SEQ ID NO: 50) |
| LIR1 | inhibitory | TTGCGCCACAGACGGCAGGGAAAGCACTGGACTAGTACGCA |
| | cytosolic | GAGGAAAGCGGACTTCCAGCATCCCGCAGGAGCCGTGGGGC |
| | domain 1 | CTGAACCCACTGATCGCGGCCTTCAATGGAGGTCTAGCCCG |
| | (LIR1) | GCGGCAGACGCACAAGAGGAAAACTTGTACGCAGCCGTTAA |
| | nucleic acid | GCACACCCAACCGGAGGACGGCGTTGAGATGGATACCCGCT |
| | | CCCCTCACGATGAAGACCCTCAAGCAGTCACTTACGCGGAA |
| | | GTAAAGCATAGCCGCCCCAGACGGGAAATGGCTAGCCCGCC |
| | | GTCCCCCCTTAGCGGGGAATTTCTGGACACTAAAGATAGGC |
| | | AGGCGGAAGAGGACCGCCAAATGGATACAGAGGCGGCGGC |
| | | AAGTGAAGCACCTCAAGACGTTACTTACGCTCAACTTCACA |
| | | GCCTTACCCTCAGGCGAGAAGCGACTGAACCACCCCCTTCC |
| | | CAAGAAGGGCCAAGCCCAGCGGTTCCTTCTATCTATGCTACT |
| | | CTTGCTATTCAC (SEQ ID NO: 54) |
| NKG2A | inhibitory | KEPASPLDKCHYTKDNGQFDQSAKQLNLEAYTIEQETALISNK |
| | cytosolic | NGKPKRQQRKPNPPLNLDSYIVGQNDM (SEQ ID NO: 93) |
| | domain 1 | |
| | (NKG2A- | |
| | reversed) | |
| | amino acid | |
| NKG2A | inhibitory | AAGGAGCCTGCGTCCCCGTTGGATAAATGCCACTATACTAA |
| | cytosolic | GGATAACGGTCAGTTCGATCAGAGTGCAAAGCAACTTAACT |
| | domain 1 | TGGAGGCTTACACTATAGAGCAAGAAACAGCGCTGATAAGT |
| | (NKG2A- | AATAAGAACGGTAAGCCAAAGCGACAGCAGAGGAAACCCA |
| | reversed) | ATCCTCCGCTTAACTTGGATAGCTACATCGTCGGGCAAAATG |
| | nucleic acid | ACATG (SEQ ID NO: 94) |
| TIGIT | inhibitory | LTRKKKALRIHSVEGDLRRKSAGQEEWSPSAPSPPGSCVQAEA |
| TIGIT (full) | cytosolic | APAGLCGEQRGEDCAELHDYFNVLSYRSLGNCSFFTETG(SEQ |
| | domain 1 | ID NO: 95) |
| | (TIGIT) | |
| | amino acid | |
| TIGIT | inhibitory | CTGACCAGAAAGAAGAAGGCCCTGAGAATCCACAGCGTGG |
| TIGIT (full) | cytosolic | AAGGCGACCTGCGGAGAAAGTCTGCCGGACAAGAAGAGTG |
| | domain 1 | GTCCCCTAGCGCTCCATCTCCACCTGGATCTTGTGTGCAGGC |
| | (TIGIT) | CGAAGCAGCTCCTGCTGGACTGTGTGGCGAACAGAGAGGCG |
| | nucleic acid | AAGATTGCGCCGAGCTGCACGACTACTTCAACGTGCTGAGC |
| | | TACAGAAGCCTGGGCAACTGCAGCTTCTTCACCGAGACAGG |
| | | A (SEQ ID NO: 96) |
| PD-1 (full) | ECD (PD-1) | FLDSPDRPWNPPTFSPALLVVTEGDNATFTCSFSNTSESFVLNW |
| | amino acid | YRMSPSNQTDKLAAFPEDRSQPGQDCRFRVTQLPNGRDFHMS |
| | | VVRARRNDSGTYLCGAISLAPKAQIKESLRAELRVTERRAEVPT |
| | | AHPSPSPRPAGQFQTLV (SEQ ID NO: 97) |
| PD-1 (full) | ECD (PD-1) | TTCCTGGACAGCCCCGACAGACCTTGGAACCCTCCTACATTC |
| | nucleic acid | AGCCCCGCTCTGCTGGTGGTTACCGAGGGCGATAATGCCAC |
| | | CTTCACCTGTAGCTTCAGCAACACCAGCGAGAGCTTCGTGCT |
| | | GAACTGGTACAGAATGAGCCCCAGCAACCAGACCGACAAG |
| | | CTGGCCGCCTTTCCTGAGGATAGATCTCAGCCCGGCCAGGA |
| | | CTGCCGGTTCAGAGTTACACAGCTGCCCAACGGCCGGGACT |
| | | TCCACATGTCTGTCGTCCGGGCCAGAAGAAACGACAGCGGC |
| | | ACATATCTGTGCGGCGCCATTTCTCTGGCCCCTAAGGCTCAG |
| | | ATCAAAGAGAGCCTGAGAGCCGAGCTGAGAGTGACAGAAA |
| | | GACGGGCCGAAGTGCCCACAGCTCACCCTTCACCTTCTCCA |
| | | AGACCTGCCGGCCAGTTTCAGACACTGGTT (SEQ ID NO: 98) |
| CTLA-4 (full) | ECD (CTLA-4) | KAMHVAQPAVVLASSRGIASFVCEYASPGKATEVRVTVLRQA |
| | amino acid | DSQVTEVCAATYMMGNELTFLDDSICTGTSSGNQVNLTIQGLR |
| | | AMDTGLYICKVELMYPPPYYLGIGNGTQIYVIDPEPCPDSDFLL |
| | | WILAAVSSGLFFYSFLLT (SEQ ID NO: 99) |
| CTLA-4 (full) | ECD (CTLA-4) | AAGGCCATGCATGTGGCTCAGCCTGCTGTGGTGCTGGCCTCT |
| | nucleic | TCTAGAGGAATCGCCAGCTTCGTGTGCGAGTACGCCTCTCCT |
| | acid | GGAAAGGCCACTGAAGTGCGCGTGACCGTTCTGAGACAGGC |
| | | CGATAGCCAAGTGACCGAAGTGTGCGCCGCCACCTACATGA |
| | | TGGGCAACGAGCTGACCTTCCTGGACGACAGCATCTGTACC |
| | | GGCACCAGCAGCGGCAATCAAGTGAACCTGACCATCCAGGG |
| | | CCTGAGAGCCATGGATACCGGCCTGTACATCTGCAAGGTGG |
| | | AACTGATGTACCCTCCTCCTTACTACCTCGGCATCGGCAACG |
| | | GCACCCAGATCTACGTGATCGACCCTGAGCCTTGTCCTGACA |
| | | GCGACTTTCTGCTGTGGATCCTGGCTGCCGTGTCCAGCGGCC |
| | | TGTTCTTCTACTCTTTTCTGCTGACC (SEQ ID NO: 100) |
| KIR3DL1 (full) | ECD | HMGGQDKPFLSAWPSAVVPRGGHVTLRCHYRHRFNNFMLYK |
| | (KIR3DL1) | EDRIHIPIFHGRIFQESFNMSPVTTAHAGNYTCRGSHPHSPTGWS |
| | amino acid | APSNPVVIMVTGNHRKPSLLAHPGPLVKSGERVILQCWSDIMF |
| | | EHFFLHKEGISKDPSRLVGQIHDGVSKANFSIGPMMLALAGTY |
| | | RCYGSVTHTPYQLSAPSDPLDIVVTGPYEKPSLSAQPGPKVQAG |
| | | ESVTLSCSSRSSYDMYHLSREGGAHERRLPAVRKVNRTFQADF |
| | | PLGPATHGGTYRCFGSFRHSPYEWSDPSDPLLVSVTGNPSSSWP |
| | | SPTEPSSKSGNPRHLH (SEQ ID NO: 101) |
| KIR3DL1 (full) | ECD | CACATGGGCGGACAGGATAAGCCTTTCCTGAGCGCCTGGCC |
| | (KIR3DL1) | TTCTGCCGTTGTTCCTAGAGGCGGACACGTGACCCTGCGGTG |
| | nucleic | TCACTACAGACACCGGTTCAACAACTTCATGCTGTACAAAG |
| | acid | AGGACCGGATTCACATCCCCATCTTCCACGGCCGGATCTTCC |
| | | AAGAGTCCTTCAACATGAGCCCCGTGACCACAGCTCACGCC |
| | | GGCAACTACACATGCAGAGGCTCTCACCCTCACAGCCCTAC |
| | | AGGCTGGAGTGCCCCTTCTAACCCCGTGGTCATCATGGTCAC |
| | | CGGCAACCACAGAAAGCCCAGCCTGCTTGCTCATCCCGGAC |
| | | CTCTGGTTAAGTCTGGCGAGCGAGTGATCCTGCAGTGTTGG |
| | | AGCGATATTATGTTCGAGCACTTCTTTCTGCACAAAGAGGGC |
| | | ATCAGCAAGGACCCCTCTAGACTCGTGGGCCAGATCCATGA |
| | | TGGCGTGTCCAAGGCCAACTTCAGCATCGGCCCTATGATGCT |
| | | GGCCCTGGCCGGCACCTATAGATGTTACGGCAGCGTGACCC |
| | | ACACACCTTACCAGCTGAGCGCCCCTAGCGACCCTCTGGAT |
| | | ATCGTGGTCACAGGCCCCTACGAGAAGCCTAGCCTGTCTGC |
| | | ACAGCCTGGACCTAAAGTGCAGGCCGGCGAAAGCGTGACAC |
| | | TGAGCTGTAGCAGCAGATCCAGCTACGACATGTACCACCTG |
| | | AGCAGAGAAGGCGGAGCCCACGAGAGAAGGCTGCCTGCCG |
| | | TCAGAAAAGTGAACCGGACCTTCCAGGCCGACTTTCCTCTG |
| | | GGACCTGCTACACACGGCGGCACCTACCGGTGTTTCGGCAG |
| | | CTTTAGACACAGCCCTTACGAGTGGAGCGACCCCTCTGATCC |
| | | TCTGCTGGTGTCTGTGACCGGCAATCCTAGCAGCAGCTGGCC |
| | | CTCTCCAACAGAGCCTTCTAGCAAGAGCGGCAACCCCAGAC |
| | | ATCTGCAC (SEQ ID NO: 102) |
| TIGIT (full) | ECD (TIGIT) | MMTGTIETTGNISAEKGGSIILQCHLSSTTAQVTQVNWEQQDQ |
| | amino acid | LLAICNADLGWHISPSFKDRVAPGPGLGLTLQSLTVNDTGEYFC |
| | | IYHTYPDGTYTGRIFLEVLESSVAEHGARFQIP |
| | | (SEQ ID NO: 103) |
| TIGIT (full) | ECD (TIGIT) | ATGATGACCGGCACCATCGAGACAACCGGCAACATCTCTGC |
| | nucleic acid | CGAGAAAGGCGGCAGCATCATCCTGCAGTGTCACCTGTCTA |
| | | GCACCACCGCTCAAGTGACCCAAGTGAACTGGGAGCAGCAG |
| | | GATCAGCTGCTGGCCATCTGCAATGCCGATCTCGGCTGGCA |
| | | CATCAGCCCCAGCTTCAAGGATAGAGTGGCCCCTGGACCTG |
| | | GCCTGGGACTGACACTTCAGAGCCTGACCGTGAACGATACC |
| | | GGCGAGTACTTCTGCATCTACCACACATACCCCGACGGCAC |
| | | CTATACCGGCCGGATCTTTCTGGAAGTGCTGGAAAGCTCTGT |
| | | GGCCGAGCACGGCGCCAGATTTCAGATTCCT (SEQ ID NO: |
| | | 104) |
| NKG2A (full) | inhibitory | MDNQGVIYSDLNLPPNPKRQQRKPKGNKNSILATEQEITYAEL |
| | cytosolic | NLQKASQDFQGNDKTYHCKDLPSAPEK (SEQ ID NO: 105) |
| | domain 1 | |
| | (NKG2A) | |
| | amino acid | |
| NKG2A (full) | inhibitory | ATGGACAACCAGGGCGTGATCTACAGCGACCTGAACCTGCC |
| | cytosolic | TCCTAATCCTAAGCGGCAGCAGAGAAAGCCCAAGGGCAACA |
| | domain 1 | AGAACAGCATCCTGGCCACCGAGCAAGAGATCACCTACGCC |
| | (NKG2A) | GAGCTGAATCTGCAGAAGGCCAGCCAGGACTTCCAGGGCAA |
| | nucleic | CGACAAGACCTACCACTGCAAGGACCTGCCTAGCGCTCCCG |
| | acid | AGAAG (SEQ ID NO: 106) |
| NKG2A (full) | TM | LIVGILGIICLILMASVVTIVVI (SEQ ID NO: 107) |
| | (NKG2A) | |
| | amino acid | |
| NKG2A (full) | TM (NKG2A) | CTGATCGTGGGAATCCTGGGCATCATCTGCCTGATCCTGATG |
| | nucleic | GCCAGCGTGGTCACCATCGTGGTCATC (SEQ ID NO: 108) |
| | acid | |
| NKG2A (full) | ECD | PSTLIQRHNNSSLNTRTQKARHCGHCPEEWITYSNSCYYIGKER |
| | (NKG2A) | RTWEESLLACTSKNSSLLSIDNEEEMKFLSIISPSSWIGVFRNSSH |
| | amino acid | HPWVTMNGLAFKHEIKDSDNAELNCAVLQVNRLKSAQCGSSII |
| | | YHCKHKL (SEQ ID NO: 109) |
| NKG2A (full) | ECD | CCCAGCACACTGATCCAGCGGCACAACAACAGCAGCCTGAA |
| | (NKG2A) | CACCAGAACACAGAAGGCCCGGCACTGCGGCCACTGTCCTG |
| | nucleic | AAGAGTGGATCACATACAGCAACAGCTGCTACTACATCGGC |
| | acid | AAAGAGCGGCGGACCTGGGAAGAATCTCTGCTGGCCTGCAC |
| | | CAGCAAGAACTCCAGCCTGCTGAGCATCGACAACGAGGAAG |
| | | AGATGAAGTTCCTGTCCATCATCAGCCCCAGCAGCTGGATC |
| | | GGCGTGTTCAGAAACAGCTCCCACCATCCTTGGGTCACCAT |
| | | GAACGGCCTGGCCTTCAAGCACGAGATCAAGGACAGCGACA |
| | | ACGCCGAACTGAACTGTGCCGTGCTGCAAGTGAACCGGCTG |
| | | AAGTCTGCCCAGTGTGGCAGCAGCATCATCTATCACTGCAA |
| | | GCACAAGCTG (SEQ ID NO: 110) |
| NKG2A (full) | Tag | NGAAEQKLISEEDL (SEQ ID NO: 111) |
| | (NGAA + | |
| | Myctag) | |
| | amino acid | |
| NKG2A (full) | Tag | AATGGGGCCGCAGAACAAAAACTCATCTCAGAAGAAGATCT |
| | (NGAA + | G (SEQ ID NO: 112) |
| | Myctag) | |
| | nucleic | |
| | acid | |

TABLE 13B
aAxl CD3z aCAR Domains

| Construct | Domain | Sequence |
|---|---|---|
| aAxl CD3z | signal | MALPVTALLLPLALLLHAARP (SEQ ID NO: 71) |
| | sequence | |
| | (CD8) | |
| | amino acid | |
| aAxl CD3z | signal | ATGGCCCTGCCTGTGACAGCTCTGCTGCTGCCTCTGGCCCTG |
| | sequence | CTGCTGCATGCTGCTAGACCT (SEQ ID NO: 123) |
| | (CD8) | |
| | nucleic acid | |
| aAxl CD3z | scFv | QVQLQESGPGLVKPSETLSLTCTVSGYSITSNYWGWIRQPPGK |
| | (aAxl 1448 | GLEWMGYITYSGSTSYNPSLKSRITISRDTSKNQFSLKLSSVTAA |
| | with (G4S)3 | DTAVYYCAITTFYYWGQGTLVTVSSGGGGSGGGGSGGGGSDI |
| | linker) | QMTQSPSSLSASVGDRVTITCRASQDIGNYLRWFQQKPGKAPK |
| | amino acid | LLISGATNLAAGVPSRFSGSGSGSDFTLTISSLQPEDFATYYCLQ |
| | | SKESPWTFGQGTKVEIKRT (SEQ ID NO: 115) |
| aAxl CD3z | scFv | CAGGTGCAGCTGCAGGAAAGCGGCCCTGGCCTCGTGAAGCC |
| | (aAxl 1448 | TAGCGAGACACTGAGCCTGACCTGCACCGTGTCCGGCTACA |
| | with (G4S)3 | GCATCACCAGCAACTACTGGGGCTGGATCAGACAGCCCCCT |
| | linker) | GGCAAGGGCCTGGAATGGATGGGCTACATCACCTACAGCGG |
| | nucleic acid | CAGCACCAGCTACAACCCCAGCCTGAAGTCCCGGATCACCA |
| | | TCAGCCGGGACACCAGCAAGAACCAGTTCTCCCTGAAGCTG |
| | | AGCAGCGTGACAGCCGCCGATACCGCCGTGTACTACTGCGC |
| | | CATCACCACCTTCTACTATTGGGGCCAGGGCACCCTCGTGAC |
| | | CGTGTCTAGCGGAGGCGGAGGATCTGGCGGCGGAGGAAGT |
| | | GGCGGAGGGGGCTCTGATATCCAGATGACCCAGAGCCCCAG |
| | | CAGCCTGTCTGCCAGCGTGGGCGACAGAGTGACCATCACCT |
| | | GTAGAGCCAGCCAGGACATCGGCAACTACCTGCGGTGGTTC |
| | | CAGCAGAAGCCAGGCAAGGCCCCCAAGCTGCTGATCTCCGG |
| | | CGCCACAAATCTGGCCGCTGGCGTGCCAAGCAGATTCAGCG |
| | | GCTCTGGCAGCGGCTCCGACTTCACCCTGACCATCTCTAGCC |
| | | TGCAGCCCGAGGACTTCGCCACCTACTACTGCCTGCAGAGC |
| | | AAAGAGAGCCCCTGGACCTTCGGACAGGGCACCAAGGTGG |
| | | AAATCAAGCGGACA (SEQ ID NO: 124) |
| aAxl CD3z | tag | EQKLISEEDLNGAA (SEQ ID NO: 125) |
| | (Myc + | |
| | NGAA | |
| | linker) | |
| | amino acid | |
| aAxl CD3z | tag | GAACAAAAACTCATCTCAGAAGAAGATCTGAATGGGGCCGC |
| | (Myc + | A (SEQ ID NO: 126) |
| | NGAA | |
| | linker) | |
| | nucleic acid | |
| aAxl CD3z | hinge | TTTPAPRPPTPAPTIALQPLSLRPEACRPAAGGAVHTRGLDFAC |
| | (CD8) | D (SEQ ID NO: 37) |
| | amino acid | |
| aAxl CD3z | hinge | ACCACGACGCCAGCGCCGCGACCACCAACACCGGCGCCCAC |
| | (CD8) | CATCGCGTTGCAGCCCCTGTCCCTGCGCCCAGAGGCGTGCC |
| | nucleic acid | GGCCAGCGGCGGGGGGCGCAGTGCACACGAGGGGGCTGGA |
| | | CTTCGCCTGTGAT (SEQ ID NO: 79) |
| aAxl CD3z | TM | IYIWAPLAGTCGVLLLSLVIT (SEQ ID NO: 127) |
| | (CD8) | |
| | amino acid | |
| aAxl CD3z | TM | ATATACATCTGGGCTCCTCTGGCTGGCACTTGCGGAGTGCTT |
| | (CD8) | CTGCTGAGTCTGGTTATTACC (SEQ ID NO: 128) |
| | nucleic acid | |
| aAxl CD3z | intracellular | RVKFSRSADAPAYKQGQNQLYNELNLGRREEYDVLDKRRGRD |
| | signaling | PEMGGKPRRKNPQEGLYNELQKDKMAEAYSEIGMKGERRRG |
| | domain | KGHDGLYQGLSTATKDTYDALHMQALPPR (SEQ ID NO: 121) |
| | (CD3Q | |
| | amino acid | |
| aAxl CD3z | intracellular | AGAGTGAAGTTCAGCAGGAGCGCAGACGCCCCCGCGTACAA |
| | signaling | GCAGGGCCAGAACCAGCTCTATAACGAGCTCAATCTAGGAC |
| | domain | GAAGAGAGGAGTACGATGTTTTGGACAAGAGACGTGGCCG |
| | (CD3Q | GGACCCTGAGATGGGGGGAAAGCCGAGAAGGAAGAACCCT |
| | nucleic acid | CAGGAAGGCCTGTACAATGAACTGCAGAAAGATAAGATGG |
| | | CGGAGGCCTACAGTGAGATTGGGATGAAAGGCGAGCGCCG |
| | | GAGGGGCAAGGGGCACGATGGCCTTTACCAGGGTCTCAGTA |
| | | CAGCCACCAAGGACACCTACGACGCCCTTCACATGCAGGCC |
| | | CTGCCCCCTCGC (SEQ ID NO: 129) |

### Results

T cells were engineered to express activating chimeric receptors (aCARS) and inhibitory chimeric receptors (iCARs) having various inhibitory domains, including specifically formats featuring only the cytosolic domain (CD) of an inhibitory receptor or also an extracellular domain of the respective inhibitory receptor (ECD; “full”).

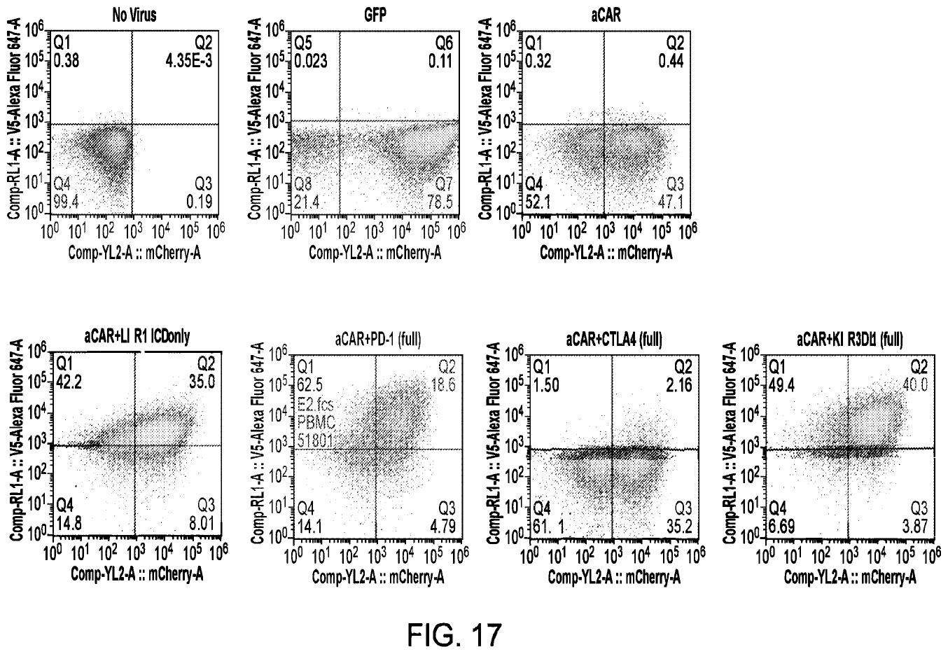

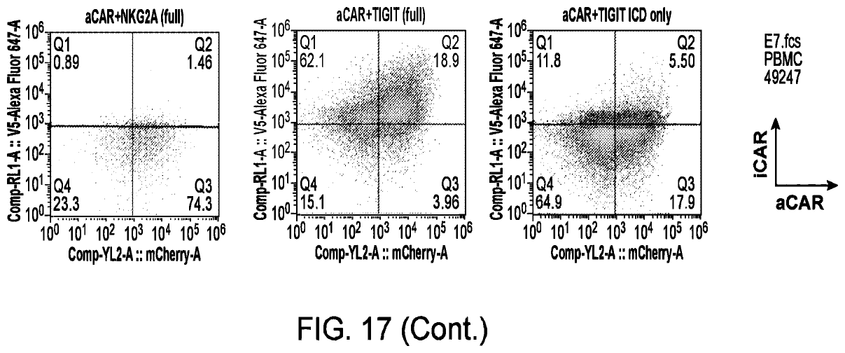

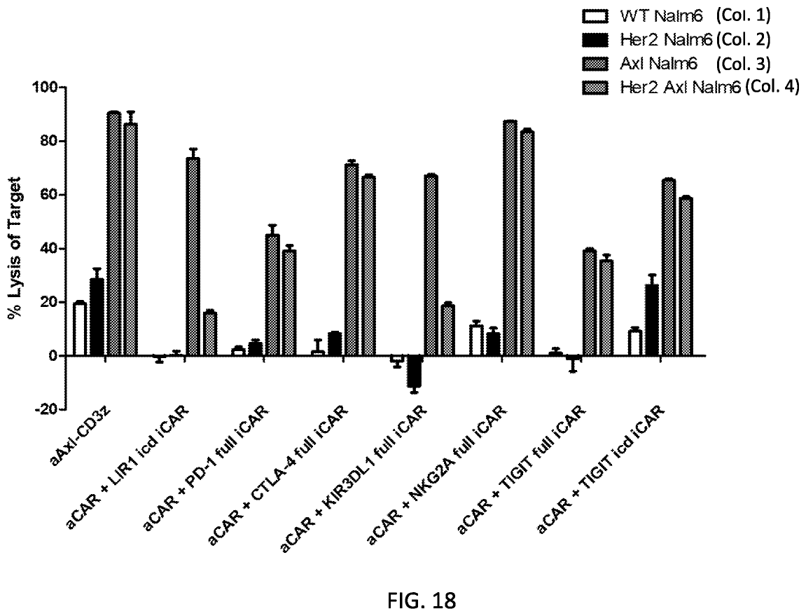

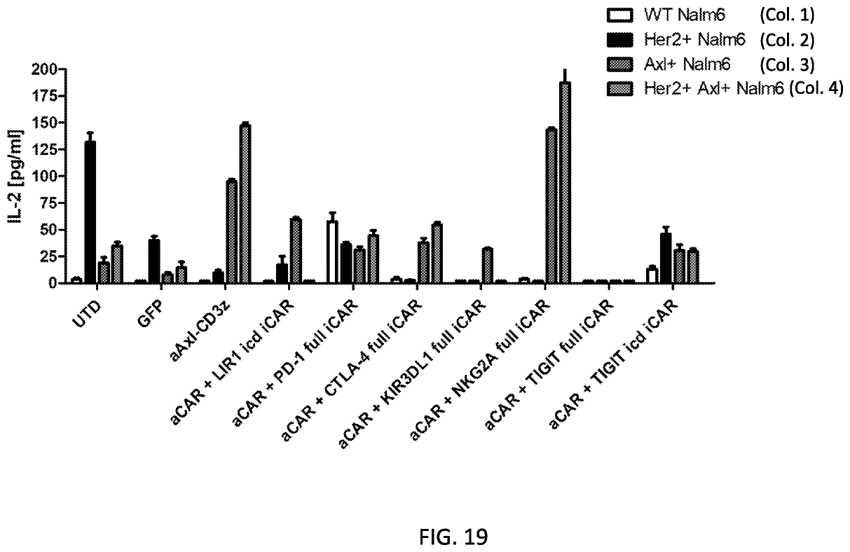

NK cells were virally transduced with aCAR only (aAxl-CD3z-mCherry), or in combination with anti-Her2 iCAR having the various inhibitory domains indicated. As shown in FIG. 17, higher percentages of cells demonstrating co-expression (aCAR+iCAR+) were observed with iCARs having only a cytosolic domain of LIR1 or a full (CD+ECD) KIR3DL1 sequence, observable but lower percentages for cells co-expression iCARs having a full (CD+ECD) PD-1 or TIGIT sequence, and minimal observable co-expression of iCARs having a full CTLA-4 sequence, a full NKG2A sequence, or a cytosolic domain of TIGIT. Engineered T cells were then assessed for iCARs reducing aCAR induced T cell activation. As shown in FIG. 18, T cells engineered to express aCAR only (“aAxal-CD3z”) or to co-express the aCAR and the various iCAR formats all demonstrated killing of target cells only expressing the aCAR antigen (Axl NALM6; column 3 each engineering condition) relative to killing of parental target cells not expressing the aCAR antigen (WT NALM6; column 1 each engineering condition) or target cells only expressing the iCAR antigen (Her2 NALM6; column 1 each engineering condition) demonstrating aCAR antigen dependent antigen-specific killing. When co-incubated with target cells expressing both aCAR and iCAR antigens (Her2 Axl NALM6; column 4 each engineering condition), T cells engineered to co-express the aCAR and iCAR for exhibited notably reduced killing relative to killing of target cells expressing only (comparing columns 4 to 3, respectively) for iCARs having only a cytosolic domain of LIR1 (“aCAR+LIR1 icd iCAR”) or a full (CD+ECD) KIR3DL1 sequence (“aCAR+KIR3DL1 full iCAR”), while other formats of iCARs exhibited more modest reductions generally in line with the aCAR only condition. As shown in FIG. 19, iCAR dependent reduction of T cell IL-2 secretion was also assessed and correlated with T cell killing. Notably, iCAR dependent reduction of T cell killing and cytokine production correlated with iCAR expression, namely the iCARs having only a cytosolic domain of LIR1 or a full (CD+ECD) KIR3DL1 sequence that demonstrated greater expression also demonstrated the greatest regulation of aCAR-mediated activation of T cells.

The results demonstrate T cells can be engineered to co-express aCARs and select iCARs successfully for select formats. In addition, iCARs demonstrated reduction of T cell-mediated killing and cytokine production in an iCAR ligand dependent manner that corresponded with co-expression in T cells.

### Example 7: Assessment of Various Inhibitory Chimeric Receptors in Reducing NK Cell Activation

### Materials and Methods

### Transduction and Expansion

NK cells are expanded for 10 days with mitomycin C-treated K562 feeder cells, followed by transduction with 7.5e5 pg of each lentivirus for aCAR and iCAR constructs. Sequences for the iCAR constructs assessed are shown in Table 14. Each iCAR construct format is from N to C terminal (except those designated as “full”): signal sequence 1-signal sequence 2-scFv-tag-hinge-TM-inhibitory cytosolic domain 1-inhibitory cytosolic domain 2 (if present). Each iCAR construct format having an ECD (designated as “full”) is from N to C terminal (except NKG2A “full”): signal sequence 1-signal sequence 2-scFv-tag-hinge-ECD-TM-inhibitory cytosolic domain 1. The NKG2A “full” iCAR format is from N to C terminal: inhibitory cytosolic domain 1-TM-ECD-hinge-tag-signal sequence 1-scFv. Anti-Axl aCAR formats aAxl CD28-CD3z or aAxl CD3z are used. Sequences for the aAxl-CD28/CD3z aCAR construct are shown in Table 10B. The aAxl CD28-CD3z format is from N to C terminal: signal sequence-tag-scFv-hinge-TM-intracellular signaling domain 1-intracellular signaling domain 2. Sequences for the aAxl CD3z aCAR construct are shown in Table 13B. The aAxl CD3z format is from N to C terminal: signal sequence-scFv-tag-hinge-TM-intracellular signaling domain. After 4 days, puromycin is added to cells for selection.

After 3 more days, cytotoxicity assays are performed by co-incubating engineered NK cells and parental target cells (WT), or targets engineered to overexpress aCAR antigens (e.g., Axl) or both aCAR antigens and iCAR antigens (e.g., both Axl and Her2). Each engineered NK cells are incubated either with (1) each target cell type separately at a ratio of 25,000 NK cells to 50,000 target cells in triplicate; or (2) as a mixture of 25,000 aCAR antigen only and 25,000 dual antigen target cells co-incubated with 25,000 NK cells of the indicated type in a 1:1:1 ratio (dual antigen targets are stained with different membrane dyes allowing them to be distinguished by flow). After overnight incubation, cells are stained with viability dyes and counted via flow cytometry. The target cell reduction is quantified as 100%×(1−No. Targets/No. Targets (NV)).

TABLE 14
Anti-Her2 iCAR Formats and Domains

| Construct | | |
|---|---|---|
| (by ICD) | Domain | Sequence |
| PD-1 | signal | MALPVTALLLPLALLLHAARP (SEQ ID NO: 71) |
| CTLA-4 | sequence 1 | |
| KIR3DL1 | (CD8) | |
| LIR1 | amino acid | |
| BTLA | | |
| NKG2A | | |
| LIR1-BTLA | | |
| LIR1-PD1 | | |
| LIR1-KIR3DL1 | | |
| KIR3DL1-LIR1 | | |
| LIR1 2x | | |
| KIR3DL1 2x | | |
| DAP10e KIR3DL1 | | |
| 28-28 KIR3DL1 | | |
| LIR1 | | |
| (codon opt.) | | |
| PD-1 (full) | | |
| CTLA-4 (full) | | |
| KIR3DL1 (full) | | |
| TIGIT | | |
| TIGIT (full) | | |
| LIR1-BTLA | | |
| LIR1-PD-1 | | |
| PD-1 | signal | ATGGCCTTACCAGTGACCGCCTTGCTCCTGCCGCTGGCCTTG |
| CTLA-4 | sequence 1 | CTGCTCCACGCCGCCAGGCCG (SEQ ID NO: 72) |
| KIR3DL1 | (CD8) | |
| LIR1 | nucleic acid | |
| BTLA | | |
| NKG2A | | |
| LIR1-BTLA | | |
| LIR1-PD1 | | |
| LIR1-KIR3DL1 | | |
| KIR3DL1-LIR1 | | |
| LIR1 2x | | |
| KIR3DL1 2x | | |
| DAP10e KIR3DL1 | | |
| 28-28 KIR3DL1 | | |
| LIR1 | | |
| (codon opt.) | | |
| PD-1 (full) | | |
| CTLA-4 (full) | | |
| KIR3DL1 (full) | | |
| TIGIT | | |
| TIGIT (full) | | |
| LIR1-BTLA | | |
| LIR1-PD-1 | | |
| PD-1 | signal | KYLLPTAAAGLLLLAAQPAMA (SEQ ID NO: 73) |
| CTLA-4 | sequence 2 | |
| KIR3DL1 | (pelB) | |
| LIR1 | amino acid | |
| BTLA | | |
| NKG2A | | |
| LIR1-BTLA | | |
| LIR1-PD1 | | |
| LIR1-KIR3DL1 | | |
| KIR3DL1-LIR1 | | |
| LIR1 2x | | |
| KIR3DL1 2x | | |
| DAP10e KIR3DL1 | | |
| 28-28 KIR3DL1 | | |
| LIR1 | | |
| (codon opt.) | | |
| PD-1 (full) | | |
| CTLA-4 (full) | | |
| KIR3DL1 (full) | | |
| TIGIT | | |
| TIGIT (full) | | |
| LIR1-BTLA | | |
| LIR1-PD-1 | | |
| NKG2A full | | |
| no ss1 | | |
| PD-1 | signal | AAATACCTATTGCCTACGGCAGCCGCTGGATTGTTATTACTC |
| CTLA-4 | sequence 2 | GCGGCCCAGCCGGCCATGGCC (SEQ ID NO: 74) |
| KIR3DL1 | (pelB) | |
| LIR1 | nucleic acid | |
| BTLA | | |
| NKG2A | | |
| LIR1-BTLA | | |
| LIR1-PD1 | | |
| LIR1-KIR3DL1 | | |
| KIR3DL1-LIR1 | | |
| LIR1 2x | | |
| KIR3DL1 2x | | |
| DAP10e KIR3DL1 | | |
| 28-28 KIR3DL1 | | |
| LIR1 | | |
| (codon opt.) | | |
| PD-1 (full) | | |
| CTLA-4 (full) | | |
| KIR3DL1 (full) | | |
| TIGIT | | |
| TIGIT (full) | | |
| LIR1-BTLA | | |
| LIR1-PD-1 | | |
| NKG2A full | | |
| no ss1 | | |
| PD-1 | scFv | QVQLVQSGAEVKKPGESLKISCKGSGYSFTSYWIAWVRQMPG |
| CTLA-4 | (aHer2H3B1 | KGLEYMGLIYPGDSDTKYSPSFQGQVTISVDKSVSTAYLQWSS |
| KIR3DL1 | with (G4S)3 | LKPSDSAVYFCARHDVGYCTDRTCAKWPEYFQHWGQGTLVT |
| LIR1 | linker) | VSSGGGGSGGGGSGGGGSQSVLTQPPSVSAAPGQKVTISCSGSS |
| BTLA | amino acid | SNIGNNYVSWYQQLPGTAPKLLIYDHTNRPAGVPDRFSGSKSG |
| NKG2A | | TSASLAISGFRS EDEAD YYCASWDYTLSGWVFGGGTKLTVLG |
| TIGIT | | (SEQ ID NO: 75) |
| LIR1-BTLA | | |
| LIR1-PD1 | | |
| LIR1-KIR3DL1 | | |
| KIR3DL1-LIR1 | | |
| LIR1 2x | | |
| KIR3DL1 2x | | |
| DAP10e KIR3DL1 | | |
| 28-28 KIR3DL1 | | |
| LIR1 | | |
| (codon opt.) | | |
| PD-1 (full) | | |
| CTLA-4 (full) | | |
| KIR3DL1 (full) | | |
| NKG2A (full) | | |
| TIGIT (full) | | |
| LIR1-BTLA | | |
| LIR1-PD-1 | | |
| PD-1 | scFv | CAGGTGCAGCTGGTGCAGTCTGGGGCAGAGGTGAAAAAGCC |
| CTLA-4 | (aHer2H3B1 | CGGGGAGTCTCTGAAGATCTCCTGTAAGGGTTCTGGATACA |
| KIR3DL1 | with (G4S)3 | GCTTTACCAGCTACTGGATCGCCTGGGTGCGCCAGATGCCC |
| LIR1 | linker) | GGGAAAGGCCTGGAGTACATGGGGCTCATCTATCCTGGTGA |
| BTLA | nucleic acid | CTCTGACACCAAATACAGCCCGTCCTTCCAAGGCCAGGTCA |
| NKG2A | | CCATCTCAGTCGACAAGTCCGTCAGCACTGCCTACTTGCAAT |
| TIGIT | | GGAGCAGTCTGAAGCCCTCGGACAGCGCCGTGTATTTTTGT |
| LIR1-BTLA | | GCGAGACATGACGTGGGATATTGCACCGACCGGACTTGCGC |
| LIR1-PD1 | | AAAGTGGCCTGAATACTTCCAGCATTGGGGCCAGGGCACCC |
| LIR1-KIR3DL1 | | TGGTCACCGTCTCCTCAGGTGGAGGCGGTTCAGGCGGAGGT |
| KIR3DL1-LIR1 | | GGCTCTGGCGGTGGCGGATCGCAGTCTGTGTTGACGCAGCC |
| LIR1 2x | | GCCCTCAGTGTCTGCGGCCCCAGGACAGAAGGTCACCATCT |
| KIR3DL1 2x | | CCTGCTCTGGAAGCAGCTCCAACATTGGGAATAATTATGTAT |
| DAP10e KIR3DL1 | | CCTGGTACCAGCAGCTCCCAGGAACAGCCCCCAAACTCCTC |
| 28-28 KIR3DL1 | | ATCTATGATCACACCAATCGGCCCGCAGGGGTCCCTGACCG |
| LIR1 | | ATTCTCTGGCTCCAAGTCTGGCACCTCAGCCTCCCTGGCCAT |
| (codon opt.) | | CAGTGGGTTCCGGTCCGAGGATGAGGCTGATTATTACTGTG |
| PD-1 (full) | | CCTCCTGGGACTACACCCTCTCGGGCTGGGTGTTCGGCGGA |
| CTLA-4 (full) | | GGGACCAAGCTGACCGTCCTAGGT (SEQ ID NO: 76) |
| KIR3DL1 (full) | | |
| NKG2A (full) | | |
| TIGIT (full) | | |
| LIR1-BTLA | | |
| LIR1-PD-1 | | |
| PD-1 | tag | GKPIPNPLLGLDSTNGAA (SEQ ID NO: 77) |
| CTLA-4 | (V5 + NGAA | |
| KIR3DL1 | linker) | |
| LIR1 | amino acid | |
| BTLA | | |
| NKG2A | | |
| TIGIT | | |
| LIR1-BTLA | | |
| LIR1-PD1 | | |
| LIR1-KIR3DL1 | | |
| KIR3DL1-LIR1 | | |
| LIR1 2x | | |
| KIR3DL1 2x | | |
| DAP10e KIR3DL1 | | |
| 28-28 KIR3DL1 | | |
| LIR1 | | |
| (codon opt.) | | |
| PD-1 (full) | | |
| CTLA-4 (full) | | |
| KIR3DL1 (full) | | |
| TIGIT (full) | | |
| LIR1-BTLA | | |
| LIR1-PD-1 | | |
| PD-1 | tag | GGGAAGCCTATCCCGAACCCTCTGTTGGGTCTCGATAGTACC |
| CTLA-4 | (V5 + NGAA | AATGGGGCCGCA (SEQ ID NO: 78) |
| KIR3DL1 | linker) | |
| LIR1 | nucleic acid | |
| BTLA | | |
| NKG2A | | |
| TIGIT | | |
| LIR1-BTLA | | |
| LIR1-PD1 | | |
| LIR1-KIR3DL1 | | |
| KIR3DL1-LIR1 | | |
| LIR1 2x | | |
| KIR3DL1 2x | | |
| DAP10e KIR3DL1 | | |
| 28-28 KIR3DL1 | | |
| LIR1 (codon opt.) | | |
| PD-1 (full) | | |
| CTLA-4 (full) | | |
| KIR3DL1 (full) | | |
| TIGIT (full) | | |
| LIR1-BTLA | | |
| LIR1-PD-1 | | |
| PD-1 | hinge | TTTPAPRPPTPAPTIALQPLSLRPEACRPAAGGAVHTRGLDFAC |
| CTLA-4 | (CD8) | D (SEQ ID NO: 37) |
| KIR3DL1 | amino acid | |
| LIR1 | | |
| BTLA | | |
| NKG2A | | |
| TIGIT | | |
| LIR1-BTLA | | |
| LIR1-PD1 | | |
| LIR1-KIR3DL1 | | |
| KIR3DL1-LIR1 | | |
| LIR1 2x | | |
| KIR3DL1 2x | | |
| DAP10e KIR3DL1 | | |
| 28-28 KIR3DL1 | | |
| LIR1 | | |
| (codon opt.) | | |
| PD-1 (full) | | |
| CTLA-4 (full) | | |
| KIR3DL1 (full) | | |
| NKG2A (full) | | |
| TIGIT (full) | | |
| LIR1-BTLA | | |
| LIR1-PD-1 | | |
| PD-1 | hinge | ACCACGACGCCAGCGCCGCGACCACCAACACCGGCGCCCAC |
| CTLA-4 | (CD8) | CATCGCGTTGCAGCCCCTGTCCCTGCGCCCAGAGGCGTGCC |
| KIR3DL1 | nucleic acid | GGCCAGCGGCGGGGGGCGCAGTGCACACGAGGGGGCTGGA |
| LIR1 | | CTTCGCCTGTGAT (SEQ ID NO: 79) |
| BTLA | | |
| NKG2A | | |
| TIGIT | | |
| LIR1-BTLA | | |
| LIR1-PD1 | | |
| LIR1-KIR3DL1 | | |
| KIR3DL1-LIR1 | | |
| LIR1 2x | | |
| KIR3DL1 2x | | |
| DAP10e KIR3DL1 | | |
| 28-28 KIR3DL1 | | |
| LIR1 (codon opt.) | | |
| PD-1 (full) | | |
| CTLA-4 (full) | | |
| KIR3DL1 (full) | | |
| NKG2A (full) | | |
| TIGIT (full) | | |
| LIR1-BTLA | | |
| LIR1-PD-1 | | |
| PD-1 | TM | VGVVGGLLGSLVLLVWVLAVI (SEQ ID NO: 60) |
| PD-1 (full) | (PD-1) | |
| | amino acid | |
| PD-1 | TM | GTTGGGGTTGTAGGTGGTCTGCTCGGCAGCCTGGTCTTGTTG |
| PD-1 (full) | (PD-1) | GTGTGGGTCTTGGCTGTGATC (SEQ ID NO: 64) |
| | nucleic acid | |
| CTLA-4 | TM | DFLLWILAAVSSGLFFYSFLLT (SEQ ID NO: 68) |
| CTLA-4 (full) | (CTLA-4) | |
| | amino acid | |
| CTLA-4 | TM | GATTTTCTGCTGTGGATTCTGGCAGCTGTGAGCTCTGGCTTG |
| CTLA-4 (full) | (CTLA-4) | TTTTTCTACAGCTTCCTCCTGACC (SEQ ID NO: 80) |
| | nucleic acid | |
| KIR3DL1 | TM | ILIGTS VVIILFILLLFFLL (SEQ ID NO: 69) |
| KIR3DL1-LIR1 | (KIR3DL1) | |
| KIR3DL1 2x | amino acid | |
| DAP10e KIR3DL1 | | |
| KIR3DL1 (full) | | |
| KIR3DL1 | TM | ATCCTGATCGGGACAAGTGTAGTAATCATACTTTTCATACTC |
| KIR3DL1-LIR1 | (KIR3DL1) | CTGCTCTTTTTTCTCTTG (SEQ ID NO: 81) |
| KIR3DL1 2x | nucleic acid | |
| DAP10e KIR3DL1 | | |
| KIR3DL1 (full) | | |
| LIR1 | TM | VIGIL VAVILLLLLLLLLFLI (SEQ ID NO: 59) |
| LIR1-KIR3DL1 | (LIR1) | |
| LIR1 2x | amino acid | |
| LIR1 | | |
| (codon opt.) | | |
| LIR1-BTLA | | |
| LIR1-PD1 | | |
| LIR1 | TM | GTTATAGGGATCCTGGTGGCTGTCATACTCCTCTTGCTCCTC |
| LIR1-KIR3DL1 | (LIR1) | TTGTTGCTGCTTTTTTTGATA (SEQ ID NO: 62) |
| LIR1 2x | nucleic acid | |
| LIR1-BTLA | | |
| LIR1-PD1 | | |
| LIR1 | TM | GTGATCGGAATTCTGGTGGCCGTGATCCTGCTGCTCCTGCTT |
| (codon opt.) | (LIR1) | CTCCTCCTGCTGTTTCTGATC (SEQ ID NO: 82) |
| | nucleic acid | |
| BTLA | TM | LLPLGGLPLLITTCFCLFCCL (SEQ ID NO: 12) |
| | (BTLA) | |
| | nucleic acid | |
| BTLA | TM | CTCTTGCCGTTGGGGGGTCTGCCACTTCTCATAACAACTTGC |
| | (BTLA) | TTCTGCCTTTTTTGCTGTTTG (SEQ ID NO: 14) |
| | nucleic acid | |
| CD28 | TM | FWVLVVVGGVLACYSLLVTVAFIIFWV (SEQ ID NO: 11) |
| | (CD28) | |
| | amino acid | |
| CD28 | TM | TTCTGGGTGCTCGTTGTTGTTGGCGGCGTGCTGGCCTGTTAT |
| | (CD28) | TCCCTGCTGGTTACCGTGGCCTTCATCATCTTTTGGGTC (SEQ |
| | nucleic acid | ID NO: 83) |
| NKG2A | TM | IVVITVVSAMLILCIIGLIGVIL (SEQ ID NO: 89) |
| | (NKG2A- | |
| | reversed) | |
| | amino acid | |
| NKG2A | TM | ATAGTGGTCATCACTGTAGTTAGTGCAATGCTTATTCTTTGT |
| | (NKG2A- | ATCATAGGGCTCATAGGGGTAATCCTG (SEQ ID NO: 90) |
| | reversed) | |
| | amino acid | |
| TIGIT | TM | LLGAMAATLVVICTAVIVVVA (SEQ ID NO: 91) |
| TIGIT (full) | (TIGIT) | |
| | amino acid | |
| TIGIT | TM | CTGCTGGGCGCCATGGCCGCCACACTGGTTGTTATCTGTACC |
| TIGIT (full) | (TIGIT) | GCCGTGATCGTGGTGGTGGCC (SEQ ID NO: 92) |
| | nucleic acid | |
| PD-1 | inhibitory | CSRAARGTIGARRTGQPLKEDPSAVPVFSVDYGELDFQWREKT |
| PD-1 (full) | cytosolic | PEPPVPCVPEQTEYATIVFPSGMGTSSPARRGSADGPRSAQPLR |
| | domain 1 | PEDGHCSWPL (SEQ ID NO: 1) |
| | (PD-1) | |
| | amino acid | |
| PD-1 | inhibitory | TGTAGCCGAGCGGCCAGAGGCACAATCGGGGCAAGACGAA |
| PD-1 (full) | cytosolic | CAGGACAGCCGCTCAAAGAGGACCCCAGTGCGGTCCCCGTT |
| | domain 1 | TTCTCCGTGGATTACGGAGAACTGGATTTCCAGTGGCGGGA |
| | (PD-1) | GAAGACACCAGAGCCCCCGGTGCCCTGCGTGCCGGAGCAGA |
| | nucleic acid | CTGAGTACGCCACGATTGTGTTTCCCTCTGGAATGGGGACTT |
| | | CATCCCCCGCTAGGCGCGGCTCAGCTGATGGCCCAAGATCC |
| | | GCTCAACCGTTGCGGCCAGAGGACGGGCATTGCAGTTGGCC |
| | | TCTG (SEQ ID NO: 51) |
| CTLA-4 | inhibitory | AVSLSKMLKKRSPLTTGVGVKMPPTEPECEKQFQPYFIPIN |
| CTLA-4 (full) | cytosolic | (SEQ ID NO: 67) |
| | domain 1 | |
| | (CTLA-4) | |
| | amino acid | |
| CTLA-4 | inhibitory | GCCGTGTCACTTAGTAAGATGCTGAAGAAGAGGTCACCACT |
| CTLA-4 (full) | cytosolic | GACGACAGGGGTTGGAGTGAAGATGCCACCCACAGAACCC |
| | domain 1 | GAATGTGAGAAGCAATTCCAGCCTTATTTCATTCCAATAAAT |
| | (CTLA-4) | (SEQ ID NO: 84) |
| | nucleic acid | |
| KIR3DL1 | inhibitory | HLWCSNKKNAAVMDQEPAGNRTANSEDSDEQDPEEVTYAQL |
| KIR3DL1-LIR1 | cytosolic | DHCVFTQRKITRPSQRPKTPPTDTILYTELPNAKPRSKVVSCP |
| KIR3DL1 2x | domain 1 | (SEQ ID NO: 66) |
| DAP10e KIR3DL1 | (KIR3DL1) | |
| 28-28 KIR3DL1 | amino acid | |
| KIR3DL1 (full) | | |
| KIR3DL1 | inhibitory | CATCTGTGGTGTTCTAATAAGAAGAATGCTGCTGTGATGGAT |
| KIR3DL1-LIR1 | cytosolic | CAAGAGCCCGCTGGTAACAGAACGGCCAACAGTGAAGATA |
| KIR3DL1 2x | domain 1 | GCGATGAGCAGGACCCAGAAGAAGTGACCTACGCCCAACTC |
| DAP10e KIR3DL1 | (KIR3DL1) | GACCACTGTGTTTTTACGCAGCGGAAAATCACTCGACCCTCT |
| 28-28 KIR3DL1 | nucleic acid | CAACGACCCAAAACGCCGCCTACGGACACCATACTCTACAC |
| KIR3DL1 (full) | | CGAACTGCCGAACGCCAAACCACGGTCCAAGGTGGTATCAT |
| | | GTCCG (SEQ ID NO: 85) |
| LIR1 | inhibitory | LRHRRQGKHWTSTQRKADFQHPAGAVGPEPTDRGLQWRSSPA |
| LIR1-KIR3DL1 | cytosolic | ADAQEENLYAAVKHTQPEDGVEMDTRSPHDEDPQAVTYAEV |
| LIR1 2x | domain 1 | KHSRPRREMASPPSPLSGEFLDTKDRQAEEDRQMDTEAAASEA |
| LIR1 | (LIR1) | PQDVTYAQLHSLTLRREATEPPPSQEGPSPAVPSIYATLAIH |
| (codon opt.) | amino acid | (SEQ ID NO: 50) |
| LIR1-BTLA | | |
| LIR1-PD1 | | |
| LIR1 | inhibitory | TTGCGCCACAGACGGCAGGGAAAGCACTGGACTAGTACGCA |
| LIR1-KIR3DL1 | cytosolic | GAGGAAAGCGGACTTCCAGCATCCCGCAGGAGCCGTGGGGC |
| LIR1 2x | domain 1 | CTGAACCCACTGATCGCGGCCTTCAATGGAGGTCTAGCCCG |
| LIR1-BTLA | (LIR1) | GCGGCAGACGCACAAGAGGAAAACTTGTACGCAGCCGTTAA |
| LIR1-PD1 | nucleic acid | GCACACCCAACCGGAGGACGGCGTTGAGATGGATACCCGCT |
| | | CCCCTCACGATGAAGACCCTCAAGCAGTCACTTACGCGGAA |
| | | GTAAAGCATAGCCGCCCCAGACGGGAAATGGCTAGCCCGCC |
| | | GTCCCCCCTTAGCGGGGAATTTCTGGACACTAAAGATAGGC |
| | | AGGCGGAAGAGGACCGCCAAATGGATACAGAGGCGGCGGC |
| | | AAGTGAAGCACCTCAAGACGTTACTTACGCTCAACTTCACA |
| | | GCCTTACCCTCAGGCGAGAAGCGACTGAACCACCCCCTTCC |
| | | CAAGAAGGGCCAAGCCCAGCGGTTCCTTCTATCTATGCTACT |
| | | CTTGCTATTCAC (SEQ ID NO: 54) |
| LIR1 | inhibitory | CTGCGGCACAGAAGGCAGGGCAAGCACTGGACAAGCACCC |
| (codon opt.) | cytosolic | AGAGAAAGGCCGATTTTCAGCACCCTGCTGGCGCCGTTGGA |
| | domain 1 | CCTGAGCCTACAGATAGAGGACTGCAGTGGCGGTCTAGCCC |
| | (LIR1) | TGCTGCCGATGCTCAAGAGGAAAACCTGTACGCCGCCGTGA |
| | nucleic acid | AGCACACCCAACCTGAAGATGGCGTGGAAATGGACACCAG |
| | codon | ATCTCCCCACGATGAGGACCCTCAGGCCGTGACATACGCTG |
| | optimized | AAGTGAAGCACTCCCGGCCTCGGAGAGAAATGGCTAGCCCT |
| | | CCAAGTCCTCTGAGCGGCGAGTTCCTGGACACCAAGGATAG |
| | | ACAGGCCGAAGAGGACCGGCAGATGGATACAGAAGCTGCC |
| | | GCCTCTGAAGCCCCACAGGATGTGACATATGCCCAGCTGCA |
| | | TAGCCTGACACTGCGGAGAGAAGCCACAGAGCCTCCACCTT |
| | | CTCAAGAGGGCCCATCTCCAGCCGTGCCTAGCATCTATGCC |
| | | ACACTGGCCATTCAC (SEQ ID NO: 86) |
| BTLA | inhibitory | RRHQGKQNELSDTAGREINLVDAHLKSEQTEASTRQNSQVLLS |
| | cytosolic | ETGIYDNDPDLCFRMQEGSEVYSNPCLEENKPGIVYASLNHSVI |
| | domain 1 | GPNSRLARNVKEAPTEYASICVRS (SEQ ID NO: 3) |
| | (BTLA) | |
| | amino acid | |
| BTLA | inhibitory | AGAAGACATCAGGGGAAGCAGAATGAACTCAGCGATACAG |
| | cytosolic | CAGGGCGAGAAATTAATTTGGTAGACGCGCATCTGAAGTCC |
| | domain 1 | GAACAGACAGAGGCTTCTACTAGACAGAACTCCCAAGTTTT |
| | (BTLA) | GTTGAGTGAGACGGGGATCTATGATAATGATCCCGATCTGT |
| | nucleic acid | GTTTTAGAATGCAGGAGGGTAGTGAAGTCTACTCAAACCCG |
| | | TGCCTGGAAGAAAATAAGCCCGGCATTGTTTACGCTAGTTT |
| | | GAATCATTCTGTAATAGGCCCGAACTCCAGACTGGCTCGCA |
| | | ATGTGAAGGAGGCCCCAACTGAGTATGCGTCCATTTGCGTG |
| | | CGGTCT (SEQ ID NO: 52) |
| NKG2A | inhibitory | KEPASPLDKCHYTKDNGQFDQSAKQLNLEAYTIEQETALISNK |
| | cytosolic | NGKPKRQQRKPNPPLNLDSYIVGQNDM (SEQ ID NO: 93) |
| | domain 1 | |
| | (NKG2A- | |
| | reversed) | |
| | amino acid | |
| NKG2A | inhibitory | AAGGAGCCTGCGTCCCCGTTGGATAAATGCCACTATACTAA |
| | cytosolic | GGATAACGGTCAGTTCGATCAGAGTGCAAAGCAACTTAACT |
| | domain 1 | TGGAGGCTTACACTATAGAGCAAGAAACAGCGCTGATAAGT |
| | (NKG2A- | AATAAGAACGGTAAGCCAAAGCGACAGCAGAGGAAACCCA |
| | reversed) | ATCCTCCGCTTAACTTGGATAGCTACATCGTCGGGCAAAATG |
| | nucleic acid | ACATG (SEQ ID NO: 94) |
| LIR1-KIR3DL1 | inhibitory | SEQ ID NO: 66 |
| KIR3DL1 2x | cytosolic | |
| | domain 2 | |
| | (KIR3DL1) | |
| | amino acid | |
| LIR1-KIR3DL1 | inhibitory | SEQ ID NO: 85 |
| KIR3DL1 2x | cytosolic | |
| | domain 2 | |
| | (KIR3DL1) | |
| | nucleic acid | |
| KIR3DL1-LIR1 | inhibitory | SEQ ID NO: 50 |
| LIR1 2x | cytosolic | |
| | domain 2 | |
| | (LIR1) | |
| | amino acid | |
| KIR3DL1-LIR1 | inhibitory | SEQ ID NO: 54 |
| LIR1 2x | cytosolic | |
| | domain 2 | |
| | (LIR1) | |
| | nucleic acid | |
| LIR1-BTLA | inhibitory | RRHQGKQNELSDTAGREINLVDAHLKSEQTEASTRQNSQVLLS |
| | cytosolic | ETGIYDNDPDLCFRMQEGSEVYSNPCLEENKPGIVYASLNHSVI |
| | domain 2 | GPNSRLARNVKEAPTEYASICVRS (SEQ ID NO: 3) |
| | (BTLA) | |
| | amino acid | |
| LIR1-BTLA | inhibitory | AGAAGACATCAGGGGAAGCAGAATGAACTCAGCGATACAG |
| | cytosolic | CAGGGCGAGAAATTAATTTGGTAGACGCGCATCTGAAGTCC |
| | domain 2 | GAACAGACAGAGGCTTCTACTAGACAGAACTCCCAAGTTTT |
| | (BTLA) | GTTGAGTGAGACGGGGATCTATGATAATGATCCCGATCTGT |
| | nucleic acid | GTTTTAGAATGCAGGAGGGTAGTGAAGTCTACTCAAACCCG |
| | | TGCCTGGAAGAAAATAAGCCCGGCATTGTTTACGCTAGTTT |
| | | GAATCATTCTGTAATAGGCCCGAACTCCAGACTGGCTCGCA |
| | | ATGTGAAGGAGGCCCCAACTGAGTATGCGTCCATTTGCGTG |
| | | CGGTCT (SEQ ID NO: 52) |
| LIR1-PD1 | inhibitory | CSRAARGTIGARRTGQPLKEDPSAVPVFSVDYGELDFQWREKT |
| | cytosolic | PEPPVPCVPEQTEYATIVFPSGMGTSSPARRGSADGPRSAQPLR |
| | domain 2 | PEDGHCSWPL (SEQ ID NO: 1) |
| | (PD-1) | |
| | amino acid | |
| LIR1-PD1 | inhibitory | TGTAGCCGAGCGGCCAGAGGCACAATCGGGGCAAGACGAA |
| | cytosolic | CAGGACAGCCGCTCAAAGAGGACCCCAGTGCGGTCCCCGTT |
| | domain 2 | TTCTCCGTGGATTACGGAGAACTGGATTTCCAGTGGCGGGA |
| | (PD-1) | GAAGACACCAGAGCCCCCGGTGCCCTGCGTGCCGGAGCAGA |
| | nucleic acid | CTGAGTACGCCACGATTGTGTTTCCCTCTGGAATGGGGACTT |
| | | CATCCCCCGCTAGGCGCGGCTCAGCTGATGGCCCAAGATCC |
| | | GCTCAACCGTTGCGGCCAGAGGACGGGCATTGCAGTTGGCC |
| | | TCTG (SEQ ID NO: 51) |
| DAP10e KIR3DL1 | hinge | TTTPAPRPPTPAPTIALQPLSLRPEACRPAAGGAVHTRGLDFAC |
| | (CD8- | DQTTPGERSSLPAFYPGTSGSCSGCGSLSLP (SEQ ID NO: 70) |
| | DAP10e) | |
| | amino acid | |
| DAP10 KIR3DL1 | hinge | ACCACGACGCCAGCGCCGCGACCACCAACACCGGCGCCCAC |
| | (CD8- | CATCGCGTTGCAGCCCCTGTCCCTGCGCCCAGAGGCGTGCC |
| | DAP10e) | GGCCAGCGGCGGGGGGCGCAGTGCACACGAGGGGGCTGGA |
| | nucleic acid | CTTCGCCTGTGATCAGACCACACCTGGCGAGAGATCTTCCCT |
| | | GCCTGCCTTCTATCCTGGCACCAGCGGCTCTTGTTCTGGCTG |
| | | TGGATCACTGAGCCTGCCT (SEQ ID NO: 87) |
| 28-28 KIR3DL1 | hinge | AAAIEVMYPPPYLDNEKSNGTIIHVKGKHLCPSPLFPGPSKP |
| | (CD28) | (SEQ ID NO: 31) |
| | amino acid | |
| 28-28 KIR3DL1 | hinge | GCCGCTGCTATCGAAGTGATGTACCCTCCTCCTTACCTGGAC |
| | (CD28) | AACGAGAAGTCCAACGGCACCATCATCCACGTGAAGGGCAA |
| | nucleic acid | GCACCTGTGTCCTTCTCCACTGTTCCCCGGACCTAGCAAGCC |
| | | T (SEQ ID NO: 88) |
| TIGIT | inhibitory | LTRKKKALRIHSVEGDLRRKSAGQEEWSPSAPSPPGSCVQAEA |
| TIGIT (full) | cytosolic | APAGLCGEQRGEDCAELHDYFNVLSYRSLGNCSFFTETG(SEQ |
| | domain 1 | ID NO: 95) |
| | (TIGIT) | |
| | amino acid | |
| TIGIT | inhibitory | CTGACCAGAAAGAAGAAGGCCCTGAGAATCCACAGCGTGG |
| TIGIT (full) | cytosolic | AAGGCGACCTGCGGAGAAAGTCTGCCGGACAAGAAGAGTG |
| | domain 1 | GTCCCCTAGCGCTCCATCTCCACCTGGATCTTGTGTGCAGGC |
| | (TIGIT) | CGAAGCAGCTCCTGCTGGACTGTGTGGCGAACAGAGAGGCG |
| | nucleic acid | AAGATTGCGCCGAGCTGCACGACTACTTCAACGTGCTGAGC |
| | | TACAGAAGCCTGGGCAACTGCAGCTTCTTCACCGAGACAGG |
| | | A (SEQ ID NO: 96) |
| PD-1 (full) | ECD (PD-1) | FLDSPDRPWNPPTFSPALLVVTEGDNATFTCSFSNTSESFVLNW |
| | amino acid | YRMSPSNQTDKLAAFPEDRSQPGQDCRFRVTQLPNGRDFHMS |
| | | VVRARRNDSGTYLCGAISLAPKAQIKESLRAELRVTERRAEVPT |
| | | AHPSPSPRPAGQFQTLV (SEQ ID NO: 97) |
| PD-1 (full) | ECD (PD-1) | TTCCTGGACAGCCCCGACAGACCTTGGAACCCTCCTACATTC |
| | nucleic acid | AGCCCCGCTCTGCTGGTGGTTACCGAGGGCGATAATGCCAC |
| | | CTTCACCTGTAGCTTCAGCAACACCAGCGAGAGCTTCGTGCT |
| | | GAACTGGTACAGAATGAGCCCCAGCAACCAGACCGACAAG |
| | | CTGGCCGCCTTTCCTGAGGATAGATCTCAGCCCGGCCAGGA |
| | | CTGCCGGTTCAGAGTTACACAGCTGCCCAACGGCCGGGACT |
| | | TCCACATGTCTGTCGTCCGGGCCAGAAGAAACGACAGCGGC |
| | | ACATATCTGTGCGGCGCCATTTCTCTGGCCCCTAAGGCTCAG |
| | | ATCAAAGAGAGCCTGAGAGCCGAGCTGAGAGTGACAGAAA |
| | | GACGGGCCGAAGTGCCCACAGCTCACCCTTCACCTTCTCCA |
| | | AGACCTGCCGGCCAGTTTCAGACACTGGTT (SEQ ID NO: 98) |
| CTLA-4 (full) | ECD (CTLA-4) | KAMHVAQPAVVLASSRGIASFVCEYASPGKATEVRVTVLRQA |
| | amino acid | DSQVTEVCAATYMMGNELTFLDDSICTGTSSGNQVNLTIQGLR |
| | | AMDTGLYICKVELMYPPPYYLGIGNGTQIYVIDPEPCPDSDFLL |
| | | WILAAVSSGLFFYSFLLT (SEQ ID NO: 99) |
| CTLA-4 (full) | ECD (CTLA-4) | AAGGCCATGCATGTGGCTCAGCCTGCTGTGGTGCTGGCCTCT |
| | nucleic | TCTAGAGGAATCGCCAGCTTCGTGTGCGAGTACGCCTCTCCT |
| | acid | GGAAAGGCCACTGAAGTGCGCGTGACCGTTCTGAGACAGGC |
| | | CGATAGCCAAGTGACCGAAGTGTGCGCCGCCACCTACATGA |
| | | TGGGCAACGAGCTGACCTTCCTGGACGACAGCATCTGTACC |
| | | GGCACCAGCAGCGGCAATCAAGTGAACCTGACCATCCAGGG |
| | | CCTGAGAGCCATGGATACCGGCCTGTACATCTGCAAGGTGG |
| | | AACTGATGTACCCTCCTCCTTACTACCTCGGCATCGGCAACG |
| | | GCACCCAGATCTACGTGATCGACCCTGAGCCTTGTCCTGACA |
| | | GCGACTTTCTGCTGTGGATCCTGGCTGCCGTGTCCAGCGGCC |
| | | TGTTCTTCTACTCTTTTCTGCTGACC (SEQ ID NO: 100) |
| KIR3DL1 (full) | ECD | HMGGQDKPFLSAWPSAVVPRGGHVTLRCHYRHRFNNFMLYK |
| | (KIR3DL1) | EDRIHIPIFHGRIFQESFNMSPVTTAHAGNYTCRGSHPHSPTGWS |
| | amino acid | APSNPVVIMVTGNHRKPSLLAHPGPLVKSGERVILQCWSDIMF |
| | | EHFFLHKEGISKDPSRLVGQIHDGVSKANFSIGPMMLALAGTY |
| | | RCYGSVTHTPYQLSAPSDPLDIVVTGPYEKPSLSAQPGPKVQAG |
| | | ESVTLSCSSRSSYDMYHLSREGGAHERRLPAVRKVNRTFQADF |
| | | PLGPATHGGTYRCFGSFRHSPYEWSDPSDPLLVSVTGNPSSSWP |
| | | SPTEPSSKSGNPRHLH (SEQ ID NO: 101) |
| KIR3DL1 (full) | ECD | CACATGGGCGGACAGGATAAGCCTTTCCTGAGCGCCTGGCC |
| | (KIR3DL1) | TTCTGCCGTTGTTCCTAGAGGCGGACACGTGACCCTGCGGTG |
| | nucleic | TCACTACAGACACCGGTTCAACAACTTCATGCTGTACAAAG |
| | acid | AGGACCGGATTCACATCCCCATCTTCCACGGCCGGATCTTCC |
| | | AAGAGTCCTTCAACATGAGCCCCGTGACCACAGCTCACGCC |
| | | GGCAACTACACATGCAGAGGCTCTCACCCTCACAGCCCTAC |
| | | AGGCTGGAGTGCCCCTTCTAACCCCGTGGTCATCATGGTCAC |
| | | CGGCAACCACAGAAAGCCCAGCCTGCTTGCTCATCCCGGAC |
| | | CTCTGGTTAAGTCTGGCGAGCGAGTGATCCTGCAGTGTTGG |
| | | AGCGATATTATGTTCGAGCACTTCTTTCTGCACAAAGAGGGC |
| | | ATCAGCAAGGACCCCTCTAGACTCGTGGGCCAGATCCATGA |
| | | TGGCGTGTCCAAGGCCAACTTCAGCATCGGCCCTATGATGCT |
| | | GGCCCTGGCCGGCACCTATAGATGTTACGGCAGCGTGACCC |
| | | ACACACCTTACCAGCTGAGCGCCCCTAGCGACCCTCTGGAT |
| | | ATCGTGGTCACAGGCCCCTACGAGAAGCCTAGCCTGTCTGC |
| | | ACAGCCTGGACCTAAAGTGCAGGCCGGCGAAAGCGTGACAC |
| | | TGAGCTGTAGCAGCAGATCCAGCTACGACATGTACCACCTG |
| | | AGCAGAGAAGGCGGAGCCCACGAGAGAAGGCTGCCTGCCG |
| | | TCAGAAAAGTGAACCGGACCTTCCAGGCCGACTTTCCTCTG |
| | | GGACCTGCTACACACGGCGGCACCTACCGGTGTTTCGGCAG |
| | | CTTTAGACACAGCCCTTACGAGTGGAGCGACCCCTCTGATCC |
| | | TCTGCTGGTGTCTGTGACCGGCAATCCTAGCAGCAGCTGGCC |
| | | CTCTCCAACAGAGCCTTCTAGCAAGAGCGGCAACCCCAGAC |
| | | ATCTGCAC (SEQ ID NO: 102) |
| TIGIT (full) | ECD (TIGIT) | MMTGTIETTGNISAEKGGSIILQCHLSSTTAQVTQVNWEQQDQ |
| | amino acid | LLAICNADLGWHISPSFKDRVAPGPGLGLTLQSLTVNDTGEYFC |
| | | IYHTYPDGTYTGRIFLEVLESSVAEHGARFQIP |
| | | (SEQ ID NO: 103) |
| TIGIT (full) | ECD (TIGIT) | ATGATGACCGGCACCATCGAGACAACCGGCAACATCTCTGC |
| | nucleic acid | CGAGAAAGGCGGCAGCATCATCCTGCAGTGTCACCTGTCTA |
| | | GCACCACCGCTCAAGTGACCCAAGTGAACTGGGAGCAGCAG |
| | | GATCAGCTGCTGGCCATCTGCAATGCCGATCTCGGCTGGCA |
| | | CATCAGCCCCAGCTTCAAGGATAGAGTGGCCCCTGGACCTG |
| | | GCCTGGGACTGACACTTCAGAGCCTGACCGTGAACGATACC |
| | | GGCGAGTACTTCTGCATCTACCACACATACCCCGACGGCAC |
| | | CTATACCGGCCGGATCTTTCTGGAAGTGCTGGAAAGCTCTGT |
| | | GGCCGAGCACGGCGCCAGATTTCAGATTCCT (SEQ ID NO: |
| | | 104) |
| NKG2A (full) | inhibitory | MDNQGVIYSDLNLPPNPKRQQRKPKGNKNSILATEQEITYAEL |
| | cytosolic | NLQKASQDFQGNDKTYHCKDLPSAPEK (SEQ ID NO: 105) |
| | domain 1 | |
| | (NKG2A) | |
| | amino acid | |
| NKG2A (full) | inhibitory | ATGGACAACCAGGGCGTGATCTACAGCGACCTGAACCTGCC |
| | cytosolic | TCCTAATCCTAAGCGGCAGCAGAGAAAGCCCAAGGGCAACA |
| | domain 1 | AGAACAGCATCCTGGCCACCGAGCAAGAGATCACCTACGCC |
| | (NKG2A) | GAGCTGAATCTGCAGAAGGCCAGCCAGGACTTCCAGGGCAA |
| | nucleic | CGACAAGACCTACCACTGCAAGGACCTGCCTAGCGCTCCCG |
| | acid | AGAAG (SEQ ID NO: 106) |
| NKG2A (full) | TM | LIVGILGIICLILMASVVTIVVI (SEQ ID NO: 107) |
| | (NKG2A) | |
| | amino acid | |
| NKG2A (full) | TM | CTGATCGTGGGAATCCTGGGCATCATCTGCCTGATCCTGATG |
| | (NKG2A) | GCCAGCGTGGTCACCATCGTGGTCATC (SEQ ID NO: 108) |
| | nucleic acid | |
| NKG2A (full) | ECD | PSTLIQRHNNSSLNTRTQKARHCGHCPEEWITYSNSCYYIGKER |
| | (NKG2A) | RTWEESLLACTSKNSSLLSIDNEEEMKFLSIISPSSWIGVFRNSSH |
| | amino acid | HPWVTMNGLAFKHEIKDSDNAELNCAVLQVNRLKSAQCGSSII |
| | | YHCKHKL (SEQ ID NO: 109) |
| NKG2A (full) | ECD | CCCAGCACACTGATCCAGCGGCACAACAACAGCAGCCTGAA |
| | (NKG2A) | CACCAGAACACAGAAGGCCCGGCACTGCGGCCACTGTCCTG |
| | nucleic acid | AAGAGTGGATCACATACAGCAACAGCTGCTACTACATCGGC |
| | | AAAGAGCGGCGGACCTGGGAAGAATCTCTGCTGGCCTGCAC |
| | | CAGCAAGAACTCCAGCCTGCTGAGCATCGACAACGAGGAAG |
| | | AGATGAAGTTCCTGTCCATCATCAGCCCCAGCAGCTGGATC |
| | | GGCGTGTTCAGAAACAGCTCCCACCATCCTTGGGTCACCAT |
| | | GAACGGCCTGGCCTTCAAGCACGAGATCAAGGACAGCGACA |
| | | ACGCCGAACTGAACTGTGCCGTGCTGCAAGTGAACCGGCTG |
| | | AAGTCTGCCCAGTGTGGCAGCAGCATCATCTATCACTGCAA |
| | | GCACAAGCTG (SEQ ID NO: 110) |
| NKG2A (full) | Tag | NGAAEQKLISEEDL (SEQ ID NO: 111) |
| | (NGAA + | |
| | Myctag) | |
| | amino acid | |
| NKG2A (full) | Tag | AATGGGGCCGCAGAACAAAAACTCATCTCAGAAGAAGATCT |
| | (NGAA + | G(SEQIDNO: 112) |
| | Myctag) | |
| | nucleic acid | |

### Results

NK cells are engineered to express activating chimeric receptors (aCARS) and inhibitory chimeric receptors (iCARs) having various inhibitory domain formats, such as various inhibitory domains derived from different inhibitory receptors, various CAR sequences (e.g., various transmembrane or hinge sequences), and/or various tandem organizations of inhibitory domains. The formats assessed are described in Table 14. NK cells are virally transduced with aCAR only or in combination with iCARs having the various inhibitory domains indicated. Engineered NK cells are assessed for iCARs reducing aCAR-induced NK cell mediated killing of target cells and NK cell cytokine production. The results demonstrate NK cells are successfully engineered to co-express aCARs and iCARs, successfully kill target cells and produce cytokines in the absence of an iCAR ligand in an aCAR ligand dependent manner, and successfully reduce NK-mediated killing and cytokine production in an iCAR ligand dependent manner.

### Example 8: Further Assessment of Various Inhibitory Chimeric Receptors in Reducing NK Cell Activation

### Materials and Methods

Individual iCAR and aCAR constructs were packaged into lentiviral particles and used to transduce primary NK cells after 10 d expansion with K562 feeder cells with 500 U/mL IL-2 and 20 ng/uL IL-15. Virus amounts were set by p24 titer (750,000 pg per transduction). iCAR constructs contained puroR cassettes and puromycin was added to NK cell cultures from day 4 to 7 post transduction, at which time expression was assessed by flow cytometry and NK cells were transferred to a microwell plate for killing assays with 12,500 NK cells and 50,000 total tumor cells. NK cells were cultured with (1) tumor cells (SEM cells) expressing aCAR antigen only, (2) tumor cells expressing both aCAR antigen and iCAR antigen, or (3) both tumor cell types mixed. After 16-18 hrs, cultures were analyzed by flow cytometry and remaining live targets cells of each type were counted. aCAR-mediated killing (basal subtracted) of a given NK cell type was quantified by first calculating total killing (reduction of targets compared to a target-only condition), and then subtracting total killing by control (iCAR-only) NK cells. iCAR-mediated protection was quantified as the change in aCAR-mediated killing between targets with or without iCAR antigen. Killing assay supernatant was analyzed for TNFα secretion, and aCAR and iCAR performance metrics were calculated analogously to killing. For expression analysis, iCARs were stained with a V5-Alexafluor 647 and aCARs with aFLAG-BV-421. Cells were assigned to 4 quadrants based on iCAR+/− and aCAR+/− expression states, allowing us to assess “% aCAR+iCAR+” and “% not aCAR+iCAR−” (aCAR+iCAR− are ungated and potentially toxic CAR-NK cells and are to be avoided). To further analyze expression level, we measured median fluorescence intensity (MFI) of aCAR and iCAR of the aCAR+iCAR+ subpopulation, which we normalized by the MFI of untransduced NK cells in the respective fluorescence channels. For each iCAR, 1-3 biological replicates were performed (shown as different points with the same marker type). X and Y error lines (where applicable): +/−standard error of the mean.

Sequences for the iCAR constructs assessed are shown in Table 15. Each iCAR construct format is from N to C terminal: signal sequence 1-signal sequence 2-scFv-tag-hinge-TM-inhibitory cytosolic domain 1-inhibitory cytosolic domain 2 (if present). NKG2A formats assessed did not include a signal sequence 2. The aCAR format uses a CD28-CD3z format from N to C terminal: signal sequence-tag-scFv-hinge-TM-intracellular signaling domain 1-intracellular signaling domain 2 (see sequences shown in Table 10B).

TABLE 15
iCAR Formats and Domains for NK Cells

| Construct | | |
|---|---|---|
| (by ICD) | Domain | Sequence |
| BTLA | signal | MALPVTALLLPLALLLHAARP (SEQ ID NO: 71) |
| LIR1 | sequence 1 | |
| LIR1 2x | (CD8) | |
| LIR1-KIR3DL1 | amino acid | |
| KIR3DL1 | | |
| KIR3DL1 2x | | |
| NKG2A | | |
| NKG2A (LIR1 TM) | | |
| BTLA | signal | ATGGCCTTACCAGTGACCGCCTTGCTCCTGCCGCTGGCCTTG |
| LIR1 | sequence 1 | CTGCTCCACGCCGCCAGGCCG (SEQ ID NO: 72) |
| LIR1 2x | (CD8) | |
| LIR1-KIR3DL1 | nucleic | |
| KIR3DL1 | acid | |
| KIR3DL1 2x | | |
| NKG2A | | |
| NKG2A (LIR1 TM) | | |
| BTLA | signal | KYLLPTAAAGLLLLAAQPAMA (SEQ ID NO: 73) |
| LIR1 | sequence 2 | |
| LIR1 2x | (pelB) | |
| LIR1-KIR3DL1 | amino acid | |
| KIR3DL1 | | |
| KIR3DL1 2x | | |
| BTLA | signal | AAATACCTATTGCCTACGGCAGCCGCTGGATTGTTATTACTC |
| LIR1 | sequence 2 | GCGGCCCAGCCGGCCATGGCC (SEQ ID NO: 74) |
| LIR1 2x | (pelB) | |
| LIR1-KIR3DL1 | nucleic | |
| KIR3DL1 | acid | |
| KIR3DL1 2x | | |
| BTLA | scFv | iCAR-antigen specific scFv with (G4S)3 linker |
| LIR1 | (iCAR- | |
| LIR1 2x | specific | |
| LIR1-KIR3DL1 | scFv with | |
| KIR3DL1 | (G4S)3 | |
| KIR3DL1 2x | linker) | |
| NKG2A | | |
| NKG2A (LIR1 TM) | | |
| BTLA | tag | GKPIPNPLLGLDSTNGAA (SEQ ID NO: 77) |
| LIR1 | (V5 + | |
| LIR1 2x | NGAA | |
| LIR1-KIR3DL1 | linker) | |
| KIR3DL1 | amino acid | |
| KIR3DL1 2x | | |
| NKG2A | | |
| NKG2A (LIR1 TM) | | |
| BTLA | tag | GGGAAGCCTATCCCGAACCCTCTGTTGGGTCTCGATAGTACC |
| LIR1 | (V5 + | AATGGGGCCGCA (SEQ ID NO: 78) |
| LIR1 2x | NGAA | |
| LIR1-KIR3DL1 | linker) | |
| KIR3DL1 | nucleic | |
| KIR3DL1 2x | acid | |
| NKG2A | | |
| NKG2A (LIR1 TM) | | |
| BTLA | hinge | TTTPAPRPPTPAPTIALQPLSLRPEACRPAAGGAVHTRGLDFAC |
| LIR1 | (CD8) | D (SEQ ID NO: 37) |
| LIR1 2x | amino acid | |
| LIR1-KIR3DL1 | | |
| KIR3DL1 | | |
| KIR3DL1 2x | | |
| NKG2A | | |
| NKG2A (LIR1 TM) | | |
| BTLA | hinge | ACCACGACGCCAGCGCCGCGACCACCAACACCGGCGCCCAC |
| LIR1 | (CD8) | CATCGCGTTGCAGCCCCTGTCCCTGCGCCCAGAGGCGTGCC |
| LIR1 2x | nucleic | GGCCAGCGGCGGGGGGCGCAGTGCACACGAGGGGGCTGGA |
| LIR1-KIR3DL1 | acid | CTTCGCCTGTGAT (SEQ ID NO: 79) |
| KIR3DL1 | | |
| KIR3DL1 2x | | |
| NKG2A | | |
| NKG2A (LIR1 TM) | | |
| KIR3DL1 | TM | ILIGTSVVIILFILLLFFLL (SEQ ID NO: 69) |
| KIR3DL1 2x | (KIR3DL1) | |
| | amino acid | |
| KIR3DL1 | TM | ATCCTGATCGGGACAAGTGTAGTAATCATACTTTTCATACTC |
| KIR3DL1 2x | (KIR3DL1) | CTGCTCTTTTTTCTCTTG (SEQ ID NO: 81) |
| | nucleic acid | |
| LIR1 | TM | VIGILVAVILLLLLLLLLFLI (SEQ ID NO: 59) |
| LIR1-KIR3DL1 | (LIR1) | |
| LIR1 2x | amino acid | |
| NKG2A (LIR1 TM) | | |
| LIR1 | TM | GTTATAGGGATCCTGGTGGCTGTCATACTCCTCTTGCTCCTC |
| LIR1-KIR3DL1 | (LIR1) | TTGTTGCTGCTTTTTTTGATA (SEQ ID NO: 62) |
| LIR1 2x | nucleic acid | |
| NKG2A (LIR1 TM) | TM | GTGATCGGCATTCTGGTGGCCGTGATTCTGCTGCTCCTGCTG |
| | (LIR1) | TTGCTGCTGCTGTTCCTGATC (SEQ ID NO: 131) |
| | nucleic acid | |
| BTLA | TM | LLPLGGLPLLTTTCFCLFCCL (SEQ ID NO: 12) |
| | (BTLA) | |
| | nucleic acid | |
| BTLA | TM | CTCTTGCCGTTGGGGGGTCTGCCACTTCTCATAACAACTTGC |
| | (BTLA) | TTCTGCCTTTTTTGCTGTTTG (SEQ ID NO: 14) |
| | nucleic acid | |
| NKG2A | TM | LIVGILGIICLILMASVVTIVVI (SEQ ID NO: 107) |
| | (NKG2A) | |
| | amino acid | |
| NKG2A | TM | CTGATCGTGGGCATCCTGGGCATCATCTGTCTGATCCTGATG |
| | (NKG2A) | GCCAGCGTGGTCACCATCGTGGTCATC (SEQ ID NO: 132) |
| | amino acid | |
| KIR3DL1 | inhibitory | HLWCSNKKNAAVMDQEPAGNRTANSEDSDEQDPEEVTYAQL |
| KIR3DL1 2x | cytosolic | DHCVFTQRKITRPSQRPKTPPTDTILYTELPNAKPRSKVVSCP |
| | domain 1 | (SEQ ID NO: 66) |
| | (KIR3DL1) | |
| | amino acid | |
| KIR3DL1 | inhibitory | CATCTGTGGTGTTCTAATAAGAAGAATGCTGCTGTGATGGAT |
| KIR3DL1 2x | cytosolic | CAAGAGCCCGCTGGTAACAGAACGGCCAACAGTGAAGATA |
| | domain 1 | GCGATGAGCAGGACCCAGAAGAAGTGACCTACGCCCAACTC |
| | (KIR3DL1) | GACCACTGTGTTTTTACGCAGCGGAAAATCACTCGACCCTCT |
| | nucleic acid | CAACGACCCAAAACGCCGCCTACGGACACCATACTCTACAC |
| | | CGAACTGCCGAACGCCAAACCACGGTCCAAGGTGGTATCAT |
| | | GTCCG (SEQ ID NO: 85) |
| LIR1 | inhibitory | LRHRRQGKHWTSTQRKADFQHPAGAVGPEPTDRGLQWRSSPA |
| LIR1-KIR3DL1 | cytosolic | ADAQEENLYAAVKHTQPEDGVEMDTRSPHDEDPQAVTYAEV |
| LIR1 2x | domain 1 | KHSRPRREMASPPSPLSGEFLDTKDRQAEEDRQMDTEAAASEA |
| | (LIR1) | PQDVTYAQLHSLTLRREATEPPPSQEGPSPAVPSIYATLAIH |
| | amino acid | (SEQ ID NO: 50) |
| LIR1 | inhibitory | TTGCGCCACAGACGGCAGGGAAAGCACTGGACTAGTACGCA |
| LIR1-KIR3DL1 | cytosolic | GAGGAAAGCGGACTTCCAGCATCCCGCAGGAGCCGTGGGGC |
| LIR1 2x | domain 1 | CTGAACCCACTGATCGCGGCCTTCAATGGAGGTCTAGCCCG |
| | (LIR1) | GCGGCAGACGCACAAGAGGAAAACTTGTACGCAGCCGTTAA |
| | nucleic acid | GCACACCCAACCGGAGGACGGCGTTGAGATGGATACCCGCT |
| | | CCCCTCACGATGAAGACCCTCAAGCAGTCACTTACGCGGAA |
| | | GTAAAGCATAGCCGCCCCAGACGGGAAATGGCTAGCCCGCC |
| | | GTCCCCCCTTAGCGGGGAATTTCTGGACACTAAAGATAGGC |
| | | AGGCGGAAGAGGACCGCCAAATGGATACAGAGGCGGCGGC |
| | | AAGTGAAGCACCTCAAGACGTTACTTACGCTCAACTTCACA |
| | | GCCTTACCCTCAGGCGAGAAGCGACTGAACCACCCCCTTCC |
| | | CAAGAAGGGCCAAGCCCAGCGGTTCCTTCTATCTATGCTACT |
| | | CTTGCTATTCAC (SEQ ID NO: 54) |
| BTLA | inhibitory | RRHQGKQNELSDTAGREINLVDAHLKSEQTEASTRQNSQVLLS |
| | cytosolic | ETGIYDNDPDLCFRMQEGSEVYSNPCLEENKPGIVYASLNHSVI |
| | domain 1 | GPNSRLARNVKEAPTEYASICVRS (SEQ ID NO: 3) |
| | (BTLA) | |
| | amino acid | |
| BTLA | inhibitory | AGAAGACATCAGGGGAAGCAGAATGAACTCAGCGATACAG |
| | cytosolic | CAGGGCGAGAAATTAATTTGGTAGACGCGCATCTGAAGTCC |
| | domain 1 | GAACAGACAGAGGCTTCTACTAGACAGAACTCCCAAGTTTT |
| | (BTLA) | GTTGAGTGAGACGGGGATCTATGATAATGATCCCGATCTGT |
| | nucleic acid | GTTTTAGAATGCAGGAGGGTAGTGAAGTCTACTCAAACCCG |
| | | TGCCTGGAAGAAAATAAGCCCGGCATTGTTTACGCTAGTTT |
| | | GAATCATTCTGTAATAGGCCCGAACTCCAGACTGGCTCGCA |
| | | ATGTGAAGGAGGCCCCAACTGAGTATGCGTCCATTTGCGTG |
| | | CGGTCT (SEQ ID NO: 52) |
| NKG2A | inhibitory | MDNQGVIYSDLNLPPNPKRQQRKPKGNKNSILATEQEITYAEL |
| NKG2A (LIR1 TM) | cytosolic | NLQKASQDFQGNDKTYHCKDLPSAPEK (SEQ ID NO: 105) |
| | domain 1 | |
| | (NKG2A) | |
| | amino acid | |
| NKG2A | inhibitory | ATGGACAACCAGGGCGTCATCTACAGCGACCTGAACCTGCC |
| NKG2A (LIR1 TM) | cytosolic | TCCTAATCCAAAGCGGCAGCAGCGGAAGCCCAAGGGCAAC |
| | domain 1 | AAGAATAGCATCCTGGCCACCGAGCAAGAGATCACCTACGC |
| | (NKG2A) | CGAGCTGAATCTGCAGAAGGCCAGCCAGGATTTCCAGGGCA |
| | nucleic acid | ACGACAAGACCTACCACTGCAAGGACCTGCCTAGCGCTCCT |
| | | GAGAAA (SEQ ID NO: 130) |
| LIR1-KIR3DL1 | inhibitory | SEQ ID NO: 66 |
| KIR3DL1 2x | cytosolic | |
| | domain 2 | |
| | (KIR3DL1) | |
| | amino acid | |
| LIR1-KIR3DL1 | inhibitory | SEQ ID NO: 85 |
| KIR3DL1 2x | cytosolic | |
| | domain 2 | |
| | (KIR3DL1) | |
| | nucleic acid | |
| LIR1 2x | inhibitory | SEQ ID NO: 50 |
| | cytosolic | |
| | domain 2 | |
| | (LIR1) | |
| | amino acid | |
| LIR1 2x | inhibitory | SEQ ID NO: 54 |
| | cytosolic | |
| | domain 2 | |
| | (LIR1) | |
| | nucleic acid | |

### Results

NK cells were engineered to express activating chimeric receptors (aCARS) and inhibitory chimeric receptors (iCARs) having various inhibitory domain formats, such as various inhibitory domains derived from different inhibitory receptors, various CAR sequences (e.g., various transmembrane or hinge sequences), and/or various tandem organizations of inhibitory domains. The formats assessed are described in Table 15. NK cells were virally transduced with aCAR only or in combination with iCARs having the various inhibitory domains indicated.

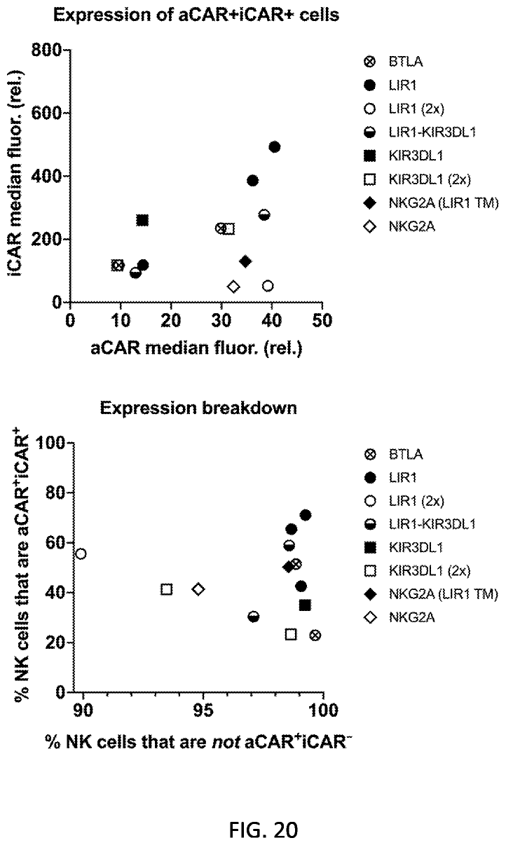

Engineered NK cells were assessed for CAR expression. As shown in FIG. 20, among aCAR+iCAR+NK cells (top panel), aCAR expression is generally greater than 10-fold above background and iCAR is generally greater than 100-fold. LIR1 constructs demonstrated notably high expression relative to other constructs. The profile of CAR expressing populations was also assessed (bottom panel) and demonstrated the total population contained fewer than 5% aCAR+iCAR− cells and had varying percentages of aCAR+iCAR+ populations for the various iCAR formats. Again, LIR1-containing iCARs notably generally demonstrated a greater proportion of aCAR+iCAR+ cells relative to other constructs.

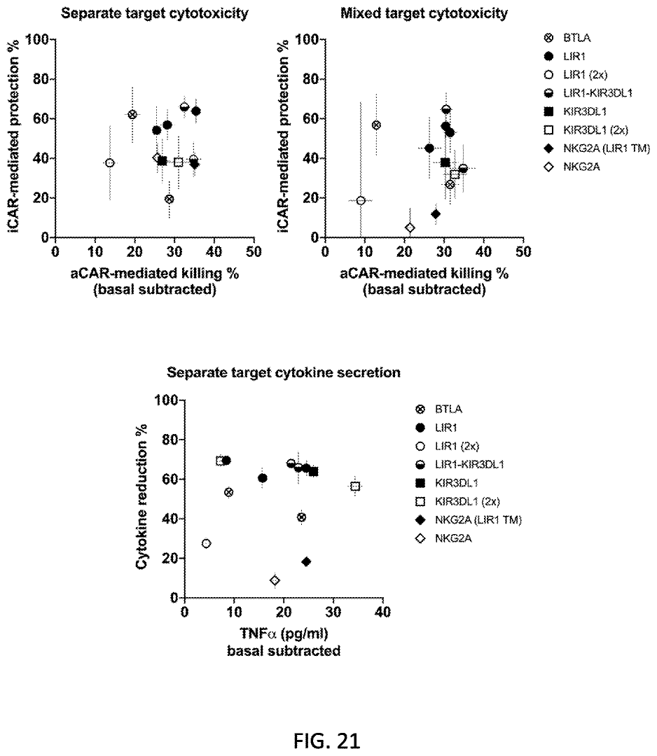

Next, iCARs reduction of aCAR-induced NK cell mediated killing of target cells and NK cell cytokine production was assessed. Reduction was assessed for each of the target SEM cells separately (“Separate”: aCAR antigen only SEM cells and aCAR/iCAR antigen co-expressing SEM cells separately) or in the context of a mixed population of target and non-target cells (“Mixed”: aCAR antigen only SEM cells and aCAR/iCAR antigen co-expressing SEM cells together in the same culture). As shown in FIG. 21, NK cells expressing LIR1, LIR1 (2×), KIR3DL1, KIR3DL1 (2×) iCAR formats demonstrated consistent aCAR-mediated performance in killing (top panels) and iCAR-mediated protection in both killing (top panels) and cytokine reduction (bottom panel), with BTLA and NKG2A constructs varying more in their performance.

The results demonstrate NK cells were successfully engineered to co-express aCARs and iCARs, successfully kill target cells and produce cytokines in the absence of an iCAR ligand in an aCAR ligand dependent manner, and successfully reduce NK-mediated killing and cytokine production in an iCAR ligand dependent manner.

### INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### EQUIVALENTS

While various specific embodiments have been illustrated and described, the above specification is not restrictive. It will be appreciated that various changes can be made without departing from the spirit and scope of the present disclosure(s). Many variations will become apparent to those skilled in the art upon review of this specification.

What is claimed is:

1. A chimeric inhibitory receptor comprising:
(a) an extracellular protein binding domain,
(b) a transmembrane domain, wherein the transmembrane domain is operably linked to the extracellular protein binding domain, wherein the transmembrane domain is selected from the group consisting of BTLA, CD8, CD3zeta, CD4, 4-IBB, OX40, ICOS, 2B4, CD25, CD7, LAX, LAT, PD-1, CTLA4, TIM3, KIR3DL1, LIR1, NKG2A, TIGIT, and LAG3,
(c) one or more intracellular signaling domains, wherein the one or more intracellular signaling domains are operably linked to the transmembrane domain;
wherein each of the one or more intracellular signaling domains is derived from a protein selected from the group consisting of BTLA, PD-1, CTLA4, TIM3, KIR3DL1, LIR1, NKG2A, TIGIT, and LAG3; and
wherein at least one of the one or more intracellular signaling domains is capable of preventing, attenuating, or inhibiting activation of a tumor-targeting chimeric receptor expressed on an immunomodulatory cell.

2. The chimeric inhibitory receptor of claim 1, wherein the transmembrane domain further comprises at least a portion of an extracellular domain of the same protein.

3. The chimeric inhibitory receptor of claim 1, wherein the one of the one or more intracellular signaling domains is derived from KIR3DL1.

4. The chimeric inhibitory receptor of claim 1, wherein the intracellular signaling domain comprises the amino acid sequence of (SEQ ID NO: 66) HLWCSNKKNAAVMDQEPAGNRTANSEDSDEQDPEEVTYAQLDHCVFTQRKITRPSQ RPKTPPTDTILYTELPNAKPRSKVVSCP.

5. The chimeric inhibitory receptor of claim 1, wherein the transmembrane domain is derived from KIR3DL1.

6. The chimeric inhibitory receptor of claim 1, wherein the transmembrane domain comprises the amino acid sequence of ILIGTSVVIILFILLLFFLL (SEQ ID NO: 69).

7. The chimeric inhibitory receptor of claim 1, wherein the protein binding domain binds a protein that is not expressed on the target tumor, or the protein binding domain binds a protein that is expressed on a non-tumor cell.

8. The chimeric inhibitory receptor of claim 1, wherein the extracellular protein binding domain comprises a ligand-binding domain, a receptor-binding domain, or an antigen-binding domain.

9. The chimeric inhibitory receptor of claim 1, wherein the chimeric inhibitory receptor further comprises a spacer region positioned between the extracellular protein binding domain and the transmembrane domain and operably linked, or physically linked, to each of the extracellular protein binding domain and the transmembrane domain, wherein the spacer region is derived from a protein selected from the group consisting of: CD8alpha, CD4, CD7, CD28, IgG1, IgG4, FcgammaRIIIalpha, LNGFR, and PDGFR; or
wherein the chimeric inhibitory receptor further comprises an intracellular spacer region positioned between the transmembrane domain and one of the one or more intracellular signaling domains and operably linked, or physically linked, to each of the transmembrane domain and the one of the one or more intracellular signaling domains, wherein the spacer region is derived from a protein selected from the group consisting of: CD8alpha, CD4, CD7, CD28, IgG1, IgG4, FcgammaRIIIalpha, LNGFR, and PDGFR.

10. The chimeric inhibitory receptor of claim 1, wherein the tumor targeting chimeric receptor is a tumor-targeting chimeric antigen receptor (CAR) or an engineered T cell receptor (TCR).

11. The chimeric inhibitory receptor of claim 1, wherein the immunomodulatory cell is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an ESC-derived cell, and an iPSC-derived cell.

12. The chimeric inhibitory receptor of claim 1, comprising a transmembrane domain derived from KIR3DL1 and one or more intracellular signaling domains derived from KIR3DL1.

13. An engineered nucleic acid encoding the chimeric inhibitory receptor of claim 1.

14. An expression vector comprising the engineered nucleic acid of claim 13.

15. An isolated immunomodulatory cell comprising the chimeric inhibitory receptor of claim 1.

16. A composition comprising:
(a) the chimeric inhibitory receptor of claim 1; and
(b) a pharmaceutically acceptable carrier, pharmaceutically acceptable excipient, or a combination thereof.

17. A method of preventing, attenuating, or inhibiting a cell-mediated immune response of an immunomodulatory cell, comprising:
engineering the immunomodulatory cell to express a chimeric inhibitory receptor on the surface of the immunomodulatory cell,
wherein upon binding of a cognate protein to the chimeric inhibitory receptor, the intracellular signaling domain prevents, attenuates, or inhibits activation of the cell-mediated immune response of the immunomodulatory cell,
wherein the chimeric inhibitory receptor comprises:
(a) an extracellular protein binding domain,
(b) a transmembrane domain, wherein the transmembrane domain is operably linked to the extracellular protein binding domain, wherein the transmembrane domain is selected from the group consisting of BTLA, CD8, CD3zeta, CD4, 4-IBB, OX40, ICOS, 2B4, CD25, CD7, LAX, LAT, PD-1, CTLA4, TIM3, KIR3DL1, LIR1, NKG2A, TIGIT, and LAG3,
(c) one or more intracellular signaling domains, wherein the one or more intracellular signaling domains are operably linked to the transmembrane domain; and
wherein each of the one or more intracellular signaling domains is derived from a protein selected from the group consisting of BTLA, PD-1, CTLA4, TIM3, KIR3DL1, LIR1, NKG2A, TIGIT, and LAG3.

18. A method of preventing, attenuating, or inhibiting activation of a tumor-targeting chimeric receptor expressed on the surface of an immunomodulatory cell, comprising:
contacting the isolated cell of claim 15 with a cognate protein of the chimeric inhibitory receptor under conditions suitable for the chimeric inhibitory receptor to bind the cognate protein,
wherein upon binding of the protein to the chimeric inhibitory receptor, the intracellular signaling domain prevents, attenuates, or inhibits activation of the tumor-targeting chimeric receptor.

19. A method of preventing, attenuating, or inhibiting a cell-mediated immune response, comprising:
engineering the immunomodulatory cell to express the chimeric inhibitory receptor of claim 1 on the surface of the immunomodulatory cell,
wherein upon binding of a cognate protein to the chimeric inhibitory receptor, the intracellular signaling domain prevents, attenuates, or inhibits activation of the cell-mediated immune response of the immunomodulatory cell.

20. A chimeric inhibitory receptor comprising:
(a) an extracellular protein binding domain,
(b) a transmembrane domain, wherein the transmembrane domain is operably linked to the extracellular protein binding domain, wherein the transmembrane domain is selected from the group consisting of BTLA, PD-1, CTLA4, TIM3, KIR3DL1, LIR1, NKG2A, TIGIT, and LAG3, and
(c) one or more intracellular signaling domains, wherein the one or more intracellular signaling domains are operably linked to the transmembrane domain, and wherein the transmembrane domain and one of the one or more intracellular signaling domains are derived from the same protein; and
wherein at least one of the one or more intracellular signaling domains is capable of preventing, attenuating, or inhibiting activation of a tumor-targeting chimeric receptor expressed on an immunomodulatory cell, optionally wherein the transmembrane domain further comprises at least a portion of an extracellular domain of the same protein.


SEQUENCE LISTING (134 sequences)

SEQ ID NO: 1 - protein, 97 aa, synthetic construct
  REGION 1..97: note = Description of Artificial Sequence: Synthetic polypeptide
CSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 2 - protein, 41 aa, synthetic construct
  REGION 1..41: note = Description of Artificial Sequence: Synthetic polypeptide
AVSLSKMLKKRSPLTTGVYVKMPPTEPECEKQFQPYFIPIN

SEQ ID NO: 3 - protein, 111 aa, synthetic construct
  REGION 1..111: note = Description of Artificial Sequence: Synthetic polypeptide
RRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 4 - DNA, 291 bp, synthetic construct
  misc_feature 1..291: note = Description of Artificial Sequence: Synthetic polynucleotide
  source 1..291: mol_type = other DNA
tgtagcagagccgccagaggaacaatcggcgccagaagaacaggccagcctctgaaagaggacccctctgccgttcctgtgttcagcgtggactatggcgagctggatttccagtggcgggaaaagacacccgagcctccagtgccttgtgtgcctgagcagacagagtacgccaccatcgtgttccctagcggcatgggcacatctagccctgccagaagaggatctgccgacggacctagatctgcccagcctcttagacctgaggacggccactgttcttggcctctt

SEQ ID NO: 5 - DNA, 123 bp, synthetic construct
  misc_feature 1..123: note = Description of Artificial Sequence: Synthetic polynucleotide
  source 1..123: mol_type = other DNA
gccgtgtctctgagcaagatgctgaagaagcggagccctctgaccaccggcgtgtacgtgaaaatgcctcctaccgagcctgagtgcgagaagcagttccagccttacttcatccccatcaac

SEQ ID NO: 6 - DNA, 333 bp, synthetic construct
  misc_feature 1..333: note = Description of Artificial Sequence: Synthetic polynucleotide
  source 1..333: mol_type = other DNA
aggagacatcaggggaagcagaatgaactcagcgatacagcagggcgagaaattaatttggtagacgcgcatctgaagtccgaacagacagaggcttctactagacagaactcccaagttttgttgagtgagacggggatctatgataatgatcccgatctgtgttttagaatgcaggagggtagtgaagtctactcaaacccgtgcctggaagaaaataagcccggcattgtttacgctagtttgaatcattctgtaataggcccgaactccagactggctcgcaatgtgaaggaggccccaactgagtatgcgtccatttgcgtgcggtct

SEQ ID NO: 7 - protein, 21 aa, synthetic construct
  REGION 1..21: note = Description of Artificial Sequence: Synthetic peptide
IFSGFAGLLAILLVVAVFCIL

SEQ ID NO: 8 - protein, 19 aa, synthetic construct
  REGION 1..19: note = Description of Artificial Sequence: Synthetic peptide
VAVAGCVFLLISVLLLSGL

SEQ ID NO: 9 - protein, 21 aa, synthetic construct
  REGION 1..21: note = Description of Artificial Sequence: Synthetic peptide
AALAVISFLLGLGLGVACVLA

SEQ ID NO: 10 - protein, 36 aa, synthetic construct
  REGION 1..36: note = Description of Artificial Sequence: Synthetic polypeptide
MEADALSPVGLGLLLLPFLVTLLAALAVRARELPVS

SEQ ID NO: 11 - protein, 27 aa, synthetic construct
  REGION 1..27: note = Description of Artificial Sequence: Synthetic peptide
FWVLVVVGGVLACYSLLVTVAFIIFWV

SEQ ID NO: 12 - protein, 21 aa, synthetic construct
  REGION 1..21: note = Description of Artificial Sequence: Synthetic peptide
LLPLGGLPLLITTCFCLFCCL

SEQ ID NO: 13 - DNA, 81 bp, synthetic construct
  misc_feature 1..81: note = Description of Artificial Sequence: Synthetic oligonucleotide
  source 1..81: mol_type = other DNA
ttttgggtgctggtggtggttggtggagtcctggcttgctatagcttgctagtaacagtggcctttattattttctgggtg

SEQ ID NO: 14 - DNA, 63 bp, synthetic construct
  misc_feature 1..63: note = Description of Artificial Sequence: Synthetic oligonucleotide
  source 1..63: mol_type = other DNA
ctcttgccgttggggggtctgccacttctcataacaacttgcttctgccttttttgctgtttg

SEQ ID NO: 15 - skipped

SEQ ID NO: 16 - protein, 6 aa, synthetic construct
  REGION 1..6: note = Description of Artificial Sequence: Synthetic peptide
GGSGGS

SEQ ID NO: 17 - protein, 9 aa, synthetic construct
  REGION 1..9: note = Description of Artificial Sequence: Synthetic peptide
GGSGGSGGS

SEQ ID NO: 18 - protein, 12 aa, synthetic construct
  REGION 1..12: note = Description of Artificial Sequence: Synthetic peptide
GGSGGSGGSGGS

SEQ ID NO: 19 - protein, 15 aa, synthetic construct
  REGION 1..15: note = Description of Artificial Sequence: Synthetic peptide
GGSGGSGGSGGSGGS

SEQ ID NO: 20 - protein, 4 aa, synthetic construct
  REGION 1..4: note = Description of Artificial Sequence: Synthetic peptide
GGGS

SEQ ID NO: 21 - protein, 8 aa, synthetic construct
  REGION 1..8: note = Description of Artificial Sequence: Synthetic peptide
GGGSGGGS

SEQ ID NO: 22 - protein, 12 aa, synthetic construct
  REGION 1..12: note = Description of Artificial Sequence: Synthetic peptide
GGGSGGGSGGGS

SEQ ID NO: 23 - protein, 16 aa, synthetic construct
  REGION 1..16: note = Description of Artificial Sequence: Synthetic peptide
GGGSGGGSGGGSGGGS

SEQ ID NO: 24 - protein, 20 aa, synthetic construct
  REGION 1..20: note = Description of Artificial Sequence: Synthetic peptide
GGGSGGGSGGGSGGGSGGGS

SEQ ID NO: 25 - protein, 5 aa, synthetic construct
  REGION 1..5: note = Description of Artificial Sequence: Synthetic peptide
GGGGS

SEQ ID NO: 26 - protein, 10 aa, synthetic construct
  REGION 1..10: note = Description of Artificial Sequence: Synthetic peptide
GGGGSGGGGS

SEQ ID NO: 27 - protein, 15 aa, synthetic construct
  REGION 1..15: note = Description of Artificial Sequence: Synthetic peptide
GGGGSGGGGSGGGGS

SEQ ID NO: 28 - protein, 20 aa, synthetic construct
  REGION 1..20: note = Description of Artificial Sequence: Synthetic peptide
GGGGSGGGGSGGGGSGGGGS

SEQ ID NO: 29 - protein, 25 aa, synthetic construct
  REGION 1..25: note = Description of Artificial Sequence: Synthetic peptide
GGGGSGGGGSGGGGSGGGGSGGGGS

SEQ ID NO: 30 - DNA, 45 bp, synthetic construct
  misc_feature 1..45: note = Description of Artificial Sequence: Synthetic oligonucleotide
  source 1..45: mol_type = other DNA
ggaggcggaggatctggtggcggaggaagtggcggaggcggttct

SEQ ID NO: 31 - protein, 42 aa, synthetic construct
  REGION 1..42: note = Description of Artificial Sequence: Synthetic polypeptide
AAAIEVMYPPPYLDNEKSNGTIIHVKGKHLCPSPLFPGPSKP

SEQ ID NO: 32 - protein, 12 aa, synthetic construct
  REGION 1..12: note = Description of Artificial Sequence: Synthetic peptide
ESKYGPPCPSCP

SEQ ID NO: 33 - protein, 12 aa, synthetic construct
  REGION 1..12: note = Description of Artificial Sequence: Synthetic peptide
ESKYGPPAPSAP

SEQ ID NO: 34 - protein, 12 aa, synthetic construct
  REGION 1..12: note = Description of Artificial Sequence: Synthetic peptide
ESKYGPPCPPCP

SEQ ID NO: 35 - protein, 12 aa, synthetic construct
  REGION 1..12: note = Description of Artificial Sequence: Synthetic peptide
EPKSCDKTHTCP

SEQ ID NO: 36 - protein, 86 aa, synthetic construct
  REGION 1..86: note = Description of Artificial Sequence: Synthetic polypeptide
AAAFVPVFLPAKPTTTPAPRPPTPAPTIASQPLSLRPEACRPAAGGAVHTRGLDFACDIYIWAPLAGTCGVLLLSLVITLYCNHRN

SEQ ID NO: 37 - protein, 45 aa, synthetic construct
  REGION 1..45: note = Description of Artificial Sequence: Synthetic polypeptide
TTTPAPRPPTPAPTIALQPLSLRPEACRPAAGGAVHTRGLDFACD

SEQ ID NO: 38 - protein, 132 aa, synthetic construct
  REGION 1..132: note = Description of Artificial Sequence: Synthetic polypeptide
ACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEADAEC

SEQ ID NO: 39 - protein, 34 aa, synthetic construct
  REGION 1..34: note = Description of Artificial Sequence: Synthetic polypeptide
ACPTGLYTHSGECCKACNLGEGVAQPCGANQTVC

SEQ ID NO: 40 - protein, 20 aa, synthetic construct
  REGION 1..20: note = Description of Artificial Sequence: Synthetic peptide
AVGQDTQEVIVVPHSLPFKV

SEQ ID NO: 41 - DNA, 126 bp, synthetic construct
  misc_feature 1..126: note = Description of Artificial Sequence: Synthetic polynucleotide
  source 1..126: mol_type = other DNA
gcagcagctatcgaggtgatgtatcctccgccctacctggataatgaaaagagtaatgggactatcattcatgtaaaagggaagcatctttgtccttctccccttttccccggtccgtctaaacct

SEQ ID NO: 42 - DNA, 36 bp, synthetic construct
  misc_feature 1..36: note = Description of Artificial Sequence: Synthetic oligonucleotide
  source 1..36: mol_type = other DNA
gaaagcaagtacggtccaccttgccctagctgtccg

SEQ ID NO: 43 - DNA, 36 bp, synthetic construct
  misc_feature 1..36: note = Description of Artificial Sequence: Synthetic oligonucleotide
  source 1..36: mol_type = other DNA
gaatccaagtacggccccccagcgcctagtgcccca

SEQ ID NO: 44 - DNA, 36 bp, synthetic construct
  misc_feature 1..36: note = Description of Artificial Sequence: Synthetic oligonucleotide
  source 1..36: mol_type = other DNA
gaatctaaatatggcccgccatgcccgccttgccca

SEQ ID NO: 45 - DNA, 36 bp, synthetic construct
  misc_feature 1..36: note = Description of Artificial Sequence: Synthetic oligonucleotide
  source 1..36: mol_type = other DNA
gaaccgaagtcttgtgataaaactcatacgtgcccg

SEQ ID NO: 46 - DNA, 258 bp, synthetic construct
  misc_feature 1..258: note = Description of Artificial Sequence: Synthetic polynucleotide
  source 1..258: mol_type = other DNA
gctgctgctttcgtacccgtgttcctccctgctaagcctacgactacccccgcaccgagaccacccacgccagcacccacgattgctagccagccccttagtttgcgaccagaagcttgtcggcctgctgctggtggcgcggtacatacccgcggccttgattttgcttgcgatatatatatctgggcgcctctggccggaacatgcggggtcctcctcctttctctggttattactctctactgtaatcacaggaat

SEQ ID NO: 47 - DNA, 396 bp, synthetic construct
  misc_feature 1..396: note = Description of Artificial Sequence: Synthetic polynucleotide
  source 1..396: mol_type = other DNA
gcctgcccgaccgggctctacactcatagcggggaatgttgtaaggcatgtaacttgggtgagggcgtcgcacagccctgcggagctaaccaaacagtgtgcgaaccctgcctcgatagtgtgacgttctctgatgttgtatcagctacagagccttgcaaaccatgtactgagtgcgttggacttcagtcaatgagcgctccatgtgtggaggcagatgatgcggtctgtcgatgtgcttacggatactaccaagacgagacaacagggcggtgcgaggcctgtagagtttgtgaggcgggctccgggctggtgttttcatgtcaagacaagcaaaatacggtctgtgaagagtgccctgatggcacctactcagacgaagcagatgcagaatgc

SEQ ID NO: 48 - DNA, 102 bp, synthetic construct
  misc_feature 1..102: note = Description of Artificial Sequence: Synthetic polynucleotide
  source 1..102: mol_type = other DNA
gcctgccctacaggactctacacgcatagcggtgagtgttgtaaagcatgcaacctcggggaaggtgtagcccagccatgcggggctaaccaaaccgtttgc

SEQ ID NO: 49 - DNA, 60 bp, synthetic construct
  misc_feature 1..60: note = Description of Artificial Sequence: Synthetic oligonucleotide
  source 1..60: mol_type = other DNA
gctgtgggccaggacacgcaggaggtcatcgtggtgccacactccttgccctttaaggtg

SEQ ID NO: 50 - protein, 168 aa, synthetic construct
  REGION 1..168: note = Description of Artificial Sequence: Synthetic polypeptide
LRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 51 - DNA, 291 bp, synthetic construct
  misc_feature 1..291: note = Description of Artificial Sequence: Synthetic polynucleotide
  source 1..291: mol_type = other DNA
tgtagccgagcggccagaggcacaatcggggcaagacgaacaggacagccgctcaaagaggaccccagtgcggtccccgttttctccgtggattacggagaactggatttccagtggcgggagaagacaccagagcccccggtgccctgcgtgccggagcagactgagtacgccacgattgtgtttccctctggaatggggacttcatcccccgctaggcgcggctcagctgatggcccaagatccgctcaaccgttgcggccagaggacgggcattgcagttggcctctg

SEQ ID NO: 52 - DNA, 333 bp, synthetic construct
  misc_feature 1..333: note = Description of Artificial Sequence: Synthetic polynucleotide
  source 1..333: mol_type = other DNA
agaagacatcaggggaagcagaatgaactcagcgatacagcagggcgagaaattaatttggtagacgcgcatctgaagtccgaacagacagaggcttctactagacagaactcccaagttttgttgagtgagacggggatctatgataatgatcccgatctgtgttttagaatgcaggagggtagtgaagtctactcaaacccgtgcctggaagaaaataagcccggcattgtttacgctagtttgaatcattctgtaataggcccgaactccagactggctcgcaatgtgaaggaggccccaactgagtatgcgtccatttgcgtgcggtct

SEQ ID NO: 53 - DNA, 333 bp, synthetic construct
  misc_feature 1..333: note = Description of Artificial Sequence: Synthetic polynucleotide
  source 1..333: mol_type = other DNA
agaaggcaccagggaaagcagaacgagctgagcgataccgccggcagagaaatcaacctggtggacgcccacctgaaaagcgagcagacagaggccagcaccagacagaatagccaggtgctgctgagcgagacaggcatctacgacaacgaccccgacctgtgcttccggatgcaagagggaagcgaggtgtacagcaacccctgcctggaagagaacaagcccggcatcgtgtacgctagcctgaaccactctgtgatcggccccaattccagactggcccggaacgtgaaagaggcccctacagagtacgccagcatctgcgtcagaagc

SEQ ID NO: 54 - DNA, 504 bp, synthetic construct
  misc_feature 1..504: note = Description of Artificial Sequence: Synthetic polynucleotide
  source 1..504: mol_type = other DNA
ttgcgccacagacggcagggaaagcactggactagtacgcagaggaaagcggacttccagcatcccgcaggagccgtggggcctgaacccactgatcgcggccttcaatggaggtctagcccggcggcagacgcacaagaggaaaacttgtacgcagccgttaagcacacccaaccggaggacggcgttgagatggatacccgctcccctcacgatgaagaccctcaagcagtcacttacgcggaagtaaagcatagccgccccagacgggaaatggctagcccgccgtccccccttagcggggaatttctggacactaaagataggcaggcggaagaggaccgccaaatggatacagaggcggcggcaagtgaagcacctcaagacgttacttacgctcaacttcacagccttaccctcaggcgagaagcgactgaaccacccccttcccaagaagggccaagcccagcggttccttctatctatgctactcttgctattcac

SEQ ID NO: 55 - DNA, 504 bp, synthetic construct
  misc_feature 1..504: note = Description of Artificial Sequence: Synthetic polynucleotide
  source 1..504: mol_type = other DNA
ctgcggcacagacggcagggcaagcactggacaagcacacagagaaaggccgactttcagcaccctgctggtgccgttggacctgagcctacagatagaggactgcagtggcggtctagccctgccgctgatgctcaagaggaaaacctgtacgccgccgtgaagcacacccaacctgaagatggcgtggaaatggacaccagatctccccacgatgaggaccctcaggccgtgacatatgccgaagtgaagcactcccggcctcggagagaaatggctagccctccaagtcctctgagcggcgagttcctggacaccaaggatagacaggccgaagaggaccggcagatggatacagaagctgccgcatctgaggccccacaggatgtgacttatgcccagctgcacagcctgacactgcggagagaagccacagagcctccaccttctcaagagggcccatctccagccgtgcctagcatctatgccacactggccatccac

SEQ ID NO: 56 - protein, 454 aa, synthetic construct
  REGION 1..454: note = Description of Artificial Sequence: Synthetic polypeptide
METDTLLLWVLLLWVPGSTGAGGSDYKDDDDKGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITTTTPAPRPPTPAPTIALQPLSLRPEACRPAAGGAVHTRGLDFACDLLPLGGLPLLITTCFCLFCCLRRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 57 - protein, 502 aa, synthetic construct
  REGION 1..502: note = Description of Artificial Sequence: Synthetic polypeptide
MALPVTALLLPLALLLHAARPEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITEQKLISEEDLNGAATTTPAPRPPTPAPTIALQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 58 - protein, 504 aa, synthetic construct
  REGION 1..504: note = Description of Artificial Sequence: Synthetic polypeptide
MALPVTALLLPLALLLHAARPQVQLVQSGAEVKKPGASVKVSCKASGYTFTNYWMHWVRQAPGQGLEWMGFITPTTGYPEYNQKFKDRVTMTADKSTSTAYMELSSLRSEDTAVYYCARRKVGKGVYYALDYWGQGTTVTVSSGGGGSGGGGSGGGGSDIQMTQSPSSLSASVGDRVTITCRASGNIHNYLAWYQQKPGKVPKLLIYNTKTLADGVPSRFSGSGSGTDYTLTISSLQPEDVATYYCQHFWSSPWTFGGGTKVEIKEQKLISEEDLNGAATTTPAPRPPTPAPTIALQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 59 - protein, 21 aa, synthetic construct
  REGION 1..21: note = Description of Artificial Sequence: Synthetic peptide
VIGILVAVILLLLLLLLLFLI

SEQ ID NO: 60 - protein, 21 aa, synthetic construct
  REGION 1..21: note = Description of Artificial Sequence: Synthetic peptide
VGVVGGLLGSLVLLVWVLAVI

SEQ ID NO: 61 - DNA, 63 bp, synthetic construct
  misc_feature 1..63: note = Description of Artificial Sequence: Synthetic oligonucleotide
  source 1..63: mol_type = other DNA
ctgctgcctcttggaggactgcccctcctgatcaccacatgcttttgcctgttctgctgtctg

SEQ ID NO: 62 - DNA, 63 bp, synthetic construct
  misc_feature 1..63: note = Description of Artificial Sequence: Synthetic oligonucleotide
  source 1..63: mol_type = other DNA
gttatagggatcctggtggctgtcatactcctcttgctcctcttgttgctgctttttttgata

SEQ ID NO: 63 - DNA, 63 bp, synthetic construct
  misc_feature 1..63: note = Description of Artificial Sequence: Synthetic oligonucleotide
  source 1..63: mol_type = other DNA
gtgatcggaattctggtggccgtgattctgctgctgctccttctgctcctgctgtttctgatt

SEQ ID NO: 64 - DNA, 63 bp, synthetic construct
  misc_feature 1..63: note = Description of Artificial Sequence: Synthetic oligonucleotide
  source 1..63: mol_type = other DNA
gttggggttgtaggtggtctgctcggcagcctggtcttgttggtgtgggtcttggctgtgatc

SEQ ID NO: 65 - DNA, 63 bp, synthetic construct
  misc_feature 1..63: note = Description of Artificial Sequence: Synthetic oligonucleotide
  source 1..63: mol_type = other DNA
gtgggagttgttggcggcctgctgggatctctggtgctgcttgtttgggtgctcgccgtgatc

SEQ ID NO: 66 - protein, 84 aa, synthetic construct
  REGION 1..84: note = Description of Artificial Sequence: Synthetic polypeptide
HLWCSNKKNAAVMDQEPAGNRTANSEDSDEQDPEEVTYAQLDHCVFTQRKITRPSQRPKTPPTDTILYTELPNAKPRSKVVSCP

SEQ ID NO: 67 - protein, 41 aa, synthetic construct
  REGION 1..41: note = Description of Artificial Sequence: Synthetic polypeptide
AVSLSKMLKKRSPLTTGVGVKMPPTEPECEKQFQPYFIPIN

SEQ ID NO: 68 - protein, 22 aa, synthetic construct
  REGION 1..22: note = Description of Artificial Sequence: Synthetic peptide
DFLLWILAAVSSGLFFYSFLLT

SEQ ID NO: 69 - protein, 20 aa, synthetic construct
  REGION 1..20: note = Description of Artificial Sequence: Synthetic peptide
ILIGTSVVIILFILLLFFLL

SEQ ID NO: 70 - protein, 75 aa, synthetic construct
  REGION 1..75: note = Description of Artificial Sequence: Synthetic polypeptide
TTTPAPRPPTPAPTIALQPLSLRPEACRPAAGGAVHTRGLDFACDQTTPGERSSLPAFYPGTSGSCSGCGSLSLP

SEQ ID NO: 71 - protein, 21 aa, synthetic construct
  REGION 1..21: note = Description of Artificial Sequence: Synthetic peptide
MALPVTALLLPLALLLHAARP

SEQ ID NO: 72 - DNA, 63 bp, synthetic construct
  misc_feature 1..63: note = Description of Artificial Sequence: Synthetic oligonucleotide
  source 1..63: mol_type = other DNA
atggccttaccagtgaccgccttgctcctgccgctggccttgctgctccacgccgccaggccg

SEQ ID NO: 73 - protein, 21 aa, synthetic construct
  REGION 1..21: note = Description of Artificial Sequence: Synthetic peptide
KYLLPTAAAGLLLLAAQPAMA

SEQ ID NO: 74 - DNA, 63 bp, synthetic construct
  misc_feature 1..63: note = Description of Artificial Sequence: Synthetic oligonucleotide
  source 1..63: mol_type = other DNA
aaatacctattgcctacggcagccgctggattgttattactcgcggcccagccggccatggcc

SEQ ID NO: 75 - protein, 255 aa, synthetic construct
  REGION 1..255: note = Description of Artificial Sequence: Synthetic polypeptide
QVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLG

SEQ ID NO: 76 - DNA, 765 bp, synthetic construct
  misc_feature 1..765: note = Description of Artificial Sequence: Synthetic polynucleotide
  source 1..765: mol_type = other DNA
caggtgcagctggtgcagtctggggcagaggtgaaaaagcccggggagtctctgaagatctcctgtaagggttctggatacagctttaccagctactggatcgcctgggtgcgccagatgcccgggaaaggcctggagtacatggggctcatctatcctggtgactctgacaccaaatacagcccgtccttccaaggccaggtcaccatctcagtcgacaagtccgtcagcactgcctacttgcaatggagcagtctgaagccctcggacagcgccgtgtatttttgtgcgagacatgacgtgggatattgcaccgaccggacttgcgcaaagtggcctgaatacttccagcattggggccagggcaccctggtcaccgtctcctcaggtggaggcggttcaggcggaggtggctctggcggtggcggatcgcagtctgtgttgacgcagccgccctcagtgtctgcggccccaggacagaaggtcaccatctcctgctctggaagcagctccaacattgggaataattatgtatcctggtaccagcagctcccaggaacagcccccaaactcctcatctatgatcacaccaatcggcccgcaggggtccctgaccgattctctggctccaagtctggcacctcagcctccctggccatcagtgggttccggtccgaggatgaggctgattattactgtgcctcctgggactacaccctctcgggctgggtgttcggcggagggaccaagctgaccgtcctaggt

SEQ ID NO: 77 - protein, 18 aa, synthetic construct
  REGION 1..18: note = Description of Artificial Sequence: Synthetic peptide
GKPIPNPLLGLDSTNGAA

SEQ ID NO: 78 - DNA, 54 bp, synthetic construct
  misc_feature 1..54: note = Description of Artificial Sequence: Synthetic oligonucleotide
  source 1..54: mol_type = other DNA
gggaagcctatcccgaaccctctgttgggtctcgatagtaccaatggggccgca

SEQ ID NO: 79 - DNA, 135 bp, synthetic construct
  misc_feature 1..135: note = Description of Artificial Sequence: Synthetic polynucleotide
  source 1..135: mol_type = other DNA
accacgacgccagcgccgcgaccaccaacaccggcgcccaccatcgcgttgcagcccctgtccctgcgcccagaggcgtgccggccagcggcggggggcgcagtgcacacgagggggctggacttcgcctgtgat

SEQ ID NO: 80 - DNA, 66 bp, synthetic construct
  misc_feature 1..66: note = Description of Artificial Sequence: Synthetic oligonucleotide
  source 1..66: mol_type = other DNA
gattttctgctgtggattctggcagctgtgagctctggcttgtttttctacagcttcctcctgacc

SEQ ID NO: 81 - DNA, 60 bp, synthetic construct
  misc_feature 1..60: note = Description of Artificial Sequence: Synthetic oligonucleotide
  source 1..60: mol_type = other DNA
atcctgatcgggacaagtgtagtaatcatacttttcatactcctgctcttttttctcttg

SEQ ID NO: 82 - DNA, 63 bp, synthetic construct
  misc_feature 1..63: note = Description of Artificial Sequence: Synthetic oligonucleotide
  source 1..63: mol_type = other DNA
gtgatcggaattctggtggccgtgatcctgctgctcctgcttctcctcctgctgtttctgatc

SEQ ID NO: 83 - DNA, 81 bp, synthetic construct
  misc_feature 1..81: note = Description of Artificial Sequence: Synthetic oligonucleotide
  source 1..81: mol_type = other DNA
ttctgggtgctcgttgttgttggcggcgtgctggcctgttattccctgctggttaccgtggccttcatcatcttttgggtc

SEQ ID NO: 84 - DNA, 123 bp, synthetic construct
  misc_feature 1..123: note = Description of Artificial Sequence: Synthetic polynucleotide
  source 1..123: mol_type = other DNA
gccgtgtcacttagtaagatgctgaagaagaggtcaccactgacgacaggggttggagtgaagatgccacccacagaacccgaatgtgagaagcaattccagccttatttcattccaataaat

SEQ ID NO: 85 - DNA, 252 bp, synthetic construct
  misc_feature 1..252: note = Description of Artificial Sequence: Synthetic polynucleotide
  source 1..252: mol_type = other DNA
catctgtggtgttctaataagaagaatgctgctgtgatggatcaagagcccgctggtaacagaacggccaacagtgaagatagcgatgagcaggacccagaagaagtgacctacgcccaactcgaccactgtgtttttacgcagcggaaaatcactcgaccctctcaacgacccaaaacgccgcctacggacaccatactctacaccgaactgccgaacgccaaaccacggtccaaggtggtatcatgtccg

SEQ ID NO: 86 - DNA, 504 bp, synthetic construct
  misc_feature 1..504: note = Description of Artificial Sequence: Synthetic polynucleotide
  source 1..504: mol_type = other DNA
ctgcggcacagaaggcagggcaagcactggacaagcacccagagaaaggccgattttcagcaccctgctggcgccgttggacctgagcctacagatagaggactgcagtggcggtctagccctgctgccgatgctcaagaggaaaacctgtacgccgccgtgaagcacacccaacctgaagatggcgtggaaatggacaccagatctccccacgatgaggaccctcaggccgtgacatacgctgaagtgaagcactcccggcctcggagagaaatggctagccctccaagtcctctgagcggcgagttcctggacaccaaggatagacaggccgaagaggaccggcagatggatacagaagctgccgcctctgaagccccacaggatgtgacatatgcccagctgcatagcctgacactgcggagagaagccacagagcctccaccttctcaagagggcccatctccagccgtgcctagcatctatgccacactggccattcac

SEQ ID NO: 87 - DNA, 225 bp, synthetic construct
  misc_feature 1..225: note = Description of Artificial Sequence: Synthetic polynucleotide
  source 1..225: mol_type = other DNA
accacgacgccagcgccgcgaccaccaacaccggcgcccaccatcgcgttgcagcccctgtccctgcgcccagaggcgtgccggccagcggcggggggcgcagtgcacacgagggggctggacttcgcctgtgatcagaccacacctggcgagagatcttccctgcctgccttctatcctggcaccagcggctcttgttctggctgtggatcactgagcctgcct

SEQ ID NO: 88 - DNA, 126 bp, synthetic construct
  misc_feature 1..126: note = Description of Artificial Sequence: Synthetic polynucleotide
  source 1..126: mol_type = other DNA
gccgctgctatcgaagtgatgtaccctcctccttacctggacaacgagaagtccaacggcaccatcatccacgtgaagggcaagcacctgtgtccttctccactgttccccggacctagcaagcct

SEQ ID NO: 89 - protein, 23 aa, synthetic construct
  REGION 1..23: note = Description of Artificial Sequence: Synthetic peptide
IVVITVVSAMLILCIIGLIGVIL

SEQ ID NO: 90 - DNA, 69 bp, synthetic construct
  misc_feature 1..69: note = Description of Artificial Sequence: Synthetic oligonucleotide
  source 1..69: mol_type = other DNA
atagtggtcatcactgtagttagtgcaatgcttattctttgtatcatagggctcataggggtaatcctg

SEQ ID NO: 91 - protein, 21 aa, synthetic construct
  REGION 1..21: note = Description of Artificial Sequence: Synthetic peptide
LLGAMAATLVVICTAVIVVVA

SEQ ID NO: 92 - DNA, 63 bp, synthetic construct
  misc_feature 1..63: note = Description of Artificial Sequence: Synthetic oligonucleotide
  source 1..63: mol_type = other DNA
ctgctgggcgccatggccgccacactggttgttatctgtaccgccgtgatcgtggtggtggcc

SEQ ID NO: 93 - protein, 70 aa, synthetic construct
  REGION 1..70: note = Description of Artificial Sequence: Synthetic polypeptide
KEPASPLDKCHYTKDNGQFDQSAKQLNLEAYTIEQETALISNKNGKPKRQQRKPNPPLNLDSYIVGQNDM

SEQ ID NO: 94 - DNA, 210 bp, synthetic construct
  misc_feature 1..210: note = Description of Artificial Sequence: Synthetic polynucleotide
  source 1..210: mol_type = other DNA
aaggagcctgcgtccccgttggataaatgccactatactaaggataacggtcagttcgatcagagtgcaaagcaacttaacttggaggcttacactatagagcaagaaacagcgctgataagtaataagaacggtaagccaaagcgacagcagaggaaacccaatcctccgcttaacttggatagctacatcgtcgggcaaaatgacatg

SEQ ID NO: 95 - protein, 82 aa, synthetic construct
  REGION 1..82: note = Description of Artificial Sequence: Synthetic polypeptide
LTRKKKALRIHSVEGDLRRKSAGQEEWSPSAPSPPGSCVQAEAAPAGLCGEQRGEDCAELHDYFNVLSYRSLGNCSFFTETG

SEQ ID NO: 96 - DNA, 246 bp, synthetic construct
  misc_feature 1..246: note = Description of Artificial Sequence: Synthetic polynucleotide
  source 1..246: mol_type = other DNA
ctgaccagaaagaagaaggccctgagaatccacagcgtggaaggcgacctgcggagaaagtctgccggacaagaagagtggtcccctagcgctccatctccacctggatcttgtgtgcaggccgaagcagctcctgctggactgtgtggcgaacagagaggcgaagattgcgccgagctgcacgactacttcaacgtgctgagctacagaagcctgggcaactgcagcttcttcaccgagacagga

SEQ ID NO: 97 - protein, 147 aa, synthetic construct
  REGION 1..147: note = Description of Artificial Sequence: Synthetic polypeptide
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 98 - DNA, 441 bp, synthetic construct
  misc_feature 1..441: note = Description of Artificial Sequence: Synthetic polynucleotide
  source 1..441: mol_type = other DNA
ttcctggacagccccgacagaccttggaaccctcctacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgtccgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgccggccagtttcagacactggtt

SEQ ID NO: 99 - protein, 147 aa, synthetic construct
  REGION 1..147: note = Description of Artificial Sequence: Synthetic polypeptide
KAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLT

SEQ ID NO: 100 - DNA, 441 bp, synthetic construct
  misc_feature 1..441: note = Description of Artificial Sequence: Synthetic polynucleotide
  source 1..441: mol_type = other DNA
aaggccatgcatgtggctcagcctgctgtggtgctggcctcttctagaggaatcgccagcttcgtgtgcgagtacgcctctcctggaaaggccactgaagtgcgcgtgaccgttctgagacaggccgatagccaagtgaccgaagtgtgcgccgccacctacatgatgggcaacgagctgaccttcctggacgacagcatctgtaccggcaccagcagcggcaatcaagtgaacctgaccatccagggcctgagagccatggataccggcctgtacatctgcaaggtggaactgatgtaccctcctccttactacctcggcatcggcaacggcacccagatctacgtgatcgaccctgagccttgtcctgacagcgactttctgctgtggatcctggctgccgtgtccagcggcctgttcttctactcttttctgctgacc

SEQ ID NO: 101 - protein, 319 aa, synthetic construct
  REGION 1..319: note = Description of Artificial Sequence: Synthetic polypeptide
HMGGQDKPFLSAWPSAVVPRGGHVTLRCHYRHRFNNFMLYKEDRIHIPIFHGRIFQESFNMSPVTTAHAGNYTCRGSHPHSPTGWSAPSNPVVIMVTGNHRKPSLLAHPGPLVKSGERVILQCWSDIMFEHFFLHKEGISKDPSRLVGQIHDGVSKANFSIGPMMLALAGTYRCYGSVTHTPYQLSAPSDPLDIVVTGPYEKPSLSAQPGPKVQAGESVTLSCSSRSSYDMYHLSREGGAHERRLPAVRKVNRTFQADFPLGPATHGGTYRCFGSFRHSPYEWSDPSDPLLVSVTGNPSSSWPSPTEPSSKSGNPRHLH

SEQ ID NO: 102 - DNA, 957 bp, synthetic construct
  misc_feature 1..957: note = Description of Artificial Sequence: Synthetic polynucleotide
  source 1..957: mol_type = other DNA
cacatgggcggacaggataagcctttcctgagcgcctggccttctgccgttgttcctagaggcggacacgtgaccctgcggtgtcactacagacaccggttcaacaacttcatgctgtacaaagaggaccggattcacatccccatcttccacggccggatcttccaagagtccttcaacatgagccccgtgaccacagctcacgccggcaactacacatgcagaggctctcaccctcacagccctacaggctggagtgccccttctaaccccgtggtcatcatggtcaccggcaaccacagaaagcccagcctgcttgctcatcccggacctctggttaagtctggcgagcgagtgatcctgcagtgttggagcgatattatgttcgagcacttctttctgcacaaagagggcatcagcaaggacccctctagactcgtgggccagatccatgatggcgtgtccaaggccaacttcagcatcggccctatgatgctggccctggccggcacctatagatgttacggcagcgtgacccacacaccttaccagctgagcgcccctagcgaccctctggatatcgtggtcacaggcccctacgagaagcctagcctgtctgcacagcctggacctaaagtgcaggccggcgaaagcgtgacactgagctgtagcagcagatccagctacgacatgtaccacctgagcagagaaggcggagcccacgagagaaggctgcctgccgtcagaaaagtgaaccggaccttccaggccgactttcctctgggacctgctacacacggcggcacctaccggtgtttcggcagctttagacacagcccttacgagtggagcgacccctctgatcctctgctggtgtctgtgaccggcaatcctagcagcagctggccctctccaacagagccttctagcaagagcggcaaccccagacatctgcac

SEQ ID NO: 103 - protein, 120 aa, synthetic construct
  REGION 1..120: note = Description of Artificial Sequence: Synthetic polypeptide
MMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIP

SEQ ID NO: 104 - DNA, 360 bp, synthetic construct
  misc_feature 1..360: note = Description of Artificial Sequence: Synthetic polynucleotide
  source 1..360: mol_type = other DNA
atgatgaccggcaccatcgagacaaccggcaacatctctgccgagaaaggcggcagcatcatcctgcagtgtcacctgtctagcaccaccgctcaagtgacccaagtgaactgggagcagcaggatcagctgctggccatctgcaatgccgatctcggctggcacatcagccccagcttcaaggatagagtggcccctggacctggcctgggactgacacttcagagcctgaccgtgaacgataccggcgagtacttctgcatctaccacacataccccgacggcacctataccggccggatctttctggaagtgctggaaagctctgtggccgagcacggcgccagatttcagattcct

SEQ ID NO: 105 - protein, 70 aa, synthetic construct
  REGION 1..70: note = Description of Artificial Sequence: Synthetic polypeptide
MDNQGVIYSDLNLPPNPKRQQRKPKGNKNSILATEQEITYAELNLQKASQDFQGNDKTYHCKDLPSAPEK

SEQ ID NO: 106 - DNA, 210 bp, synthetic construct
  misc_feature 1..210: note = Description of Artificial Sequence: Synthetic polynucleotide
  source 1..210: mol_type = other DNA
atggacaaccagggcgtgatctacagcgacctgaacctgcctcctaatcctaagcggcagcagagaaagcccaagggcaacaagaacagcatcctggccaccgagcaagagatcacctacgccgagctgaatctgcagaaggccagccaggacttccagggcaacgacaagacctaccactgcaaggacctgcctagcgctcccgagaag

SEQ ID NO: 107 - protein, 23 aa, synthetic construct
  REGION 1..23: note = Description of Artificial Sequence: Synthetic peptide
LIVGILGIICLILMASVVTIVVI

SEQ ID NO: 108 - DNA, 69 bp, synthetic construct
  misc_feature 1..69: note = Description of Artificial Sequence: Synthetic oligonucleotide
  source 1..69: mol_type = other DNA
ctgatcgtgggaatcctgggcatcatctgcctgatcctgatggccagcgtggtcaccatcgtggtcatc

SEQ ID NO: 109 - protein, 140 aa, synthetic construct
  REGION 1..140: note = Description of Artificial Sequence: Synthetic polypeptide
PSTLIQRHNNSSLNTRTQKARHCGHCPEEWITYSNSCYYIGKERRTWEESLLACTSKNSSLLSIDNEEEMKFLSIISPSSWIGVFRNSSHHPWVTMNGLAFKHEIKDSDNAELNCAVLQVNRLKSAQCGSSIIYHCKHKL

SEQ ID NO: 110 - DNA, 420 bp, synthetic construct
  misc_feature 1..420: note = Description of Artificial Sequence: Synthetic polynucleotide
  source 1..420: mol_type = other DNA
cccagcacactgatccagcggcacaacaacagcagcctgaacaccagaacacagaaggcccggcactgcggccactgtcctgaagagtggatcacatacagcaacagctgctactacatcggcaaagagcggcggacctgggaagaatctctgctggcctgcaccagcaagaactccagcctgctgagcatcgacaacgaggaagagatgaagttcctgtccatcatcagccccagcagctggatcggcgtgttcagaaacagctcccaccatccttgggtcaccatgaacggcctggccttcaagcacgagatcaaggacagcgacaacgccgaactgaactgtgccgtgctgcaagtgaaccggctgaagtctgcccagtgtggcagcagcatcatctatcactgcaagcacaagctg

SEQ ID NO: 111 - protein, 14 aa, synthetic construct
  REGION 1..14: note = Description of Artificial Sequence: Synthetic peptide
NGAAEQKLISEEDL

SEQ ID NO: 112 - DNA, 42 bp, synthetic construct
  misc_feature 1..42: note = Description of Artificial Sequence: Synthetic oligonucleotide
  source 1..42: mol_type = other DNA
aatggggccgcagaacaaaaactcatctcagaagaagatctg

SEQ ID NO: 113 - protein, 20 aa, synthetic construct
  REGION 1..20: note = Description of Artificial Sequence: Synthetic peptide
METDTLLLWVLLLWVPGSTG

SEQ ID NO: 114 - DNA, 60 bp, synthetic construct
  misc_feature 1..60: note = Description of Artificial Sequence: Synthetic oligonucleotide
  source 1..60: mol_type = other DNA
atggaaaccgacacactgctgctgtgggtgctgcttctttgggtgcccggatctacaggt

SEQ ID NO: 115 - protein, 237 aa, synthetic construct
  REGION 1..237: note = Description of Artificial Sequence: Synthetic polypeptide
QVQLQESGPGLVKPSETLSLTCTVSGYSITSNYWGWIRQPPGKGLEWMGYITYSGSTSYNPSLKSRITISRDTSKNQFSLKLSSVTAADTAVYYCAITTFYYWGQGTLVTVSSGGGGSGGGGSGGGGSDIQMTQSPSSLSASVGDRVTITCRASQDIGNYLRWFQQKPGKAPKLLISGATNLAAGVPSRFSGSGSGSDFTLTISSLQPEDFATYYCLQSKESPWTFGQGTKVEIKRT

SEQ ID NO: 116 - DNA, 711 bp, synthetic construct
  misc_feature 1..711: note = Description of Artificial Sequence: Synthetic polynucleotide
  source 1..711: mol_type = other DNA
caggtccagctgcaagaatctggaccaggcctcgtgaagcccagcgagacactgtctctgacctgtaccgtgtccggctacagcatcaccagcaactactggggctggatcagacagcctcctggcaaaggccttgagtggatgggctacatcacctacagcggcagcaccagctacaaccccagcctgaagtcccggatcaccatcagcagagacaccagcaagaaccagttctccctgaagctgagcagcgtgacagccgccgatacagccgtgtactactgtgccatcaccaccttctactattggggccagggcaccctggtcacagtttctagcggaggcggaggatctggtggcggaggaagtggcggaggcggttctgatatccagatgacacagagccccagcagcctgtctgcctctgtgggagacagagtgaccatcacctgtagggccagccaggacatcggcaactacctgagatggttccagcagaagcctggcaaggcccctaagctgctgattagcggcgccacaaatctggctgctggcgtgccaagcagattttccggctctggcagcggctccgatttcaccctgaccatatctagcctgcagcctgaggacttcgccacctactactgcctgcagagcaaagagagcccctggacatttggacagggcaccaaggtggaaatcaagcggacc

SEQ ID NO: 117 - protein, 15 aa, synthetic construct
  REGION 1..15: note = Description of Artificial Sequence: Synthetic peptide
AGGSDYKDDDDKGGS

SEQ ID NO: 118 - DNA, 45 bp, synthetic construct
  misc_feature 1..45: note = Description of Artificial Sequence: Synthetic oligonucleotide
  source 1..45: mol_type = other DNA
gccggcggaagcgactacaaggacgacgatgacaaaggcggcagc

SEQ ID NO: 119 - protein, 41 aa, synthetic construct
  REGION 1..41: note = Description of Artificial Sequence: Synthetic polypeptide
RSKRSRLLHSDYMNMTPRRPGPTRKHYQPYAPPRDFAAYRS

SEQ ID NO: 120 - DNA, 123 bp, synthetic construct
  misc_feature 1..123: note = Description of Artificial Sequence: Synthetic polynucleotide
  source 1..123: mol_type = other DNA
cgaagcaagcggagccggctgctgcacagcgattacatgaacatgacccctcggaggcccggacctaccagaaagcactaccagccttacgctcctcctagagatttcgccgcctaccggtcc

SEQ ID NO: 121 - protein, 112 aa, synthetic construct
  REGION 1..112: note = Description of Artificial Sequence: Synthetic polypeptide
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 122 - DNA, 336 bp, synthetic construct
  misc_feature 1..336: note = Description of Artificial Sequence: Synthetic polynucleotide
  source 1..336: mol_type = other DNA
agagtgaagttcagcagatccgccgatgctcccgcctataagcagggccagaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggactgagcaccgccaccaaggatacctatgacgccctgcacatgcaggccctgcctccaaga

SEQ ID NO: 123 - DNA, 63 bp, synthetic construct
  misc_feature 1..63: note = Description of Artificial Sequence: Synthetic oligonucleotide
  source 1..63: mol_type = other DNA
atggccctgcctgtgacagctctgctgctgcctctggccctgctgctgcatgctgctagacct

SEQ ID NO: 124 - DNA, 711 bp, synthetic construct
  misc_feature 1..711: note = Description of Artificial Sequence: Synthetic polynucleotide
  source 1..711: mol_type = other DNA
caggtgcagctgcaggaaagcggccctggcctcgtgaagcctagcgagacactgagcctgacctgcaccgtgtccggctacagcatcaccagcaactactggggctggatcagacagccccctggcaagggcctggaatggatgggctacatcacctacagcggcagcaccagctacaaccccagcctgaagtcccggatcaccatcagccgggacaccagcaagaaccagttctccctgaagctgagcagcgtgacagccgccgataccgccgtgtactactgcgccatcaccaccttctactattggggccagggcaccctcgtgaccgtgtctagcggaggcggaggatctggcggcggaggaagtggcggagggggctctgatatccagatgacccagagccccagcagcctgtctgccagcgtgggcgacagagtgaccatcacctgtagagccagccaggacatcggcaactacctgcggtggttccagcagaagccaggcaaggcccccaagctgctgatctccggcgccacaaatctggccgctggcgtgccaagcagattcagcggctctggcagcggctccgacttcaccctgaccatctctagcctgcagcccgaggacttcgccacctactactgcctgcagagcaaagagagcccctggaccttcggacagggcaccaaggtggaaatcaagcggaca

SEQ ID NO: 125 - protein, 14 aa, synthetic construct
  REGION 1..14: note = Description of Artificial Sequence: Synthetic peptide
EQKLISEEDLNGAA

SEQ ID NO: 126 - DNA, 42 bp, synthetic construct
  misc_feature 1..42: note = Description of Artificial Sequence: Synthetic oligonucleotide
  source 1..42: mol_type = other DNA
gaacaaaaactcatctcagaagaagatctgaatggggccgca

SEQ ID NO: 127 - protein, 21 aa, synthetic construct
  REGION 1..21: note = Description of Artificial Sequence: Synthetic peptide
IYIWAPLAGTCGVLLLSLVIT

SEQ ID NO: 128 - DNA, 63 bp, synthetic construct
  misc_feature 1..63: note = Description of Artificial Sequence: Synthetic oligonucleotide
  source 1..63: mol_type = other DNA
atatacatctgggctcctctggctggcacttgcggagtgcttctgctgagtctggttattacc

SEQ ID NO: 129 - DNA, 336 bp, synthetic construct
  misc_feature 1..336: note = Description of Artificial Sequence: Synthetic polynucleotide
  source 1..336: mol_type = other DNA
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 130 - DNA, 210 bp, synthetic construct
  misc_feature 1..210: note = Description of Artificial Sequence: Synthetic polynucleotide
  source 1..210: mol_type = other DNA
atggacaaccagggcgtcatctacagcgacctgaacctgcctcctaatccaaagcggcagcagcggaagcccaagggcaacaagaatagcatcctggccaccgagcaagagatcacctacgccgagctgaatctgcagaaggccagccaggatttccagggcaacgacaagacctaccactgcaaggacctgcctagcgctcctgagaaa

SEQ ID NO: 131 - DNA, 63 bp, synthetic construct
  misc_feature 1..63: note = Description of Artificial Sequence: Synthetic oligonucleotide
  source 1..63: mol_type = other DNA
gtgatcggcattctggtggccgtgattctgctgctcctgctgttgctgctgctgttcctgatc

SEQ ID NO: 132 - DNA, 69 bp, synthetic construct
  misc_feature 1..69: note = Description of Artificial Sequence: Synthetic oligonucleotide
  source 1..69: mol_type = other DNA
ctgatcgtgggcatcctgggcatcatctgtctgatcctgatggccagcgtggtcaccatcgtggtcatc

SEQ ID NO: 133 - protein, 4 aa, synthetic construct
  REGION 1..4: note = Description of Artificial Sequence: Synthetic peptide
NGAA

SEQ ID NO: 134 - protein, 4 aa, synthetic construct
  REGION 1..4: note = Description of Artificial Sequence: Synthetic peptide
AGGS